(19) United States
(12) Patent Application Publication
(10) Pub. No.: US 2022/0409713 A1
(43) Pub. Date: Dec. 29, 2022

(54) COMPOSITIONS AND METHODS FOR THE PROPHYLAXIS AND TREATMENT OF BABESIOSIS

(71) Applicant: Tufts Medical Center, Inc., Boston, MA (US)

(72) Inventors: Edouard VANNIER, Boston, MA (US); Joseph J. CAMPO, JR., Irvine, CA (US)

(21) Appl. No.: 17/831,012

(22) Filed: Jun. 2, 2022

Related U.S. Application Data
(60) Provisional application No. 63/195,935, filed on Jun. 2, 2021.

(51) Int. Cl. A61K 39/018; C07K 16/20; G01N 33/569

(52) U.S. Cl. CPC A61K 39/018; C07K 16/20; G01N 33/56905; G01N 2333/44; G01N 2469/20; G01N 2800/52; G01N 2800/26; A61K 2039/53

(57) ABSTRACT

Described herein are compositions that comprise one or more Babesia microti antigens, one or more Babesia microti nucleic acid molecules, or one or more anti-Babesia microti antibodies and uses thereof in methods for the prophylaxis of babesiosis, the treatment of babesiosis and the monitoring of individuals undergoing prophylactic or therapeutic administration of the compositions of the invention.

### SEQUENCE LISTING

The instant application contains a Sequence Listing which has been submitted electronically in ASCII format and is hereby incorporated by reference in its entirety. Said ASCII copy, created on Jun. 2, 2022, is named 00398-562002_Sequence_Listing_5_18_22_ST25 and is 173,543 bytes in size.

### FIELD OF THE INVENTION

This invention relates generally to the field of Babesia immunology.

### BACKGROUND OF THE INVENTION

Human babesiosis caused by the tick-borne parasite Babesia microti (Bm) is an emerging infectious disease in the United States. In 2019, approximately 2,400 cases were reported to the Centers for Disease Control and Prevention. Given that Lyme disease, another tick-borne disease, is underreported by a factor of 10, one can reasonably estimate the number of annual cases of babesiosis to be on the order of 24,000. Approximately one-half of cases are severe enough to warrant hospital admission for at least one day.

Babesiosis can be difficult to treat, particularly in individuals who are immunocompromised due to age, comorbidity, or treatment of comorbidity. The recommended treatment regimen consists of a combination of two antimicrobial agents, including atovaquone and azithromycin. Despite antimicrobial therapy, babesiosis can be complicated by severe anemia, renal insufficiency or failure, acute respiratory distress or failure, and hepatic compromise. Other complications include disseminated intravascular coagulation, congestive heart failure, and splenic infarct and/or rupture. In some cases, babesiosis can lead to death. Accordingly, there is a need for novel approaches to treat severe babesiosis. There is also a need for prophylactic measures as neither a vaccine nor a prophylactic antibiotic regimen is available for human babesiosis.

### SUMMARY OF THE INVENTION

The invention provides compositions containing (a) one or more (e.g., two or more) Babesia microti (Bm) antigens that each include an amino acid sequence having at least 80%, e.g., at least 90%, sequence identity to the amino acid sequence of any one of SEQ ID NOs: 1-24, or antigenic fragments thereof; and (b) a pharmaceutically acceptable carrier or diluent.

In some embodiments, the one or more (e.g., two or more) Bm antigens each include an amino acid sequence having at least 90% sequence identity to the amino acid sequence of any one of SEQ ID NOs: 1-3, or antigenic fragments thereof.

In some embodiments, the composition further contains one or more Bm antigens each including an amino acid sequence having at least 90% sequence identity to the amino acid sequence of any one of SEQ ID NOs: 4-9, or one or more antigenic fragments thereof.

In some embodiments, the one or more (e.g., two or more) Bm antigens each include an amino acid sequence having at least 90% sequence identity to the amino acid sequence of any one of SEQ ID NOs: 1-9, or antigenic fragments thereof.

In some embodiments, the composition includes at least two, at least three, at least four, at least five, at least six, at least seven, at least eight, at least nine, or at least twenty-four Bm antigens or antigenic fragments thereof.

In some embodiments, the composition comprises a Bm antigen of SEQ ID NO: 49, or an antigenic variant and/or antigenic fragment thereof; a Bm antigen of SEQ ID NO: 50, or an antigenic variant and/or antigenic fragment thereof; a Bm antigen of SEQ ID NO: 51, or an antigenic variant and/or antigenic fragment thereof; a Bm antigen of SEQ ID NO: 52, or an antigenic variant and/or antigenic fragment thereof; a Bm antigen of SEQ ID NO: 53, or an antigenic variant and/or antigenic fragment thereof; and/or a Bm antigen of SEQ ID NO: 54, or an antigenic variant and/or antigenic fragment thereof.

In some embodiments, the composition comprises two or more of said Bm antigens, or antigenic variants and/or antigenic fragments thereof.

In some embodiments, the composition comprises a Bm antigen of SEQ ID NO: 49, or an antigenic variant and/or antigenic fragment thereof; and/or a Bm antigen of SEQ ID NO: 50, or an antigenic variant and/or antigenic fragment thereof.

In some embodiments, the one or more (e.g., two or more) Bm antigens each include an amino acid sequence having at least 95% sequence identity to the amino acid sequence of any one of SEQ ID NOs: 1-24, or antigenic fragments thereof.

In some embodiments, the one or more (e.g., two or more) or more Bm antigens each include an amino acid sequence having at least 95% sequence identity to the amino acid sequence of any one of SEQ ID NOs: 1-3, or antigenic fragments thereof.

In some embodiments, the composition further comprises one or more Bm antigens each including an amino acid sequence having at least 95% sequence identity to the amino acid sequence of any one of SEQ ID NOs: 4-9, or antigenic fragments thereof.

In some embodiments, the one or more (e.g., two or more) Bm antigens each include an amino acid sequence having at least 95% sequence identity to the amino acid sequence of any one of SEQ ID NOs: 1-9, or antigenic fragments thereof.

In some embodiments, the composition includes at least two, at least three, at least four, at least five, at least six, at least seven, at least eight, at least nine, or at least twenty-four Bm antigens or antigenic fragments thereof.

In some embodiments, the one or more (e.g., two or more) or more Bm antigens each include an amino acid sequence independently selected from any one of SEQ ID NOs: 1-24.

In some embodiments, the one or more (e.g., two or more) Bm antigens each include an amino acid sequence independently selected from any one of SEQ ID NOs: 1-3.

In some embodiments, the composition further comprises one or more Bm antigens each including an amino acid sequence independently selected from any one of SEQ ID NOs: 4-9.

In some embodiments, the one or more (e.g., two or more) Bm antigens each include an amino acid sequence independently selected from any one of SEQ ID NOs: 1-9.

The invention also provides compositions comprising (a) one or more (e.g., two or more) nucleic acid molecules encoding one or more (e.g., two or more) or more Bm antigens, or antigenic fragments thereof, and optionally (b) a pharmaceutically acceptable carrier or diluent, wherein the one or more (e.g., two or more) Bm antigens, or antigenic fragments thereof, are as described above or elsewhere herein.

In some embodiments, the one or more (e.g., two or more) nucleic acid molecules comprise an RNA molecule or a DNA molecule, which optionally comprises a sequence of any one or more (e.g., two or more) of SEQ ID NOs: 25-48, fragments thereof, or a codon-optimized version thereof (with the understanding that T's are replaced with U's or similar nucleotides (e.g., pseudouridines) in RNA molecules, and that modified nucleotides and/or linkages can optionally be used, as is understood in the art).

In some embodiments, the composition further includes an adjuvant.

In some embodiments, the composition is formulated for administration by the oral route or a parenteral route, such as a parenteral route selected from the group consisting of the intravenous, intraperitoneal, subcutaneous, intramuscular, and topical routes.

The invention also provides compositions containing one or more antibodies that each specifically bind to one or more Bm antigens that each include an amino acid sequence having at least 90% sequence identity to the amino acid sequence of any one of SEQ ID NOs: 1-24, or antigenic fragments thereof.

In some embodiments, the one or more antibodies each specifically bind to one or more Bm antigens that each include an amino acid sequence having at least 90% sequence identity to the amino acid sequence of any one of SEQ ID NOs: 1-3, or antigenic fragments thereof.

In some embodiments, the composition further contains one or more antibodies that each specifically bind to one or more Bm antigens that each include an amino acid sequence having at least 90% sequence identity to the amino acid sequence of any one of SEQ ID NOs: 4-9, or antigenic fragments thereof.

In some embodiments, the one or more antibodies each specifically bind to one or more Bm antigens that each include an amino acid sequence having at least 90% sequence identity to the amino acid sequence of any one of SEQ ID NOs: 1-9, or antigenic fragments thereof.

In some embodiments, the composition further includes a pharmaceutically acceptable carrier or diluent.

The invention additionally provides methods of immunizing or conferring protective immunity against babesiosis to a subject, the methods including administering the composition as described above and elsewhere herein to the subject.

In some embodiments, the subject does not experience babesiosis. In some embodiments, treatment reduces the parasite burden upon a subsequent challenge. In some embodiments, treatment reduces the severity of babesiosis upon a subsequent challenge.

In some embodiments, e.g., when the composition comprises one or more antibodies, the subject experiences babesiosis. In some embodiments, treatment reduces the parasite burden. In some embodiments, treatment reduces the severity of babesiosis.

In some embodiments, the subject experiences mild or severe babesiosis, including persistent or relapsing babesiosis.

The invention additionally provides methods of supplementing an immune response in a subject experiencing babesiosis, the methods including administering to the subject a composition including one or more antibodies as described above or elsewhere herein.

The invention also provides methods of treating babesiosis in a subject in need thereof, the methods including administering to the subject a composition including one or more antibodies or antigens as described above or elsewhere herein.

In some embodiments, the etiology of babesiosis is Babesia microti.

The invention further provides methods for determining whether a subject, prior to administration of a composition as described above or elsewhere herein, has protective immunity against Bm-induced babesiosis, the methods including: (a) applying a body fluid sample from the subject to a solid support, wherein the solid support include one or more (e.g., two or more) Bm antigens including an amino acid sequence having at least 90% sequence identity to the amino acid sequence of any one of SEQ ID NOs: 1-24 and 49-54, or antigenic fragments thereof; (b) applying an antibody detection reagent to the solid support of (a); and (c) identifying the subject as likely to have protective immunity against Bm-induced babesiosis if the fluid sample from the subject is determined to have a sufficient titer of IgG antibodies that specifically react with one or more (e.g., two or more) Bm antigens of (a) or antigenic fragments thereof of, that is, a titer in the fluid sample above a cutoff titer for the one or more (e.g., two or more) Bm antigens. In some embodiments, the solid support includes two or more antigens from one of the compositions 1-112 as described herein, or a variant or fragment thereof, as described herein.

The invention additionally provides methods for determining whether a subject, following administration of a composition as described above or elsewhere herein, has acquired protective immunity against Bm-induced babesiosis, the method including: (a) applying a body fluid sample from the subject to a solid support, wherein the solid support include one or more (e.g., two or more) Bm antigens including an amino acid sequence having at least 90% sequence identity to the amino acid sequence of any one of SEQ ID NOs: 1-24 and 49-54, or antigenic fragments thereof; (b) applying an antibody detection reagent to the solid support of (a); and (c) identifying the subject as likely to have protective immunity against Bm-induced babesiosis if the fluid sample from the subject is determined to have a sufficient titer of IgG antibodies that specifically react with one or more (e.g., two or more) Bm antigens of (a) or antigenic fragments thereof of, that is, a titer in the fluid sample above a cutoff titer for the one or more (e.g., two or more) Bm antigens. In some embodiments, the solid support includes two or more antigens of one of the compositions 1-112 as described herein, or a variant or fragment thereof, as described herein.

The invention also provides methods for identifying a patient who experiences babesiosis and is likely to benefit from an anti-Bm antibody-based therapy, the methods including: (a) applying a body fluid sample from the subject to a solid support, wherein the solid support include one or more Bm antigens including an amino acid sequence having at least 90% sequence identity to the amino acid sequence of any one of SEQ ID NOs: 1-24 and 49-54, or antigenic fragments thereof; (b) applying an antibody detection reagent to the solid support of (a); and (c) identifying the patient as likely to benefit from an anti-Bm antibody based therapy if the fluid sample from the subject is determined to have an insufficient titer of IgG antibodies that specifically react with one or more Bm antigens of (a) or antigenic fragments thereof, that is, a titer in the fluid sample below a cutoff titer for the one or more Bm antigens. In some embodiments, the solid support includes antigens of one of the compositions 1-112 as described herein, or a variant or fragment thereof, as described herein.

The invention further provides methods of optimizing the administration or therapeutic efficacy of an anti-Bm antibody-based therapy to a subject experiencing babesiosis, the methods including: (a) applying a body fluid sample from the subject to a solid support, wherein the solid support include one or more Bm antigens including an amino acid sequence having at least 90% sequence identity to the amino acid sequence of any one of SEQ ID NOs: 1-24 and 49-54, or antigenic fragments thereof; (b) applying an antibody detection reagent to the solid support of (a); and (c) administering to the subject a composition including one or more antibodies as described above or elsewhere herein if the sample from the subject is determined to have an insufficient titer of IgG antibodies that specifically react with one or more Bm antigens of (a) or antigenic fragments thereof, that is, a titer below a cutoff titer for the one or more Bm antigens. In some embodiments, the solid support includes antigens of one of the compositions 1-112 as described herein, or a variant or fragment thereof, as described herein.

In some embodiments, the methods further include administering to the subject one or more antibodies as described above or elsewhere herein, wherein the subject has been determined as likely to benefit from an anti-Bm antibody based therapy or from a modified dose or dosage of anti-Bm antibody based therapy.

In some embodiments, of the methods described herein the subject is a human.

The invention also provides kits containing (a) one or more Bm antigens including an amino acid sequence having at least 90% sequence identity to the amino acid sequence of any one of SEQ ID NOs: 1-24 and 49-54, or antigenic fragments thereof, wherein one or more Bm antigens are immobilized on one or more solid supports; (b) an antibody detection reagent; and (c) a package insert including instructions for using the one or more Bm antigens and the antibody detection reagent in accordance with a method described above or elsewhere herein. In some embodiments, the kits include antigens of one of the compositions 1-112 as described herein, or a variant or fragment thereof, as described herein.

The invention provides kits containing (a) a composition including one or more antigens as described above or elsewhere herein, and (b) a package insert including instructions for using the composition in accordance with a method described above or elsewhere herein.

The invention also provides kits containing (a) a composition including one or more antibodies as described above or elsewhere herein, and (b) a package insert including instructions for using the composition in accordance with a method described above or elsewhere herein.

It is to be understood that when reference is made to one or more antigens that each have a specified sequence, the sequence of each of the antigens can be independently selected from the sequence options noted. Thus, if one or more Bm antigens are noted as each including the amino acid sequence of any one of SEQ ID NOs: 1-24 and 49-54, this can include, e.g., 30 different antigens, with there being a separate antigen for each of SEQ ID NOs: 1-24 and 49-54.

The invention also provides a composition selected from the group consisting of compositions 1-112 of Tables 2-9, wherein the composition comprises each of the Bm antigens indicated as being present in the composition or one or more antigenic variants and/or antigenic fragments thereof.

In some embodiments, the composition comprises one or more antigenic variant of one or more Bm antigen indicated as being present in the composition, and the sequence of the one or more variant has at least 80%, 85%, 90%, 95%, 97%, or 99% identity to the sequence of the corresponding Bm antigen indicated as being present in the composition, or an antigenic fragment thereof.

In some embodiments, one or more (e.g., each) Bm antigen of the composition comprises a sequence having 100% identity to the sequence of a Bm antigen indicated as being present in the composition or an antigenic fragment thereof.

In some embodiments, the composition further comprises a Bm antigen selected from the group consisting of Bm antigen of SEQ ID NO: 4, 8-24, 51-54, or an antigenic variant and/or antigenic fragment thereof.

In some embodiments, the composition further comprises 1, 2, 3, 4, or 5 Bm antigens selected from the group consisting of Bm antigen(s) of SEQ ID NO: 4, 8-24, 51-54, or antigenic variant(s) and/or antigenic fragment(s) thereof.

The invention also provides a composition comprising a nucleic acid molecule corresponding to each Bm antigen, antigenic variant, or antigenic fragment of a composition described above.

The invention further provides a composition comprising an antibody that specifically binds to each Bm antigen, or antigenic variant and/or antigenic fragment thereof, of a composition described above.

In some embodiments, the composition further comprises one or more adjuvant, carrier, diluent, excipient, or preservative.

In some embodiments, the composition is formulated for administration by the oral route or a parenteral route, such as a parenteral route selected from the group consisting of the intravenous, intraperitoneal, subcutaneous, intramuscular, and topical routes.

The invention also provides a method of immunizing or conferring protective immunity against babesiosis to a subject, the method comprising administering a composition described above to the subject.

In some embodiments, the subject does not experience babesiosis.

In some embodiments, the treatment reduces the severity of Babesia infection (e.g., parasitemia or parasite burden) upon a subsequent challenge.

In some embodiments, the treatment reduces the severity of babesiosis upon a subsequent challenge.

In some embodiments, the subject experiences babesiosis.

In some embodiments, the treatment reduces the severity of Babesia infection (e.g., parasitemia or parasite burden).

In some embodiments, the treatment reduces the severity of babesiosis.

In some embodiments, the subject experiences mild or severe babesiosis, including persistent or relapsing babesiosis.

The invention also provides a method of supplementing an immune response in a subject experiencing babesiosis, the method comprising administering a composition of including antibodies as described above to the subject.

The invention also provides methods of treating babesiosis in a subject in need thereof, the method comprising administering a composition described above to the subject.

In some embodiments, the etiology of babesiosis is Babesia microti.

The invention also provides a method for determining whether a subject, prior to administration of a composition described above, has protective immunity against Bm-induced babesiosis, the method comprising:

a. applying a body fluid sample from the subject to a solid support, wherein the solid support comprises two or more Bm antigens comprising an amino acid sequence having at least 80% sequence identity to the amino acid sequence of any one of SEQ ID NOs: 1-24 or 49-54, or one or more antigenic fragments thereof;

b. applying an antibody detection reagent to the solid support of (a); and

c. identifying the subject as likely to have protective immunity against Bm-induced babesiosis if the fluid sample from the subject is determined to have a sufficient titer of IgG antibodies that specifically react with two or more Bm antigens of (a) or antigenic fragments thereof of, that is, a titer in the fluid sample above a cutoff titer for the two or more Bm antigens.

The invention also provides a method for determining whether a subject, following administration of a composition described above, has acquired protective immunity against Bm-induced babesiosis, the method comprising:

a. applying a body fluid sample from the subject to a solid support, wherein the solid support comprise two or more Bm antigens comprising an amino acid sequence having at least 80% sequence identity to the amino acid sequence of any one of SEQ ID NOs: 1-24 or 49-54, or one or more antigenic fragments thereof;

b. applying an antibody detection reagent to the solid support of (a); and

c. identifying the subject as likely to have protective immunity against Bm-induced babesiosis if the fluid sample from the subject is determined to have a sufficient titer of IgG antibodies that specifically react with two or more Bm antigens of (a) or antigenic fragments thereof of, that is, a titer in the fluid sample above a cutoff titer for the two or more Bm antigens.

The invention also provides a method for identifying a patient who experiences babesiosis and is likely to benefit from an anti-Bm antibody-based therapy, the method comprising:

a. applying a body fluid sample from the subject to a solid support, wherein the solid support comprise two or more Bm antigens comprising an amino acid sequence having at least 80% sequence identity to the amino acid sequence of any one of SEQ ID NOs: 1-24 or 49-54, or one or more antigenic fragments thereof;

b. applying an antibody detection reagent to the solid support of (a); and

c. identifying the patient as likely to benefit from a composition comprising antibodies as described above if the fluid sample from the subject is determined to have an insufficient titer of IgG antibodies that specifically react with two or more Bm antigens of (a) or antigenic fragments thereof, that is, a titer in the fluid sample below a cutoff titer for the two or more Bm antigens.

The invention also provides a method of optimizing the administration or therapeutic efficacy of an anti-Bm antibody-based therapy to a subject experiencing babesiosis, the method comprising:

a. applying a body fluid sample from the subject to a solid support, wherein the solid support comprise two or more Bm antigens comprising an amino acid sequence having at least 80% sequence identity to the amino acid sequence of any one of SEQ ID NOs: 1-24 or 49-54, or one or more antigenic fragments thereof;

b. applying an antibody detection reagent to the solid support of (a); and

c. administering to the subject the composition comprising antibodies as described above if the sample from the subject is determined to have an insufficient titer of IgG antibodies that specifically react with two or more Bm antigens of (a) or antigenic fragments thereof, that is, a titer below a cutoff titer for the two or more Bm antigens.

In some embodiments, the method further comprises administering to the subject the composition described herein, wherein the subject has been determined as likely to benefit from an anti-Bm antibody-based therapy or from a modified dose or dosage of anti-Bm antibody-based therapy.

The invention also provides a kit comprising (a) two or more Bm antigens comprising an amino acid sequence having at least 80% (e.g., at least 85%, 90%, 92%, 95%, 97%, 98%, 99%, or 100%) sequence identity to the amino acid sequence of any one of SEQ ID NOs: 1-24 or 49-54, or one or more antigenic fragments thereof, wherein two or more Bm antigens are optionally immobilized on one or more solid supports; (b) optionally an antibody detection reagent; and/or (c) optionally a package insert comprising instructions for using the two or more Bm antigens and the antibody detection reagent in accordance with a method described herein. In some embodiments, the two or more Bm antigens is comprised within one of compositions 1-112.

In some embodiments, the kit comprises one or more Bm antigen comprising an amino acid sequence having at least 80% (e.g., at least 85%, 90%, 92%, 95%, 97%, 98%, 99%, or 100%) sequence identity to the amino acid sequence of any one of SEQ ID NOs: 49-54, or one or more antigenic fragments thereof. The invention also provides a kit comprising (a) the composition described above and (b) a package insert comprising instructions for using the composition in accordance with a method described herein.

### Definitions

In this application, unless otherwise clear from context, (i) the term “a” may be understood to mean “at least one;” (ii) the term “or” may be understood to mean “and/or;” and (iii) the terms “including” and “includes” may be understood to encompass itemized components or steps whether presented by themselves or together with one or more additional components or steps.

As used herein, the term “about” refers to a value that is no more than 10% above or below the value being described. For example, the term “about 5 nM” indicates a concentration that ranges from 4.5 nM to 5.5 nM.

By “adjuvant” is meant one or more substances that cause stimulation of the immune system. In the context of this application, an adjuvant is used to enhance an immune response to one or more Bm antigens. Enhancement of humoral immunity can be determined by, for example, an increase in the titer of antibodies specific for an antigen. Enhancement of cellular immunity can be measured by, for example, a delayed type hypersensitivity response, the proliferation of antigen-specific T cells or the production of cytokines (e.g., IFN-gamma, IL-2) by T cells upon exposure to the antigen. An adjuvant may be administered to a subject before, in combination with, or after administration of one or more Bm antigens. Examples of adjuvants include, but are not limited to, aluminum compounds (e.g., alum or ALHYDROGEL®), oil formulations, polymers, immune stimulating complexes (e.g., ISCOM® matrix), vitamins (e.g., vitamin A, vitamin B12, vitamin E), minerals (e.g., selenium), saponins (e.g., Quil-A™, QS21), bacterial and fungal cell wall components (e.g., Freund's complete adjuvant, Freund's incomplete adjuvant, lipopolysaccharides, lipoproteins, and glycoproteins), hormones, cytokines, toxins, micelle forming adjuvants, DDA (dimethyldioctadecylammonium bromide), bacterial DNA such as CpG DNA, and encapsulating adjuvants such as liposome formulations.

As used herein, the term “administration” refers to the administration of a composition (e.g., a pharmaceutical preparation that includes a prophylactic or therapeutic agent as described herein) to a subject. Administration to a subject (e.g., to a human or mouse) may use any appropriate route (e.g., oral or parenteral, including, for example, the intravenous, intraperitoneal, subcutaneous, intramuscular, and topical routes.

As used herein, the term “antibody” refers to an immunoglobulin molecule that specifically binds to, or is reactive with, a particular antigen (e.g., a Bm antigen as described herein), and includes monoclonal, genetically engineered, and otherwise modified forms of antibodies, including, for example, chimeric antibodies, humanized antibodies, heteroconjugate antibodies (e.g., bi-, tri-, and quadri-specific antibodies), and antigen-binding fragments of antibodies including Fab, Fab′, F(ab′)₂, Fv, scFv, tandem scFv, diabody, triabody, tetrabody, small modular immunopharmaceutical (SMIP), nanobody or other single domain antibody.

By “antigen” is meant a molecule to which an antibody can selectively bind. A target antigen can be a protein, carbohydrate, nucleic acid, lipid, hapten, or other naturally occurring or synthetic compound. A target antigen, if a protein, can be a polypeptide, peptide, or peptide mimic. An “antigenic fragment” or an “antibody-binding fragment” is able to complex with the same antigen-binding molecule, e.g., an antibody, as the antigen from which it is derived. An antigen or antigenic fragment can be administered to a subject to generate an immune response. Antigens of the invention are “immunogens,” which are capable of inducing an immune response in a subject to which they are administered. Examples of immunogens include polypeptides of any one of SEQ ID NOs: 1-24 and 49-54, immunogenic fragments thereof, and variants of these polypeptides and immunogenic fragments.

The portion of an antigen that specifically interacts with the antigen-binding domain of an antibody is an “antigenic determinant” or “epitope.” An antigen can comprise a single epitope, but typically comprise at least two epitopes, and can include any number of epitopes, depending on the size, conformation, and type of antigen.

The term “antigen-binding fragment,” as used herein, refers to one or more fragments of an antibody that retain the ability to specifically bind to a target antigen (e.g., a Bm antigen). Antibody fragments can be a Fab, Fab′, F(ab′)₂, Fv, scFv, tandem scFv, diabody, triabody, tetrabody, small modular immunopharmaceutical (SMIP), nanobody, or other single domain antibody. Examples of antibody fragments encompassed of the term “antigen-binding fragment” of an antibody include, but are not limited to: (i) a Fab fragment, a monovalent fragment consisting of one variable domain (V_{L}, V_{H}) and one constant domain (C_{L}, C_{H}1) of each of the light (L) and heavy (H) chains; (ii) a Fab′ fragment, a monovalent Fab fragment which includes reduced sulfide thiols; (iii) a F(ab′)₂fragment, a bivalent fragment comprising two Fab′ fragments linked by a disulfide bridge at the hinge region; (iv) a Fv fragment, which consists of the V_{L} and V_{H} domains of a single arm of an antibody; (v) a single-chain (sc) Fv, a Fv fragment for which the V_{L} and V_{H} domains are connected by a small peptide linker and fold onto each other; (vi) a tandem scFv, two scFv which are connected by a small peptide in tandem; (vii) a diabody, a dimer of scFv for which the peptide linker between the V_{L} and V_{H} domains is so small that one domain cannot fold onto the other domain of the same scFv but rather dimerizes with the other domain of the other scFv; (viii) a triabody, a trimer of scFv; (ix) a tetrabody, a tetramer of scFv; (x) a small modular immunopharmaceutical (SMIP), a modified scFv connected to a tandem of CH domains by the hinge region of an immunoglobulin; (xi) a nanobody, a single domain antibody comprising the V_{H} domain of a heavy chain antibody or a common antibody, (xii) an isolated complementarity determining region (CDR); and (xiii) a combination of two or more isolated CDRs which can optionally be joined by a synthetic linker. In addition, an antigen-binding fragment also refers to an antibody mimic such as an affibody, a structure which typically consists of three alpha helixes and recognizes the target antigen otherwise recognized by an intact antibody. Antigen-binding fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in some embodiments, by chemical peptide synthesis procedures known in the art.

As used herein, “babesiosis” is the disease caused by any protozoan parasites of the genus Babesia. The genus “Babesia” includes pathogenic species that are capable of infecting humans including, for example, Babesia microti, Babesia duncani, Babesia divergens, Babesia venatorum, Babesia bovis, Babesia bigemina, and Babesia crassa. Babesia microti is a species complex for which isolates have been grouped into 4 clades. The term “Babesia microti”, as used herein, encompasses isolates from all 4 of these clades.

As used herein, “experiencing babesiosis” or “experiences babesiosis” refers to a subject who presents with at least one clinical manifestation or symptom known to be evoked by babesiosis and is diagnosed as infected with one or more Babesia species.

A “binding molecule” or “antigen-binding molecule” of the present application refers, in its broadest sense, to a molecule that specifically binds to an antigen (e.g., to an antigenic determinant of an antigen). In some embodiments, a binding molecule is an antibody or an antigen-binding fragment thereof.

In certain embodiments, the phrase “specifically binds” or “specific immunoreactivity” refers to a binding reaction which is determinative of the presence of an antigen (e.g., a particular Bm antigen) in a heterogeneous population of proteins and other biological molecules that is recognized, e.g., by an antibody or antigen-binding fragment thereof, with particularity. An antibody or antigen-binding fragment thereof that specifically binds to an antigen will bind to the antigen with a K_{D} of less than 100 nM. For example, an antibody or antigen-binding fragment thereof that specifically binds to an antigen will bind to the antigen with a K_{D} of up to 100 nM (e.g., between 1 pM and 100 nM). An antibody or antigen-binding fragment thereof that does not exhibit specific binding to a particular antigen or epitope thereof will exhibit a K_{D} of greater than 100 nM (e.g., greater than 500 nm, 1 μM, 100 μM, 500 μM, or 1 mM) for that particular antigen or epitope thereof. A variety of immunoassay formats may be used to select antibodies or antigen-binding fragments which are specifically immunoreactive with a particular antigen. For example, ELISA assays are routinely used to identify antibodies which specifically bind to an antigen, such as a protein or carbohydrate. See, Harlow & Lane, Antibodies, A Laboratory Manual, Cold Spring Harbor Press, New York (1988) and Harlow & Lane, Using Antibodies, A Laboratory Manual, Cold Spring Harbor Press, New York (1999), for a description of immunoassay formats and conditions that can be used to determine specific immunoreactivity.

As used herein, the terms “bi-, tri-, and quadri-specific antibodies” refer to monoclonal, often human or humanized antibodies which have binding specificities for two, three, or four different antigens, respectively.

“CD4” is an abbreviation for “cluster of differentiation 4,” a glycoprotein expressed on the surface of T lymphocytes, as well as certain other cells. Human CD4 is encoded by the CD4 gene whereas mouse CD4 is encoded by the cd4 gene. The amino acid sequence of an exemplary protein encoded by human CD4 is shown under UniProt Accession No. P01730. The nucleic acid sequence of exemplary human CD4 is shown under NCBI Reference Sequence: NM_000616.5. The term “CD4” also refers to natural variants of the CD4 protein, such as proteins having at least 85% identity (e.g., 85%, 86%, 87%, 88%, 89%, 90%, 91%, 92%, 93%, 94%, 95%, 96%, 97%, 98%, 99%, 99.9% identity, or more) to the amino acid sequence of the exemplary CD4 protein.

As used herein, the terms “cd4-deficient” and “cd4 knockout” (cd4 KO) mean a mouse or genetic background lacking the cd4 gene and therefore lacking expression of the CD4 molecule.

“Conferring protective immunity” refers to providing a subject (e.g., a human or mouse) or a population of subjects with antigens or antibodies specific for these antigens in order to protect against a disease (e.g., babesiosis) caused by exposure to a pathogen (e.g., Babesia microti) such that the clinical manifestations, pathology, or symptoms of disease are less severe in the treated subject than in a non-treated subject, or such that the rate of infection or clinical manifestations, pathology, or symptoms of disease is lower in the treated population than in the non-treated population.

As used herein, the terms “conservative mutation” and “conservative substitution” refer to a mutation or substitution of one or more amino acids for one or more different amino acids which exhibit similar physicochemical properties, such as polarity, electrostatic charge, and steric volume. It is appreciated that amino acid families include (i) G, A, V, L and I; (ii) D and E; (iii) C, S and T; (iv) H, K and R; (v) N and Q; and (vi) F, Y and W. A conservative mutation or substitution is therefore one that substitutes one amino acid for a member of the same amino acid family (e.g., a substitution of Arg (A) for Lys (L) or Ser (S) for Thr (T)).

As used herein, the terms “effective amount,” “therapeutically effective amount,” and a “sufficient amount” of a composition described herein refer to a quantity sufficient to, when administered to the subject, effect beneficial or desired results, including clinical improvement. As such, an “effective amount” or synonym thereto depends upon the context in which it is being applied. In the context of babesiosis, it is an amount of the composition sufficient to reduce or eliminate clinical manifestations, pathology and/or symptoms associated with babesiosis. The amount of a given composition required to achieve such protection will depend upon several factors, including the molecule(s) of the composition that confer protection, the pharmaceutical formulation, the route of administration, the severity of disease, the characteristics of the subject (e.g., age, gender, health status, or weight), and the like. These various factors can be easily determined by one skilled in the art and should be taken into consideration to set or adjust doses and dosages so that the optimum therapeutic response is achieved.

As used herein, the term “IgG” is an abbreviation for immunoglobulin G antibody.

As used herein, “immunize” means to administer a clinically relevant dose of a molecule or composition of molecules, or multiple doses over time in a dosage regimen, to achieve an immunized state. “Immunized,” as used herein, qualifies the state of a subject who is protected, partially or in full, from a pathogen. In the context of babesiosis, a subject may be immunized with a composition comprising one or more Bm antigens (or antigenic fragments thereof), a composition comprising one or more nucleic acid molecules (e.g., RNA or DNA molecules) encoding one or more Bm antigens (or antigenic fragments thereof), or a composition comprising one or more Bm-specific antibodies.

“Immune response” in the context of babesiosis means a response in the subject to the introduction of one or more Bm antigens which is generally characterized by, for example, expansion of Bm-specific T cells and/or production of Bm-specific antibodies. Generally, an immune response includes a cellular response such as the expansion of a population of CD4+ T cells specific for Bm antigens, a humoral response such as the production of Bm-specific antibodies, or both. In some embodiments, the immune response elicited by a composition of the invention comprising one or more Bm antigens includes, but is not limited to, responses to the protein components of the composition. In certain embodiments, upon subsequent challenge with Bm parasites, the immune response induced by the introduction of one of more Bm antigens prevents the expansion of the parasite population that causes babesiosis.

As used herein, “immunoreactive” qualifies an antigen which reacts with an antigen-binding molecule (e.g., an intact antibody or antigen-binding fragment thereof). Immunoreactivity can be referred to as “seroreactivity” when an antigen reacts with one or more antibodies present in serum or plasma prepared from the blood of a subject. “Immunoreactivity” and “reactivity” are used interchangeably herein to mean the detection of an antigen-binding molecule (e.g., an antibody (e.g., an IgG antibody)) in a sample (e.g., a body fluid sample). As used herein, “differential immunoreactivity” or “differential reactivity” means a difference in immunoreactivity of an antigen with an antigen-binding molecule (e.g., an antibody (e.g., an IgG antibody)) in a sample (e.g., a body fluid sample) between two experimental conditions, wherein p-values are less than 0.05 as calculated using parametric or non-parametric hypothesis testing methods (e.g., two-tailed Student's t-test and Wilcoxon's rank-sum test).

As used herein, “lacking” or “to lack” means being deficient in, or not having a gene or a molecule.

As used herein, “parasitemia” refers to the frequency of red blood cells infected with a parasite (e.g., Babesia microti) in the blood of an infected subject. The terms “parasitemia” and “infection” may be used interchangeably, when the latter is considered in the context of the parasites causing the infection.

“Percent (%) sequence identity” with respect to a reference polynucleotide or polypeptide sequence is defined as the percentage of nucleotides or amino acids in a candidate sequence that are identical to the nucleotides or amino acids in the reference polynucleotide or polypeptide sequence, after aligning the sequences and introducing gaps, if necessary, to achieve the maximum sequence alignment. Alignment for the purpose of determining percent sequence identity can be achieved in various ways that are within the capabilities of one of skill in the art, for example, using publicly available computer software such as BLAST, BLAST-2, or MegAlign. Those skilled in the art can determine appropriate parameters for aligning sequences, including any algorithms needed to achieve maximal alignment over the full length of the sequences being compared. As an illustration, the percent sequence identity of a given nucleic or amino acid sequence, A, to, with, or against a given nucleic or amino acid sequence, B, (which can alternatively be phrased as a given nucleic or amino acid sequence, A that has a certain percent sequence identity to, with, or against a given nucleic or amino acid sequence, B) is calculated as follows:

100 multiplied by the fraction X/Y

where X is the number of nucleic or amino acids scored as identical matches by a sequence alignment program (e.g., BLAST) in that program's alignment of A and B, and where Y is the total number of nucleic or amino acids in B. It will be appreciated that, where the length of nucleic or amino acid sequence A is not equal to the length of nucleic or amino acid sequence B, the percent sequence identity of A to B will not equal the percent sequence identity of B to A. Percent sequence identity can be used to qualify variant sequences (e.g., variants of a polypeptide of any one of SEQ ID NOs: 1-24 and 49-54, or fragments thereof).

As used herein, the term “pharmaceutically acceptable” refers to those compounds, materials, compositions and/or dosage forms, which are suitable for contact with the tissues of a subject without inducing excessive toxicity, irritation, allergic response and other complications commensurate with a reasonable benefit/risk ratio.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” also refers to the products of post-expression modifications, including, without limitation, glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology but is not necessarily translated from a designated nucleic acid sequence. It may be generated in any manner, including by chemical synthesis.

A polypeptide “fragment,” “variant,” “analog,” or “derivative” of the present application encompasses any polypeptide that retains at least some of the properties of the intact polypeptide of the application. Polypeptide fragments of the present application include proteolytic fragments and deletion fragments, as well as antibody-binding fragments discussed elsewhere herein. Polypeptide variants of the present application include polypeptides and fragments thereof for which the amino acid sequence has been altered by one or more amino acid substitutions, deletions, or insertions. Variants may occur naturally or non-naturally. Non-naturally occurring variants may be produced using mutagenesis techniques known to those skilled in the art. Polypeptide variants may comprise conservative or non-conservative amino acid substitutions. For example, variants include polypeptides that have at least 80% sequence identity to an amino acid sequence recited herein. Polypeptide variants may also be referred to herein as “polypeptide analogs.” As used herein, polypeptide derivatives refer to polypeptides having one or more amino acid residues chemically derivatized by reaction of a functional side group. Also included as “derivatives” are polypeptides that contain one or more naturally occurring amino acid derivatives of the twenty standard amino acids. Derivatives of the present application may include polypeptides that have been altered so as to exhibit features not found in the reference polypeptides of the application.

The term “polynucleotide” is intended to encompass a singular “polynucleotide” as well as plural “polynucleotides,” and refers to a molecule composed of monomers (nucleic acids) linearly linked by conventional phosphodiester bonds or non-conventional bonds (e.g., an amide bond, such as found in peptide nucleic acids). A “polynucleotide” can be derived from a natural biological source or produced by recombinant technology. The term “nucleic acid” refers to any one or more nucleic acid segments present in a polynucleotide (e.g., DNA or RNA). The term “nucleic acid” additionally encompasses polynucleotides (e.g., DNA or RNA) that contain modified nucleotides or modified nucleotide linkages. For example, the term encompasses RNA molecules that contain pseudouridines and/or 2′-O-methylated nucleosides.

By “isolated” polynucleotide or nucleic acid is intended a polynucleotide molecule or nucleic acid segment that has been removed from its native environment. For example, a recombinant polynucleotide encoding a polypeptide or antigenic fragment thereof contained in a vector is considered isolated for the purposes of the present invention. Further examples of an isolated polynucleotide include recombinant polynucleotides maintained in heterologous host cells or purified (partially or substantially) polynucleotides in solution. Isolated RNA molecules include transcripts (e.g., messenger RNA) of polynucleotides of the present invention that are generated in vivo or in vitro. Isolated nucleic acids or polynucleotides of the present invention also include such molecules produced synthetically. An “isolated” nucleic acid or polynucleotide may include a regulatory element such as a promoter, transcription terminator, or ribosome binding site.

As used herein, “probing” with respect to a body fluid sample means exposing the sample to one or more antigens and measuring the reactivities of these antigens to antigen-binding molecules (e.g., antibodies) contained within the sample.

“Resolution,” as used herein, refers to the process or processes by which a subject clears a pathogen (e.g., Babesia microti) and overcomes some of the clinical manifestations, pathology, or symptoms caused by the pathogen. A “partial resolution,” as used herein, refers to a reduction of parasitemia of 10% or more that is not complete (also referred to as full), whereas a “complete resolution” or “a full resolution” refers to a reduction of parasitemia to levels below or near the limit of detection.

The term “sample,” as used herein, refers to a composition that is obtained or derived from a subject. Samples include, e.g., blood (e.g., whole blood, serum, or plasma), urine, saliva, sputum, tears, perspiration, mucus, and combinations thereof.

A “test sample” is a sample obtained from a subject after administration of one or more Bm antigens or one or more antibodies specific for Bm antigens. A “reference sample” or “control sample” is a sample that is used for the purpose of comparison to a test sample. In various examples, a reference sample is obtained from one or more subjects prior to administration of one or more Bm antigens or one or more antibodies specific for Bm antigens. “Reference levels,” “control levels,” or “cutoff levels” of Bm-specific antibodies, for example, can be based on the levels of Bm-specific antibodies measured in the “reference samples” or “control samples.”

As used herein, “solid support” is defined as any surface to which molecules, also referred to as “ligands”, can be attached through either covalent or non-covalent bonds. Solid support includes, but is not limited to, membranes, plastics, paramagnetic beads, charged paper, nylon, Langmuir-Bodgett films, functionalized glass, germanium, silicon, polytetrafluoroethylene, polystyrene, gallium arsenide, gold and silver. Any other material known in the art that is capable of having functional groups such as amino, carboxyl, thiol or hydroxyl incorporated on its surface, is also contemplated. Such material includes any surface with any topology, including, for example, spherical surfaces, grooved surfaces, and cylindrical surfaces (e.g., columns). Multiple ligands, each specific for a target, can be attached to specific locations (“addresses”) on the surface of a solid support in an addressable format to form an array, also referred to as a “microarray” or “chip.” By way of non-limiting example only, an array can be formed with a planar solid support, the surface of which ligands are attached to. By way of non-limiting example only, an array also can be formed by attaching ligands to beads and placing the beads in an array format that uses another solid support, such as a microtiter plate.

As used herein, the terms “subject,” “individual,” and “patient” refer to an organism that experiences or is at risk of experiencing a particular disease or condition as described herein (e.g., babesiosis). Examples of subjects, individuals, and patients include mammals, such as humans, non-human primates, and mice, among others. As used herein, and as examples only, human subjects at risk of experiencing babesiosis are those who are older than 40 years of age and reside in babesiosis endemic areas or travel to such endemic areas. Additionally included are those who, regardless of age, are transfused with blood products. As used herein, and as examples only, subjects who experience or are at risk of experiencing severe babesiosis, including persistent or relapsing babesiosis, are those who are or were recently treated with rituximab for a B cell lymphoma or an autoimmune disease, those for whom the CD4 T cell compartment has been depleted by HIV infection or an immunosuppressive therapy for stem cell or solid organ transplantation, and those who are immunosuppressed by treatment of comorbidity, particularly malignancy in the context of asplenia.

By “treating” a disease, disorder, or condition is meant reducing at least one symptom of the disease, disorder, or condition by administrating a therapeutic agent to the subject which has been diagnosed with the disease, disorder, or condition.

By “treating prophylactically” a disease, disorder, or condition is meant reducing the incidence of a disease, disorder or condition in a population or reducing the severity of a disease, disorder or condition in a subject or population of subjects by administering a prophylactic agent to the subject or subjects prior to the onset of said disease, disorder, or condition.

As used herein, the term “wild-type” and the abbreviation “WT” means a subject or a group of subjects which do not lack a particular gene (e.g., the cd4 gene) and therefore do not lack the molecule encoded by this particular gene (e.g., the CD4 molecule).

Unless defined otherwise, all technical and scientific terms used herein have the same meaning as commonly understood to one of ordinary skill in the art to whom this disclosure belongs. For any term presented in the art which is identical to any term expressly defined in this disclosure, the term's definition presented in this disclosure will control in all respects. The details of one or more embodiments of the invention are set forth in the description below. Other features, objects, and advantages of the invention will be apparent from the description and from the claims.

### BRIEF DESCRIPTION OF THE DRAWINGS

FIG. 1 is a pair of graphs showing that B cells do not contribute to the resistance of wild-type mice to Babesia microti (B. microti) (left panel) but are critical for resolution of B. microti infection in cd4-deficient mice (right panel). Parasitemia values are reported as mean+SEM. N=5-7 per group.

FIG. 2 is a series of graphs showing that resolution of B. microti infection in cd4-deficient mice is concomitant with an accumulation of B. microti-specific IgGs in blood. Parasitemia values and B. microti specific antibody titers are reported as mean+SEM. For a given time point, differences in antibody titers were tested for statistical significance using an unpaired Student's t-test (*=P<0.05; **=P<0.01; ***=P<0.001). N=6-7 per group.

FIG. 3 is a graph showing that activation-induced cytidine deaminase, the enzyme required for antibody class switch and somatic hypermutation, is required for complete resolution of B. microti infection in cd4-deficient mice. Parasitemia values are reported as mean+/−SEM. N=7-8 per group.

FIG. 4 is a graph showing that Fc receptors, whether activating or inhibitory, are dispensable for resolution of B. microti infection in cd4-deficient mice. Parasitemia values are reported as mean+SEM for 1 of the 4 groups; for clarity, SEM is omitted for 3 of the 4 groups. N=5-7 per group.

FIG. 5 is a graph showing that the complement component C3 promotes clearance of B. microti organisms at time of peak infection in cd4-deficient mice but does not contribute to the resistance of wild-type mice to B. microti. Parasitemia values are reported as mean+/−SEM. For a given time point, differences in parasitemia values were tested for statistical significance using an unpaired Student's t-test (*=p<0.05; **=p<0.01; ***=P<0.001). N=6-8 per group.

FIG. 6 is a graph showing the time points at which blood was collected from wild-type mice and cd4-deficient mice infected with B. microti. Specifically, blood samples were obtained at times (arrows) of peak infection (day 18), partial resolution of infection (day 24), and full resolution of infection (day 30) in cd4-deficient mice. Note that on day 24 post-infection, the infection was fully resolved in WT mice. N=6 for the WT group; N=10 for the cd4-/- group.

FIG. 7A is a heat map of IgG antibody reactivity stratified by mouse strain and time post-infection. Each column of the heat map represents a mouse sample, and each row represents a B. microti protein. Each grey header encompasses several columns and denotes a mouse strain at a given time point. B. microti proteins (n=74) are those significantly reactive to IgG contained in plasma from B. microti-infected wild-type or cd4-deficient mice and are ranked in decreasing order by mean normalized signal intensity for all samples across strains and time points. The grey scale (top-left corner) represents normalized signal intensity on the log₂ scale, i.e., values of 1.0 signify a 2.0-fold increase over background, values of 2.0 signify a 4.0-fold increase over background, values of 3.0 signify an 8.0-fold increase over background, etc.

FIG. 7B is a Venn diagram showing the numbers of IgG reactive B. microti proteins at times of peak infection (inner circle, n=16), partial resolution of infection (intermediate circle, n=35), and full resolution of infection (outer circle, n=52) in cd4-deficient mice.

FIG. 8 is a series of graphs showing the IgG repertoire against B. microti antigens in wild-type mice (top panel) and cd4-deficient mice (middle panel). Dashed lines indicate the cutoff for IgG reactivity (2-fold above background). The lower panel depicts strain differences in IgG reactivity. In this panel, dashed lines depict strain differences (gain or loss) in IgG reactivity of 2.0-fold.

FIG. 9 is a pair of graphs showing the IgG reactivity against B. microti antigens in cd4-deficient mice at time of full resolution of infection (top panel) and time of peak infection (bottom panel). Dashed lines indicate the cutoff for IgG reactivity (2-fold above background). In both panels, B. microti antigens are ranked in decreasing order of IgG reactivity at time of full resolution. Solid grey bars in the bottom panel denote antigens for which the IgG reactivity is above the cutoff defined above. Below each grey bar is reported the rank of the antigen in regard to IgG reactivity at time of full resolution.

FIG. 10 is a table that lists B. microti antigens for which IgG reactivity is detected at time of peak infection in cd4-deficient mice. Antigens are considered immunoreactive when IgG binding is above that of background by at least 2-fold (1.0 on a log 2 scale). Antigens are identified by their gene ID and are ranked in decreasing order of log 2 IgG reactivity. A brief description of each antigen, when available, is provided. This description includes the Plasmodium falciparum ortholog in the clone PF3D7, when available. The rank of each antigen in regard to IgG reactivity at time of full resolution is reported in the most right column.

FIG. 11 is a series of graphs showing and comparing the IgG reactivity against B. microti antigens in cd4-deficient mice at times of peak infection (top panel) and partial resolution of infection (middle panel). In the top and middle panels, dashed lines indicate the cutoff for IgG reactivity (2-fold above background). The lower panel depicts differences (e.g., gains and losses) in IgG reactivity from time of peak infection to time of partial resolution. In this panel, the dashed line depicts gains in IgG reactivity of 2-fold. Grey bars denote antigens that gained reactivity from time of peak infection to time of partial resolution. Solid grey bars denote antigens that were already reactive at time of peak infection, whereas hatched grey bars denote antigens that were not reactive at time of peak infection. Below each grey bar is reported the rank of the antigen in regard to IgG reactivity at time of full resolution.

FIG. 12 is a volcano plot showing gains in log 2 IgG reactivity (from time of peak infection to time of partial resolution) against −log 10 p values. The larger dots depict the 20 antigens for which gains in IgG reactivity reached the highest degree of significance as assessed by use of a paired Student's t-test. The horizontal dashed line indicates the threshold for significance (p=0.05). Numbers denote antigens for which the gain in reactivity was significant and greater than 2-fold (vertical dashed line). Numbers highlighted by a circle denote antigens that were already reactive at time of peak infection, whereas numbers highlighted by a hexagon denote antigens that were not reactive at time of peak infection. Each number corresponds to the rank of the antigen in regard to IgG reactivity at time of full resolution.

FIG. 13 is a table that lists B. microti antigens for which IgG reactivity increased by more than 2-fold from time of peak infection to time of partial resolution in cd4-deficient mice. Antigens are identified by their gene ID and ranked in decreasing order of the gain in log 2 IgG reactivity. A brief description of each antigen, when available, is provided. This description includes the Plasmodium falciparum ortholog in the clone PF3D7, when available. The rank of each antigen in regard to IgG reactivity at time of full resolution is reported in the most right column. Antigens denoted in grey were already reactive at time of peak infection, whereas antigens denoted in black were not reactive at time of peak infection.

FIG. 14 is a series of graphs showing and comparing the IgG reactivity against B. microti antigens in cd4-deficient mice at times of partial infection (top panel) and full resolution of infection (middle panel). In the top and middle panels, dashed lines indicate the cutoff for IgG reactivity (2-fold above background). The lower panel depicts differences (e.g., gains and losses) in IgG reactivity from time of partial resolution to time of full resolution. In this panel, the dashed line depicts gains in IgG reactivity of 2-fold. Grey columns denote antigens that gained reactivity from time of partial resolution to time of full resolution. Solid grey columns denote antigens that were already reactive at time of peak infection, whereas hatched grey columns denote antigens that were not reactive at time of peak infection but gained reactivity from time of peak infection to time of partial resolution. The dotted grey column denotes the single antigen that was not reactive at time of peak infection, did not gain reactivity from time of peak infection to time of partial resolution but gained reactivity from time of partial resolution to time of full resolution. Below each grey bar is reported the rank of the antigen in regard to IgG reactivity at time of full resolution.

FIG. 15 is a volcano plot showing gains in log 2 IgG reactivity (from time of partial resolution to time of full resolution) against −log 10 p values. The larger dots depict the 20 antigens for which gains in IgG reactivity reached the highest degree of significance as assessed by use of a paired Student's t-test. The horizontal dashed line indicates the threshold for significance (p=0.05). Numbers denote antigens for which the gain in reactivity was significant and greater than 2-fold (vertical dashed line). Numbers highlighted by a circle denote antigens that were already reactive at time of peak infection, whereas numbers highlighted by a hexagon denote antigens that were not reactive at time of peak infection but gained reactivity from time of peak infection to time of partial resolution. The number highlighted by a rectangle denotes the single antigen that was not reactive at time of peak infection, did not gain reactivity from time of peak infection to time of partial resolution but gained reactivity from time of partial resolution to time of full resolution. Each number corresponds to the rank of the antigen in regard to IgG reactivity at time of full resolution.

FIG. 16 is a table that lists B. microti antigens for which IgG reactivity increased by more than 2-fold from time of partial resolution to time of full resolution in cd4-deficient mice. Antigens are identified by their gene ID and ranked in decreasing order of the gain in log 2 IgG reactivity. A brief description of each antigen, when available, is provided. This description includes the Plasmodium falciparum ortholog in the clone PF3D7, when available. The rank of each antigen in regard to IgG reactivity at time of full resolution is reported in the most right column. Antigens denoted in grey were reactive at time of peak infection and/or gained reactivity from time of peak infection to time of partial resolution. The antigen denoted in black was not reactive at time of peak infection, did not gain reactivity from time of peak infection to time of partial resolution but gained reactivity from time of partial resolution to time of full resolution.

FIG. 17 is a series of graphs showing the inverse relationship between parasitemia and IgG reactivity against each of two B. microti antigens at time of peak infection in cd4-deficient mice. These 2 antigens, namely BMR1_01G03280 and BMR1_01G00985, ranked first and tenth when considering their IgG reactivity at time of full resolution in cd4-deficient mice (top panel).

FIG. 18 is a series of graphs showing the relationship between parasitemia and IgG reactivity against each of four B. microti antigens at time of partial resolution in cd4-deficient mice. Three antigens, namely BMR1_01G03280, BMR1_04G05532, and BMR1_04G07360, had IgG titers at time of partial resolution which were inversely correlated with parasitemia at time of partial resolution (left bottom panels). A fourth antigen, namely BMR1_01G02100, had IgG titers at time of partial resolution which were positively correlated with parasitemia at time of partial resolution (most right bottom panel). Note that BMR1_01G03280 and BMR1_04G07360 were already reactive at time of peak infection (solid grey) whereas BMR1_04G05532 and BMR1_01G02100 were not reactive at time of peak infection but gained reactivity from time peak of infection to time of partial resolution (hatched grey).

FIG. 19 is a schematic that classifies the 24 B. microti antigens which displayed IgG reactivity during the resolution of B. microti infection in cd4-deficient mice. Sixteen antigens were identified as IgG reactive at time of peak infection. Of the antigens identified as gaining IgG reactivity from time of peak infection to time of partial resolution, only 7 were not reactive at time of peak infection. Of the antigens identified as gaining IgG reactivity from time of partial resolution to time of full resolution, only 1 antigen was not reactive at time of peak infection or had not gained reactivity by time of partial resolution. Among these 24 distinct antigens, 15 are predicted to lack a signal peptide and are referred to as “group #3 antigens.” Of the 9 antigens predicted to contain a signal peptide, only 3 had an IgG reactivity which was inversely correlated with parasitemia at time of peak infection or time of partial resolution. These 3 antigens are referred to as “group #1 antigens.” The other 6 antigens are referred to as group #2 antigens as they are predicted to contain a signal peptide but displayed IgG reactivity at time of peak infection or partial resolution which was not inversely correlated with parasitemia measured at the respective time point.

FIG. 20 is a table that lists the 24 B. microti antigens identified as displaying or gaining IgG reactivity during the resolution of B. microti infection in cd4-deficient mice. Antigens are identified by their gene ID. A brief description of each antigen, when available, is provided. This description includes the Plasmodium falciparum ortholog in the clone PF3D7, when available. Antigens are classified into three groups using two criteria, namely the predicted presence of a signal peptide, and an inverse relationship between IgG reactivity and parasitemia at time of peak infection and/or partial resolution of infection. Within each group, antigens are ranked in decreasing order of their highest IgG reactivity or gain in IgG reactivity (grey boxes). For the purpose of this application, a sequence ID number (SEQ ID NO) is attributed to each of the 24 antigens.

FIG. 21 is a graph showing that in the absence of interferon-gamma activity, resolution of B. microti infection requires B cells. The solid triangles indicated in the upper portion of the graph correspond to ifngr1-/-+18B12, while the solid triangles indicated in the lower portion of the graph correspond to B6.

FIG. 22 is a graph showing an experimental design to probe the humoral response in ifngr1-deficient mice. The d22 indicator is plasma obtained from wild-type (wt) mice, while the d0, d16, d24, and d35 indicators are for plasma obtained from ifngr1-deficient mice.

FIG. 23 is a series of graphs showing that lack of interferon-gamma activity alters the range of cognate antigens recognized by IgG antibodies.

FIG. 24 is a series of graphs showing accrual of IgG reactivity during resolution of B. microti infection in ifngr1-deficient mice.

FIG. 25 is a series of graphs showing an inverse relationship between IgG titers and parasitemia at the time of mid-resolution.

FIG. 26 is a table that lists 18 B. microti antigens identified by a screen of ifngr1-deficient mice.

### DETAILED DESCRIPTION OF THE INVENTION

The invention is based, in part, on the findings that a) resolution of B. microti infection in cd4-deficient mice requires antibody-producing B cells and antibody class switching, b) such resolution is concomitant with the accumulation of B. microti-specific IgG antibodies in blood, and c) these IgG antibodies target a restricted set of B. microti polypeptides. The invention is further based on studies in a mouse model of interferon gamma receptor type 1 (ifngr1) deficiency.

Accordingly, the invention provides compositions for use in methods for the prophylaxis of babesiosis, the monitoring of prophylaxis efficacy, the treatment of babesiosis, and the monitoring of treatment regimens in a subject (e.g., a mammalian subject, such as a human), as well as such methods. The invention also provides kits that can be used to carry out the methods of the invention. The compositions, methods, and kits of the invention are described further, as follows.

### Compositions

In general, the present invention provides compositions comprising one or more B. microti (Bm) antigens for use in methods for the prophylaxis of babesiosis, the monitoring of prophylaxis efficacy, and the monitoring of treatment regimens for babesiosis that comprise one or more anti-Bm antibodies. The present invention also provides compositions comprising one or more nucleic acid molecules (e.g., RNA or DNA) encoding one or more Bm antigens for use in methods for the prophylaxis of babesiosis. The present invention further provides compositions comprising one or more anti-Bm antibodies for use in methods for the prophylaxis and the treatment of babesiosis.

These compositions and methods can be used to generate or provide immunity to B. microti and thereby to confer partial, enhanced, or full protection in humans and other mammalian subjects who are at risk of exposure to Bm and at risk of developing babesiosis (e.g., mild or severe babesiosis, including persistent or relapsing babesiosis). In various examples, the compositions are useful to (a) reduce the chance of a subject to become ill when infected with Bm, (b) reduce the severity and/or duration of illness in an infected subject, (c) reduce the parasite burden in an infected subject, and/or (d) reduce the chance of dying from babesiosis. In various examples, the compositions are vaccines.

Babesia Microti Antigen-Based Compositions

Compositions of the invention, which can be used, e.g., in the methods and kits described herein, include one or more Bm antigens and optionally a pharmaceutically acceptable adjuvant, carrier or diluent.

Exemplary Bm antigens for use in the compositions of the invention include BMR1_01G03280 (SEQ ID NO: 1), BMR1_04G05532 (SEQ ID NO: 2), BMR1_01G00985 (SEQ ID NO: 3), BMR1_02G01760 (SEQ ID NO: 4), BMR1_03G00365 (SEQ ID NO: 5), BMR1_03G04695 (SEQ ID NO: 6), BMR1_03G03430 (SEQ ID

NO: 7), BMR1_04G06070 (SEQ ID NO: 8), BMR1_04G09385 (SEQ ID NO: 9), BMR1_03G04485 (SEQ ID NO: 10), BMR1_03G01645 (SEQ ID NO: 11), BMR1_03G01960 (SEQ ID NO: 12), BMR1_04G05080 (SEQ ID NO: 13), BMR1_03G00820 (SEQ ID NO: 14), BMR1_01G02100 (SEQ ID NO: 15), BMR1_04G07360 (SEQ ID NO: 16), BMR1_02G02185 (SEQ ID NO: 17), BMR1_04G08260 (SEQ ID NO: 18), BMR1_03G02345 (SEQ ID NO: 19), BMR1_02G02960 (SEQ ID NO: 20), BMR1_01G02545 (SEQ ID NO:

21), BMR1_03G04110 (SEQ ID NO: 22), BMR1_02G02560 (SEQ ID NO: 23), BMR1_04G05635 (SEQ ID NO: 24), BMR1_02G01795 (SEQ ID NO: 49), BMR1_04G07915_s2 (SEQ ID NO: 50), BMR1_01G01620 (SEQ ID NO: 51), BMR1_04G05940 (SEQ ID NO: 52), BMR1_02G02565 (SEQ ID NO: 53), and BMR1_04G06705 (SEQ ID NO: 54). The amino acid sequences for the 24 full-length antigens or their ectodomains are provided in Table 1.

TABLE 1
Amino acid sequences of exemplary
full-length B. microti antigens or their ectodomain

| SEQ ID | | |
|---|---|---|
| NO. | Amino Acid Sequence | Gene ID |
| 1 | DVYEISSGNPPDIEPTSTSLETNVVTNYIPEPNADSESVHVEIQEHDNINPQDACDSE | BMR1_01G03280 |
| | PLEQMDSDTRVLPESLDEGVPHQFSRLGHHSDMASDINDEEPSFKIGENDIIQPPW | ectodomain |
| | EDTAPYHSIDDEELDNLMRLTAQETSDDHEEGNGKLNTNKSEKTERKSHDTQTPQ | |
| | EIYEELDNLLRLTAQEIYEERKEGHGKPNTNKSEKAERKSHDTQTTQEICEECEEGHD | |
| | KINKNKSGNAGIKSYDTQTPQETSDAHEEGHDEINTNKSEKAERKSHDTQTTQEIC | |
| | EECEEGHDKINKNKSGNAGIKSYDTQTPQETSDAHEEEHGNLNKNKSGKAGIKSH | |
| | NTQTPLKKKDFCKEGCHGCNNKPEDNERDPSSPDDDGGCECGMTNHFVFDYKTT | |
| | LLLKSLKTET | |
| 2 | IITDGLSAIGSVASEVGNTVKDVSSEALLGELQQIADGGKIIRDGTESNFVNSIANTV | BMR1_04G05532 |
| | MKNVVGTAVLKASSGITHNGDFAFYNFMYPENDDYPWACICDESDYEEYIKGKKD | ectodomain |
| | KVRCRNYIDSSLQNAVLYCNPANHNSSINDNANNNPPKQIDNHVSIPQTAPANHT | |
| | TVLSTEVDTNHNEQKQPNSPSVPSESQNSVSAPKDESVSSTVEGAKSSS | |
| 3 | TLVSKITTPNNLPGIDTCSNWEDVSVCTTKNTRNCISGEGKPKDCFAIGKSLFKTFPN | BMR1_01G00985 |
| | CYEGVIIDQVTFSGFETLEIHYCDIDKILHKANEVVKSIHELKEKTNQLTEKIKDIPDLID | ectodomain |
| | KVIKVNSEISKIFHQDKIKHMEKEANDFKNALKTTRNYIISYDSLDKTKQSNLLTSLGK | |
| | LMNKIKTKLSEMDKTLHSTLDTNNTIIDLVNNNSSHVKHPNDFNKTMELYNETITK | |
| | ADAIKKNIEKLKEHRKISTHKTIFSNNIDKLIDNLTDYFENINRSIDAVRDKLSKYQLET | |
| | GKMVLLFKNVNEIQKHIKNTDMHIRTCNYDFSDIEQKYSLITAKITVEDGQSITTSNK | |
| | STVDIPEEKVDRVNVNVDKAENSDNETSQENTSVKPTDHKEIEDSASEENAIGENG | |
| | DYDSDEDIDTNDVKEDHENAIDSEYTVSSTGDVLEDEVVEENAIGENGDYDSDEDI | |
| | DTNDVKEDHENAIDSEYTVSSTGDVLEDEVVEENAIGENGDYDSDEDIDTNDVKED | |
| | HENAIDSEYTVSSTGDVLEDEVVEENAIGENGDYDSDEDIDTNDVKEDHENAIDSE | |
| | YTVSSTGDVLEDEVVEENAIGENGDYDSDEDIDTNDVKEDHENAIDSEYLVSSTGD | |
| | VLEDEWEENAIGENGDYDSDEDIDTNDVKEDHEDAIDSEYLVSLTGDVLEDEVVEE | |
| | NAIGENGDHDSDEDIDIEEVNEEDHEDAIDSDNSISNSENVDTTPTENVDTIPTKNA | |
| | NTTPTKNANATPTKNVDTIPTKNVDTIPTKNANTTPTKNVVTTPTKNANTTPTKNV | |
| | VTTPTKNANTTPTKNVVTTPTKNANTTPTKNVVTTSTGNVRTKHTSTNSHVLAPDT | |
| | DEYPAQISQHKTIDKYYQSLELEDEENADISSSDKPVSPINLEDKNSTYDHMHKTDN | |
| | VKSAGIASYD | |
| 4 | NRSCPGNNGVGGGSGDNNSGIIPNDPHPCCNNLRQKPQYQTKPENELVNDDRDL | BMR1_02G01760 |
| | NFNKIRGGKQIITFTVPSIDDLKNKRLSDSEFILSEKANPLISSGDSKNVIVFEVKNDNE | ectodomain |
| | KLMGSVEVGQWEVTITTSCIRRIVIFDSNEVSDNIPMYIYIVDYFEGGNSTVSKFFFA | |
| | NNRWNDFTNHTPNAA | |
| 5 | FLLNRSEFKWFKVGLIITTIFPFKHSFDYNLTHIFLFSICTLIFCVKPVDEESGAKKEGFD | BMR1_03G00365 |
| | FKKMVPDKFKKYT | ectodomain |
| 6 | DSGNSSPQTPPETSSPINGVIGDENNGLEHLSSSGLEVDDNLPELLKTSPFSGQNSD | BMR1_03G04695 |
| | VQSASTPVEPTTPVHSNDQSNPITNKVDTNSNDHTDIKNEGSSHRTSSNNSSVTTN | ectodomain |
| | TNNEIRNGGGPLDQNEDKAEDEGETDAEGRGWNERTKNKPTFNATNRDFVDDN | |
| | LPELLKTSPFSGQNSDVQSASTPVEPTTPVHSNDQSNPITNKVDTNSNDHTDIKNE | |
| | GSSHRTSSNNSSVTTNTNNEIRNGGGPLDQNEDKAEDEGETDAEGRGWNERTKN | |
| | KPTFNATNRASPDGIGKMNMEEKQLENFINVSSNALELDISIGRDNFATKFLAQHV | |
| | NIFGDRISGLSAAYVEGYNNLAKIMYNSHSVLFDRKFNGAVISDNLIGNIADFGSYFL | |
| | EISPNTTRTNRSDYLKSVVLSKVQYLLSADFSTTDNIQRLTNLALALGYNNVKENNP | |
| | GNSQHSITTSLSTELFWSFGNNIFLFGHLATLMLAYLESNAYFTSGATRPFFSWQTL | |
| | VSTGGNEKFDKLDSMCGVIRGSKYSRKNNGFIKPHYKRLRRKTLLEGEPRLLCSMLE | |
| | EALDTVDKAIKFKGEELNSQGANIENSVSNDINSKRLQAKLCSNLNDSLINVSCDFRS | |
| | SKLDKHNKKLREAFDLLLACGNLNTGKKEAFPEYLRLISNPFEYGDIFSMTMWWDP | |
| | REFDGKQGWVEIYKKLRKNIMKPELKNVDMQLKYDSAISYYKQLKESETYPKKNIP | |
| | WARLYLYMSVIMSRSNAMSWAEDALRSFSNLYRMKPSLVMRGEGLETLLNYCAP | |
| | DPVALSHIFLYHFLTKKDAGKDLEKDLRRLEKGTLLSRIVNSSSIFIPNKLKKFLKMGA | |
| | RGFFNKKLNTLRAKSTLLRLFPKNLLHSALGAIEFTTHSLATLQISKNMDMWESLAQ | |
| | TKNLDAGGFPGEIDSLFNHWSESGGYSGYITGKLENGDDLTGDDIKKMNIKAPINN | |
| | DSLNWQKYINKKISEHFGKFLNLPFIQASGSQKNYIYQLVRDSKANLDDNLEQTVFF | |
| | GKVLPPGKTNNVIKKLKRIADSFTSMLLRSSARPVDHAVWVGVKINVPIVIHITKKLY | |
| | MIQRDMPRKEAWNLESAFLDLLQDLVIMVTNPGKRSPIGFETIGGNPGLPEISIRYP | |
| | HMSIEERKIEFQHSQCADHCISIWRSLIAFTLNTLNNPAAIKQFEKSLSSNSSLNDMS | |
| | KPEYINSFKYILKGDSVLHMYDNMLPRKVKREIKALKYGK | |
| 7 | LPDILSQNNTFKSFLEVNNVDQEDLICNKALCKSTDSINRNTSSYCYKYKLCSKCSVS | BMR1_03G03430 |
| | NVPDHPVCYLLDNDHNYIHLMEGHLGSQPIGSANSHDNSSHDEHSSHSNNGDM | ectodomain |
| | MDEHEEENFLQEYESKSMKFIPTSNMSDFDHARRSCAVDSKGNVMISVRLIIQWY | |
| | MSKDKSNNQQHHGNDDDSQNYDANYLQLTPMYSDDSVNSSMLEMDHDDSESS | |
| | NSHKSRMANMAKNFQVLKNIHKSAVKRYKSPKAKIYLIFSNPKINSCRHPVIYNGKI | |
| | SPSSMFVAKLESTISQIDLTQDLIKSSIETIVSCEACDKLKYNSCIQVTCAKNTPGAASL | |
| | AMGSAVYVPMTNTTIGVNAHNPNAVVAAGIPMGKIPVIPHPAAISGGNVGHLNN | |
| | GLHKAVNNAVMMPNGTSLPVQSGVVIKSLYNCLAFLLTILYLNF | |
| 8 | NPIFASATSAPSRNRAQDMTKAECIELLKGIVKSQEETKIVMKKLTSDLINNPLRLEQ | BMR1_04G06070 |
| | VYTKASQMQPEDPMEQWGVTVIDLDHLIEKYQHDPIVKDYILKIMNSPGINDTTLS | ectodomain |
| | DDVRNITISQILAIHEYMLSELESVVREFKSLQNRQTMEIKTLTIAAQAIVAAKVEEKF | |
| | NLTSDQVESAVIINHAELTASHAFTRLTMQMQTEMSELIGCQFPGW | |
| 9 | STNGKGGVDSVSKKSFVIELEDSTFERKTQASSGGTSGVWFVKFYAPWCGHCRSM | BMR1_04G09385 |
| | ENDWNELANILGKQINVAKIDATKHSVTAKRFGITSFPTLLLLKDGNFYQYENNNRT | ectodomain |
| | ADALKQFALHGYKQVKSKPVPKEWSYFVRFKFFIKSGFYEVKRIYQLAYPGFIT | |
| 10 | MAKLHESTSSASASFDPEKSDYDDTYVLTETTPTYIRHGFVRKVFAILFAQLLVTLGFSL | BMR1_03G04485 |
| | ICYFYRESVHSFISKNIWIFPTLAILSFITSLILIFSPSLSRRYPLNYAILVIETLYFSFIVGL | full sequence |
| | SCAFTKSPTAIVLSVSITLGIILLVVLFTLQTKIDFTRYIIYFILFSFVTLVFGFIGIFVPFDT | |
| | PLRMFYYGLGVLGYSLWMVLDLQLIIGGKTYEWTVDDYVPASLSLYTDVIGIFLNVH | |
| | GMFSDR | |
| 11 | MTITLNIKVNSETNFTVEAEPSFTVKELKILCESQSNIEAQNQRLICKGKLLKDTDILSD | BMR1_03G01645 |
| | VGAVDGATVYLVRSQVNKTQSAAPKQNTVPQPTLQTTNQPAGQTQTSGLGFQQ | full sequence |
| | QGFQQGFSGSQQPGFQANPFQSMLAGGFPNLDPTQMMEILNSPMAQEAMQR | |
| | LSQNPEVLRNILQNSSLMTPMLEQNPMLSEMLSNPELMRSMLRPEVLQAGLQM | |
| | HQAMQQQQQQQPGTQTNPIGSQNPDFSNMMRQMMNVFQQNPSVAQPTAP | |
| | QVYTDPRPPAERFATQLQALAEMGFIDTEKNITALIATNGDLNATVTRLLESNF | |
| 12 | MEEAERKFKEFNWADSQGWRIYWDNLYPTPPLSKVDKFKRSWFKRNVDSNISST | BMR1_03G01960 |
| | PLSEVGKQTQQTTPNQYRSQGNFAILTLEAGVRLLYLLITAPLFVLPLIGVRLSRYIYY | full sequence |
| | HYYIDIALLLIFLLSGIVRERGLPKMDSVWLASAFYSDMTQYIMYTCILMMSAPRPV | |
| | YLILPFLTCLIGLNSLAETNLSKLPKWLENIVGEIFKYTKDNIYWLMQTRGDVECYLLF | |
| | YIVFGFLTKSSAVITLMAYINFMKLRIGIGDPFIMSAFSKLHGCIVKLLSYDMFGHIPL | |
| | RMYLKISELLTKYMSGPRPQY | |
| 13 | MEDQNTTNESISNLHMENYFPKDIFSNLDNQKNLKTYHSKTFEKYFESAAKDNVER | BMR1_04G05080 |
| | CRTSAVKEWLNRNLPSEYNERHKPTLKLNLSPNHLNSTYNKQISDYTRNAYNRIEQL | full sequence |
| | KKLQEAYSLLRQKLQEKRRASCHKEVSEAEASVLNTQRLIISLKREDKDKIATELLIQN | |
| | AEELGIPSKDLETLNGYTFNEALKTIIDIISNMINNKFMTNLKDRCESARRKAGSEYR | |
| | DEMERIKVDLDMYKTKSEKLESTISTLNSYADSSKQNAEKVMELQHSLEDSNRQIN | |
| | QLKDSLYNMAKVNEELEKNEVKLEESLVELERYKLESQKLESVIKDLELLNQQKHDN | |
| | EELIKMLHDELDQCKSKSLQLNKKIEDLENSNKENLIPLNNELAQAYQKLSELEHSYE | |
| | QIEHQKNEADKKLESSIDEIEKQKQHSEELEQSITKLKQTIEEMEKETTDQVNDLQTC | |
| | LIDANCKIESLEGTISQLKQLNLELEGGEHRIQELQSKLTESNSTIEKLEKTITELENVSS | |
| | KYIDYDEKMDNLQKQLKEYTEKITVLENNASEFNYKDQAETLKTELDKSQLKIMELE | |
| | TMIKENSEKTERVPSPRKDNEEVDRLTSEILQLKNQVDILQNEKTDLEQKLSQQSPR | |
| | KDNEEVDRLTSEILQLKNQVDILQNEKTDLEQKLSQQSPRKDNEEVDRLTSEILQLK | |
| | NQVDILQNEKTDLEQKLSQQSPRKDNEEVDRLTSEILQLKNQVDILQNEKTDLEQKL | |
| | SQQSPRKDNEEVDRLTSEILQLKNQVDILQNEKTDLEQKLSQISAVIEENRQYKEKIE | |
| | LLERKLNEINKEKPKDSFTNVEVINAAFEDVRSLPINSSNASDWTAMPSELELEEHKL | |
| | YKKKSSRKGKKKSSREKETSRSDISSRSTSRHSKKDKPTIVDNNSTDVEASDSNEQMI | |
| | SDPVESITDQLNLVKQSTIQLTELGYDSISNVGSYLSDYIFGGN | |
| 14 | MDDLPGSLHQSTPNEKPMAPPTAPKSAPHVPISVESKELSKEIPKESPMTKEDKKD | BMR1_03G00820 |
| | VAKSKVSAAVTEKKVVKEPVVPKITIEMKKFSMSRESTQYSIFIIFNLIFALLYIFKVRL | full sequence |
| | MAIFCNLIIVAISIGAILSSIDPNRRKVDETNVNIIFISPTTVSDLAIVITAKLNQYISYFR | |
| | RILLWQDFILSTRFTLCVYIMGILFKIIPLVALIYIMCWAFYLYMFICKEMADQLLDIIM | |
| | VYLKRVSGNFDEFCCNIPKMKDVGKEL | |
| 15 | MFNENEIPQFQRPFTPSKDTEIDTSINFPANSILNNISFQKLLDWLDECTEGLPIVESF | BMR1_01G02100 |
| | ARRFNVTRGHIAAIFSAILILYFIFGWKINIFCNTIGLVYPAFKSHKVLLIHRAMTESPK | full sequence |
| | ATATITTGGKDKENDTNPQMPTCLNGIQGEIMFWLRYWIVYSLYLFISILIFPLISWL | |
| | PLISIVRVGFILYLYHPYTRGANAIYYLVISPLLSKNQKIIEQAIDTLERVAMGEIRKFTE | |
| | KQMKLH | |
| 16 | MDSIEECNKLVDAVTKLATSFDYQAQEYLYNLTRDENAINIALSFLLSNKYVLNYSSS | BMR1_04G07360 |
| | YSNEILQYITDVRGPSSCCIWDNIQLDENTSRHLGHITSILFKEHMHIVLCFDDAKLIA | full sequence |
| | LIDTLVTIWQLQSTISLVTQSCFADNATDDHITRVISMIIFKKPQLLTHFINILNTPEVT | |
| | DKYVADFSMKFKLRNCDRDDIYVTRYRHMIVYVLAQVLENFVSSNVTDKISFLPINF | |
| | SDLVILSGNCNNRYLYSCALLIVNSLNFHISEELLHQLLVIVEKSGFDCDILEMWNFAI | |
| | SHLQYLKVDNFMMKSFRLVQNGLIESTNPINIVPVLNGITVYYVNNSSVPTEIVRAY | |
| | RQLLSVESLDIPIEIGELFAAVRNACTESLITYDKIASFVSQFDIRMISSRLIELCNPNM | |
| | QCWSWLNVNCCVEEFPTDYDIYIDSMKNVFKDLFFILPEQLLDTIISEILQSVNENTE | |
| | TSLIALICYDCLEVRHALAILDKIKSLFTLKLSSNYDFILTCMFCQSIFVPHLHRIKDILFQ | |
| | RFVINMKISNIWSKRLAKCIAILNDRDSVHSVIECVQYILETSKSKGVLSQNHYPSILSL | |
| | SNLLDTLPQMPCDVNLTGSNYIFCKCLLALSISTRQLSQILPKIALNLDILYEGDVDND | |
| | LFCLSTSDSQAAVNLLMQLYIQNKVTDIPNELHEIRHPGLLMLCNLNENFEQFRMN | |
| | KCVEFMSTVSQESCCHCAWACIAYASYVIEQKFNFEFSMEMVKVIYRVLPCLIKMV | |
| | KSKNEPLHWDLDAILEYRINEIHPSIKCLKGTIWNIYYDGTVSVGSDACKYLSLVNKY | |
| | APRIIKSLSNPNVLQLVYQISMCTGSIEVIANKIAISL | |
| 17 | MSCILKCNNEDELVVDGEKPKVVEYVEKPSVIYKPTTVVPPNSLIEITAPKDLPQNPT | BMR1_02G02185 |
| | FFPTIDTFFDNDVKQIVLLMELPGFVAGDIDLEVGEGEVCVCGPRSKEELYEKYGQN | full sequence |
| | LDIHIRERKVGYFYRRFKLPHNALDNTVKASYQNGILEVRITCTEFSPKTRVEITS | |
| 18 | MNGSVEELLNRLSAINDRCYLLVDEIKNYMSNKLYHELTLALIELFTMSEISCNDRLLL | BMR1_04G08260 |
| | FEMIVHPIKNDLNILKFSHILRLSSEHLEPLASLDQLSKYDNYLSTDTQASFIKIAKSYH | full sequence |
| | HTRNQSYDQSLKLLEEVKPEIESGFGLDITVISAYYKVSANLNKATHKYNSWYQDSL | |
| | MYLNYTPLDSISPTERDELALDIAIASIAAPDNYNFGAVLIQPLINTCLKQHSTFGWV | |
| | YAILMALNDGDFTQYDEIISKYKVQISHSELNHHKEQLQRKITLMAFLKLVFRKAKK | |
| | QRIFTFEEISQNCRIPIDEVEYLLLKAMCNNVVKGKINQVEQIVSFTWVQPRIIDSTKL | |
| | TVLLDGVNEWNQQLKALINKLKEITPELLVS | |
| 19 | MSGLFGQSQQIGGGLFGQSNQQSGGGLFGSTSQQPTQTCSGLFGSSPAPANSSIF | BMR1_03G02345 |
| | GSNTQSAASSGGIFGSSTAPVNSGGGIFGQSNTNVSSGSGLFGGGNTTGQSGGGI | full sequence |
| | FGSSTTSAPASGGGLFGQTGTTTSGGGGLFTSSFAPAPSSGGLFGQPSTPATSGTGL | |
| | FSSTSTTQPSSGAGLFGSSTTPASGSGGLFGQPSTSTTTSGGIFGSSTTSAPASGGGL | |
| | FGQTGTPASGSSGIFGSTNTTTSASGTGLFGSTSTTPQPGSGSGLFGGGNTTGQSG | |
| | GGIFGSSTTSAPASGGGLFGQTGTTTSGGNLFGTTSTTTPAPASGGLFGSSSTTSTT | |
| | TPTQATTTVGGGGLFGTASATTAPASGGLFGTTSTTTPAPASGGLFGSSSTTSTTPT | |
| | QATTTVGGGGLFGTASATTAPVSGGLFGTTSTTTPAPANNTTPANTTTVPTAILTTS | |
| | TPSPATDGLFGSTDVTTTTDSTTKLVGTSPFEKQTDSGPDVTAATNDTPNAFITDKP | |
| | TAGGDLKGQVELSFSSVEHECVQDLLSNWEKRMEVKIQRFTEFAQDIQRIDRDLIL | |
| | QTEKLQVLLDEQATVQERQNQVQEMIQVIEKEQQQVLESLDIMDTALETLLGPDK | |
| | KITSKSGETIDFVSNKLRDLEAQLKAAHDVVDSVVKASQPEPLANVAKVFAFHQDTI | |
| | ENIQLQTSEIEKKLDAIKQQQAV | |
| 20 | MASLLKVSPQDNIEFPLVLYTPLNANLLLENLSGVHVAFKIKTTAPKGYLVRPSTGTI | BMR1_02G02960 |
| | KPGEALTVQIILQPLSEVPNVVNDRFLVQCTAIANDELVSKDFWTTLDKASIQDHRL | full sequence |
| | NVTFKKDIGLNIQTSQSNIGVPPHIAARILTPLGPNAGVAELRQKYEELVSYCLTAEK | |
| | QKAALVKDNEKLRQRLHLGPNDPASGNKWPLEGWHLPVMVIILVIILKAIGYW | |
| 21 | MSQGPAIGIDLGTTYSCVGVWKNETVEIIANDQGNRTTPSYVAFTDVERLVGDAA | BMR1_01G02545 |
| | KNQDARNPENTVFDAKRLIGRKINDPCIQSDIKHWPFTVAAGPNDKPVIKVQFQG | full sequence |
| | ETKSFHPEEISSMVLTKMKEIAESYLGKTISNAVITVPAYFNDSQRQATKDAGTIAGL | |
| | NVMRIINEPTAAAIAYGMDKKGTSEKNVLIFDLGGGTFDVSILTIEDGIFEVKATQG | |
| | DTHLGGEDFDNRLVNFCVDDFKRKNGGKNISTNRRALRRLRTQCERAKRTLSHST | |
| | QATIVVEAIFDGIDYSCNITRARFEELCAEMFKNTLIPVEKALADADMDKKQIHEVV | |
| | LVGGSTRIPKIQQLIKDFFNGKEPCKSINPDEAVAYGAAVQAAILTGEQSSKVQDLLL | |
| | LDVTPLSLGLETAGGVMTVLIPRNTTIPAKKEQEFTTNENNQTGVMIQVFEGERSM | |
| | TCDNNLLGKFHLTGIPPAPRGVPQIKVTFDIDANGILTVSAADKSTGKTEHVTITND | |
| | KGRLSQQDIDRMVAEAEKFREDDEKKKRCVESKNELENYCYSMKNALEEEGVKSKL | |
| | SSSELSEAQKLLQNTFSWIESNQLAEKEEFEAKLKEVQAVCTPLTAKLYQAGGGVPG | |
| | GAAPGGFNAGGAAPSGPTVEEVD | |
| 22 | MKLIACTLKNVETCVEVDPSDTVDALTNKIGSSLNNASASKMRLIHAGKILKMEQKI | BMR1_03G04110 |
| | SDYSDIKDGDKIIVLFSKQSEASTIANPTPAPTSTPIADANTSPPKPIPTTDPNALLMG | full sequence |
| | EELEKAINGIVEMGFDVESVKAAMSAAFNNPNRAIELLTRHEVDVSDHDTHQSVQ | |
| | TTGVLDELRQHPMFEQMRAIVRSNPQTLPQILSLIGQSDPSLLQAITENQEEFIQLLS | |
| | EPVLGTSGDFIDAQSITLTPEEMESINRLEGLGFSRPAAVEAFLACDKNEEMAANYL | |
| | LENIADYVSDNDN | |
| 23 | MDGQTEQQLIEENIERFRILYQLHLDNNEPSPTAQFNYACALVCSNQRSHNDTAIY | BMR1_02G02560 |
| | LLDELVRIRYESEECFYQLALAHMKRRSFVKSKEYLDRIIALEGSNQRVMALKSVVVS | full sequence |
| | LLAQDTFMGGLLGATAAFAIILFFTMKRNT | |
| 24 | MNQNSLCCSKGFTMFAGGAFFLVSFKPETVITNPLLLRPLLNVSWGYIFGSHLWAA | BMR1_04G05635 |
| | ISTYNKKYWENRIIPDYANSPSREIMQSRLKINESRIYRIYLENLIQTNVLANGILLVTT | full sequence |
| | SALAPSNKFLRICSGAALLLSIGNVVFALPEDEKDDDVGVVKTSSTSCFLSEVLSFCTF | |
| | GAIVPYVFA | |
| 49 | RNPRHTKFHKKHTPITDISPTANNLDDYELITYGNDEGLHDEPGLGSIVTDIEIRTPA | BMR1_02G01795 |
| | NFDGTAGNKGRKSKRTDKPVKKAKPVKTRNPVNITEINNANDEDTIDADLDEDLED | ectodomain |
| | DTYTDKPTGFFM | |
| 50 | SIDSQKSDNDVANTSETSEKLNYYDARDDFLHTMDMVNLVGGSCTLIDKANQPSD | BMR1_04G07915 |
| | FKQLLNASIFGYGRISALLTQTKSIYDVTVASTLAAVFGKLMQINLDDEGLAVEQWS | ectodomain |
| | ARVCSMLEIAEASLLSTSFKILADRLTKHCGRIAEVFLSQKENDPIDKNKSSDSINDYS | |
| | SGEGVDVVWSVSTECLMEFIAPNSPGGFTSKWTFVTFKHLLMQLSISIIMCEYQLR | |
| | DPKLLDDEIEGVENLLFRSWDVLEHYSNIQFYTQLDGASGIMTFAQSNLMPLVKRD | |
| | GDKINVSWNLQNEFEADPSSNSSGDLGLAAGDNASSDGEKPVATDPPFYSRRPRT | |
| | YSMLSDYKRRLSLTYEMSDEDTPDEDDDFEREDEEVIVEKETESTPNKQIKNAKNVF | |
| | GRRKTPQNKVFRPKLYPHDTVSTSSVTYAIKTNNENQIQFKSGAAQPIDSDIDSVPR | |
| | AKASNPSIDSGNDSVPRAKASNPSIDSGNDSVPRAKASNPSNGSDIDSVPRAKASN | |
| | PSNGPGNDSVPRAQASNPSNGPGNDSVPRAKASKPSNGSDIDSVPTAPASKPSN | |
| | GSDIDSVPTAPASTSKSVRGTTGTGSNSKWSSVRNSVLKNKTEENDNNEGPSKASG | |
| | SAGGGLKGEISFSDTVSILRFRSEPLRWTENTKVEANMHRKMVNKCQVEQLRPLSI | |
| | DYYNKISPPNLGHPVMGRATASSSSTLLPKFMGIGAKVSKTSNNPSMDTLSNFWG | |
| | MQRHDKELSGIRKLDKTVSQMAILDRFSRLADTCWDVASGRPSGYISPETMYRLG | |
| | GNRKDDYIHHVTRVYPFNLVKAYRFLVTKEEPQVQSNGDDNFEFSLFLQAPEFVSIL | |
| | PNGDRAEALVIEQKLVSTIRTLNTNSTEDPPISVCGYVYKRNDVDRVLIGEFGVKLPS | |
| | TILEHVYKIHTPYTILIHAKFTDSKFNKLKAVMQFLQGTHFGDFTLKFVYKGMVPSK | |
| | NVVKDSCHECLIGHY | |
| 51 | MDNEFTDFSLDKFGTTEYYINNHNHNSNDTVEDPFYLTHNHDSNDNVKPSILNPA | BMR1_01G01620 |
| | NASSCIPASYWQQQMEYNDSHLDNSRSSMMNENLIIKELGVDDSPQNDNEITSID | full sequence |
| | DITKLTEGELTTHTHEPYNLNYDSKTDLQNAFDTFYHDFSLFTPTETLPESQPCTIDSS | |
| | GHLFNHKFDISNNDSYYQSNEYLNTLPQSFGHWTRTSQTNNCSMEDKHRDDDN | |
| | NKLSTQLSTFLNPINSDNVDNSGHLYLDINASNTMATEYMHSNQPSGKPFDPLTLL | |
| | NRVKLRDSTAQIALAQIFNEAQEQKQTLNNVLDSLFDNVGYEGNLLEGDFKFPFISD | |
| | SLLTATISYSVRTANDEILSILVCYAITNKVNLRADLVADIIDIFFKSLRYSDAYKLIDYCY | |
| | NDKTKLDSILKTDNFQHMFNMCKEITVDNKISFEIYQYLLQCNGEKEILCKIVESIDE | |
| | AIDSDSHLASLLRTSSIPFIIRLFVERGDLNKLSYYLKIYLSKPSPSTHDLVEIIDVILETNR | |
| | AQLQSVVQQISESNSFDLTILLYIFTFLHLKNKSNIIHMIYADAIDKQIQNIPSDLKAVL | |
| | TSLSIPIFCFDFAPQYISDLLDSILRIYDTCNNSLVKRARAITSVHPATAMNEVDISND | |
| | VANEGDFDDLAISELKRMSLTPSGVDFAEIKSLIFSDDFLPRIIPLLTNENIFCLLQLSIA | |
| | SFDINSIEKISYSISLKDSNDLIIVAMINNALVNYGYVEKSKSLLKRAEKLICNVRLRLN | |
| | NKYPSMPTGTGSPISDSSGIGVRTGGTKEDNFNFGVSDCSGSHLPLKLRDISWVSSI | |
| | SNLLCSKDLSNSESFHLLSFISSLYSHPFIVSTVLKKFIVAHHILIRMLKSKLHINQAMIS | |
| | AICQVLNNTCCINQLQQLLMMTDLLQYHMSNDFYSAILDACIRINSPDHLLESVNK | |
| | YKKFGFHPDLQTYGLLIKFFSSSDNVMECFHLWNEMTSLYGYELNEVTYGCMFDAL | |
| | VSNNMLDEALSLLKDMKKNSNIKPNTIIYSTLIKGFGQTKQLNKALNIYLTMLDEGV | |
| | VPNTITYNSIIDACARVGDMNKAANLLEDMLNNNIEPDLITFSTVIKGYCVQSNMD | |
| | RSLQLLRAMSERGIKPDGILYNSLLDGCVKSGRPWLCQQLWDEMQENGIAPSNFT | |
| | LTILIKMYGRLGQLDKAFQLMDELPRKYNIQTNTHVYTCLMSACITNGKYKMALDV | |
| | FNCMNGNGIVPDSKTYETIIFGAIKGRLLYQVIDIIKAAYTLMSRGNGTGGRMNKSI | |
| | FKIESRILKLFAQKVEASGDPLLLQQAQSLAENLKKFNIILPIRSNLVTQKTQIKKVSNS | |
| | RAGHYDSFLLKNVTQSLRNDCTNNYRRNSVGATVFANNDGFKDNSVSDFREGINC | |
| | FGSDDNLNSFKDEHFNAHTQRMHTFASDITCNYRNDERNFQKENRSASGGTTLK | |
| | GVMWDKDIGGNINNAAYRFDASSIPYSAENAAIGSINSVFGGSYAGNGEDHFLSK | |
| | NPQYSMQFNPAMVPASNCGNRRGKLKF | |
| 52 | MAQLAVEELSPFERQELLCVYSSLLLYDDELEISKENINKVLNSAGAKVEPYLPMLFA | BMR1_04G05940 |
| | KALKGKDLNALFGSVASIGAPVASHAAATTSAAVDAPQAGKAQESNVEEEEDDDD | full sequence |
| | MGFSLFD | |
| 53 | MRWKPIYVFTSALIRLFCIFSCNITFSECIRQSHGVDRRNINGLSLCTPTNVLFSTFAC | BMR1_02G02565 |
| | YIRPSKIGKSYNNLNKYKLGTSSSEVDGNEQSDISSGSDVERGNLIQGKRLKNYYRIL | full sequence |
| | NLDKYASGEDIKNQYENLIESLKPLENVDSNVIDMINEAYRILSDENTRRIYDELVAK | |
| | KSLEKESGYNDSNIDQFYHDDGDYFPENLYNVLESDYDFTSDSDEMVLEMSTDDD | |
| | YSDEDSNEMISLIPKLIKPNGTKLHTTLTIPFERAIMGGNETVTISRLENCKCLENLTTC | |
| | KSCNGYGLDGKDKLGTGFVSSKECSECGGIGKSRAKKCDLCDNTGQVKVDNATIQ | |
| | VQVPRNVYDGARLLIRNQGNVYGTNGKAGDLVVTLRVKEHDHMYRMGKNIYSD | |
| | VTVPYAAAILGTTIKLETCGGVVSLEIPPGTQHGDEIQVPNESIPMKHICRIEVALPKS | |
| | VDKQERSLLEKIIQLK | |
| 54 | MDQLKDSIDQSNIDRKAALEAIESFLSSKIQAANAGSIRGKKNPAVNDFNNPPIVQS | BMR1_04G06705 |
| | YLSEDEGVTSISEYKNGISDPFYNIAVGTNHSISSAKNIPVSYQPNHTDTNRETPLIQR | full sequence |
| | SIAYFKNKTKFLSKMDTFTSALIPAAITLVITNSAQPLLIFLLRKYGGTPVGAYFFLFPTY | |
| | LGMICVGLYPTKKPVWKENWIYPGVLAGIDFLHQLIEKAGLLYCGPCLYTVASSSNT | |
| | LFLALFTSIYLNVKITRNTAISLTIISIAVSFSGSGKLCEINSTHLIGFTLNAFNFVIGEIILK | |
| | ENKIEGPNLVCIMGFISFISLTLWTCVWTIPNWSTIAHHTEMNVYAIFIILFVLFISNFI | |
| | RSSVYWILIKRAGSLFTGVLKALRIVIVIVISHILFSHIDPMQRITFTKIFTALLCSTGIVIY | |
| | SIDNNNKIDNYKNEMKGEREAVEDGEDEKIMVNDDV | |

In some embodiments, the compositions include one Bm antigen or one or more antigenic fragments thereof. In some embodiments, the composition includes a Bm antigen that comprises an amino acid sequence having at least 80% sequence identity (e.g., at least 80%, 85%, 90%, 95%, 97%, 99%, or greater) to the amino acid sequence of SEQ ID NO: 1, or one or more antigenic fragments thereof. In some embodiments, the composition includes a Bm antigen that comprises an amino acid sequence having at least 80% sequence identity (e.g., at least 80%, 85%, 90%, 95%, 97%, 99%, or greater) to the amino acid sequence of SEQ ID NO: 2, or one or more antigenic fragments thereof. In some embodiments, the composition includes a Bm antigen that comprises an amino acid sequence having at least 80% sequence identity (e.g., at least 80%, 85%, 90%, 95%, 97%, 99%, or greater) to the amino acid sequence of SEQ ID NO: 3, or one or more antigenic fragments thereof. In some embodiments, the composition includes a Bm antigen that comprises an amino acid sequence having at least 80% sequence identity (e.g., at least 80%, 85%, 90%, 95%, 97%, 99%, or greater) to the amino acid sequence of SEQ ID NO: 4, or one or more antigenic fragments thereof. In some embodiments, the composition includes a Bm antigen that comprises an amino acid sequence having at least 80% sequence identity (e.g., at least 80%, 85%, 90%, 95%, 97%, 99%, or greater) to the amino acid sequence of SEQ ID NO: 5, or one or more antigenic fragments thereof. In some embodiments, the composition includes a Bm antigen that comprises an amino acid sequence having at least 80% sequence identity (e.g., at least 80%, 85%, 90%, 95%, 97%, 99%, or greater) to the amino acid sequence of SEQ ID NO: 6, or one or more antigenic fragments thereof. In some embodiments, the composition includes a Bm antigen that comprises an amino acid sequence having at least 80% sequence identity (e.g., at least 80%, 85%, 90%, 95%, 97%, 99%, or greater) to the amino acid sequence of SEQ ID NO: 7, or one or more antigenic fragments thereof. In some embodiments, the composition includes a Bm antigen that comprises an amino acid sequence having at least 80% sequence identity (e.g., at least 80%, 85%, 90%, 95%, 97%, 99%, or greater) to the amino acid sequence of SEQ ID NO: 8, or one or more antigenic fragments thereof. In some embodiments, the composition includes a Bm antigen that comprises an amino acid sequence having at least 80% sequence identity (e.g., at least 80%, 85%, 90%, 95%, 97%, 99%, or greater) to the amino acid sequence of SEQ ID NO: 9, or one or more antigenic fragments thereof. In some embodiments, the composition includes one Bm antigen that comprises an amino acid sequence having at least 80% sequence identity (e.g., at least 80%, 85%, 90%, 95%, 97%, 99%, or greater) to the amino acid sequence of any one of SEQ ID NOs: 1-3. In some embodiments, the composition includes one Bm antigen that comprises an amino acid sequence having at least 80% sequence identity (e.g., at least 80%, 85%, 90%, 95%, 97%, 99%, or greater) to the amino acid sequence of any one of SEQ ID NOs: 1-9, or one or more antigenic fragments thereof. In some embodiments, the composition includes one Bm antigen that comprises an amino acid sequence having at least 80% sequence identity (e.g., at least 80%, 85%, 90%, 95%, 97%, 99%, or greater) to the amino acid sequence of any one of SEQ ID NOs: 1-24, or one or more antigenic fragments thereof.

In some embodiments, the compositions include one Bm antigen that comprises an amino acid sequence having at least 80% sequence identity (e.g., at least 80%, 85%, 90%, 95%, 97%, 99%, or greater) to the amino acid sequence of any one of SEQ ID NOs: 1-3, or one or more antigenic fragments thereof, and also include one or more Bm antigens that each comprise an amino acid sequence having at least 80% sequence identity (e.g., at least 80%, 85%, 90%, 95%, 97%, 99%, or greater) to the amino acid sequence of any one of SEQ ID NOs: 4-9, or one or more antigenic fragments thereof.

In some embodiments, the compositions include two Bm antigens, one or more antigenic fragments thereof, or a mixture thereof. In some embodiments, the compositions include two Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-3, or one or more antigenic fragments thereof. An exemplary composition includes the following two Bm antigens: BMR1_01G03280 (SEQ ID NO: 1) and BMR1_04G05532 (SEQ ID NO: 2). Another exemplary composition includes BMR1_01G03280 (SEQ ID NO: 1) and BMR1_01G00985 (SEQ ID NO: 3). In some embodiments, the compositions include one Bm antigen that comprises an amino acid sequence having at least 80% sequence identity (e.g., at least 80%, 85%, 90%, 95%, 97%, 99%, or greater) to the amino acid sequence of any one of SEQ ID NOs: 1-3, or one or more antigenic fragments thereof, and one Bm antigen that comprises an amino acid sequence having at least 80% sequence identity (e.g., at least 80%, 85%, 90%, 95%, 97%, 99%, or greater) to the amino acid sequence of any one of SEQ ID NOs: 4-9, or one or more antigenic fragments thereof. An exemplary composition includes the following two Bm antigens: BMR1_01G03280 (SEQ ID NO: 1) and BMR1_02G01760 (SEQ ID NO: 4). In some embodiments, the compositions include two or more Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-9, or one or more antigenic fragments of each of these two antigens. In some embodiments, the compositions include two Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-24, or two or more antigenic fragments thereof.

In some embodiments, the compositions include three Bm antigens, one or more antigenic fragments thereof, or a mixture thereof. In some embodiments, the compositions include three Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-3, or one or more antigenic fragments of each of these three antigens. In some embodiments, the compositions include two Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-3, and one Bm antigen that comprises an amino acid sequence having at least 80% sequence identity (e.g., at least 80%, 85%, 90%, 95%, 97%, 99%, or greater) to the amino acid sequence of any one of SEQ ID NOs: 4-9, or one or more antigenic fragments thereof. An exemplary composition includes the following three Bm antigens: BMR1_01G03280 (SEQ ID NO: 1), BMR1_04G05532 (SEQ ID NO: 2) and BMR1_02G01760 (SEQ ID NO: 4). Another exemplary composition includes BMR1_01G03280 (SEQ ID NO: 1), BMR1_01G00985 (SEQ ID NO: 3), and BMR1_02G01760 (SEQ ID NO: 4). In some embodiments, the compositions include one Bm antigen that comprises an amino acid sequence having at least 80% sequence identity (e.g., at least 80%, 85%, 90%, 95%, 97%, 99%, or greater) to the amino acid sequence of any one of SEQ ID NOs: 1-3, or one or more antigenic fragments thereof, and two Bm antigens that each comprise an amino acid sequence having at least 80% sequence identity (e.g., at least 80%, 85%, 90%, 95%, 97%, 99%, or greater) to the amino acid sequence of any one of SEQ ID NOs: 4-9, or one or more antigenic fragments thereof. An exemplary composition includes the following three Bm antigens: BMR1_01G03280 (SEQ ID NO: 1), BMR1_03G00365 (SEQ ID NO: 5) and BMR1_03G04695 (SEQ ID NO: 6). In some embodiments, the compositions include three Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-9, or one or more antigenic fragments of each of these three antigens. In some embodiments, the compositions include three Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-24, or one or more antigenic fragments thereof.

In some embodiments, the compositions include four Bm antigens, one or more antigenic fragments thereof, or a mixture thereof. In some embodiments, the compositions include four Bm antigens that each comprises an amino acid sequence having at least 80% sequence identity (e.g., at least 80%, 85%, 90%, 95%, 97%, 99%, or greater) to the amino acid sequence of any one of SEQ ID NOs: 1-3, or one or more antigenic fragments of each of these four antigens. In some embodiments, the compositions include three Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-3, or one or more antigenic fragments thereof, and one Bm antigen that comprises an amino acid sequence having at least 80% sequence identity (e.g., at least 80%, 85%, 90%, 95%, 97%, 99%, or greater) to the amino acid sequence of any one of SEQ ID NOs: 4-9, or one or more antigenic fragments thereof. In some embodiments, the compositions include at least one Bm antigen that comprises an amino acid sequence having at least 80% sequence identity (e.g., at least 80%, 85%, 90%, 95%, 97%, 99%, or greater) to the amino acid sequence of any one of SEQ ID NOs: 1-3, or one or more antigenic fragments thereof, and at least one Bm antigen comprises an amino acid sequence having at least 80% sequence identity (e.g., at least 80%, 85%, 90%, 95%, 97%, 99%, or greater) to the amino acid sequence of any one of SEQ ID NOs: 4-9, or one or more antigenic fragments thereof. An exemplary composition include the following four Bm antigens: BMR1_01G03280 (SEQ ID NO: 1), BMR1_04G05532 (SEQ ID NO: 2), BMR1_03G00365 (SEQ ID NO: 5), and BMR1_03G04695 (SEQ ID NO: 6). Another exemplary composition includes BMR1_01G03280 (SEQ ID NO: 1), BMR1_01G00985 (SEQ ID NO: 3), BMR1_03G00365 (SEQ ID NO: 5), and BMR1_03G04695 (SEQ ID NO: 6). In some embodiments, the compositions include four Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-9, or one or more antigenic fragments of each of these four antigens. In some embodiments, the compositions include four Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-24, or one or more antigenic fragments thereof.

In some embodiments, the compositions include five Bm antigens, one or more antigenic fragments thereof, or a mixture thereof. In some embodiments, the compositions include five Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-3, or one or more antigenic fragments of each of these five antigens. In some embodiments, the compositions include three Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-3, or one or more antigenic fragments thereof, and two Bm antigens that each comprise an amino acid sequence having at least 80% sequence identity (e.g., at least 80%, 85%, 90%, 95%, 97%, 99%, or greater) to the amino acid sequence of any one of SEQ ID NOs: 4-9, or one or more antigenic fragments thereof. In some embodiments, the compositions include at least one Bm antigen that comprises an amino acid sequence having at least 80% sequence identity (e.g., at least 80%, 85%, 90%, 95%, 97%, 99%, or greater) to the amino acid sequence of any one of SEQ ID NOs: 1-3, or one or more antigenic fragments thereof, and at least one Bm antigen that comprises an amino acid sequence having at least 80% sequence identity (e.g., at least 80%, 85%, 90%, 95%, 97%, 99%, or greater) to the amino acid sequence of any one of SEQ ID NOs: 4-9, or one or more antigenic fragments thereof. An exemplary composition includes the following five Bm antigens: BMR1_01G03280 (SEQ ID NO: 1), BMR1_04G05532 (SEQ ID NO: 2), BMR1_02G01760 (SEQ ID NO: 4), BMR1_03G00365 (SEQ ID NO: 5), and BMR1_03G04695 (SEQ ID NO: 6). Another exemplary composition includes BMR1_01G03280 (SEQ ID NO: 1), BMR1_01G00985 (SEQ ID NO: 3), BMR1_02G01760 (SEQ ID NO: 4), BMR1_03G00365 (SEQ ID NO: 5), and BMR1_03G04695 (SEQ ID NO: 6). In some embodiments, the compositions include five Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-9, or one or more antigenic fragments thereof. In some embodiments, the compositions include five Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-24, or one or more antigenic fragments thereof.

In some embodiments, the compositions include six Bm antigens, one or more antigenic fragments thereof, or a mixture thereof. In some embodiments, the compositions include six Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-3, or one or more antigenic fragments of each of these six antigens. In some embodiments, the compositions include three Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-3, or one or more antigenic fragments thereof, and three Bm antigens that each comprise an amino acid sequence having at least 80% sequence identity (e.g., at least 80%, 85%, 90%, 95%, 97%, 99%, or greater) to the amino acid sequence of any one of SEQ ID NOs: 4-9, or one or more antigenic fragments thereof. In some embodiments, the compositions include at least one Bm antigen that comprises an amino acid sequence having at least 80% sequence identity (e.g., at least 80%, 85%, 90%, 95%, 97%, 99%, or greater) to the amino acid sequence of any one of SEQ ID NOs: 1-3, or one or more antigenic fragments thereof, and at least one Bm antigen that comprises an amino acid sequence having at least 80% sequence identity (e.g., at least 80%, 85%, 90%, 95%, 97%, 99%, or greater) to the amino acid sequence of any one of SEQ ID NOs: 4-9, or one or more antigenic fragments thereof. An exemplary composition includes the following six Bm antigens: BMR1_01G03280 (SEQ ID NO: 1), BMR1_04G05532 (SEQ ID NO: 2), BMR1_02G01760 (SEQ ID NO: 4), BMR1_03G00365 (SEQ ID NO: 5), BMR1_03G04695 (SEQ ID NO: 6), and BMR1_03G03430 (SEQ ID NO: 7). Another exemplary composition includes BMR1_01G03280 (SEQ ID NO: 1), BMR1_01G00985 (SEQ ID NO: 3), BMR1_02G01760 (SEQ ID NO: 4), BMR1_03G00365 (SEQ ID NO: 5), BMR1_03G04695 (SEQ ID NO: 6), and BMR1_03G03430 (SEQ ID NO: 7). In some embodiments, the compositions include six Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-9, or one or more antigenic fragments thereof. In some embodiments, the compositions include six Bm antigens that each comprise an amino acid sequence having at 80% sequence identity (e.g., at least 80%, 85%, 90%, 95%, 97%, 99%, or greater) to the amino acid sequence of any one of SEQ ID NOs: 1-24, or one or more antigenic fragments thereof.

In some embodiments, the compositions include seven Bm antigens, one or more antigenic fragments thereof, or a mixture thereof. In some embodiments, the compositions include seven Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-3, or one or more antigenic fragments of each of these seven antigens. In some embodiments, the compositions include seven Bm antigens that each comprises an amino acid sequence having at least 80% sequence identity (e.g., at least 80%, 85%, 90%, 95%, 97%, 99%, or greater) to the amino acid sequence of any one of SEQ ID NOs: 1-9, or one or more antigenic fragments of each of these seven antigens. In some embodiments, the compositions include three Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-3, or one or more antigenic fragments thereof, and four Bm antigens that each comprise an amino acid sequence having at least 80% sequence identity (e.g., at least 80%, 85%, 90%, 95%, 97%, 99%, or greater) to the amino acid sequence of any one of SEQ ID NOs: 4-9, or one or more antigenic fragments thereof. In some embodiments, the compositions include at least one Bm antigen that comprises an amino acid sequence having at least 80% sequence identity (e.g., at least 80%, 85%, 90%, 95%, 97%, 99%, or greater) to the amino acid sequence of any one of SEQ ID NOs: 1-3, or one or more antigenic fragments thereof, and at least one Bm antigen that comprises an amino acid sequence having at least 80% sequence identity (e.g., at least 80%, 85%, 90%, 95%, 97%, 99%, or greater) to the amino acid sequence of any one of SEQ ID NOs: 4-9, or one or more antigenic fragments thereof. An exemplary composition includes the following Bm antigens: BMR1_01G03280 (SEQ ID NO: 1), BMR1_04G05532 (SEQ ID NO: 2), BMR1_02G01760 (SEQ ID NO: 4), BMR1_03G00365 (SEQ ID NO: 5), BMR1_03G04695 (SEQ ID NO: 6), BMR1_03G03430 (SEQ ID NO: 7) and BMR1_04G06070 (SEQ ID NO: 8). Another exemplary composition includes BMR1_01G03280 (SEQ ID NO: 1), BMR1_01G00985 (SEQ ID NO: 3), BMR1_02G01760 (SEQ ID NO: 4), BMR1_03G00365 (SEQ ID NO: 5), BMR1_03G04695 (SEQ ID NO: 6), BMR1_03G03430 (SEQ ID NO: 7) and BMR1_04G06070 (SEQ ID NO: 8). In some embodiments, the compositions include seven Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-24, or one or more antigenic fragments thereof.

In other embodiments, the compositions include eight Bm antigens, one or more antigenic fragments thereof, or a mixture thereof. In some embodiments, the compositions include eight Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-3, or one or more antigenic fragments of each of these eight antigens. In some embodiments, the compositions include eight Bm antigens that each comprises an amino acid sequence having at least 80% sequence identity (e.g., at least 80%, 85%, 90%, 95%, 97%, 99%, or greater) to the amino acid sequence of any one of SEQ ID NOs: 1-9, or one or more antigenic fragments of each of these eight antigens. In some embodiments, the compositions include three Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-3, or one or more antigenic fragments thereof, and five Bm antigens that each comprise an amino acid sequence having at least 80% sequence identity (e.g., at least 80%, 85%, 90%, 95%, 97%, 99%, or greater) to the amino acid sequence of any one of SEQ ID NOs: 4-9, or one or more antigenic fragments thereof. An exemplary composition includes the following eight Bm antigens: BMR1_01G03280 (SEQ ID NO: 1), BMR1_04G05532 (SEQ ID NO: 2), BMR1_01G00985 (SEQ ID NO: 3), BMR1_02G01760 (SEQ ID NO: 4), BMR1_03G00365 (SEQ ID NO: 5), BMR1_03G04695 (SEQ ID NO: 6), BMR1_03G03430 (SEQ ID NO: 7), and BMR1_04G06070 (SEQ ID NO: 8). In some embodiments, the compositions include two Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-3, or one or more antigenic fragments thereof, and six Bm antigens that each comprise an amino acid sequence having at least 80% sequence identity (e.g., at least 80%, 85%, 90%, 95%, 97%, 99%, or greater) to the amino acid sequence of any one of SEQ ID NOs: 4-9, or one or more antigenic fragments thereof. In some embodiments, the compositions include eight Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-24, or one or more antigenic fragments thereof.

In other embodiments, the compositions include nine Bm antigens, one or more antigenic fragments thereof, or a mixture thereof. In some embodiments, the compositions include nine Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-9, or one or more antigenic fragments thereof. In some embodiments, the compositions include the following nine Bm antigens: BMR1_01G03280 (SEQ ID NO: 1), BMR1_04G05532 (SEQ ID NO: 2), BMR1_01G00985 (SEQ ID NO: 3), BMR1_02G01760 (SEQ ID NO: 4), BMR1_03G00365 (SEQ ID NO: 5), BMR1_03G04695 (SEQ ID NO: 6), BMR1_03G03430 (SEQ ID NO: 7), BMR1_04G06070 (SEQ ID NO: 8), BMR1_04G09385 (SEQ ID NO: 9). In some embodiments, the compositions include nine Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-24, or one or more antigenic fragments thereof.

In some embodiments, the compositions include one or more (e.g., 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, or 24 or more) Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-24, or one or more antigenic fragments thereof. In some embodiments, the one or more Bm antigens each comprise an amino acid sequence having at least 90% sequence identity (e.g., at least 90%, 95%, 97%, 99%, or greater) to the amino acid sequence of any one of SEQ ID NOs: 1-24, or one or more antigenic fragments thereof. In some embodiments, the one or more Bm antigens each comprise an amino acid sequence having at least 95% sequence identity (e.g., at least 95%, 97%, 99%, or greater) to the amino acid sequence of any one of SEQ ID NOs: 1-24, or one or more antigenic fragments thereof. In some embodiments, the one or more Bm antigens each comprise an amino acid sequence having at least 99% sequence identity (e.g., at least 99%, or greater) to the amino acid sequence of any one of SEQ ID NOs: 1-24, or one or more antigenic fragments thereof. In some embodiments, the one or more Bm antigens each comprise an amino acid sequence of any one of SEQ ID NOs: 1-24. In other embodiments, the compositions include twenty-four Bm antigens or twenty-four or more antigenic fragments thereof. In some embodiments, the compositions include twenty-four or more Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-24, or twenty-four or more antigenic fragments thereof. In certain embodiments, the compositions include the following twenty-four Bm antigens: BMR1_01G03280 (SEQ ID NO: 1), BMR1_04G05532 (SEQ ID NO: 2), BMR1_01G00985 (SEQ ID NO: 3), BMR1_02G01760 (SEQ ID NO: 4), BMR1_03G00365 (SEQ ID NO: 5), BMR1_03G04695 (SEQ ID NO: 6), BMR1_03G03430 (SEQ ID NO: 7), BMR1_04G06070 (SEQ ID NO: 8), BMR1_04G09385 (SEQ ID NO: 9), BMR1_03G04485 (SEQ ID NO: 10), BMR1_03G01645 (SEQ ID NO: 11), BMR1_03G01960 (SEQ ID NO: 12), BMR1_04G05080 (SEQ ID NO: 13), BMR1_03G00820 (SEQ ID NO: 14), BMR1_01G02100 (SEQ ID NO: 15), BMR1_04G07360 (SEQ ID NO: 16), BMR1_02G02185 (SEQ ID NO: 17), BMR1_04G08260 (SEQ ID NO: 18), BMR1_03G02345 (SEQ ID NO: 19), BMR1_02G02960 (SEQ ID NO: 20), BMR1_01G02545 (SEQ ID NO: 21), BMR1_03G04110 (SEQ ID NO: 22), BMR1_02G02560 (SEQ ID NO: 23), and BMR1_04G05635 (SEQ ID NO: 24).

In some embodiments, the compositions include one Bm antigen that comprises an amino acid sequence having at least 80% sequence identity (e.g., at least 80%, 85%, 90%, 95%, 97%, 99%, or greater), but less than 100% identity, to a reference sequence as set forth herein.

In some embodiments, a composition comprises one or more of a protein of SEQ ID NO: 49, 50, 51, 52, 53, or 54, or an antigenic variant or fragment of any thereof. In some embodiments, a composition comprises a protein of SEQ ID NO: 49 or an antigenic variant or fragment thereof. In some embodiments, a composition comprises a protein of SEQ ID NO: 50 or an antigenic variant or fragment thereof. In some embodiments, a composition comprises a protein of SEQ ID NO: 51 or an antigenic variant or fragment thereof. In some embodiments, a composition comprises a protein of SEQ ID NO: 52 or an antigenic variant or fragment thereof. In some embodiments, a composition comprises a protein of SEQ ID NO: 53 or an antigenic variant or fragment thereof. In some embodiments, a composition comprises a protein of SEQ ID NO: 54 or an antigenic variant or fragment thereof. In some embodiments of the compositions described in this paragraph, the protein(s) of the composition have at least 80% (e.g., at least 85%, 90%, 95%, 97%, 99%, or 100%) identity to the reference sequence noted or to an antigenic fragment thereof.

In some embodiments, a composition comprises two or more of a protein of SEQ ID NO: 49, 50, 51, 52, 53, or 54, or an antigenic variant or fragment of any thereof. In some embodiments, a composition comprises three or more of a protein of SEQ ID NO: 49, 50, 51, 52, 53, or 54, or an antigenic variant or fragment of any thereof. In some embodiments, a composition comprises four or more of a protein of SEQ ID NO: 49, 50, 51, 52, 53, or 54, or an antigenic variant or fragment of any thereof. In some embodiments, a composition comprises five or more of a protein of SEQ ID NO: 49, 50, 51, 52, 53, or 54, or an antigenic variant or fragment of any thereof. In some embodiments, a composition comprises all five proteins of SEQ ID NO: 49, 50, 51, 52, 53, and 54, or an antigenic variant or fragment of any thereof. In some embodiments of the compositions described in this paragraph, the protein(s) of the composition have at least 80% (e.g., at least 85%, 90%, 95%, 97%, 99%, or 100%) identity to the reference sequence noted or to an antigenic fragment thereof.

In some embodiments, one or more Bm antigen selected from SEQ ID NOs: 49-54, or an antigenic variant or fragment thereof, is comprised within a composition comprising one or more Bm antigen selected from SEQ ID NOs: 1-24, or an antigenic variant or fragment thereof. In some embodiments of the compositions described in this paragraph, the protein(s) of the composition have at least 80% (e.g., at least 85%, 90%, 95%, 97%, 99%, or 100%) identity to the reference sequence noted or to an antigenic fragment thereof.

In some embodiments, the compositions include one Bm antigen that comprises an amino acid sequence having at least 80% sequence identity (e.g., at least 80%, 85%, 90%, 95%, 97%, 99%, or greater), but less than 100% identity, to a reference sequence as set forth herein.

Exemplary compositions of the invention are presented in Tables 2-9, below, with each indicated composition comprising the Bm antigens indicated with an “X” or antigenic variants and/or fragments thereof. Accordingly, each indicated composition can comprise Bm antigens (or antigenic variants and/or fragments thereof) that consist of those indicated or, optionally, the compositions can comprise additional components (e.g., additional Bm antigens, antigenic variants, and/or antigenic fragments thereof). In some embodiments, therefore, each indicated composition may be considered as a base or core composition to which additional components (e.g., additional Bm antigens, antigenic variants, and/or antigenic fragments thereof) are optionally added.

Each composition can also optionally include one or more adjuvant, carrier, diluent, excipient, and/or preservative, e.g., as described herein.

Furthermore, instead of the indicated Bm antigens (or antigenic variants and/or antigenic fragments thereof), the compositions can each alternatively comprise or consist of corresponding nucleic acid (e.g., mRNA) molecules or modified versions thereof as described herein (see, e.g., Table 10).

In some embodiments, one or more (e.g., 1, 2, 3, 4, 5, or 6) of the indicated Bm antigens (or corresponding nucleic acids) comprise a sequence that is 100% identical to that indicated in Table 1 (or a corresponding nucleic acid sequence), or an antigenic fragment thereof as described herein (see below). In some embodiments, the one or more (e.g., 1, 2, 3, 4, 5, or 6) Bm antigens or antigenic fragments (or corresponding nucleic acids) are in a combination as set forth in a composition of one of Tables 2-9. In other embodiments, one or more (e.g., 1, 2, 3, 4, 5, or 6) of the indicated Bm antigens (or an antigenic variant and/or fragment thereof, or a corresponding nucleic acid) comprise an amino acid sequence that is at least 80% (e.g., at least 85%, 90%, 95%, 97%, or 99%) identical to a sequence in one or more of Table 1 (or a corresponding nucleic acid sequence), or an antigenic fragment thereof as described herein (see below). In some embodiments, the one or more (e.g., 1, 2, 3, 4, 5, or 6) Bm antigens, antigenic variants, and/or antigenic fragments (or corresponding nucleic acids) are in a combination as set forth in a composition of one of Tables 2-9.

Additional Bm antigens (or corresponding nucleic acid molecules) that can optionally be included in the compositions include those of any one or more of SEQ ID NOs: 4, 8-24, 51-54, or antigenic variants and/or fragments thereof as described herein (or corresponding nucleic acid sequences; see, e.g., Table 10).

As a specific example, each of the compositions listed below can each optionally include a Bm antigen of SEQ ID NO: 4, or an antigenic variant or fragment thereof.

In some embodiments, use of the combinations present in the compositions provide additive, synergistic, or otherwise improved results as compared to the use of the individual components alone.

TABLE 2

| Composition No.: | 1 | 2 | 3 | 4 | 5 | 6 | 7 | 8 | 9 | 10 | 11 | 12 | 13 | 14 | 15 | 16 |
|---|---|---|---|---|---|---|---|---|---|---|---|---|---|---|---|---|
| BMR1_01G03280 | X | X | X | X | X | X | X | X | X | X | X | X | X | X | X | X |
| (SEQ ID NO: 1) | | | | | | | | | | | | | | | | |
| BMR1_04G05532 | | X | | | | X | X | X | | | | X | X | X | | X |
| (SEQ ID NO: 2) | | | | | | | | | | | | | | | | |
| BMR1_01G00985 | X | X | X | X | X | X | X | X | X | X | X | X | X | X | X | X |
| (SEQ ID NO: 3) | | | | | | | | | | | | | | | | |
| BMR1_03G00365 | | | X | | | X | | | X | X | | X | X | | X | X |
| (SEQ ID NO: 5) | | | | | | | | | | | | | | | | |
| BMR1_03G04695 | | | | X | | | X | | X | | X | X | | X | X | X |
| (SEQ ID NO: 6) | | | | | | | | | | | | | | | | |
| BMR1_03G03430 | | | | | X | | | X | | X | X | | X | X | X | X |
| (SEQ ID NO: 7) | | | | | | | | | | | | | | | | |

TABLE 3

| Composition No.: | 17 | 18 | 19 | 20 | 21 | 22 | 23 | 24 | 25 | 26 | 27 | 28 | 29 | 30 | 31 |
|---|---|---|---|---|---|---|---|---|---|---|---|---|---|---|---|
| BMR1_01G03280 | X | X | X | X | X | X | X | X | X | X | X | X | X | X | X |
| (SEQ ID NO: 1) | | | | | | | | | | | | | | | |
| BMR1_04G05532 | X | X | X | X | X | X | X | X | | | | | | | |
| (SEQ ID NO: 2) | | | | | | | | | | | | | | | |
| BMR1_01G00985 | | | | | | | | | | | | | | | |
| (SEQ ID NO: 3) | | | | | | | | | | | | | | | |
| BMR1_03G00365 | | X | | | X | X | | X | X | X | X | X | | | |
| (SEQ ID NO: 5) | | | | | | | | | | | | | | | |
| BMR1_03G04695 | | | X | | X | | X | X | | X | | X | X | X | |
| (SEQ ID NO: 6) | | | | | | | | | | | | | | | |
| BMR1_03G03430 | | | | X | | X | X | X | | | X | X | | X | X |
| (SEQ ID NO: 7) | | | | | | | | | | | | | | | |

TABLE 4

| Composition No.: | 32 | 33 | 34 | 35 | 36 | 37 | 38 | 39 | 40 | 41 | 42 | 43 | 44 | 45 | 46 |
|---|---|---|---|---|---|---|---|---|---|---|---|---|---|---|---|
| BMR1_01G03280 | | | | | | | | | | | | | | | |
| (SEQ ID NO: 1) | | | | | | | | | | | | | | | |
| BMR1_04G05532 | X | X | X | X | X | X | X | X | | | | | | | |
| (SEQ ID NO: 2) | | | | | | | | | | | | | | | |
| BMR1_01G00985 | X | X | X | X | X | X | X | X | X | X | X | X | X | X | X |
| (SEQ ID NO: 3) | | | | | | | | | | | | | | | |
| BMR1_03G00365 | | X | | | X | X | | X | X | X | X | X | | | |
| (SEQ ID NO: 5) | | | | | | | | | | | | | | | |
| BMR1_03G04695 | | | X | | X | | X | X | | X | | X | X | X | |
| (SEQ ID NO: 6) | | | | | | | | | | | | | | | |
| BMR1_03G03430 | | | | X | | X | X | X | | | X | X | | X | X |
| (SEQ ID NO: 7) | | | | | | | | | | | | | | | |

TABLE 5

| Composition No.: | 47 | 48 | 49 | 50 | 51 | 52 | 53 | 54 | 55 | 56 |
|---|---|---|---|---|---|---|---|---|---|---|
| BMR1_01G00985 | X | X | X | X | X | X | | | | |
| (SEQ ID NO: 3) | | | | | | | | | | |
| BMR1_03G00365 | X | | | X | X | | X | X | X | |
| (SEQ ID NO: 5) | | | | | | | | | | |
| BMR1_03G04695 | | X | | X | | X | X | | X | X |
| (SEQ ID NO: 6) | | | | | | | | | | |
| BMR1_03G03430 | | | X | | X | X | | X | X | X |
| (SEQ ID NO: 7) | | | | | | | | | | |

TABLE 6

| Composition No.: | 57 | 58 | 59 | 60 | 61 | 62 | 63 | 64 | 65 | 66 | 67 | 68 | 69 | 70 | 71 | 72 |
|---|---|---|---|---|---|---|---|---|---|---|---|---|---|---|---|---|
| BMR1_01G00985 | X | X | X | X | X | X | X | X | X | X | X | X | X | X | X | X |
| (SEQ ID NO: 3) | | | | | | | | | | | | | | | | |
| BMR1_03G00365 | X | X | X | X | X | X | X | X | X | X | X | X | X | X | X | X |
| (SEQ ID NO: 5) | | | | | | | | | | | | | | | | |
| BMR1_01G03280 | | X | | | | X | X | X | | | | X | X | X | | X |
| (SEQ ID NO: 1) | | | | | | | | | | | | | | | | |
| BMR1_03G04695 | | | X | | | X | | | X | X | | X | X | | X | X |
| (SEQ ID NO: 6) | | | | | | | | | | | | | | | | |
| BMR1_02G01795 | | | | X | | | X | | X | | X | X | | X | X | X |
| (SEQ ID NO: 49) | | | | | | | | | | | | | | | | |
| BMR1_04G07915 | | | | | X | | | X | | X | X | | X | X | X | X |
| (SEQ ID NO: 50) | | | | | | | | | | | | | | | | |

TABLE 7

| Composition No.: | 73 | 74 | 75 | 76 | 77 | 78 | 79 | 80 | 81 | 82 | 83 | 84 | 85 | 86 | 87 |
|---|---|---|---|---|---|---|---|---|---|---|---|---|---|---|---|
| BMR1_01G00985 | X | X | X | X | X | X | X | X | X | X | X | X | X | X | X |
| (SEQ ID NO: 3) | | | | | | | | | | | | | | | |
| BMR1_03G00365 | | | | | | | | | | | | | | | |
| (SEQ ID NO: 5) | | | | | | | | | | | | | | | |
| BMR1_01G03280 | X | X | X | X | X | X | X | X | | | | | | | |
| (SEQ ID NO: 1) | | | | | | | | | | | | | | | |
| BMR1_03G04695 | | X | | | X | X | | X | X | X | X | X | | | |
| (SEQ ID NO: 6) | | | | | | | | | | | | | | | |
| BMR1_02G01795 | | | X | | X | | X | X | | X | | X | X | X | |
| (SEQ ID NO: 49) | | | | | | | | | | | | | | | |
| BMR1_04G07915 | | | | X | | X | X | X | | | X | X | | X | X |
| (SEQ ID NO: 50) | | | | | | | | | | | | | | | |

TABLE 8

| Composition No.: | 88 | 89 | 90 | 91 | 92 | 93 | 94 | 95 | 96 | 97 | 98 | 99 | 100 | 101 | 102 |
|---|---|---|---|---|---|---|---|---|---|---|---|---|---|---|---|
| BMR1_01G00985 | | | | | | | | | | | | | | | |
| (SEQ ID NO: 3) | | | | | | | | | | | | | | | |
| BMR1_03G00365 | X | X | X | X | X | X | X | X | X | X | X | X | X | X | X |
| (SEQ ID NO: 5) | | | | | | | | | | | | | | | |
| BMR1_01G03280 | X | X | X | X | X | X | X | X | | | | | | | |
| (SEQ ID NO: 1) | | | | | | | | | | | | | | | |
| BMR1_03G04695 | | X | | | X | X | | X | X | X | X | X | | | |
| (SEQ ID NO: 6) | | | | | | | | | | | | | | | |
| BMR1_02G01795 | | | X | | X | | X | X | | X | | X | X | X | |
| (SEQ ID NO: 49) | | | | | | | | | | | | | | | |
| BMR1_04G07915 | | | | X | | X | X | X | | | X | X | | X | X |
| (SEQ ID NO: 50) | | | | | | | | | | | | | | | |

TABLE 9

| Composition No.: | 103 | 104 | 105 | 106 | 107 | 108 | 109 | 110 | 111 | 112 |
|---|---|---|---|---|---|---|---|---|---|---|
| BMR1_01G03280 | X | X | X | X | X | X | | | | |
| (SEQ ID NO: 1) | | | | | | | | | | |
| BMR1_03G04695 | X | | | X | X | | X | X | X | |
| (SEQ ID NO: 6) | | | | | | | | | | |
| BMR1_02G01795 | | X | | X | | X | X | | X | X |
| (SEQ ID NO: 49) | | | | | | | | | | |
| BMR1_04G07915 | | | X | | X | X | | X | X | X |
| (SEQ ID NO: 50) | | | | | | | | | | |

In some embodiments, the antigenic fragment may comprise a portion of the Bm antigen of at least 10 (e.g., at least 10, 25, 50, 75, 100, 150, 200, 500, 600, 700, 800, 900, 1000, or more) amino acid residues of a Bm antigen described herein. In other embodiments, the antigenic fragment may comprise a portion of the Bm antigen of at least 100 (e.g., at least 100, 150, 200, 500, 600, 700, 800, 900, 1000, or more) amino acid residues of a Bm antigen described herein. In further embodiments, the antigenic fragment may comprise a portion of the Bm antigen of at least 500 (e.g., at least 500, 600, 700, 800, 900, 1000, or more) amino acid residues of a Bm antigen described herein. Antigenic fragments may be of any length sufficient to induce an immune response when administered to a subject.

Exemplary Bm antigens useful in conjunction with the compositions, methods, and kits described herein include those for which the amino acid sequence displays at least 80% sequence identity (e.g., at least 80%, 85%, 90%, 95%, 97%, 99%, or greater) to one of the foregoing sequences, and in which the sequence changes are conservative amino acid substitutions. Further Bm antigens useful in conjunction with the compositions, methods, and kits described herein include those for which the amino acid sequence has one or more (e.g., 1, 2, 3, 4, 5, 6, 7, 8, 9, 10, 1-50, 2-40, 3-30, 4-25, 5-20, or 10-15) conservative amino acid substitutions with respect to one or more of the foregoing sequences.

In some embodiments, the Bm antigens used in the compositions of the invention may be purified from the parasite or artificially manufactured molecules, e.g., recombinant proteins, synthetic peptides, proteins or peptides encoded by DNA plasmids or produced by recombinant viruses or bacteria. In some embodiments, the Bm antigens, antigenic fragments thereof, or nucleic acid molecules encoding them (e.g., RNA or DNA molecules) are in purified or isolated form. In some embodiments, the Bm antigens or antigenic fragments thereof (or combinations thereof, e.g., as described herein), are at least 50%, 60%, 70%, 75%, 80%, 85%, 90%, 95%, 96%, 97%, 98%, 99%, or 100% free of other molecules with which they may be naturally associated in nature. In some embodiments, the compositions of the invention are pharmaceutical compositions and do not exist in nature even if specified in general terms, e.g., a composition comprising the antigen or antigenic fragment and a pharmaceutically acceptable carrier or diluent.

A composition may comprise one or more Bm antigens or antigenic fragments together with other components such as an excipient, preservative, diluent, or carrier. Exemplary excipients include sugars and sugar alcohols, such as lactose, sucrose, glucose, mannitol, and sorbitol, as well as gelatin, cellulose, cellulose derivatives, polyvinylpyrrolidine, starch, and polyethylene glycol. Exemplary preservatives include thimerosal, 2-phenoxyethanol, and formaldehyde. Exemplary diluents include water, saline (e.g., phosphate-buffered saline), and sucrose solutions. Exemplary carriers include human serum albumin.

A composition may further comprise one or more adjuvants, as readily understood by those skilled in the art. Suitable adjuvants for compositions of the present invention include adjuvants that are capable of enhancing the immune response to the Bm antigens of the present invention (e.g., Bm antigens described herein). Adjuvants are well known in the art (see, e.g., Vaccine Design-The Subunit and Adjuvant Approach, 1995, Pharmaceutical Biotechnology, Volume 6, Eds. Powell and Newman, Plenum Press, New York and London, hereby incorporated by reference).

Exemplary adjuvants for use in the compositions of the present invention include aluminum salts (e.g., aluminum oxyhydroxide and aluminum hydroxyphosphate) and calcium salts (e.g., calcium phosphate). A well-known example of aluminum oxyhydroxide is Alhydrogel™, whereas a well-known example of aluminum hydroxyphosphate is AdjuPhos™.

Another adjuvant for use in the compositions of the present invention is an emulsion. An emulsion can be a water-in-oil-in-water emulsion or a water-in-oil emulsion. The oil phase may consist of squalene and squalane mixed in one of several commonly used ratios. A well-known example of such adjuvant oil is Montanide™ ISA-720 (Seppic, Castres, France), which contains both squalene and squalane, with squalene predominating. In addition to the water phase and the oil phase, emulsions contain a surfactant emulsifier. Examples of surfactant emulsifiers include mono- and di-fatty acid (C₁₂-C₂₄) esters of sorbitan or mannide, such as sorbitan monostearate, sorbitan monooleate, mannide monostearate, and mannide monooleate. An exemplary water-in-oil emulsion used in the composition of the invention may consist one or more Bm antigens or antigenic fragments thereof dissolved in the water phase and emulsified in Montanide™ ISA-720 using mannide monooleate (Arlacel™ A) as surfactant emulsifier. Oil-in-water emulsion adjuvants include those disclosed in WO 95/17210 and EP 0399842, incorporated herein by reference. An exemplary oil-in-water emulsion is MF59 which uses squalene as adjuvant oil (Chiron Corp; e.g., see, U.S. Pat. Nos. 5,709,879 and 6,086,901).

Small molecules can be used as adjuvants. Such adjuvants include 7-substituted-8-sulfo-guanosine derivatives and 7-substituted-8-oxo-guanosine derivatives (e.g., 7-allyl-8-oxoguanosine (loxoribine)), as described in U.S. Pat. Nos. 4,539,205; 4,643,992; 5,011,828; and 5,093,318; which are each incorporated herein by reference.

Lipid products of bacterial origin also can be used as adjuvants, including a monophosphoryl lipid A (MPL) (Corixa Corp.; see, U.S. Pat. No. 4,987,237), and a 3-O-deacylated derivative of MPL (3D-MPL). Cell wall proteoglycans of bacterial origin that can be used as adjuvants include muramyl dipeptide analogues (e.g., as described in U.S. Pat. No. 4,767,842), such as N-acetyl-muramyl-L-threonyl-D-isoglutamine (Thr-MDP; U.S. Pat. No. 4,606,918), N-acetyl-nor-muramyl-L-alanyl-D-isoglutamine (nor-MDP; CGP 11637), and N-acetyl-muramyl-L-alanyl-D-isoglutaminyl-L-alanyl-2-(1′,2′-dipalmitoyl-sn-glycero-3′-hydroxyphosphoryloxy)-ethylamide (MTP-PE; CGP 1983A).

Other exemplary adjuvants include the saponin fractions derived from the bark of the South American tree Quillaja saponaria Molina (e.g., Quil-A™). Derivatives of Quil-A and methods for its production are described in U.S. Pat. No. 5,057,540. In addition to QS21 (also known as QA21), other fractions such as QS17 (also known as QA17) are described.

Other exemplary adjuvants include RC-529, CpG oligodeoxynucleotides (Pfizer), SBAS2 (SmithKline Beecham), GM-CSF, Complete Freund's Adjuvant (CFA), and Incomplete Freund's Adjuvant (IFA). Yet another class of exemplary adjuvants consists of glycolipid analogues such as N-glycosylamides, N-glycosylureas, and N-glycosylcarbamates, each of which is substituted in the sugar residue by an amino acid.

Adjuvant mixtures that can be used in the invention include, e.g., combinations of 3D-MPL and QS21 (see, e.g., EP0671948 B1), oil-in-water emulsions including 3D-MPL and QS21 (see, e.g., WO 95/17210 and PCT/EP98/05714), 3D-MPL formulated with other carriers (see, e.g., EP 0689454 B1), QS21 formulated in cholesterol-containing liposomes (see, e.g., WO 96/33739), and immunostimulatory oligonucleotides (see, e.g., WO 96/02555). Alternative adjuvants include those described in, e.g., WO 99/52549, and non-particulate suspensions of polyoxyethylene ether (see, e.g., UK Patent Application No. 9807805.8).

Adjuvants are utilized in various amounts, which can vary depending upon the type of adjuvant, the components of the composition (e.g., compositions including one or more Bm antigens or antigenic fragments thereof), and the subject to which the composition is administered. Typical amounts can vary from about 1 μg to about 50 mg per dosage. Those skilled in the art can readily determine appropriate concentrations and amounts to use.

Babesia Microti Nucleic Acid-Based Compositions

Compositions of the invention, which can be used, e.g., in the methods and kits described herein, can include one or more nucleic acid molecules (e.g., DNA or RNA (e.g., mRNA)) encoding one or more Bm antigens (or one or more antigenic fragments thereof) and optionally a pharmaceutically acceptable adjuvant, carrier or diluent.

The nucleic acid molecules can encode any one or more of the Bm antigens or antigenic fragments described above in the subsection entitled “Babesia microti antigen-based compositions” or elsewhere herein. Accordingly, the nucleic acid molecules can encode any one or more (e.g., 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, or 24 or more) of SEQ ID NOs: 1-24 and 49-54, or one or more antigenic fragments thereof (e.g., a fragment of at least 10, 25, 50, 75, 100, 150, 200, 500, 600, 700, 800, 900, 1000, or more amino acids). The nucleic acid molecules can further encode polypeptides having at least 80%, 85%, 90%, 95%, 97%, 99%, or greater sequence identity to any one of SEQ ID NOs: 1-24 and 49-54, or antigenic fragments thereof, consistent with the description provided above.

In some embodiments, the nucleic acid molecules (e.g., RNA or DNA molecules) are in purified or isolated form. In some embodiments, nucleic acid molecules (e.g., RNA or DNA molecules) are at least 50%, 60%, 70%, 75%, 80%, 85%, 90%, 95%, 96%, 97%, 98%, 99%, or 100% free of other molecules with which they may be naturally associated in nature. In some embodiments, the compositions of the invention are pharmaceutical compositions and do not exist in nature even if specified in general terms, e.g., a composition comprising the nucleic acid (e.g., RNA or DNA) and a pharmaceutically acceptable carrier or diluent.

The nucleic acid molecules can comprise DNA, RNA (e.g., mRNA), or modified forms thereof. In some embodiments, the nucleic acid molecules comprise DNA that is designed to encode and express an RNA molecule that, in turn, encodes a Bm antigen or antigenic fragment thereof as described herein. Such DNA molecules can optionally be present in the context of a vector (e.g., a plasmid or an expression vector) that is administered to a subject.

In some embodiments, the nucleic acid molecules comprise an RNA molecule that is administered to a subject. In some embodiments, the RNA comprises one or more modified nucleosides (e.g., pseudouridines and/or 2′-O-methylated nucleosides). In some embodiments, the RNA molecule is non-replicating RNA. In some embodiments, the RNA is self-replicating. In some embodiments, the RNA is present in a lipid nanoparticle. In some embodiments, the RNA is present in an RNA virus (e.g., a retrovirus, lentivirus, alphavirus, or rhabdovirus). In some embodiments, the RNA is introduced into dendritic cells ex vivo, and the dendritic cells are then administered to a subject (e.g., a subject from whom the dendritic cells were obtained).

Nucleic acid molecule sequences encoding each of SEQ ID NOs: 1-24 and 49-54 are provided below in Table 10 as SEQ ID NOs: 25-48 and 55-60, respectively. DNA coding sequences are shown. As is well known in the art, in the case of RNA molecules, each “T” is replaced with a “U” or an equivalent nucleotide (e.g., pseudouridine). Also as known in the art, nucleic acid sequences can be codon optimized for use in the species in which expression is desired.

TABLE 10
Nucleic acid sequences encoding exemplary
full-length B. microti antigens or their ectodomain

| SEQ ID | | |
|---|---|---|
| NO. | Nucleic Acid Sequence | Gene ID |
| 25 | GATGTATATGAGATATCTTCTGGTAATCCACCCGACATAGAGCCAACATCTACT | BMR1_01G03280 |
| | TCTCTAGAAACAAATGTAGTTACCAACTATATTCCAGAACCCAATGCGGATTCA | ectodomain |
| | GAATCTGTACATGTTGAAATCCAGGAACATGATAACATCAATCCACAAGACGCT | |
| | TGCGATAGTGAGCCGCTCGAACAAATGGATTCTGATACCAGGGTGTTGCCCGA | |
| | AAGTTTGGATGAGGGGGTACCACACCAATTCTCTAGATTAGGGCACCACTCAG | |
| | ACATGGCATCTGATATAAATGATGAAGAACCATCATTTAAAATCGGCGAGAAT | |
| | GACATAATTCAACCACCCTGGGAAGATACAGCTCCATACCATTCAATAGATGAT | |
| | GAAGAGCTTGACAACTTAATGAGACTAACGGCGCAAGAAACAAGTGACGATCA | |
| | TGAAGAAGGGAATGGCAAACTCAATACGAATAAAAGTGAGAAGACTGAAAGA | |
| | AAATCGCATGATACTCAGACACCGCAAGAAATATATGAAGAGCTTGACAACTTA | |
| | CTGAGACTAACGGCACAAGAAATATATGAAGAGCGTAAAGAAGGGCATGGCA | |
| | AACCCAATACGAATAAAAGTGAGAAGGCTGAAAGAAAATCGCATGATACTCAG | |
| | ACAACGCAAGAAATATGTGAAGAGTGTGAAGAAGGGCATGACAAAATCAATA | |
| | AGAATAAAAGTGGAAATGCTGGAATAAAATCGTATGATACTCAGACACCGCAG | |
| | GAAACAAGTGACGCTCATGAAGAAGGGCATGACGAAATCAATACGAATAAAA | |
| | GTGAGAAGGCTGAAAGAAAATCGCATGATACTCAGACAACGCAAGAAATATGT | |
| | GAAGAGTGTGAAGAAGGGCATGACAAAATCAATAAGAATAAAAGTGGAAATG | |
| | CTGGAATAAAATCGTATGATACTCAGACACCGCAGGAAACAAGTGACGCTCAT | |
| | GAAGAAGAGCATGGCAATCTCAATAAGAATAAAAGTGGGAAGGCTGGAATAA | |
| | AATCGCATAATACTCAGACACCGCTGAAAAAAAAAGACTTTTGTAAAGAAGGG | |
| | TGTCATGGTTGCAATAATAAGCCCGAGGATAATGAAAGAGACCCGTCGTCGCC | |
| | TGATGATGATGGTGGCTGCGAATGCGGCATGACGAATCACTTTGTCTTTGACTA | |
| | CAAGACAACACTCTTGTTAAAGAGCCTCAAGACTGAAACA | |
| 26 | ATAATTACGGATGGATTGTCCGCTATTGGCTCTGTGGCGAGTGAAGTGGGAAA | BMR1_04G05532 |
| | TACAGTGAAGGATGTTTCCAGTGAAGCGCTCTTAGGAGAATTACAACAAATTG | ectodomain |
| | CAGACGGTGGCAAAATTATAAGAGACGGCACTGAAAGCAATTTTGTGAATTCA | |
| | ATAGCTAATACGGTTATGAAAAATGTAGTTGGCACTGCAGTTCTCAAAGCTTCA | |
| | TCTGGTATTACACATAATGGTGATTTCGCCTTTTACAACTTTATGTACCCGGAAA | |
| | ATGATGATTATCCATGGGCATGTATCTGTGATGAATCTGATTATGAAGAGTATA | |
| | TTAAGGGCAAGAAGGACAAGGTTAGGTGCAGGAACTATATTGATTCATCCTTA | |
| | CAAAATGCAGTTTTATATTGTAACCCAGCAAATCATAACTCTTCCATAAATGACA | |
| | ATGCCAATAACAATCCCCCAAAGCAGATTGATAACCATGTGTCCATACCACAAA | |
| | CAGCGCCAGCAAATCATACCACAGTTTTATCTACGGAAGTCGACACTAATCACA | |
| | ATGAACAAAAACAACCAAATTCGCCTTCAGTTCCAAGTGAATCTCAAAATTCAG | |
| | TGTCCGCTCCGAAAGATGAATCTGTTAGTAGCACCGTAGAAGGAGCCAAATCA | |
| | AGTTCA | |
| 27 | ACACTTGTATCTAAAATTACAACTCCAAACAATTTACCCGGAATAGATACTTGCT | BMR1_01G00985 |
| | CGAACTGGGAAGATGTATCAGTGTGTACTACTAAAAACACCAGAAATTGCATTT | ectodomain |
| | CAGGTGAAGGCAAACCAAAGGATTGTTTTGCGATTGGGAAATCCTTATTTAAA | |
| | ACATTTCCAAATTGTTATGAAGGTGTTATAATTGATCAAGTCACGTTTTCTGGAT | |
| | TTGAGACACTAGAAATCCATTATTGCGATATAGATAAGATCTTACACAAAGCAA | |
| | ATGAAGTTGTTAAATCAATACACGAATTGAAAGAGAAAACAAATCAATTAACC | |
| | GAGAAAATTAAGGACATACCTGATTTGATTGATAAAGTTATTAAGGTAAATTCG | |
| | GAAATTTCAAAAATATTTCATCAAGATAAAATTAAACATATGGAAAAGGAGGCC | |
| | AACGATTTCAAAAACGCCTTGAAAACCACTAGAAACTACATAATCAGTTATGAT | |
| | TCCTTAGATAAAACCAAGCAAAGCAATCTACTAACTTCACTTGGAAAATTAATG | |
| | AATAAGATTAAAACAAAACTATCAGAAATGGATAAGACATTACATTCTACACTT | |
| | GACACAAACAATACCATTATAGATCTTGTCAATAACAATAGTTCACACGTTAAA | |
| | CATCCTAATGATTTTAACAAAACAATGGAACTCTACAATGAAACTATTACTAAA | |
| | GCTGATGCGATTAAAAAAAATATTGAAAAACTTAAAGAACATAGGAAAATATC | |
| | TACTCACAAAACAATATTTTCAAACAATATAGATAAGTTGATTGACAATTTGACA | |
| | GATTATTTTGAAAATATAAATCGCTCCATTGATGCTGTTAGAGACAAACTTTCCA | |
| | AATACCAACTTGAAACGGGCAAGATGGTTTTATTGTTTAAGAACGTGAATGAA | |
| | ATTCAAAAGCATATTAAAAATACAGATATGCATATAAGAACGTGCAATTATGAT | |
| | TTCTCAGATATCGAACAAAAATACTCACTCATTACTGCAAAAATTACTGTCGAA | |
| | GATGGACAATCAATCACAACTTCAAATAAATCAACTGTGGATATACCAGAAGA | |
| | GAAGGTAGATAGGGTGAATGTCAATGTTGACAAAGCAGAGAACTCTGATAATG | |
| | AAACTTCTCAGGAAAATACATCTGTTAAACCGACTGATCACAAAGAAATTGAGG | |
| | ATTCAGCTTCCGAAGAAAATGCCATTGGAGAAAATGGTGATTATGATTCAGAT | |
| | GAAGATATTGATACAAATGACGTTAAAGAAGATCACGAAAATGCAATTGATTC | |
| | CGAATACACCGTTTCATCGACTGGGGATGTATTAGAAGATGAGGTAGTTGAGG | |
| | AAAATGCCATTGGAGAAAATGGTGATTATGATTCAGATGAAGATATTGATACA | |
| | AATGACGTTAAAGAAGATCACGAAAATGCAATTGATTCCGAATACACCGTTTCA | |
| | TCGACTGGGGATGTATTAGAAGATGAGGTAGTTGAGGAAAATGCCATTGGAG | |
| | AAAATGGTGATTATGATTCAGATGAAGATATTGATACAAATGACGTTAAAGAA | |
| | GATCACGAAAATGCAATTGATTCCGAATACACCGTTTCATCGACTGGGGATGTA | |
| | TTAGAAGATGAGGTAGTTGAGGAAAATGCCATTGGAGAAAATGGTGATTATGA | |
| | TTCAGATGAAGATATTGATACAAATGACGTTAAAGAAGATCACGAAAATGCAA | |
| | TTGATTCCGAATACACCGTTTCATCGACTGGGGATGTATTAGAAGATGAGGTAG | |
| | TTGAGGAAAATGCCATTGGAGAAAATGGTGATTATGATTCAGATGAAGATATT | |
| | GATACAAATGACGTTAAAGAAGATCACGAAAATGCAATTGATTCCGAATACCTC | |
| | GTGTCATCGACTGGGGATGTATTAGAAGATGAGGTAGTTGAGGAAAATGCCAT | |
| | TGGAGAAAATGGTGATTATGATTCAGATGAAGATATTGATACAAATGACGTTA | |
| | AAGAAGATCACGAGGATGCAATTGATTCCGAATACCTCGTGTCATTGACTGGG | |
| | GATGTATTAGAAGATGAGGTAGTTGAGGAAAATGCCATTGGAGAAAATGGTG | |
| | ATCATGATTCAGATGAAGATATTGATATAGAAGAAGTTAACGAAGAGGATCAT | |
| | GAAGATGCGATTGATTCTGATAACTCCATATCAAATAGCGAAAATGTAGATACT | |
| | ACACCAACTGAGAATGTAGATACTATACCAACTAAGAATGCAAATACTACACCA | |
| | ACTAAGAATGCAAATGCTACACCGACTAAGAATGTAGATACTATACCAACTAAG | |
| | AATGTAGATACTATACCAACTAAGAATGCAAATACTACACCAACTAAGAATGTA | |
| | GTTACGACACCAACTAAGAATGCAAATACTACACCAACTAAGAATGTAGTTACG | |
| | ACACCAACTAAGAATGCAAATACTACACCAACTAAGAATGTAGTTACGACACCA | |
| | ACTAAGAATGCAAATACTACACCAACTAAGAATGTAGTTACGACATCAACTGGA | |
| | AATGTAAGAACCAAACACACATCGACAAATTCTCATGTTTTAGCTCCGGATACA | |
| | GACGAATACCCAGCACAAATTTCACAACATAAGACGATTGATAAATATTATCAA | |
| | TCGTTGGAATTGGAGGATGAAGAAAATGCAGATATATCAAGCTCAGATAAACC | |
| | AGTTTCTCCAATAAATTTGGAAGATAAAAATTCGACATATGATCATATGCATAA | |
| | AACCGATAATGTTAAAAGCGCCGGTATTGCTTCATATGAT | |
| 28 | AACCGCTCTTGTCCAGGAAACAATGGCGTTGGCGGTGGATCTGGTGATAATAA | BMR1_02G01760 |
| | CAGTGGCATAATTCCAAATGATCCACACCCCTGTTGTAACAATCTTAGACAAAA | ectodomain |
| | ACCCCAATACCAAACCAAGCCAGAAAATGAACTAGTCAATGATGATAGAGATTT | |
| | GAACTTTAATAAGATCAGAGGTGGCAAGCAAATCATCACCTTTACTGTCCCTTC | |
| | CATAGATGATCTCAAGAATAAAAGATTATCCGACTCTGAATTCATTTTATCAGA | |
| | AAAGGCAAATCCATTGATTTCCTCCGGCGACAGTAAAAACGTTATTGTATTCGA | |
| | AGTAAAAAACGATAATGAGAAGTTAATGGGTAGTGTTGAAGTTGGTCAATGGG | |
| | AAGTTACTATCACCACATCATGCATCAGACGTATCGTTATTTTCGATTCCAATGA | |
| | AGTTTCAGATAACATTCCCATGTATATATATATCGTGGACTACTTTGAAGGAGG | |
| | TAATAGCACTGTTTCAAAATTCTTTTTCGCGAATAATAGATGGAACGCTGATTTC | |
| | ACCAATCACACACCTAATGCTGCT | |
| 29 | TTCCTACTGAACCGCAGCGAATTTAAGTGGTTTAAAGTGGGTCTAATAATTACC | BMR1_03G00365 |
| | ACGATATTCCCTTTTAAGCATTCATTTGACTATAATTTGACACACATATTTCTATT | ectodomain |
| | TTCAATATGCACGTTAATATTTTGTGTAAAACCTGTGGATGAGGAGAGCGGTGC | |
| | CAAAAAGGAGGGATTTGACTTCAAGAAGATGGTGCCAGATAAGTTCAAAAAGT | |
| | ATACC | |
| 30 | GATAGTGGTAATTCTTCCCCCCAAACGCCGCCAGAAACTAGCAGTCCTATAAAT | BMR1_03G04695 |
| | GGTGTAATCGGCGATGAAAATAATGGATTGGAACATTTGAGTAGTTCTGGTCT | ectodomain |
| | GGAAGTTGATGATAACCTGCCCGAATTACTCAAAACTTCTCCATTTTCAGGCCA | |
| | AAATTCAGATGTTCAATCTGCTTCAACACCAGTTGAACCCACTACTCCTGTTCAT | |
| | TCCAATGACCAATCTAATCCTATCACCAATAAAGTTGATACCAATTCTAATGACC | |
| | ATACTGATATTAAGAATGAAGGTTCATCCCATCGTACTTCATCCAACAATTCTTC | |
| | TGTCACAACCAATACTAATAATGAGATTAGAAATGGCGGAGGACCATTAGATC | |
| | AGAATGAAGATAAGGCTGAAGATGAAGGTGAAACTGATGCCGAAGGGAGAG | |
| | GATGGAATGAGAGAACGAAAAATAAACCTACATTTAATGCCACAAATCGCGAT | |
| | TTTGTTGATGATAACCTGCCCGAATTACTCAAAACTTCTCCATTTTCAGGCCAAA | |
| | ATTCAGATGTTCAATCTGCTTCAACACCAGTTGAACCCACTACTCCTGTTCATTC | |
| | CAATGACCAATCTAATCCTATCACCAATAAAGTTGATACCAATTCTAATGACCAT | |
| | ACTGATATTAAGAATGAAGGTTCATCCCATCGTACTTCATCCAACAATTCTTCTG | |
| | TCACAACCAATACTAATAATGAGATTAGAAATGGCGGAGGACCATTAGATCAG | |
| | AATGAAGATAAGGCTGAAGATGAAGGTGAAACTGATGCCGAAGGGAGAGGAT | |
| | GGAATGAGAGAACGAAAAATAAACCTACATTTAATGCCACAAATCGCGCTTCTC | |
| | CCGATGGAATTGGCAAAATGAACATGGAAGAAAAACAGTTGGAGAATTTTATA | |
| | AATGTATCATCCAACGCTCTTGAATTAGATATCTCAATTGGCCGGGATAATTTTG | |
| | CTACTAAATTCCTAGCACAACACGTTAATATTTTTGGAGACAGGATAAGCGGCT | |
| | TGAGTGCGGCGTATGTAGAGGGCTATAATAACTTGGCTAAAATTATGTACAAT | |
| | AGTCACTCTGTTTTATTTGATAGAAAATTCAATGGAGCAGTTATTTCCGACAATT | |
| | TGATTGGAAATATTGCAGATTTTGGTTCATACTTCTTAGAGATATCTCCAAATAC | |
| | AACTAGAACCAATAGAAGTGATTATCTCAAATCAGTAGTACTTTCCAAAGTTCA | |
| | GTATCTGTTGTCTGCTGATTTTTCCACCACTGACAACATCCAAAGGCTCACAAAT | |
| | TTGGCACTTGCCCTAGGCTATAACAATGTCAAAGAAAATAATCCTGGCAATTCC | |
| | CAACATTCAATAACAACCTCCCTCTCAACTGAGCTATTTTGGTCCTTTGGCAACA | |
| | ATATATTCTTATTTGGACACTTGGCAACTCTAATGCTTGCTTATCTAGAATCTAA | |
| | TGCATATTTCACATCTGGTGCGACCAGGCCTTTCTTTTCATGGCAAACTCTGGTT | |
| | TCCACTGGTGGTAATGAGAAATTTGATAAACTGGACTCTATGTGTGGAGTGATT | |
| | CGTGGATCAAAGTATTCGCGAAAAAACAATGGATTTATCAAGCCACACTACAAA | |
| | AGATTACGAAGGAAGACTTTGCTCGAAGGTGAACCGCGTTTGTTGTGCAGCAT | |
| | GCTGGAAGAGGCTTTGGATACTGTGGATAAAGCTATTAAATTTAAGGGCGAAG | |
| | AGCTAAATTCACAAGGCGCAAATATTGAAAATTCGGTCTCCAATGACATCAACA | |
| | GTAAACGTTTACAGGCGAAACTTTGCTCAAATTTGAATGATTCACTTATAAACG | |
| | TGAGCTGTGATTTTAGATCATCAAAATTGGACAAACACAATAAGAAATTAAGA | |
| | GAGGCATTTGACTTATTATTAGCTTGTGGCAATTTGAACACTGGTAAAAAGGAG | |
| | GCATTTCCGGAATACTTGAGATTGATCTCTAACCCCTTCGAATACGGCGATATTT | |
| | TTTCCATGACTATGTGGTGGGATCCTAGGGAGTTTGATGGCAAACAGGGCTGG | |
| | GTCGAAATATACAAAAAGTTGAGGAAGAATATTATGAAACCTGAACTGAAAAA | |
| | TGTGGACATGCAACTCAAATACGACTCGGCAATTTCTTATTACAAACAACTGAA | |
| | GGAATCGGAAACCTACCCCAAGAAAAACATTCCATGGGCTAGATTATACCTTTA | |
| | CATGTCAGTAATCATGTCAAGATCGAACGCTATGAGTTGGGCTGAGGACGCCC | |
| | TCCGCTCATTTAGCAATCTATATCGCATGAAACCTTCATTAGTGATGAGAGGGG | |
| | AAGGGTTAGAGACTTTGCTCAATTACTGCGCTCCTGATCCAGTGGCTTTATCAC | |
| | ACATATTCCTCTACCACTTTTTGACAAAGAAAGATGCCGGTAAAGATTTGGAAA | |
| | AAGACTTGAGAAGACTTGAGAAAGGGACACTCCTCTCGCGCATTGTGAATTCA | |
| | TCGTCAATATTCATACCTAATAAACTAAAGAAATTCCTTAAAATGGGCGCTCGC | |
| | GGATTTTTCAATAAAAAACTTAACACTTTGAGAGCAAAGAGCACCCTTCTACGA | |
| | TTATTCCCCAAGAATTTACTACATTCTGCATTGGGCGCAATTGAATTCACCACAC | |
| | ATTCACTAGCCACGTTACAAATATCTAAGAATATGGATATGTGGGAGAGTTTGG | |
| | CCCAGACAAAGAATTTAGATGCAGGCGGTTTCCCTGGCGAAATCGATTCACTGT | |
| | TTAATCATTGGTCGGAAAGTGGTGGATACAGTGGGTATATTACCGGCAAATTA | |
| | GAGAATGGAGATGATCTAACGGGCGATGATATCAAAAAAATGAACATAAAGG | |
| | CCCCTATTAACAATGATTCACTCAATTGGCAAAAGTATATCAATAAAAAAATATC | |
| | CGAGCATTTTGGCAAATTTCTAAACCTTCCATTTATCCAAGCCTCCGGTTCACAA | |
| | AAGAACTACATATATCAGCTTGTACGGGATAGTAAGGCAAACCTTGACGATAA | |
| | TTTGGAACAAACTGTGTTCTTTGGCAAAGTGCTTCCCCCAGGAAAAACTAATAA | |
| | TGTTATTAAGAAATTGAAAAGAATTGCAGATTCATTTACCAGCATGCTGTTACG | |
| | TTCTTCTGCTCGTCCTGTTGACCATGCTGTGTGGGTTGGAGTGAAAATCAACGT | |
| | ACCTATTGTAATTCATATTACTAAAAAGTTGTACATGATACAGCGTGATATGCCT | |
| | AGGAAGGAAGCGTGGAATTTGGAAAGTGCTTTCTTGGACCTGTTGCAGGATCT | |
| | GGTGATAATGGTTACAAACCCGGGGAAGAGATCTCCGATAGGGTTTGAAACGA | |
| | TTGGGGGCAATCCTGGATTACCAGAAATTAGCATTAGATACCCTCATATGTCGA | |
| | TTGAGGAAAGGAAGATTGAATTTCAACATTCACAATGTGCAGATCATTGTATAT | |
| | CAATTTGGAGATCCCTAATCGCATTTACACTTAACACACTAAACAATCCTGCGGC | |
| | AATAAAGCAATTTGAGAAATCCTTATCCAGTAATAGTTCTCTAAACGACATGTC | |
| | GAAGCCCGAATATATCAACAGCTTCAAGTATATACTAAAAGGCGACTCGGTGTT | |
| | GCATATGTATGATAACATGTTGCCTAGGAAGGTGAAAAGGGAAATAAAGGCAC | |
| | TAAAGTATGGTAAG | |
| 31 | CTGCCTGACATTCTATCGCAGAATAACACGTTTAAATCTTTTCTCGAAGTAAATA | BMR1_03G03430 |
| | ACGTGGATCAGGAAGATTTGATTTGTAATAAGGCACTGTGTAAATCCACTGACT | ectodomain |
| | CTATCAACAGAAATACAAGTTCTTATTGTTATAAATACAAACTGTGTAGTAAGT | |
| | GTAGCGTATCCAACGTCCCAGATCACCCTGTTTGCTATCTTCTGGATAATGACCA | |
| | CAATTACATCCACTTAATGGAGGGGCATTTAGGCTCTCAGCCCATAGGATCAGC | |
| | GAATAGTCACGATAATTCTTCTCATGACGAACATTCATCGCATTCGAATAATGG | |
| | AGATATGATGGATGAGCATGAAGAGGAAAATTTTCTGCAGGAATACGAATCAA | |
| | AATCAATGAAATTCATACCTACTAGCAACATGAGTGACTTTGATCATGCTAGGC | |
| | GATCTTGTGCCGTGGATTCCAAAGGCAATGTAATGATAAGTGTTAGATTGATCA | |
| | TCCAATGGTATATGTCAAAGGATAAATCTAATAATCAGCAGCACCATGGCAATG | |
| | ATGATGATTCTCAAAATTATGACGCAAATTATTTACAACTCACCCCTATGTATTC | |
| | TGACGACTCTGTTAATTCTTCTATGTTAGAGATGGACCACGACGATAGTGAATC | |
| | ATCAAATTCGCATAAATCTAGGATGGCAAATATGGCCAAAAACTTCCAAGTTTT | |
| | GAAAAACATCCATAAAAGTGCAGTTAAGCGTTATAAATCTCCAAAGGCCAAGA | |
| | TATATCTCATATTTTCTAATCCAAAGATCAACAGCTGTAGACACCCGGTAATATA | |
| | CAACGGTAAAATCTCCCCCTCAAGCATGTTTGTAGCGAAACTTGAGTCAACAAT | |
| | ATCACAAATTGATTTGACACAAGATCTGATCAAATCGTCAATAGAAACTATTGT | |
| | CTCTTGTGAAGCTTGTGATAAGTTAAAATACAACAGTTGTATACAGGTTACCTG | |
| | TGCCAAAAATACACCAGGCGCAGCGTCTTTAGCTATGGGTAGTGCTGTATACGT | |
| | TCCAATGACGAATACTACGATTGGAGTAAATGCCCACAACCCTAATGCGGTAGT | |
| | AGCTGCCGGTATCCCTATGGGTAAAATACCTGTAATCCCCCATCCAGCAGCTAT | |
| | AAGTGGTGGAAATGTTGGTCATTTGAATAATGGTTTGCACAAGGCGGTTAATA | |
| | ATGCTGTTATGATGCCAAATGGAACATCATTGCCAGTGCAAAGCGGAGTTGTTA | |
| | TAAAATCATTATACAATTGTCTCGCTTTTTTACTGACAATTTTGTATCTCAATTTC | |
| 32 | AACCCTATATTTGCATCTGCAACCTCAGCTCCAAGTAGGAATAGAGCTCAAGAC | BMR1_04G06070 |
| | ATGACCAAAGCGGAATGTATAGAACTATTAAAAGGCATAGTGAAATCTCAAGA | ectodomain |
| | GGAAACCAAAATTGTTATGAAGAAGTTGACCTCAGACCTGATAAACAATCCACT | |
| | GCGCCTAGAACAGGTATATACCAAGGCCAGTCAAATGCAGCCAGAGGATCCTA | |
| | TGGAGCAATGGGGAGTCACTGTAATTGATCTCGACCATTTGATTGAAAAGTATC | |
| | AACATGATCCTATTGTGAAGGATTACATTTTGAAGATCATGAATTCGCCCGGCA | |
| | TAAACGATACAACTCTGAGTGACGATGTACGCAATATTACTATTAGTCAAATAT | |
| | TAGCTATCCATGAATATATGCTCAGCGAATTGGAGTCTGTAGTCCGCGAATTCA | |
| | AATCGCTGCAGAACAGACAAACTATGGAGATTAAAACGTTAACTATTGCTGCG | |
| | CAAGCAATCGTAGCTGCCAAAGTAGAGGAAAAATTCAATTTAACATCAGATCA | |
| | GGTTGAATCGGCTGTGATAATAAACCATGCCGAATTAACTGCTAGCCATGCATT | |
| | CACCAGATTGACTATGCAAATGCAGACGGAGATGAGTGAGTTGATTGGGTGCC | |
| | AATTTCCCGGGTGG | |
| 33 | AGTACGAATGGTAAGGGGGGTGTAGACTCTGTGTCGAAGAAATCTTTCGTCAT | BMR1_04G09385 |
| | TGAACTTGAAGACTCCACTTTTGAAAGAAAGACCCAAGCTTCTTCTGGTGGCAC | ectodomain |
| | TTCTGGTGTATGGTTTGTCAAGTTTTATGCACCATGGTGTGGACATTGTAGATC | |
| | AATGGAGAATGATTGGAATGAGTTGGCCAATATTTTGGGCAAACAAATTAATG | |
| | TAGCAAAAATTGATGCTACAAAACACAGTGTTACTGCGAAAAGATTTGGAATTA | |
| | CAAGTTTCCCAACACTTTTGTTGCTGAAAGATGGAAATTTCTATCAATATGAAAA | |
| | TAATAATAGAACTGCTGATGCTCTTAAACAATTCGCTTTGCATGGCTATAAACA | |
| | GGTTAAATCAAAGCCTGTTCCTAAAGAATGGAGTTATTTTGTTCGGTTCAAATTC | |
| | TTTATCAAATCTGGATTTTATGAAGTTAAGCGCATATACCAGTTAGCATATCCTG | |
| | GGTTCATAACA | |
| 34 | ATGGCGAAATTACATGAGAGTACATCAAGTGCAAGTGCTAGCTTCGACCCAGA | BMR1_03G04485 |
| | GAAGTCGGATTATGACGATACTTATGTTTTAACAGAGACAACGCCAACTTACAT | full sequence |
| | AAGGCATGGATTTGTTCGAAAGGTCTTTGCTATTTTGTTTGCTCAATTACTAGTG | |
| | ACCCTTGGATTTTCATTGATCTGTTATTTCTACCGAGAATCTGTTCATTCGTTCAT | |
| | ATCCAAAAATATCTGGATTTTTCCAACCTTAGCTATATTATCATTTATAACATCCC | |
| | TTATACTCATATTTTCACCTTCACTCTCTAGGAGATATCCGTTGAATTACGCTATT | |
| | CTCGTTATAGAAACATTGTATTTCTCATTCATTGTCGGATTGTCATGCGCGTTTA | |
| | CTAAAAGTCCGACTGCCATAGTATTATCCGTCTCTATTACTCTTGGGATCATTCT | |
| | TCTTGTTGTTCTGTTCACACTCCAGACCAAAATTGACTTCACTAGATATATTATCT | |
| | ATTTTATTTTATTTAGTTTTGTAACGTTGGTTTTTGGCTTTATTGGTATCTTTGTCC | |
| | CATTTGACACCCCTCTCAGGATGTTTTATTACGGTTTAGGCGTACTTGGTTATTC | |
| | ACTTTGGATGGTACTTGATCTTCAACTGATTATCGGTGGCAAGACTTACGAATG | |
| | GACGGTTGATGATTACGTCCCTGCATCACTCTCGCTTTATACAGATGTTATTGGA | |
| | ATTTTCTTAAACGTTCATGGCATGTTTTCTGATAGA | |
| 35 | ATGACTATAACCTTGAATATAAAGGTTAATTCTGAGACAAATTTTACGGTAGAG | BMR1_03G01645 |
| | GCTGAACCTTCCTTTACTGTAAAGGAATTAAAGATATTATGTGAATCACAATCG | full sequence |
| | AATATTGAGGCGCAAAATCAACGCTTAATTTGTAAGGGCAAGCTTTTGAAGGA | |
| | TACAGACATTCTTTCCGATGTAGGGGCAGTTGATGGTGCCACGGTTTACCTGGT | |
| | TCGTAGCCAAGTCAACAAAACGCAATCAGCTGCTCCTAAGCAAAATACTGTCCC | |
| | TCAACCCACATTACAGACTACAAATCAACCTGCTGGTCAAACACAAACGTCTGG | |
| | ACTTGGATTTCAACAACAAGGGTTTCAGCAAGGATTTTCTGGATCTCAGCAACC | |
| | AGGATTTCAAGCTAACCCCTTCCAAAGCATGCTTGCTGGTGGATTTCCCAATTTA | |
| | GATCCCACTCAAATGATGGAGATTTTGAATAGTCCAATGGCACAGGAAGCTAT | |
| | GCAGAGATTGAGCCAGAATCCAGAAGTTTTGAGGAATATTTTGCAGAACTCAT | |
| | CTTTGATGACTCCCATGCTTGAACAAAATCCTATGCTATCAGAAATGCTATCAAA | |
| | CCCGGAATTGATGAGAAGTATGTTGAGACCCGAGGTTTTACAAGCCGGATTGC | |
| | AAATGCACCAAGCCATGCAGCAGCAGCAACAACAACAACCTGGGACTCAAACC | |
| | AATCCAATAGGTTCACAAAATCCAGACTTCAGCAACATGATGAGGCAAATGAT | |
| | GAATGTATTTCAGCAAAATCCATCTGTTGCACAGCCCACAGCACCACAGGTATA | |
| | TACCGACCCGAGGCCTCCGGCTGAAAGATTTGCAACTCAGCTACAGGCATTGG | |
| | CTGAGATGGGATTCATTGACACTGAAAAAAATATCACTGCACTTATTGCGACCA | |
| | ATGGCGATCTCAACGCTACAGTGACCAGGCTGCTTGAATCCAATTTT | |
| 36 | ATGGAAGAGGCTGAACGCAAATTCAAGGAATTTAATTGGGCCGATAGTCAGG | BMR1_03G01960 |
| | GATGGCGAATTTACTGGGACAACTTGTATCCAACTCCCCCCTTATCCAAAGTAG | full sequence |
| | ATAAGTTCAAACGTTCCTGGTTCAAGAGAAACGTAGATTCTAATATATCTTCAA | |
| | CACCACTGTCTGAGGTTGGCAAACAAACTCAACAAACTACTCCAAATCAATATC | |
| | GTTCGCAGGGCAACTTTGCTATTTTAACTCTCGAGGCCGGTGTTAGATTGTTGT | |
| | ACCTGCTTATTACAGCACCTTTATTTGTTCTACCACTAATAGGAGTCAGGCTCAG | |
| | TAGATATATATACTACCACTACTACATAGACATAGCGCTGTTATTGATCTTTCTA | |
| | TTGTCTGGCATCGTAAGGGAAAGAGGTCTACCGAAAATGGATTCCGTGTGGCT | |
| | GGCATCTGCTTTCTATTCAGACATGACACAATATATAATGTATACATGCATCCTG | |
| | ATGATGTCCGCACCAAGGCCTGTTTATCTGATTTTACCCTTTCTAACTTGTCTAAT | |
| | CGGGCTAAATTCTCTGGCCGAAACAAATTTATCCAAACTGCCCAAATGGCTTGA | |
| | AAACATAGTGGGTGAAATATTTAAGTATACTAAGGATAATATATATTGGTTAAT | |
| | GCAAACTCGTGGCGATGTTGAGTGTTATTTGTTGTTTTATATTGTATTCGGGTTC | |
| | CTAACGAAATCAAGCGCAGTTATCACTTTAATGGCCTACATAAACTTTATGAAG | |
| | TTAAGGATTGGCATTGGTGATCCCTTTATAATGTCTGCCTTCTCTAAATTGCATG | |
| | GCTGCATAGTGAAGTTGTTATCGTATGATATGTTTGGACATATACCTTTGAGGA | |
| | TGTACTTGAAGATATCAGAGCTCTTAACTAAATATATGTCGGGGCCAAGGCCGC | |
| | AATAT | |
| 37 | ATGGAGGACCAAAATACTACAAATGAAAGCATTTCAAATTTGCACATGGAAAA | BMR1_04G05080 |
| | CTACTTCCCCAAAGATATTTTTTCCAACCTAGATAATCAAAAAAACTTAAAAACA | full sequence |
| | TATCATTCAAAGACGTTTGAGAAATATTTCGAGTCTGCTGCCAAAGATAATGTT | |
| | GAAAGGTGTCGTACATCTGCTGTCAAAGAATGGCTGAATAGAAATCTTCCTAGT | |
| | GAATATAATGAAAGGCACAAGCCCACACTTAAACTAAACCTGAGCCCCAACCAC | |
| | TTAAACTCAACTTACAACAAACAAATTTCCGATTACACAAGGAATGCCTATAATC | |
| | GCATAGAACAGTTGAAGAAGCTTCAAGAAGCTTACTCTCTGTTAAGACAGAAA | |
| | CTACAAGAGAAACGCAGGGCCTCTTGTCATAAGGAGGTCTCTGAGGCAGAGGC | |
| | TTCAGTTCTTAATACTCAAAGATTGATAATTAGCTTAAAGAGAGAAGACAAAGA | |
| | TAAAATCGCCACAGAGTTACTTATACAAAATGCAGAAGAGTTAGGTATACCATC | |
| | CAAAGATTTGGAAACTCTAAACGGCTACACATTTAATGAGGCTCTCAAAACTAT | |
| | AATCGATATAATTTCTAACATGATAAACAACAAATTTATGACAAATTTGAAAGA | |
| | TAGGTGTGAATCAGCTAGGCGTAAGGCAGGATCTGAATATCGCGATGAAATGG | |
| | AAAGGATAAAAGTCGATTTGGACATGTACAAAACAAAGTCTGAAAAACTCGAA | |
| | TCCACAATATCTACATTGAATTCTTATGCAGATTCTAGCAAACAAAATGCTGAAA | |
| | AGGTCATGGAATTGCAGCATTCGCTAGAAGATTCCAATAGACAAATTAATCAGC | |
| | TAAAAGATTCACTGTACAATATGGCCAAAGTTAATGAGGAGTTGGAAAAAAAC | |
| | GAAGTTAAGCTAGAGGAAAGTTTGGTAGAGTTGGAGAGGTATAAGTTGGAAT | |
| | CTCAAAAGCTTGAGTCAGTCATTAAAGATTTAGAGCTTTTGAACCAACAAAAAC | |
| | ATGATAATGAAGAACTCATTAAAATGCTGCATGATGAGCTAGATCAGTGTAAG | |
| | TCTAAGTCACTGCAATTGAATAAAAAGATTGAAGATCTTGAAAATTCAAACAAG | |
| | GAAAACCTAATACCCTTGAACAATGAATTGGCCCAGGCTTATCAGAAACTATCC | |
| | GAATTGGAGCATTCATATGAACAGATTGAACACCAGAAGAATGAAGCAGATAA | |
| | AAAACTTGAATCTTCAATTGATGAGATTGAGAAACAGAAACAACACTCCGAAG | |
| | AATTGGAGCAATCCATCACTAAATTGAAACAAACAATTGAAGAAATGGAAAAG | |
| | GAAACCACGGACCAAGTAAATGATCTGCAAACGTGCCTAATAGATGCGAATTG | |
| | TAAAATCGAATCGCTAGAGGGAACGATTTCTCAGTTGAAACAGCTAAACTTAG | |
| | AGTTGGAAGGGGGAGAACATAGGATACAGGAATTACAATCCAAGCTTACCGA | |
| | ATCAAATTCCACAATTGAAAAACTTGAAAAGACGATCACAGAATTGGAAAATGT | |
| | GTCTAGTAAATATATCGACTACGATGAGAAGATGGATAATTTGCAAAAGCAGC | |
| | TGAAAGAATACACTGAAAAGATCACAGTGCTGGAAAACAACGCCTCTGAATTC | |
| | AACTATAAAGACCAAGCTGAAACGTTGAAGACGGAACTGGATAAGTCGCAACT | |
| | AAAGATAATGGAACTGGAGACTATGATTAAGGAGAACAGCGAAAAAACGGAA | |
| | AGAGTGCCATCTCCCAGAAAGGACAACGAGGAGGTTGATAGATTAACTTCGGA | |
| | AATATTACAGTTGAAGAATCAGGTGGACATTCTGCAAAATGAAAAGACTGACC | |
| | TTGAACAAAAATTATCACAACAATCTCCCAGAAAGGACAACGAGGAGGTTGAT | |
| | AGATTAACTTCGGAAATATTACAGTTGAAGAATCAGGTGGACATTCTGCAAAAT | |
| | GAAAAGACTGACCTTGAACAAAAATTATCACAACAATCTCCCAGAAAGGACAA | |
| | CGAGGAAGTTGATAGATTAACTTCGGAAATATTACAGTTGAAGAATCAGGTGG | |
| | ACATTCTGCAAAATGAAAAGACTGACCTTGAACAAAAATTATCACAACAATCTC | |
| | CCAGAAAGGACAACGAGGAAGTTGATAGATTAACTTCGGAAATATTACAGTTG | |
| | AAGAATCAGGTGGACATTCTGCAAAATGAAAAGACTGACCTTGAACAAAAATT | |
| | ATCACAACAATCTCCCAGAAAGGACAACGAGGAGGTTGATAGATTAACTTCGG | |
| | AAATATTACAGTTGAAGAATCAGGTGGACATTCTGCAAAATGAAAAGACTGAC | |
| | CTTGAACAAAAATTATCACAAATTAGTGCGGTTATTGAAGAAAATAGACAATAC | |
| | AAGGAAAAAATCGAATTACTCGAGAGAAAATTGAATGAAATAAATAAAGAGA | |
| | AACCAAAAGACTCATTTACAAACGTAGAAGTTATCAATGCAGCTTTTGAAGATG | |
| | TAAGATCCCTCCCAATCAATTCCTCCAATGCCTCTGATTGGACCGCAATGCCCAG | |
| | TGAGCTGGAGCTGGAAGAGCATAAATTGTACAAAAAAAAATCTAGTAGAAAG | |
| | GGGAAAAAGAAATCATCAAGGGAGAAGGAGACTAGCAGAAGTGACATATCAT | |
| | CCAGGTCAACTAGCAGGCACTCAAAGAAGGATAAGCCTACAATCGTTGACAAC | |
| | AACTCTACTGATGTAGAAGCATCAGATTCAAATGAACAGATGATTAGTGATCCA | |
| | GTTGAATCGATTACAGACCAGTTGAACTTGGTTAAACAATCAACAATACAACTA | |
| | ACTGAGCTGGGGTACGACAGCATATCCAACGTTGGGTCGTACTTGTCTGACTAC | |
| | ATATTTGGCGGTAAT | |
| 38 | ATGGATGATCTACCCGGTTCATTACATCAATCAACACCAAATGAGAAGCCAATG | BMR1_03G00820 |
| | GCTCCACCCACAGCACCAAAATCTGCTCCACACGTTCCTATTTCTGTCGAATCAA | full sequence |
| | AGGAGTTATCAAAAGAAATTCCAAAAGAATCTCCTATGACTAAGGAGGACAAG | |
| | AAAGACGTGGCCAAGAGTAAAGTTTCTGCTGCTGTAACTGAGAAAAAAGTTGT | |
| | TAAGGAACCAGTGGTACCTAAAATAACAATTGAGATGAAAAAATTTTCTATGTC | |
| | CAGAGAATCCACCCAATACTCAATCTTCATTATATTTAACCTTATCTTTGCACTAT | |
| | TATACATTTTCAAAGTAAGATTGATGGCCATATTTTGCAATCTAATTATTGTGGC | |
| | TATTTCAATTGGAGCTATCCTTTCCTCCATAGACCCTAATAGACGCAAAGTTGAT | |
| | GAGACAAATGTTAATATTATATTTATAAGCCCGACAACAGTTTCAGATTTAGCA | |
| | ATCGTAATCACGGCAAAACTGAACCAGTACATTTCATACTTTAGAAGAATTTTG | |
| | TTATGGCAGGATTTTATTCTTTCAACCAGATTTACTTTGTGTGTCTACATTATGG | |
| | GAATCCTTTTCAAAATTATACCGCTGGTTGCTTTAATATATATAATGTGTTGGGC | |
| | ATTTTATCTTTATATGTTCATATGCAAGGAGATGGCAGATCAGTTGTTGGACATT | |
| | ATAATGGTTTATTTGAAAAGGGTGTCGGGAAATTTCGACGAGTTCTGCTGCAAT | |
| | ATCCCCAAGATGAAGGATGTGGGTAAGGAACTG | |
| 39 | ATGTTTAACGAAAACGAGATCCCTCAGTTCCAGCGCCCTTTCACCCCCTCCAAA | BMR1_01G02100 |
| | GACACGGAGATTGACACTAGCATCAATTTCCCTGCCAATTCAATTCTAAATAAC | full sequence |
| | ATATCATTTCAAAAATTATTGGATTGGTTAGATGAATGCACAGAAGGCTTACCA | |
| | ATCGTGGAAAGTTTTGCGAGACGATTCAACGTTACTAGGGGTCATATAGCGGC | |
| | AATATTTTCTGCTATATTAATTCTGTACTTTATTTTCGGCTGGAAAATTAACATCT | |
| | TTTGCAATACAATAGGGTTGGTATATCCAGCATTTAAATCTCATAAGGTTTTACT | |
| | GATTCATAGAGCCATGACTGAAAGTCCAAAAGCAACAGCTACTATCACAACTG | |
| | GGGGAAAGGACAAAGAAAATGACACAAACCCTCAAATGCCTACTTGTCTCAAT | |
| | GGAATCCAAGGGGAAATTATGTTTTGGCTTAGGTACTGGATTGTTTACTCGCTT | |
| | TATTTATTTATTTCGATATTAATTTTCCCCTTGATATCATGGTTGCCACTAATATC | |
| | TATAGTTCGGGTTGGTTTTATCCTATATCTCTACCATCCATACACTAGAGGGGCC | |
| | AACGCGATCTACTACCTCGTAATATCACCTCTGCTAAGCAAAAACCAGAAGATC | |
| | ATAGAACAGGCCATTGACACCCTCGAAAGGGTTGCGATGGGTGAAATCAGGA | |
| | AATTTACAGAAAAACAAATGAAGCTACAC | |
| 40 | ATGGATAGCATTGAAGAGTGTAATAAATTGGTAGATGCGGTGACCAAATTGGC | BMR1_04G07360 |
| | TACATCATTTGATTACCAAGCGCAAGAATATCTATACAATCTTACCCGCGATGA | full sequence |
| | AAATGCAATTAACATTGCATTATCCTTTCTATTATCCAATAAATACGTGTTGAAC | |
| | TATTCAAGCAGTTACAGCAACGAAATTTTACAATATATAACCGATGTTCGTGGC | |
| | CCAAGCAGTTGTTGCATCTGGGACAATATCCAATTGGACGAAAATACCAGTAG | |
| | GCATCTGGGCCACATAACGTCTATTTTGTTTAAGGAGCATATGCATATAGTTTT | |
| | GTGTTTCGACGATGCAAAGCTTATTGCACTTATTGATACATTGGTTACTATTTGG | |
| | CAATTACAATCAACTATATCACTAGTAACTCAAAGCTGTTTCGCTGATAATGCTA | |
| | CTGATGATCATATAACGAGAGTTATTTCTATGATTATATTCAAAAAGCCGCAATT | |
| | ATTAACCCACTTCATCAACATACTAAACACTCCCGAGGTAACTGATAAGTATGT | |
| | GGCTGATTTCTCAATGAAATTTAAATTAAGAAATTGCGATAGAGATGATATTTA | |
| | TGTCACAAGGTATAGGCATATGATTGTCTATGTGTTAGCTCAGGTGCTTGAAAA | |
| | TTTTGTGTCTTCGAACGTTACTGATAAGATATCTTTTTTGCCAATTAACTTCTCAG | |
| | ATTTAGTCATATTGTCGGGAAATTGTAACAATAGGTATCTTTACTCATGCGCGTT | |
| | ATTAATTGTAAATTCATTGAACTTTCACATTTCAGAAGAGTTATTACATCAATTA | |
| | TTGGTAATTGTTGAGAAATCTGGATTTGACTGCGATATACTTGAGATGTGGAAT | |
| | TTTGCAATCAGTCATTTACAATATTTGAAAGTTGACAATTTCATGATGAAATCTT | |
| | TCAGGTTAGTCCAAAACGGGCTCATTGAATCCACAAATCCAATAAACATTGTGC | |
| | CTGTATTAAACGGTATAACAGTATACTATGTTAATAATTCATCAGTTCCAACTGA | |
| | GATCGTAAGAGCTTACAGGCAATTGCTTTCCGTCGAATCCCTTGACATTCCCATT | |
| | GAAATCGGTGAGTTATTTGCAGCTGTCCGAAATGCTTGCACTGAAAGTCTGATT | |
| | ACGTATGATAAAATTGCTTCATTTGTGTCGCAATTTGACATAAGAATGATTTCAA | |
| | GTAGGTTAATTGAACTTTGCAATCCTAACATGCAATGTTGGTCTTGGTTGAATG | |
| | TAAATTGTTGTGTAGAAGAATTTCCCACGGATTATGACATATACATTGATTCTAT | |
| | GAAAAATGTATTTAAAGACCTATTTTTCATCTTACCAGAACAACTTTTAGACACT | |
| | ATAATAAGTGAGATATTACAATCTGTAAATGAAAATACGGAAACGTCACTAATA | |
| | GCATTAATTTGCTATGATTGCCTAGAAGTAAGGCATGCATTGGCAATTCTAGAC | |
| | AAGATTAAATCATTGTTTACGCTGAAATTGTCTTCAAACTACGATTTCATCCTTA | |
| | CTTGTATGTTTTGCCAATCCATATTTGTGCCTCATTTGCACAGGATAAAAGATAT | |
| | ATTATTCCAGAGATTTGTTATCAACATGAAAATATCGAATATTTGGTCTAAAAG | |
| | ACTGGCTAAATGTATAGCAATATTGAACGATAGGGATTCCGTTCATAGCGTGAT | |
| | TGAATGTGTTCAGTATATATTGGAAACTTCTAAATCCAAGGGTGTATTGTCACA | |
| | AAATCACTATCCTTCAATTCTATCATTGTCAAATCTTTTAGATACGTTGCCACAAA | |
| | TGCCTTGTGATGTTAATTTGACTGGATCCAATTATATTTTTTGCAAATGCTTGCT | |
| | CGCGTTAAGTATTTCTACTAGACAATTATCACAAATATTGCCAAAAATCGCTCTC | |
| | AATTTGGATATACTATATGAAGGTGATGTGGACAACGATTTGTTTTGTTTATCA | |
| | ACATCTGATAGCCAAGCTGCAGTTAACTTATTGATGCAACTATATATACAAAAT | |
| | AAGGTAACCGATATACCTAATGAATTGCACGAGATACGTCACCCCGGGCTTTTA | |
| | ATGTTATGTAATTTGAATGAAAATTTTGAACAATTCAGGATGAATAAGTGTGTA | |
| | GAGTTCATGTCAACGGTCAGCCAAGAATCTTGCTGTCACTGTGCATGGGCCTGT | |
| | ATTGCCTATGCATCTTATGTTATCGAACAGAAATTCAACTTTGAATTTTCAATGG | |
| | AGATGGTTAAAGTGATATATAGGGTGTTACCATGTTTAATTAAAATGGTTAAGT | |
| | CCAAGAATGAGCCTTTACATTGGGATTTAGATGCAATTTTGGAGTACAGAATTA | |
| | ATGAAATACATCCTTCAATTAAGTGTCTAAAGGGGACAATTTGGAATATTTACT | |
| | ATGATGGTACAGTTAGTGTCGGCTCTGACGCTTGTAAATATTTATCACTAGTAA | |
| | ATAAATATGCTCCAAGAATCATTAAAAGTTTATCAAATCCAAATGTATTGCAGTT | |
| | GGTGTATCAAATTTCAATGTGTACTGGATCCATTGAAGTTATAGCAAATAAAAT | |
| | CGCAATTTCACTA | |
| 41 | ATGAGCTGCATATTGAAGTGTAACAACGAGGATGAATTGGTTGTGGATGGGGA | BMR1_02G02185 |
| | AAAACCCAAGGTAGTAGAGTACGTCGAGAAACCATCGGTAATTTACAAGCCTA | full sequence |
| | CAACCGTTGTCCCCCCTAACAGCCTGATCGAAATTACTGCTCCCAAGGATTTGCC | |
| | TCAAAATCCAACATTCTTCCCAACCATAGATACATTTTTCGACAATGACGTCAAG | |
| | CAGATCGTTTTGTTGATGGAATTGCCCGGATTTGTGGCGGGAGATATTGATTTG | |
| | GAGGTTGGTGAAGGTGAAGTTTGTGTTTGCGGCCCTAGGTCTAAGGAGGAACT | |
| | ATATGAGAAGTATGGTCAAAATTTAGATATACACATTCGAGAACGAAAGGTTG | |
| | GCTACTTTTACCGTCGCTTCAAGCTCCCGCACAATGCTCTTGACAACACCGTAAA | |
| | GGCATCGTATCAAAATGGGATTCTCGAAGTGCGCATTACCTGTACAGAATTTTC | |
| | ACCGAAAACGCGTGTGGAGATCACATCT | |
| 42 | ATGAACGGGTCGGTGGAAGAACTATTAAATCGCTTATCGGCGATTAACGACAG | BMR1_04G08260 |
| | GTGTTATCTGCTGGTGGATGAAATAAAAAATTACATGTCAAACAAACTTTACCA | full sequence |
| | TGAGTTAACCCTAGCACTCATCGAGTTGTTTACCATGTCTGAAATATCTTGCAAT | |
| | GACAGACTTCTTTTATTTGAAATGATAGTGCATCCTATCAAAAATGATCTGAACA | |
| | TTTTGAAGTTCTCACATATTTTACGCCTGTCCTCTGAACATCTAGAGCCTTTAGC | |
| | GTCACTAGATCAATTGTCTAAATATGACAATTACTTATCAACAGACACTCAAGCC | |
| | AGTTTTATGATTAAAATTGCAAAGTCATACCACCATACTCGGAATCAATCCTACG | |
| | ACCAAAGTTTGAAGCTTTTGGAGGAAGTAAAACCTGAGATAGAATCCGGATTT | |
| | GGCTTGGACATAACGGTAATCTCAGCCTATTACAAAGTGTCAGCGAATCTAAAT | |
| | AAGGCAACGCACAAATACAATTCATGGTACCAGGACTCACTCATGTACCTAAAC | |
| | TACACTCCTCTGGATAGTATCAGTCCAACTGAACGTGATGAATTGGCACTGGAT | |
| | ATAGCAATTGCCTCGATTGCAGCGCCAGATAACTACAATTTTGGTGCTGTTCTA | |
| | ATTCAGCCACTAATCAACACGTGTTTGAAACAGCACTCCACATTTGGCTGGGTA | |
| | TACGCAATTTTAATGGCTCTTAATGATGGCGACTTCACACAATACGATGAGATA | |
| | ATTTCAAAATATAAAGTCCAAATCAGCCATTCCGAATTAAATCATCACAAAGAG | |
| | CAACTCCAAAGAAAAATTACACTTATGGCATTCCTGAAACTCGTGTTTAGAAAG | |
| | GCTAAGAAACAACGCATATTTACATTCGAAGAAATATCCCAGAATTGTCGGATT | |
| | CCAATAGATGAGGTGGAATACTTGCTTCTAAAAGCTATGTGCAACAATGTGGTT | |
| | AAGGGTAAAATTAACCAGGTTGAGCAGATCGTCAGTTTTACGTGGGTTCAACCT | |
| | AGGATAATCGACTCAACAAAATTGACAGTTCTCCTAGATGGGGTTAACGAGTG | |
| | GAATCAACAACTAAAAGCGCTCATAAACAAGCTCAAAGAAATTACTCCAGAGTT | |
| | ACTAGTGTCG | |
| 43 | ATGTCGGGACTGTTTGGTCAATCGCAGCAGATTGGTGGTGGATTATTTGGCCA | BMR1_03G02345 |
| | ATCTAACCAACAGTCTGGGGGAGGGTTATTCGGGTCAACATCGCAACAGCCAA | full sequence |
| | CCCAAACATGCAGCGGATTATTTGGATCATCTCCAGCTCCCGCAAATAGTAGTA | |
| | TTTTCGGTTCAAATACACAGTCTGCTGCTTCTAGCGGTGGGATTTTTGGTTCATC | |
| | CACAGCTCCAGTTAATAGCGGAGGCGGCATTTTTGGTCAATCTAACACTAATGT | |
| | TAGCAGTGGTAGTGGATTATTTGGCGGTGGAAATACTACAGGTCAATCTGGTG | |
| | GGGGTATTTTTGGTTCATCAACTACTTCGGCTCCAGCATCAGGAGGAGGGTTAT | |
| | TCGGCCAAACTGGAACCACTACCAGTGGAGGAGGTGGATTATTTACCTCATCTT | |
| | TTGCGCCAGCGCCAAGTAGTGGCGGACTATTCGGGCAGCCATCCACACCTGCG | |
| | ACAAGCGGCACTGGATTGTTTTCTAGTACCAGTACGACTCAACCAAGTTCGGGA | |
| | GCGGGGCTATTTGGTTCAAGTACTACTCCAGCTAGTGGAAGCGGTGGTCTTTTTT | |
| | GGTCAACCTAGCACTTCAACGACAACTAGTGGTGGTATTTTTGGTTCATCAACT | |
| | ACTTCGGCTCCAGCATCAGGAGGAGGGTTATTTGGCCAAACAGGAACCCCTGC | |
| | CAGTGGAAGCAGTGGCATATTTGGCTCAACAAATACCACAACCTCGGCCTCAG | |
| | GGACTGGATTGTTCGGTTCGACGTCAACAACTCCACAGCCTGGCAGTGGTAGT | |
| | GGATTATTTGGCGGTGGAAATACTACAGGTCAATCTGGTGGGGGTATTTTTGG | |
| | TTCATCAACTACTTCGGCTCCAGCATCAGGAGGAGGGTTATTCGGCCAAACTGG | |
| | AACCACTACCAGTGGAGGTAATTTATTTGGCACCACCAGCACTACAACACCGGC | |
| | GCCTGCTAGCGGTGGACTATTCGGTTCTTCTTCTACTACGTCCACAACAACTCCA | |
| | ACACAAGCAACAACTACAGTAGGAGGGGGAGGGTTGTTTGGCACTGCAAGTG | |
| | CAACTACTGCGCCGGCGTCTGGTGGATTATTTGGCACCACCAGCACCACAACAC | |
| | CGGCGCCTGCTAGCGGTGGACTATTCGGTTCTTCTTCTACTACGTCCACAACTCC | |
| | AACACAAGCAACAACTACAGTAGGAGGGGGAGGGTTGTTTGGCACTGCAAGT | |
| | GCAACTACTGCGCCGGTGTCTGGTGGATTATTTGGCACCACCAGCACTACAACA | |
| | CCGGCGCCTGCTAACAACACTACTCCTGCTAACACTACAACTGTGCCAACTGCC | |
| | ATACTAACAACTAGTACACCGTCCCCTGCTACCGATGGTTTATTTGGATCGACC | |
| | GACGTTACAACTACTACTGATTCCACAACTAAACTCGTTGGCACTAGCCCTTTTG | |
| | AAAAACAAACAGATTCGGGGCCGGATGTCACTGCCGCTACTAACGATACGCCT | |
| | AACGCATTTATCACAGATAAGCCAACAGCTGGAGGAGATCTCAAAGGCCAAGT | |
| | TGAGTTGTCATTTTCGTCTGTGGAGCATGAATGTGTGCAAGACCTACTTTCTAA | |
| | CTGGGAGAAGAGAATGGAAGTCAAGATACAAAGGTTTACTGAGTTTGCTCAAG | |
| | ACATACAGAGGATCGATCGCGATCTAATACTACAAACAGAAAAGTTACAAGTA | |
| | TTGTTGGATGAGCAAGCAACTGTTCAGGAAAGACAGAACCAAGTACAGGAGAT | |
| | GATCCAAGTTATTGAGAAGGAACAGCAACAGGTGCTAGAATCGTTGGATATAA | |
| | TGGATACTGCGCTTGAGACGTTGTTGGGTCCAGATAAGAAAATAACCAGTAAG | |
| | AGTGGCGAAACTATAGATTTCGTCTCCAACAAATTGCGGGATTTAGAAGCTCAA | |
| | TTAAAGGCCGCACATGATGTGGTGGATTCTGTGGTTAAAGCTTCGCAACCGGA | |
| | GCCCCTGGCTAATGTTGCTAAAGTGTTTGCATTCCACCAGGACACTATTGAAAA | |
| | TATCCAGCTTCAGACATCGGAGATTGAGAAGAAACTTGACGCTATTAAGCAGC | |
| | AGCAAGCAGTC | |
| 44 | ATGGCATCGCTGCTGAAAGTATCTCCACAGGACAATATCGAGTTTCCACTGGTG | BMR1_02G02960 |
| | TTATACACGCCTTTAAATGCGAACTTATTACTGGAAAACTTATCTGGTGTGCATG | full sequence |
| | TGGCCTTTAAAATCAAGACCACAGCACCCAAGGGCTATCTAGTCCGCCCCTCAA | |
| | CAGGGACAATTAAACCTGGAGAGGCACTTACTGTTCAAATCATTTTGCAGCCTC | |
| | TTAGTGAGGTTCCAAATGTGGTCAACGACAGGTTCCTCGTCCAGTGCACTGCCA | |
| | TCGCAAATGACGAATTGGTTTCCAAGGATTTCTGGACTACACTCGACAAAGCCA | |
| | GTATCCAAGATCACCGCCTCAATGTAACCTTTAAGAAGGATATTGGTCTCAACA | |
| | TACAGACAAGCCAATCCAACATTGGCGTCCCGCCTCACATCGCCGCCAGAATTC | |
| | TCACCCCCTTGGGACCCAATGCAGGCGTTGCAGAACTAAGACAAAAGTACGAG | |
| | GAGCTAGTATCATACTGTCTAACGGCAGAAAAGCAAAAGGCTGCTCTGGTTAA | |
| | AGACAACGAGAAACTTCGTCAGCGCCTCCACCTGGGCCCCAACGACCCTGCCTC | |
| | TGGAAACAAATGGCCCCTAGAAGGTTGGCATCTACCCGTAATGGTGATCATATT | |
| | AGTAATTATTCTCAAGGCCATTGGCTACTGG | |
| 45 | ATGTCACAAGGACCTGCTATTGGGATTGATTTGGGAACCACTTACTCATGTGTT | BMR1_01G02545 |
| | GGCGTTTGGAAAAATGAAACTGTAGAAATTATAGCCAATGATCAAGGCAACCG | full sequence |
| | TACAACTCCGTCTTATGTTGCCTTCACTGATGTGGAGCGATTGGTTGGCGACGC | |
| | CGCCAAAAATCAGGATGCTAGAAATCCTGAAAACACAGTTTTTGATGCAAAAA | |
| | GATTGATAGGGAGAAAGATCAATGATCCCTGCATCCAAAGTGATATAAAACAC | |
| | TGGCCATTTACTGTTGCGGCTGGACCAAATGATAAGCCTGTGATCAAGGTACAA | |
| | TTTCAGGGTGAAACTAAATCCTTCCACCCAGAGGAAATATCTTCTATGGTCCTCA | |
| | CCAAGATGAAGGAAATTGCAGAATCTTATTTGGGAAAGACAATCTCTAATGCT | |
| | GTTATCACTGTACCCGCTTATTTTAACGATTCTCAACGACAGGCCACTAAGGAC | |
| | GCTGGTACCATTGCTGGGTTAAATGTTATGCGTATTATCAATGAACCAACTGCT | |
| | GCCGCAATTGCCTATGGTATGGACAAGAAAGGTACTTCTGAAAAAAATGTGTT | |
| | GATTTTCGATTTGGGCGGTGGCACTTTCGATGTATCAATTTTAACTATCGAAGA | |
| | TGGCATTTTTGAGGTAAAGGCCACACAAGGTGATACCCACTTGGGCGGTGAGG | |
| | ACTTTGACAACAGATTGGTCAACTTTTGCGTAGATGACTTTAAGCGAAAGAATG | |
| | GCGGGAAGAATATTTCAACCAATAGACGTGCATTACGTAGACTTAGAACACAA | |
| | TGTGAACGTGCTAAACGCACCTTATCCCACTCAACACAGGCCACGATTGTTGTA | |
| | GAAGCTATATTTGATGGCATCGACTACAGTTGCAACATCACTAGGGCCAGATTC | |
| | GAGGAGCTCTGTGCCGAAATGTTCAAAAACACTTTAATCCCAGTCGAAAAAGC | |
| | CTTAGCTGATGCAGATATGGATAAGAAGCAGATCCATGAAGTGGTACTTGTAG | |
| | GAGGTTCGACCCGTATCCCTAAGATACAGCAATTGATTAAGGACTTTTTTCAATG | |
| | GCAAAGAACCCTGCAAATCAATTAACCCAGACGAAGCAGTAGCCTATGGCGCG | |
| | GCTGTTCAGGCTGCAATTTTAACTGGCGAACAATCTAGCAAGGTCCAAGATTTA | |
| | CTGTTACTAGATGTCACTCCACTGTCACTAGGACTTGAGACCGCCGGCGGTGTT | |
| | ATGACCGTGCTGATACCCAGAAATACTACTATCCCCGCCAAGAAGGAACAAGA | |
| | ATTTACAACCAATGAAAATAACCAAACGGGGGTTATGATCCAGGTCTTCGAAG | |
| | GAGAACGTTCTATGACATGTGACAACAATTTACTAGGCAAATTCCATTTGACTG | |
| | GCATACCTCCTGCTCCAAGAGGAGTTCCCCAGATTAAAGTCACCTTTGACATCG | |
| | ATGCCAATGGTATATTGACAGTGTCTGCTGCCGACAAGTCGACAGGAAAGACT | |
| | GAACATGTAACTATAACAAATGACAAGGGAAGACTTTCACAGCAAGACATTGA | |
| | TAGGATGGTTGCTGAAGCAGAGAAGTTTAGGGAAGATGATGAAAAGAAGAAG | |
| | AGGTGTGTTGAATCCAAGAATGAACTTGAGAACTATTGTTATTCAATGAAAAAT | |
| | GCCTTGGAAGAGGAGGGTGTTAAATCTAAATTGTCATCTTCTGAGCTTTCCGAG | |
| | GCTCAGAAACTGTTACAAAACACTTTCAGTTGGATAGAATCTAATCAATTGGCT | |
| | GAGAAAGAGGAATTTGAAGCTAAACTCAAGGAGGTACAAGCTGTATGTACACC | |
| | TTTGACTGCTAAATTGTACCAAGCTGGTGGTGGCGTCCCAGGTGGCGCTGCCCC | |
| | AGGTGGATTTAATGCTGGCGGTGCAGCTCCCTCAGGACCAACTGTGGAAGAAG | |
| | TTGAT | |
| 46 | ATGAAACTTATCGCCTGTACCCTTAAGAATGTTGAGACCTGTGTCGAAGTAGAC | BMR1_03G04110 |
| | CCATCTGACACCGTAGATGCTTTAACCAATAAAATTGGATCGAGCCTGAACAAT | full sequence |
| | GCTAGTGCCAGCAAAATGCGATTAATCCATGCTGGTAAAATTTTGAAAATGGA | |
| | ACAAAAGATATCGGACTATTCTGACATCAAGGATGGGGATAAAATTATTGTTCT | |
| | GTTCAGCAAACAATCCGAAGCAAGTACAATAGCTAATCCTACACCTGCCCCTAC | |
| | CTCTACTCCCATTGCAGATGCCAACACCAGTCCCCCGAAACCCATCCCTACCACG | |
| | GACCCTAATGCCTTACTGATGGGCGAGGAGCTAGAAAAAGCCATAAACGGTAT | |
| | AGTAGAAATGGGTTTTGATGTTGAATCAGTCAAAGCGGCCATGAGCGCAGCAT | |
| | TCAACAACCCTAACAGGGCTATAGAACTCCTCACGCGTCATGAGGTAGACGTTT | |
| | CAGACCATGATACGCATCAATCTGTTCAAACGACGGGCGTGTTGGATGAGCTG | |
| | CGACAGCACCCTATGTTTGAGCAGATGCGGGCGATTGTGCGCAGTAATCCACA | |
| | GACGCTGCCACAAATTTTATCGCTCATAGGGCAGTCAGACCCATCATTACTCCA | |
| | AGCCATCACTGAAAATCAGGAAGAATTTATCCAGCTACTGAGTGAACCGGTACT | |
| | AGGCACAAGTGGTGATTTTATTGACGCCCAGTCTATAACTTTGACACCAGAGGA | |
| | AATGGAGTCTATAAACAGACTGGAGGGGCTCGGGTTTTCGAGACCGGCAGCTG | |
| | TCGAGGCATTTTTGGCATGCGATAAAAATGAGGAGATGGCAGCAAATTATTTG | |
| | CTTGAAAACATAGCAGATTATGTCTCTGACAATGATAAT | |
| 47 | ATGGATGGACAAACGGAACAACAATTGATTGAGGAAAATATCGAAAGATTTAG | BMR1_02G02560 |
| | GATACTTTATCAATTGCATTTGGACAACAATGAACCCTCTCCCACCGCTCAATTT | full sequence |
| | AATTATGCATGTGCTCTAGTCTGCTCCAATCAGCGATCACATAATGACACAGCT | |
| | ATATATCTCTTAGATGAATTGGTTCGCATTAGATATGAAAGTGAAGAATGTTTTT | |
| | ATCAACTCGCTCTTGCACATATGAAACGCAGAAGTTTCGTCAAGTCAAAGGAAT | |
| | ACTTGGATCGTATCATTGCACTGGAGGGTTCTAATCAACGTGTAATGGCACTTA | |
| | AATCAGTAGTCGTTTCATTACTAGCTCAGGATACTTTCATGGGCGGATTACTTG | |
| | GCGCCACTGCAGCGTTCGCAATAATTCTCTTCTTCACAATGAAAAGGAATACC | |
| 48 | ATGAACCAGAATTCACTCTGTTGTTCCAAGGGTTTTACCATGTTTGCAGGTGGT | BMR1_04G05635 |
| | GCCTTTTTCCTAGTTTCATTTAAGCCGGAAACTGTTATAACGAATCCACTGCTGT | full sequence |
| | TACGGCCGCTGTTGAATGTGAGCTGGGGGTATATATTTGGATCCCATCTATGG | |
| | GCAGCAATATCTACCTATAACAAAAAATATTGGGAAAACCGGATAATTCCTGAT | |
| | TATGCTAATTCACCATCACGGGAGATTATGCAAAGCAGACTTAAGATTAACGAA | |
| | TCGCGCATTTATCGTATATACCTAGAGAATCTAATCCAAACAAACGTTTTAGCG | |
| | AATGGGATATTATTAGTTACAACAAGCGCATTGGCGCCTTCAAACAAATTTCTA | |
| | AGGATTTGCTCGGGAGCAGCACTGCTTTTATCCATAGGAAATGTAGTATTTGCG | |
| | TTGCCTGAAGACGAAAAGGATGATGATGTGGGAGTTGTAAAGACATCTTCTAC | |
| | TTCATGTTTCCTGTCTGAAGTACTCTCTTTTTGCACATTCGGCGCCATAGTCCCTT | |
| | ATGTATTTGCA | |
| 55 | AGGAACCCACGACACACTAAATTTCACAAAAAACACACTCCAATAACTGACATA | BMR1_02G01795 |
| | TCACCCACAGCTAATAATTTGGATGATTATGAATTAATTACTTATGGAAATGAC | ectodomain |
| | GAAGGTCTACACGATGAACCTGGCCTTGGTAGTATAGTTACAGATATCGAGAT | |
| | AAGGACACCCGCCAATTTTGATGGGACAGCTGGTAATAAGGGGAGAAAGAGC | |
| | AAGCGCACTGATAAACCAGTGAAAAAGGCAAAGCCAGTGAAAACGAGGAACC | |
| | CAGTGAACATCACCGAGATAAATAACGCTAATGATGAAGATACTATCGACGCA | |
| | GACTTAGACGAAGATCTAGAGGATGATACTTATACTGATAAGCCCACCGGTTTC | |
| | TTTATG | |
| 56 | TCCATAGACTCACAAAAGTCCGATAATGATGTCGCAAACACTAGTGAAACAAGT | BMR1_04G07915 |
| | GAGAAATTGAATTATTACGATGCGCGAGATGATTTCCTCCATACTATGGATATG | ectodomain |
| | GTTAATCTAGTCGGTGGCTCCTGCACTTTGATAGATAAGGCAAATCAGCCCAGT | |
| | GACTTTAAACAACTACTAAATGCTTCTATCTTCGGCTACGGTAGAATCTCAGCCC | |
| | TTCTCACCCAAACCAAGTCCATCTACGACGTAACAGTGGCCAGTACATTGGCCG | |
| | CTGTGTTTGGTAAACTGATGCAAATTAATTTGGATGATGAAGGGCTGGCGGTT | |
| | GAGCAATGGTCTGCCAGGGTTTGTAGTATGTTGGAGATAGCCGAGGCGAGTCT | |
| | TCTTAGTACATCATTCAAGATCCTGGCTGATAGGCTTACCAAACACTGTGGGAG | |
| | GATCGCTGAGGTGTTTTTGTCGCAAAAGGAAAATGATCCCATCGATAAAAACA | |
| | AATCATCAGATTCTATAAATGATTATTCGTCCGGTGAAGGCGTGGACGTTGTTT | |
| | GGAGTGTGAGTACGGAATGCCTAATGGAATTCATCGCCCCAAACAGCCCAGGC | |
| | GGGTTCACTTCTAAATGGACCTTTGTGACATTCAAACACCTACTAATGCAGCTGT | |
| | CCATCTCAATCATAATGTGTGAATATCAGTTGCGTGATCCAAAATTGTTGGATG | |
| | ATGAAATTGAAGGCGTAGAAAATTTGTTATTTCGCTCATGGGATGTGCTGGAG | |
| | CATTATAGCAACATTCAGTTCTACACGCAATTAGATGGTGCATCTGGGATAATG | |
| | ACTTTTGCCCAAAGCAATCTAATGCCCCTTGTGAAACGGGATGGGGATAAAATT | |
| | AACGTGTCATGGAACCTACAGAATGAATTTGAAGCTGATCCATCGTCTAATAGC | |
| | AGTGGGGATTTGGGCTTGGCCGCCGGTGATAATGCTAGCAGCGATGGCGAAA | |
| | AGCCCGTTGCTACAGACCCACCCTTCTACAGCAGGAGGCCCAGGACCTATAGC | |
| | ATGTTGTCAGACTATAAAAGGAGGCTTTCTCTTACCTACGAGATGTCTGACGAG | |
| | GACACGCCTGATGAGGATGATGATTTTGAGCGTGAAGATGAGGAAGTAATAGT | |
| | TGAGAAGGAAACGGAATCAACTCCTAATAAACAAATTAAAAATGCAAAAAACG | |
| | TGTTTGGCAGAAGAAAAACTCCGCAAAATAAGGTATTTAGACCTAAACTGTATC | |
| | CACATGATACTGTATCCACTTCTTCTGTGACTTATGCCATTAAAACCAATAATGA | |
| | GAATCAAATCCAGTTCAAGTCGGGGGCCGCCCAGCCTATTGACTCAGACATCG | |
| | ATTCAGTCCCTAGGGCTAAAGCTTCAAATCCATCTATTGACTCAGGCAACGATT | |
| | CAGTCCCTAGGGCTAAAGCTTCAAATCCATCTATTGACTCAGGCAACGATTCAG | |
| | TCCCTAGGGCTAAAGCTTCAAACCCATCTAATGGCTCAGACATCGATTCAGTCC | |
| | CTAGGGCTAAAGCTTCAAATCCATCTAATGGCCCAGGCAACGATTCAGTCCCTA | |
| | GGGCTCAAGCTTCAAATCCATCTAATGGCCCAGGCAACGATTCAGTCCCTAGGG | |
| | CTAAAGCTTCAAAACCATCTAATGGCTCAGACATCGATTCAGTCCCTACGGCTC | |
| | CAGCTTCAAAACCATCTAATGGCTCAGACATCGATTCAGTCCCTACGGCTCCAG | |
| | CTTCAACGTCCAAATCTGTAAGAGGCACTACCGGAACTGGCAGTAACAGTAAA | |
| | TGGAGCAGTGTCAGAAACTCAGTGCTGAAGAACAAAACTGAAGAGAATGACA | |
| | ACAACGAGGGCCCTTCCAAGGCTAGTGGTAGTGCTGGTGGTGGGTTGAAAGG | |
| | TGAGATATCCTTCTCGGACACAGTATCGATACTCAGGTTCAGGTCAGAACCTCT | |
| | GCGGTGGACCGAAAATACTAAGGTAGAGGCTAACATGCATAGGAAGATGGTC | |
| | AATAAGTGCCAGGTGGAGCAGCTTCGTCCCCTCTCAATAGACTACTACAACAAG | |
| | ATCTCCCCGCCCAACCTGGGCCATCCAGTCATGGGACGGGCCACCGCTAGTTCA | |
| | TCGTCGACCCTTCTACCAAAGTTCATGGGTATCGGTGCCAAGGTATCCAAAACG | |
| | TCTAACAACCCTTCGATGGACACGCTGTCAAACTTTTGGGGGATGCAACGCCAC | |
| | GATAAGGAGCTGTCAGGGATTCGTAAATTGGATAAAACTGTTAGCCAAATGGC | |
| | CATTCTTGACCGCTTTAGTAGACTAGCTGACACCTGCTGGGATGTTGCCAGTGG | |
| | GAGGCCTTCAGGGTATATTTCTCCGGAGACCATGTACCGCCTAGGCGGTAACA | |
| | GGAAAGATGATTACATCCATCATGTAACTAGGGTCTATCCCTTCAATTTAGTTAA | |
| | GGCCTACCGTTTCTTAGTGACCAAAGAGGAGCCCCAAGTGCAGTCAAATGGGG | |
| | ACGATAATTTTGAATTCTCCCTCTTTTTACAGGCTCCAGAATTTGTCTCGATTCTA | |
| | CCCAATGGAGATAGGGCCGAAGCTCTGGTTATTGAACAAAAACTTGTCTCAACT | |
| | ATTAGGACCCTCAACACTAATAGCACAGAAGACCCACCTATAAGCGTCTGTGGA | |
| | TACGTTTACAAGCGAAACGACGTGGATAGGGTGCTCATTGGTGAGTTTGGCGT | |
| | TAAATTACCGTCCACTATCCTAGAACACGTCTATAAAATACATACGCCATACACT | |
| | ATACTCATTCACGCCAAGTTCACCGATAGCAAGTTCAATAAGCTCAAGGCCGTC | |
| | ATGCAGTTTTTGCAGGGGACACACTTTGGTGACTTCACTTTAAAATTTGTCTACA | |
| | AGGGTATGGTGCCCAGCAAAAATGTCGTAAAGGACAGTTGCCATGAGTGTTTG | |
| | ATAGGTCATTAC | |
| 57 | ATGGACAACGAATTCACTGATTTCTCTTTGGATAAATTTGGTACCACGGAATATT | BMR1_01G01620 |
| | ACATTAATAACCATAATCACAATTCAAATGATACTGTAGAAGATCCATTCTATCT | full sequence |
| | TACACATAACCATGATTCTAATGATAACGTAAAACCGAGCATACTAAACCCTGC | |
| | TAATGCTAGTTCTTGCATCCCTGCTAGTTATTGGCAGCAGCAAATGGAATACAA | |
| | CGATTCTCATTTGGACAATTCCCGATCTTCCATGATGAACGAGAATCTCATAATC | |
| | AAAGAACTGGGCGTCGATGATTCTCCTCAGAATGATAATGAGATAACTTCTATT | |
| | GATGATATTACTAAACTCACTGAAGGTGAACTCACTACCCATACCCATGAACCA | |
| | TACAATCTAAATTACGATTCTAAAACGGACCTACAAAATGCATTTGATACATTTT | |
| | ACCATGATTTTTCACTGTTTACTCCTACGGAAACTTTACCTGAATCACAGCCATG | |
| | CACTATTGATTCCTCTGGCCACCTTTTCAACCACAAATTTGATATATCAAACAAT | |
| | GACTCGTATTACCAATCCAACGAATACCTCAACACATTGCCACAATCCTTCGGG | |
| | CATTGGACCAGAACTTCTCAAACTAACAACTGTTCAATGGAAGATAAGCATCGG | |
| | GATGATGACAACAATAAATTATCTACCCAGCTTTCCACATTTCTAAATCCAATAA | |
| | ACTCCGACAACGTGGACAACAGTGGTCATTTATACCTTGATATCAACGCATCGA | |
| | ATACGATGGCAACAGAGTATATGCACTCAAATCAACCATCTGGCAAACCATTTG | |
| | ACCCCCTAACCCTTCTCAATAGAGTCAAGTTACGCGATTCAACAGCGCAAATTG | |
| | CATTGGCGCAGATTTTTAACGAAGCACAAGAACAGAAACAAACATTGAACAAT | |
| | GTACTGGATTCATTATTTGATAATGTAGGATATGAAGGTAATCTTTTGGAGGGC | |
| | GACTTCAAATTTCCTTTCATATCGGATAGTTTGTTAACTGCAACTATTTCATATTC | |
| | AGTAAGGACGGCAAATGACGAAATCCTTTCAATACTTGTATGTTACGCAATCAC | |
| | TAATAAGGTTAATTTACGTGCAGATTTGGTGGCAGATATAATTGATATTTTCTTC | |
| | AAATCATTAAGGTACTCTGACGCCTACAAACTCATAGATTATTGTTATAATGACA | |
| | AAACAAAGCTCGACAGCATCCTCAAAACTGATAATTTTCAGCACATGTTCAACA | |
| | TGTGCAAGGAAATTACAGTTGATAACAAGATTAGTTTTGAAATATATCAATATT | |
| | TGTTACAGTGCAATGGTGAAAAGGAGATTTTATGTAAGATAGTTGAATCCATTG | |
| | ATGAGGCAATTGATTCTGATTCTCATTTGGCTTCACTGCTCAGGACATCATCCAT | |
| | TCCATTTATTATCAGATTATTTGTGGAAAGGGGGGATTTGAATAAGTTATCATA | |
| | TTATTTGAAGATATATTTGTCGAAGCCAAGCCCTTCTACACACGATTTGGTTGAG | |
| | ATAATCGACGTCATACTGGAGACGAATAGAGCCCAACTTCAGTCTGTTGTACAA | |
| | CAAATATCAGAGTCTAACTCTTTTGATTTGACCATTTTGCTGTATATTTTCACATT | |
| | TTTGCATTTGAAAAATAAGTCCAATATCATTCATATGATCTACGCCGACGCCATT | |
| | GATAAGCAAATCCAAAACATTCCATCTGACCTTAAAGCAGTACTTACCAGCCTTT | |
| | CTATACCAATATTTTGTTTTGATTTTGCGCCTCAGTATATTTCCGACTTGCTAGAT | |
| | TCAATTCTGAGGATCTATGATACATGCAATAATAGTTTGGTCAAACGCGCCCGG | |
| | GCCATAACCTCGGTTCATCCTGCAACGGCTATGAATGAGGTCGACATTTCAAAT | |
| | GATGTGGCAAACGAAGGAGATTTTGATGATTTAGCGATATCCGAGCTGAAACG | |
| | AATGTCATTGACTCCTTCCGGAGTTGATTTTGCTGAGATTAAATCTTTGATATTT | |
| | TCGGACGATTTTTTGCCTCGAATCATACCTTTGTTAACGAACGAAAATATATTTT | |
| | GCCTACTTCAACTATCAATAGCTTCTTTTGACATAAATTCAATTGAAAAAATAAG | |
| | CTATTCAATCTCGCTTAAGGACAGCAATGATTTGATTATTGTTGCCATGATTAAC | |
| | AATGCGTTGGTCAACTACGGTTATGTTGAGAAGTCAAAGTCACTGCTTAAAAG | |
| | GGCAGAGAAGCTGATTTGCAATGTAAGACTAAGGTTGAATAATAAGTATCCAT | |
| | CCATGCCAACTGGTACTGGTTCTCCAATTAGCGATTCCTCTGGTATTGGGGTCC | |
| | GCACTGGTGGTACGAAAGAAGATAATTTCAATTTTGGCGTCTCTGACTGTTCTG | |
| | GTTCACACTTACCATTGAAACTACGTGATATATCATGGGTTTCATCTATAAGTAA | |
| | CTTGTTGTGTTCCAAAGACCTAAGCAATTCTGAGTCATTTCACCTGTTATCTTTTA | |
| | TCTCCAGTTTGTACAGTCATCCCTTCATCGTAAGCACTGTGCTCAAGAAGTTCAT | |
| | TGTGGCGCATCACATTTTGATACGTATGCTCAAAAGTAAATTGCACATAAATCA | |
| | AGCAATGATATCTGCCATATGTCAAGTATTGAATAATACCTGTTGCATAAATCA | |
| | ACTGCAACAACTACTTATGATGACCGATTTGTTACAGTACCACATGTCAAATGA | |
| | CTTTTATTCGGCTATTTTGGATGCTTGTATAAGAATTAATTCACCTGATCACCTTT | |
| | TAGAGTCTGTTAATAAATATAAAAAATTTGGATTCCACCCTGACCTCCAGACAT | |
| | ATGGCTTGTTGATCAAATTCTTCAGTTCATCAGACAATGTGATGGAGTGTTTCC | |
| | ACCTTTGGAACGAAATGACGTCCCTTTACGGTTATGAGCTGAATGAAGTAACCT | |
| | ATGGTTGCATGTTTGATGCATTAGTCTCCAACAACATGTTGGACGAGGCGTTGA | |
| | GTTTGCTCAAAGACATGAAGAAGAACTCTAATATTAAGCCAAATACAATTATTT | |
| | ATTCAACACTTATAAAGGGGTTTGGGCAGACAAAGCAGTTAAACAAGGCGTTG | |
| | AATATCTATTTGACCATGTTGGATGAAGGAGTAGTCCCCAATACTATTACGTAT | |
| | AATTCTATAATTGATGCATGTGCTCGTGTTGGGGACATGAATAAAGCTGCCAAT | |
| | TTGCTTGAGGATATGTTAAACAATAACATAGAACCTGACTTAATTACTTTCTCAA | |
| | CCGTCATTAAGGGATATTGCGTACAATCTAACATGGATCGCTCCTTGCAACTAC | |
| | TTAGGGCGATGTCTGAAAGAGGGATTAAACCCGATGGAATACTATACAATTCA | |
| | CTTCTCGATGGATGCGTAAAATCTGGCAGGCCTTGGTTATGCCAACAACTTTGG | |
| | GATGAAATGCAGGAGAACGGTATAGCACCAAGTAACTTTACCCTCACTATACTG | |
| | ATTAAAATGTATGGAAGATTAGGCCAACTAGACAAGGCGTTCCAGCTTATGGA | |
| | TGAGTTGCCCAGAAAATATAACATCCAAACTAATACGCATGTATATACGTGCCT | |
| | AATGTCTGCCTGCATCACGAATGGCAAATACAAAATGGCTTTGGACGTGTTCAA | |
| | TTGCATGAACGGCAATGGCATAGTTCCCGATTCAAAGACTTATGAGACAATAAT | |
| | ATTTGGCGCAATAAAGGGCCGTTTGCTCTACCAGGTGATCGATATTATCAAGGC | |
| | TGCCTATACCCTGATGAGCCGAGGTAATGGCACAGGTGGGCGGATGAACAAAT | |
| | CTATTTTCAAGATAGAATCTAGGATACTTAAGTTGTTTGCACAGAAAGTGGAGG | |
| | CCAGTGGCGACCCTCTGCTGCTACAACAAGCCCAATCATTGGCAGAGAATTTGA | |
| | AAAAGTTCAACATCATTCTTCCCATTCGTAGCAATTTGGTCACACAAAAAACACA | |
| | GATTAAAAAGGTATCTAACAGTAGGGCTGGCCATTACGATTCGTTTTTATTGAA | |
| | GAATGTTACGCAGTCACTTAGGAATGATTGTACCAACAACTATCGCCGAAACAG | |
| | CGTTGGTGCCACAGTATTTGCAAATAATGATGGGTTTAAGGATAATAGTGTGA | |
| | GTGATTTTAGGGAAGGAATAAATTGTTTTGGAAGCGATGATAATTTAAACAGTT | |
| | TCAAAGATGAACATTTTAACGCACACACGCAAAGAATGCACACTTTTGCAAGTG | |
| | ACATTACATGCAATTACAGAAATGATGAGAGGAATTTCCAAAAGGAAAACCGG | |
| | TCGGCCTCCGGGGGAACTACGTTAAAGGGAGTCATGTGGGATAAGGATATAG | |
| | GTGGTAATATTAACAATGCGGCTTATAGATTTGACGCCTCGTCTATACCATACA | |
| | GCGCTGAAAACGCTGCTATAGGCTCCATAAACAGCGTGTTCGGGGGTTCATAT | |
| | GCGGGTAATGGTGAGGACCATTTTTTATCGAAGAACCCGCAGTATTCCATGCA | |
| | GTTTAACCCCGCCATGGTCCCTGCTTCGAATTGTGGCAATCGCAGGGGTAAGCT | |
| | TAAATTTTGA | |
| 58 | ATGGCGCAATTAGCCGTAGAGGAGCTTAGCCCTTTTGAAAGACAAGAACTGTT | BMR1_04G05940 |
| | ATGTGTATATTCATCACTACTATTGTACGATGATGAATTGGAGATATCTAAGGA | full sequence |
| | AAACATAAACAAAGTGCTAAACAGTGCCGGTGCTAAGGTGGAGCCCTATCTTC | |
| | CAATGCTTTTTGCCAAAGCTTTGAAGGGTAAGGATTTGAATGCTTTGTTTGGTT | |
| | CAGTAGCCTCTATTGGTGCTCCTGTTGCTTCCCATGCTGCTGCCACTACTAGTGC | |
| | AGCGGTAGATGCGCCTCAGGCTGGAAAGGCCCAGGAATCAAATGTTGAAGAA | |
| | GAAGAGGATGATGATGACATGGGATTTTCACTCTTTGACTAA | |
| 59 | ATGAGGTGGAAACCAATTTACGTATTTACATCTGCTTTAATACGCCTCTTTTGCA | BMR1_02G02565 |
| | TATTTAGTTGTAATATAACATTTTCCGAGTGTATAAGGCAATCGCATGGTGTGG | full sequence |
| | ATAGGAGGAATATAAATGGTTTGTCTTTATGCACTCCTACCAATGTTCTATTCTC | |
| | GACTTTCGCATGCTATATACGACCATCAAAGATTGGCAAGAGCTACAACAATTT | |
| | GAATAAGTATAAACTTGGCACAAGTTCCAGTGAGGTTGATGGTAATGAACAAT | |
| | CAGATATATCATCTGGTTCTGATGTTGAGCGTGGAAATTTAATACAAGGGAAAA | |
| | GGCTGAAGAATTACTATCGAATTTTAAATTTGGACAAGTATGCATCTGGAGAAG | |
| | ACATCAAGAATCAGTATGAAAATCTGATTGAATCACTTAAACCGCTTGAAAATG | |
| | TAGATTCCAATGTTATAGATATGATAAATGAAGCCTATAGGATCCTATCTGATG | |
| | AAAATACTCGACGCATTTACGATGAGTTGGTGGCAAAAAAATCTCTAGAGAAG | |
| | GAAAGCGGATATAATGATAGTAATATTGACCAATTTTATCACGATGATGGTGAT | |
| | TACTTCCCAGAAAATCTTTACAACGTTTTAGAGTCAGATTACGACTTTACCAGTG | |
| | ATTCTGACGAAATGGTTCTAGAGATGAGCACAGATGATGATTATTCTGATGAA | |
| | GATTCTAATGAAATGATCAGCTTGATACCCAAATTGATTAAACCAAACGGAACA | |
| | AAATTGCATACAACACTTACCATCCCCTTTGAAAGAGCAATTATGGGTGGAAAT | |
| | GAGACAGTGACAATTTCGAGGCTTGAAAATTGTAAATGTTTGGAAAATTTAACT | |
| | ACATGTAAGAGTTGCAATGGATATGGTCTGGATGGGAAGGATAAATTGGGAA | |
| | CGGGATTCGTATCATCAAAGGAATGTTCGGAATGTGGAGGAATTGGTAAATCT | |
| | AGGGCGAAAAAATGTGATTTATGTGACAATACTGGACAAGTTAAAGTTGATAA | |
| | TGCAACTATTCAAGTGCAGGTTCCGCGAAATGTATATGACGGCGCCAGGCTTTT | |
| | GATTAGGAACCAGGGAAATGTATATGGCACTAATGGCAAAGCTGGTGATTTAG | |
| | TTGTAACTTTACGAGTAAAAGAGCATGATCATATGTATAGAATGGGTAAAAATA | |
| | TATATTCCGACGTCACAGTGCCATATGCAGCTGCTATACTCGGTACTACTATAAA | |
| | ATTGGAAACATGTGGAGGAGTTGTATCGCTTGAAATCCCTCCCGGTACTCAACA | |
| | TGGAGATGAAATACAAGTTCCAAACGAGTCTATTCCCATGAAACACATTTGTAG | |
| | AATTGAGGTTGCGCTGCCTAAATCTGTAGATAAACAAGAAAGGAGTTTGTTGG | |
| | AAAAGATAATCCAGCTTAAGTAG | |
| 60 | ATGGATCAATTAAAAGATTCCATTGATCAATCAAATATTGATCGAAAGGCAGCT | BMR1_04G06705 |
| | CTGGAGGCTATTGAATCCTTTCTTTCCTCTAAAATACAAGCCGCTAATGCGGGG | full sequence |
| | AGTATTCGTGGGAAAAAGAATCCAGCTGTTAACGATTTTAATAACCCGCCAATA | |
| | GTACAGTCATATTTATCCGAGGATGAGGGGGTAACCAGTATATCTGAATATAA | |
| | AAATGGAATATCCGACCCCTTTTACAATATTGCCGTAGGTACCAATCATTCCATT | |
| | TCCTCGGCGAAAAATATACCTGTATCGTATCAGCCGAATCACACTGATACAAAT | |
| | AGAGAAACGCCCCTGATACAACGTAGTATTGCCTATTTTAAAAATAAAACTAAA | |
| | TTCCTCTCCAAAATGGACACGTTTACCTCGGCGTTGATCCCCGCTGCCATTACCC | |
| | TGGTCATTACTAACTCAGCACAACCACTACTTATATTCTTGCTTAGAAAATATGG | |
| | AGGGACTCCGGTGGGTGCCTACTTCTTCCTATTCCCAACCTATCTCGGCATGATC | |
| | TGCGTCGGCCTATATCCAACCAAAAAACCCGTATGGAAGGAAAATTGGATTTAC | |
| | CCCGGAGTATTAGCTGGAATTGACTTCTTGCATCAGCTTATTGAAAAGGCTGGG | |
| | TTGCTATACTGCGGTCCATGCCTATACACTGTGGCCAGCAGCAGCAATACTCTG | |
| | TTCCTAGCGCTATTTACTTCCATTTATTTAAATGTAAAAATAACTAGAAATACTG | |
| | CAATTTCATTGACTATTATTTCAATTGCAGTGTCATTTAGTGGTTCGGGAAAATT | |
| | GTGCGAAATAAACTCAACACATTTGATCGGCTTTACATTGAACGCATTCAACTTT | |
| | GTGATAGGAGAGATCATACTCAAAGAAAATAAAATCGAAGGCCCAAACCTCGT | |
| | CTGTATAATGGGATTCATATCCTTTATCTCTCTCACCCTCTGGACCTGCGTTTGG | |
| | ACCATTCCAAACTGGAGTACAATCGCTCATCATACGGAAATGAATGTATATGCC | |
| | ATTTTCATTATTTTATTTGTCCTGTTTATATCAAATTTCATACGGTCTTCTGTATAT | |
| | TGGATACTCATAAAACGTGCTGGATCTCTATTCACAGGTGTGCTAAAGGCGCTC | |
| | CGAATTGTTATTGTTATTGTAATAAGTCACATTTTGTTCTCCCACATAGATCCCAT | |
| | GCAGAGGATCACATTCACCAAAATCTTCACCGCTCTGCTGTGTTCAACCGGTAT | |
| | CGTGATCTATTCAATAGACAACAATAATAAGATTGATAACTATAAGAATGAAAT | |
| | GAAGGGGGAGAGAGAAGCGGTTGAGGATGGCGAAGACGAAAAAATCATGGT | |
| | AAATGATGATGTTTAA | |

Anti-Babesia Microti Antibody-Based Compositions

Also provided herein are compositions that include one or more (e.g., 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, or 24 or more) antibodies or antigen-binding fragments thereof which specifically bind to one or more Bm antigens or antigenic fragments thereof. These compositions optionally include a pharmaceutically acceptable excipient, preservative, carrier or diluent.

In some embodiments, the one or more antibodies bind to one or more Bm antigens that each comprise an amino acid sequence having at least 80% sequence identity (e.g., at least 80%, 85%, 90%, 95%, 97%, 99%, or greater) to the amino acid sequence of SEQ ID NO: 1, or one or more antigenic fragments thereof. In some embodiments, the one or more antibodies bind to one or more Bm antigens that each comprise an amino acid sequence having at least 80% sequence identity (e.g., at least 80%, 85%, 90%, 95%, 97%, 99%, or greater) to the amino acid sequence of SEQ ID NO: 2, or one or more antigenic fragments thereof. In some embodiments, the one or more antibodies bind to one or more Bm antigens that each comprise an amino acid sequence having at 80% sequence identity (e.g., at least 80%, 85%, 90%, 95%, 97%, 99%, or greater) to the amino acid sequence of SEQ ID NO: 3, or one or more antigenic fragments thereof. In some embodiments, the one or more antibodies bind to one or more Bm antigens that each comprise an amino acid sequence having at least 80% sequence identity (e.g., at least 80%, 85%, 90%, 95%, 97%, 99%, or greater) to the amino acid sequence of SEQ ID NO: 4, or one or more antigenic fragments thereof. In some embodiments, the one or more antibodies bind to one or more Bm antigens that each comprise an amino acid sequence having at least 80% sequence identity (e.g., at least 80%, 85%, 90%, 95%, 97%, 99%, or greater) to the amino acid sequence of SEQ ID NO: 5, or one or more antigenic fragments thereof. In some embodiments, the one or more antibodies bind to one or more Bm antigens that each comprise an amino acid sequence having at least 80% sequence identity (e.g., at least 80%, 85%, 90%, 95%, 97%, 99%, or greater) to the amino acid sequence of SEQ ID NO: 6, or one or more antigenic fragments thereof. In some embodiments, the one or more antibodies bind to one or more Bm antigens that each comprise an amino acid sequence having at least 80% sequence identity (e.g., at least 80%, 85%, 90%, 95%, 97%, 99%, or greater) to the amino acid sequence of SEQ ID NO: 7, or one or more antigenic fragments thereof. In some embodiments, the one or more antibodies bind to one or more Bm antigens that each comprise an amino acid sequence having at least 80% sequence identity (e.g., at least 80%, 85%, 90%, 95%, 97%, 99%, or greater) to the amino acid sequence of SEQ ID NO: 8, or one or more antigenic fragments thereof. In some embodiments, the one or more antibodies bind to one or more Bm antigens that each comprise an amino acid sequence having at least 80% sequence identity (e.g., at least 80%, 85%, 90%, 95%, 97%, 99%, or greater) to the amino acid sequence of SEQ ID NO: 9, or one or more antigenic fragments thereof.

In some embodiments, the one or more antibodies bind to one or more Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-3, or one or more antigenic fragments thereof. In some embodiments, the one or more antibodies bind to one or more Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-3, or one or more antigenic fragments thereof, and to one or more Bm antigens that each comprise an amino acid sequence having at least 80% sequence identity (e.g., at least 80%, 85%, 90%, 95%, 97%, 99%, or greater) to the amino acid sequence of any one of SEQ ID NOs: 4-9, or one or more antigenic fragments thereof. In some embodiments, the one or more antibodies bind to one or more Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-9, or one or more antigenic fragments thereof. In some embodiments, the one or more antibodies bind to one or more Bm antigens that each comprise an amino acid sequence having at least 80% sequence identity (e.g., at least 80%, 85%, 90%, 95%, 97%, 99%, or greater) to the amino acid sequence of any one of SEQ ID NOs: 1-24, or one or more antigenic fragments thereof.

Antibodies and antigen-binding fragments, variants, or derivatives thereof include, but are not limited to, monoclonal, genetically engineered, and otherwise modified forms of antibodies, including, for example, chimeric antibodies, humanized antibodies, primatized antibodies, heteroconjugate antibodies (e.g., bi-, tri-, and quadri-specific antibodies), and antigen-binding fragments of antibodies including Fab, Fab′, F(ab′)₂, Fv, scFv, tandem scFv, diabody, triabody, tetrabody, small modular immunopharmaceutical (SMIP), nanobody or other single domain antibody. Also included are affibodies, isolated CDRs, and a combination of two or more isolated CDRs.

Antibodies can be generated using any of the numerous methods for making antibodies known in the art. Monoclonal antibodies that specifically bind to one or more Bm antigens can be prepared using standard hybridoma technology (see, e.g., Kohler et al., Nature 256:495-7, 1975; Kohler et al., Eur. J. Immunol. 6:511-9, 1976; Kohler et al., Eur. J. Immunol. 6:292-5, 1976; Hammerling et al., Monoclonal Antibodies and T Cell Hybridomas, Elsevier, N.Y., 1981). Antigen-binding fragments can be produced by recombinant DNA techniques, enzymatic or chemical cleavage of intact antibodies, or, in certain cases, by chemical peptide synthesis procedures known in the art. Antigen-binding fragments also can be identified by screening a phage display library (Vaughan et al., Nat. Biotechnol. 14:309-14, 1996) using a polypeptide of the invention described above. See, for example, the references cited herein above, as well as Zhiqiang An (Editor), Therapeutic Monoclonal Antibodies: From Bench to Clinic. 1st Edition. Wiley 2009, and also Greenfield (Ed.), Antibodies: A Laboratory Manual. (Second edition) Cold Spring Harbor Laboratory Press 2013, for methods of making recombinant antibodies, including antibody engineering, use of degenerate oligonucleotides, 5′-RACE, phage display, and mutagenesis; antibody testing and characterization; antibody pharmacokinetics and pharmacodynamics; antibody purification and storage; and screening and labeling techniques.

Useful antibodies can be identified using one of several screening assays, including ELISA, immunoprecipitation or Western blot analysis. In one example, antibodies are assayed by ELISA to determine whether they are specific for the immunizing antigen (e.g., one or more Bm polypeptides as described herein). Using standard techniques, some wells of a plate are coated with the immunogen whereas other wells are coated with irrelevant Bm antigens. An aliquot of the antibody sample is added to each well of the plate. The unbound material is washed, and the bound antibody detected by use of a second antibody specific for the immunoglobulin of the species in which the antibody was generated. Antigen-binding fragments can be screened for utility in the same manner as intact antibodies.

In some embodiments, the anti-Bm antibodies used in the therapeutic or prophylactic methods of the invention can be purified from cells known to produce monoclonal antibodies, e.g., hybridomas as understood by those of skill in the art. In other embodiments, the anti-Bm antibodies used in the therapeutic or prophylactic methods of the invention can be artificially manufactured molecules, e.g., recombinant antibodies. In some embodiments, a therapeutic or prophylactic antibody may comprise monoclonal antibodies along with other components such as excipient, diluent, or carrier (e.g., human serum albumin). A therapeutic or prophylactic antibody may further comprise preservatives or combinations thereof, as will be readily understood by those of skill in the art.

Pharmaceutical Formulations

The Bm antigens, the Bm nucleic acid molecules, and the Bm-specific antibodies described herein can be formulated into pharmaceutical compositions that are biologically compatible and suited for administration to a subject

Compositions may be administered to a subject in a variety of forms which depend on the route of administration, as will be understood by those skilled in the art. The compositions described herein may be administered by the oral route or a parenteral route, including, for example, the intravenous, intraperitoneal, subcutaneous, intradermal, intramuscular, and topical routes.

A composition described herein may be orally administered, for example, with an inert diluent or an edible carrier. The composition may be enclosed in hard- or soft-shell gelatin capsules, or compressed into tablets, or incorporated directly with the food of the diet. A composition described herein may be incorporated with an excipient and used in the form of ingestible tablets, buccal tablets, troches, capsules, elixirs, suspensions, syrups, and wafers. Compositions suitable for buccal or sublingual absorption include tablets, lozenges, and pastilles, where the active ingredient is formulated with a carrier, such as sugar, acacia, tragacanth, gelatin, and glycerin. Nucleic acid-based compositions can optionally be formulated in a drug delivery vehicle such as, e.g., a lipid nanoparticle or a dendritic cell, in the case of an RNA vaccine. DNA vaccines, e.g., plasmid vaccines, can be formulated in saline or sucrose solutions, as is known in the art.

A composition described herein may also be administered parenterally. Solutions of a composition described herein can be prepared in water suitably mixed with a surfactant, such as hydroxypropylcellulose. Dispersions can also be prepared in glycerol, liquid polyethylene glycols, DMSO, and mixtures thereof with or without alcohol, and in oils. Under ordinary conditions of storage and use, these preparations may contain a preservative. Conventional procedures and ingredients for the selection and preparation of suitable formulations are described, for example, in Remington's Pharmaceutical Sciences (2012, 22nd ed.) and in The United States Pharmacopeia: The National Formulary (USP 41 NF 36), published in 2018. The pharmaceutical forms suitable for injectable use include dispersions or aqueous solutions, and powders for the extemporaneous preparation of injectable dispersions or aqueous solutions. In all cases, the forms must be sterile and must be fluid to the extent that they may be easily administered via syringe. Compositions for injectable use may be administered by continuous infusion, single or multiple boluses, or through the use of microneedles. Compositions suitable for topical administration include lotions, creams, ointments, gels, foam, transdermal patches, pastes, and tinctures. Nucleic acid-based compositions, such as DNA vaccines, can be administered by injection (e.g., intramuscular or intradermal injection). DNA vaccine administration can optionally include the use of a gene gun and, optionally, gold or tungsten microparticles onto which the DNA has been absorbed.

The compositions described herein may be administered to a subject, e.g., a human, alone or in combination with pharmaceutically acceptable carriers, as noted herein, the proportion of which is determined by the solubility and chemical nature of the composition, the route of administration, and standard pharmaceutical practice.

### Methods

The invention provides methods for determining whether a subject has natural protective immunity against Bm; immunizing a subject against Bm; determining whether a prophylactic regimen has conferred protective immunity against Bm; identifying a subject who is likely to benefit from an anti-Bm antibody based therapy; treating a subject having babesiosis with an anti-Bm antibody based therapy; and optimizing administration of an anti-Bm antibody regimen. The methods described herein are based on the findings that a) resolution of B. microti infection in cd4-deficient mice requires antibody-producing B cells and antibody class switching, b) such resolution is concomitant with the accumulation of B. microti-specific IgG antibodies in blood, and c) these IgG antibodies target a restricted set of B. microti polypeptides.

Methods for Monitoring Immune Protection

The invention provides methods for detecting particular combinations of a subset of Bm-specific antibodies that can serve as indicators of protection against Bm, including protection acquired naturally during the course of an infection with Bm, protection induced by a Bm antigen-containing composition, a Bm antigen-encoding composition, or an anti-Bm antibody composition so as to achieve prophylaxis against Bm, and protection induced by an anti-Bm antibody composition so as to treat a subject experiencing babesiosis.

The invention provides methods for determining whether a subject has protective immunity against Bm. In certain instances, the methods include the use of immunoassays. In one example, the methods include applying a body fluid sample from a subject to a solid support containing one or more Bm antigens or antigenic fragments thereof (e.g., as described herein); applying an antibody detection agent to the solid support; and determining whether the fluid sample contains IgG antibodies that are specifically reactive to the one or more Bm antigens or antigenic fragments thereof. In certain embodiments, the methods further include identifying the subject as likely to have protective immunity against Bm if the sample from this subject is determined to have a sufficient titer of one or more IgG antibodies that bind to one or more Bm antigens or antigenic fragments thereof, that is, a titer in the fluid sample that is above a cutoff titer.

The invention also provides methods for identifying a patient experiencing babesiosis as likely to benefit from an anti-Bm antibody-based therapy. In certain instances, the methods include the use of immunoassays. In one example, the methods include applying a body fluid sample from a subject to a solid support containing one or more Bm antigens or antigenic fragments thereof (e.g., as described herein); applying an antibody detection agent to the solid support; and determining whether the fluid sample contains antibodies that are specifically reactive to the one or more Bm antigens or antigenic fragments thereof. In certain embodiments, the methods further include identifying the subject as likely to benefit from an anti-Bm antibody based therapy if the sample from the subject is determined to have an insufficient titer of one or more IgG antibodies specific for one or more Bm antigens or antigenic fragments thereof, that is, a titer in the fluid sample that is below a cutoff titer. In some embodiments, the methods further include administering to the subject an effective amount of a composition comprising one or more anti-Bm antibodies if the subject has been identified as likely to benefit from administration of the one or more anti-Bm antibodies.

The invention further provides methods for optimizing the administration of an anti-Bm antibody-based therapy to a subject experiencing babesiosis. In certain instances, the methods include the use of immunoassays. In one example, the methods include applying a body fluid sample from a subject to a solid support containing one or more Bm antigens or antigenic fragments thereof (e.g., as described herein); applying an antibody detection agent to the solid support; and determining whether the fluid sample contains antibodies that are specifically reactive to the one or more Bm antigens or antigenic fragments thereof. In certain embodiments, the methods further include administering to the subject an effective amount of a composition comprising one or more anti-Bm antibodies if the sample from the subject is determined to have an insufficient titer of one or more IgG antibodies specific for one or more Bm antigens or antigenic fragments thereof, that is, a titer in the fluid sample that is below a cutoff titer.

Any of the compositions described herein may be used in conjunction with any of the above-described methods. In certain embodiments, the one or more Bm antigens or antigenic fragments thereof (e.g., as described herein) are provided as an array affixed to a solid phase. In other embodiments, antibody reactivity is determined by ELISA, western blot analysis, or rapid diagnostic tests such as a lateral flow assay, or an assay employing a microfluidic device.

In some embodiments, the body fluid is a blood sample or a cell-free fraction thereof (e.g., a serum or plasma sample). In certain embodiments, the subject from whom body fluid is acquired was previously immunized with a Bm antigen-containing composition, a Bm antigen-encoding composition, or an anti-Bm antibody composition (e.g., a composition described herein). In certain embodiments, the detection of certain Bm-specific antibodies in body fluids of previously immunized individuals is used to ascertain immune protection against Bm. In other embodiments, the subject from whom body fluid is acquired was previously treated with an anti-Bm antibody composition (e.g., a composition described herein). In some embodiments, the detection of certain Bm-specific antibodies in body fluids of individuals previously treated with therapeutic antibodies is used to ascertain immune protection against babesiosis.

Methods for Conferring Prophylaxis

The invention provides methods for immunizing or conferring protective immunity against Bm in a subject who does not experience babesiosis. In some embodiments, the methods include administering to the subject a composition including one or more Bm antigens or antigenic fragments thereof (e.g., as described herein). In some embodiments, the methods include administering to the subject a composition including one or more nucleic acid molecules (e.g., DNA or RNA) encoding one or more Bm antigens or antigenic fragments (e.g., as described herein). In other embodiments, the methods include administering to the subject a composition including one or more antibodies or antigen-binding fragments thereof that specifically bind to one or more Bm antigens or antigenic fragments thereof (e.g., as described herein).

In some instances, the subject has been determined to be at risk of Bm infection or risk of experiencing babesiosis. In certain instances, the subject has been determined to have a titer of IgG antibodies specific for one or more Bm antigens (e.g., as described herein) that is below a cutoff titer in accordance with the diagnostic methods described herein. In some embodiments, administration of a composition as described herein for prophylaxis against Bm reduces duration and/or severity of a future episode of babesiosis.

Methods for Treating Babesiosis

The invention provides methods for treating a subject who experiences babesiosis (e.g., mild or severe, including persistent or relapsing babesiosis). In some embodiments, the methods include administering to the subject an effective amount of a composition comprising one or more anti-Bm antibodies or antigen-binding fragments thereof that are specific for one or more Bm antigens (e.g., as described herein).

The invention also provides methods for supplementing an immune response against Bm in a subject who experiences babesiosis (e.g., mild or severe, including persistent or relapsing babesiosis). In some embodiments, the methods include administering to the subject an effective amount of a composition comprising one or more anti-Bm antibodies or antigen-binding fragments thereof that are specific for one or more Bm antigens (e.g., as described herein). In certain embodiments, prior to administration, the subject has been determined to have insufficient titers of IgG antibodies specific for one or more Bm antigens, that is, titers that are below cutoff titers for these Bm antigens.

In some embodiments of any of the above-described methods, treatment reduces duration and/or severity of symptoms of babesiosis. In other embodiments, treatment reduces duration of infection with the etiologic agent of babesiosis. In some embodiments, the etiologic agent of babesiosis is Babesia microti.

Selection of Subjects

Subjects who may benefit from the methods described herein are subjects who experience or are at risk of experiencing babesiosis (e.g., mild or severe babesiosis, including persistent or relapsing babesiosis). Patients at risk of experiencing mild babesiosis include otherwise healthy individuals of age 40 years and above. Patients at risk of experiencing severe babesiosis include those who lack a spleen; those who suffer from anatomical or functional hyposplenism; those who are or were recently treated with rituximab or any other antibody that depletes mature B cells, including those diagnosed with a B cell lymphoma or an autoimmune disease; those for whom the CD4 T cell compartment has been depleted, including those experiencing HIV/AIDS or treated with an immunosuppressive therapy for stem cell or solid organ transplantation; and those who are immunosuppressed by treatment of comorbidity, including those who suffer from a malignancy and those who are treated from a chronic inflammatory disorder. Patients at risk of developing mild or severe babesiosis are individuals who are exposed to ticks and those who are transfused with blood products, particularly packed red blood cells.

Subjects who may be treated using the methods described herein include those who received a diagnosis of babesiosis that was made following the standard and routine diagnostic procedures known in the art. An exemplary diagnostic procedure is the microscopic evaluation of thick or thin blood smears after exposure to the Giemsa stain or the Wright stain. Another exemplary diagnostic procedure is the amplification of Babesia genomic DNA by use of the polymerase chain reaction and of Babesia specific primers. In certain embodiments, a subject has already undergone treatment for babesiosis but such treatment has not been sufficient to achieve cure. In other embodiments, the subject has yet to be treated for babesiosis.

Doses and Dosages

A composition of the invention is administered to a subject (e.g., a mammal, such as a human or mouse) in an effective amount, which is an amount that produces a desirable effect in the treated subject. Effective and optimal doses and dosages for the compositions of the invention can be determined using methods known in the art. Single or multiple administrations of the compositions of the invention can be carried out to attain the desirable effect. Doses and dosages can be selected by the treating physician. It is anticipated that optimal doses and dosages will vary with the age, weight, and health of the subject. It is also anticipated that optimal doses and dosages will vary with the mode of administration, that is, a lower amount of the composition will be needed to attain the desirable effect when administered by the intravenous route as compared with other parental routes such as the intradermal, subcutaneous and intramuscular routes.

Guidance to identify effective and optimized doses and dosages of the composition is provided in the exemplified approach described herein. For example, in the context of prophylaxis, subjects are immunized with varying doses at varying intervals, and are evaluated for the titers of antibodies that are specific for one or more Bm antigens. Immunized subjects also are evaluated for their protection from babesiosis caused by a subsequent challenge with a Babesia species. Such results can be used to optimize doses and dosages required for effective immunization of subjects, e.g., humans, mice and other mammals. Results can be extrapolated by persons having skill in the art.

In the context of prophylaxis, compositions of the invention are administered to a subject (e.g., a human or mouse) in an amount sufficient to delay, reduce, or preferably prevent the onset of the disorder (e.g., babesiosis). By way of example, a composition comprising one or more Bm antigens or antigenic fragments thereof, or one or more nucleic acid molecules encoding one or more Bm antigens or antigenic fragments thereof, may be administered to a subject at a dose in a range of 1 μg/kg to 1,000 μg/kg (e.g., 1 to 1,000 μg/kg, 5 to 1,000 μg/kg, 10 to 1,000 μg/kg, 1 to 750 μg/kg, 5 to 750 μg/kg, 10 to 750 μg/kg, 1 to 500 μg/kg, 5 to 500 μg/kg, 10 to 500 μg/kg, 1 to 100 μg/kg, 5 to 100 μg/kg, 10 to 100 μg/kg, 1 to 50 μg/kg, 5 to 50 μg/kg, or 10 to 50 μg/kg).

In therapeutic applications, compositions of the invention are administered to a subject (e.g., a human or mouse) already suffering from babesiosis in an amount sufficient to achieve cure, reduce the severity of one or more symptoms associated with babesiosis, or prevent or reduce the complications of babesiosis. By way of example, a composition comprising one or more anti-Bm antibodies or antigen-binding fragments thereof may be administered to a subject at a dose in a range of 50 mg/kg to 2,000 mg/kg (e.g., 50 to 2,000 mg/kg, 100 to 1,500 mg/kg, 200 to 1,000 mg/kg, 300 to 750 mg/kg, or 400 to 500 mg/kg).

Combination Treatments

A pharmaceutical composition including one or more anti-Bm antibodies or antigen-binding fragments thereof that specifically bind one or more Bm antigens as described herein, can be administered alone or in combination with one or more therapeutic agents. For example, a pharmaceutical composition including one or more anti-Bm antibodies or antigen-binding fragments thereof that specifically bind one or more Bm antigens as described herein can be administered in combination with standard-of-care antibiotics used in the treatment of babesiosis. Antibiotic regimens used to treat babesiosis include two-drug regimens (e.g., atovaquone and azithromycin; atovaquone and clindamycin; or clindamycin and quinine), three-drug regimens (e.g., atovaquone, azithromycin, and clindamycin; or atovaquone, proguanil, and azithromycin), four-drug regimens (e.g., atovaquone, azithromycin, clindamycin, and quinine; or atovaquone, proguanil, azithromycin, and clindamycin). In combination treatments, one or more of the therapeutic agents may be administered at a lower dose or dosage than the standard dose and dosage used when administered alone. If combined, therapeutic agents should be administered at doses and dosages that provide a therapeutic effect. Doses and dosages may be determined empirically from combinations and permutations or may be deduced by isobolographic analysis (e.g., Black et al., Neurology 65:S3-S6, 2005).

Evaluation of Efficacy

The efficacy of the methods and compositions of the invention in preventing or treating babesiosis can be readily ascertained by those of skill in the art by means of (a) evaluating clinical manifestations associated with Babesia infection, including elevated temperature, chills, sweats, anorexia, headache, or (b) detecting abnormal laboratory parameters associated with Babesia infection, including low red blood counts, low platelet counts, elevated liver enzymes, or (c) monitoring the presence of Babesia parasites by microscopic evaluation of peripheral blood smears or PCR-based amplification of Babesia DNA. Thus, according to the methods of the present invention, a subject shows little to no clinical manifestations and/or few to no laboratory parameter abnormalities when titers of IgG antibodies specific for one or more Bm antigens are above cutoff titers for these Bm antigens.

### Kits

The invention also provides kits or articles of manufacture containing materials useful for the prevention of babesiosis, treatment of babesiosis, and/or monitoring of individuals undergoing prophylactic or therapeutic administration of the compositions of the invention.

Any of the compositions described herein can be provided in a kit for use in accordance with any of the prophylactic and therapeutic methods described herein. By way of example, a kit of the invention may include one or more Bm antigens or antigenic fragments thereof, as described herein. By way of another example, a kit of the invention may include one or more nucleic acid molecules (e.g., DNA or RNA) that encode one or more Bm antigens or an antigenic fragment thereof. By way of another example, a kit of the invention may include one or more antibodies or antigen-binding fragments thereof that are specific for one or more Bm antigens, as described herein. These kits can include a package insert that instructs a user of the kit, such as a physician, to perform the methods described herein. These kits may optionally include a syringe and a needle or another device for administering the composition.

Any of the composition comprising of one or more Bm antigens can be provided in a kit for use in accordance with the methods described herein to monitor individuals undergoing prophylactic or therapeutic administration of any one of the compositions of the invention. In one example, such a kit includes (a) one or more Bm antigens or antigenic fragments thereof, as described herein, optionally immobilized on one or more solid supports; (b) an antibody detection reagent; and (c) a package insert comprising instructions for using the one or more Bm antigens and the antibody detection reagent in accordance with any of the methods described herein.

### EXAMPLES

The following examples illustrate certain aspects of the invention and are not to be considered as limiting the scope thereof.

### Example 1: Host IgG Antibodies are Required for Resolution of Bm Infection in cd4-Deficient Mice

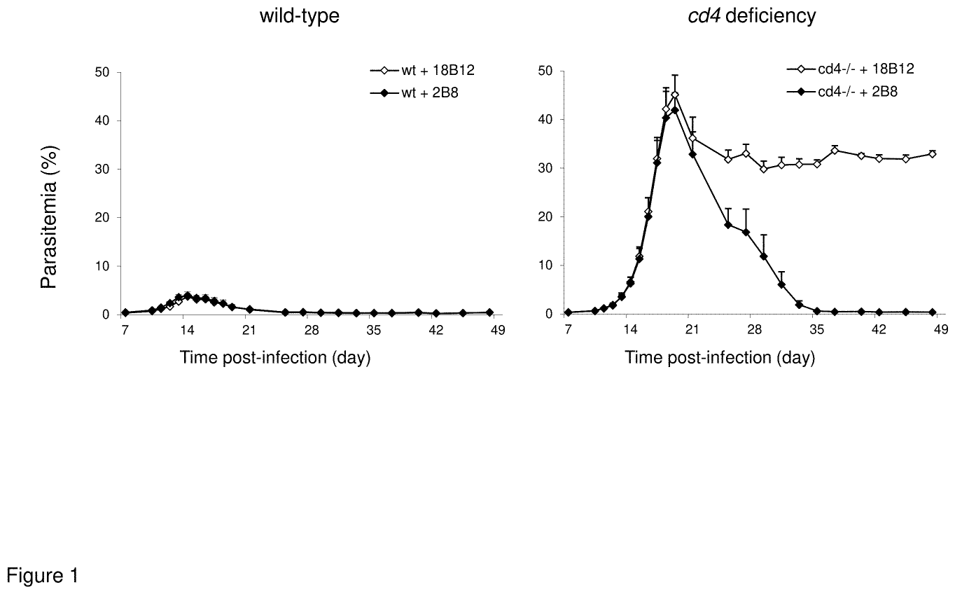

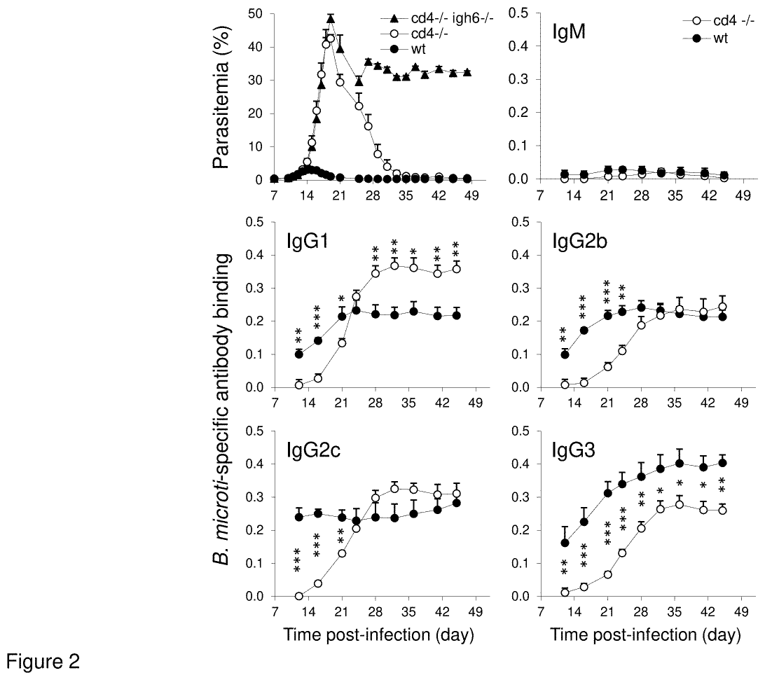

Mice expressing the cd4 gene (“WT mice”) or lacking the cd4 gene (“cd4-deficient mice” or “cd4-/- mice”) were infected with Bm and monitored over time for Bm parasitemia, i.e., the frequency of red blood cells infected with Bm (FIG. 1). Cd4-deficient mice experienced higher peak parasitemia than WT mice but mice from both strains resolved the infection. Depletion of B cells, antibody-producing cells, by administration of 18B12, a monoclonal antibody directed against mouse CD20, did not alter the inherent resistance of WT mice but prevented the resolution of Bm infection in cd4-deficient mice (FIG. 1). Administration of 2B8, an irrelevant monoclonal antibody that recognizes human CD20 but not mouse CD20, failed to alter the course of parasitemia in WT mice and in cd4-deficient mice (FIG. 1). The importance of B cells to the resolution of Bm parasitemia in cd4-deficient mice was confirmed by the persistence of Bm parasitemia in cd4-deficient mice that lacked the igh6 gene (FIG. 2).

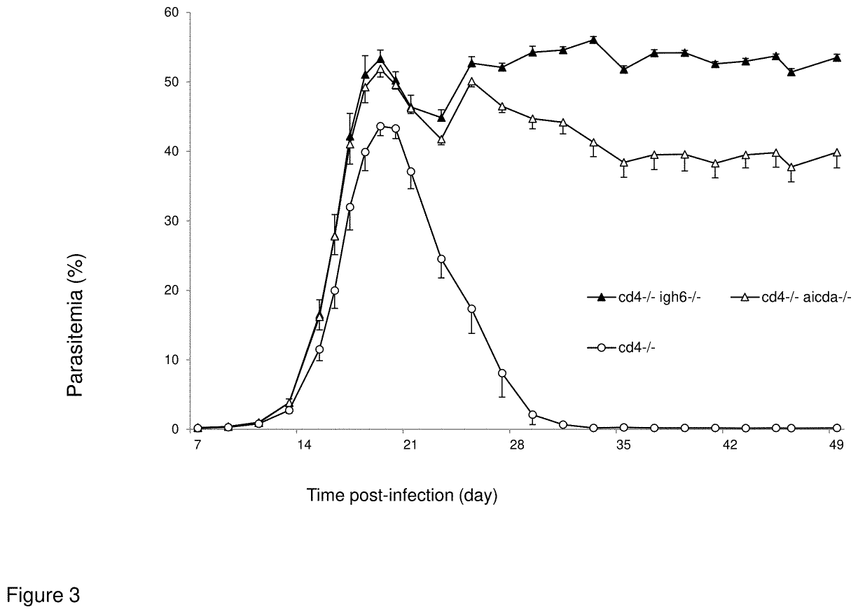

In cd4-deficient mice, resolution of Bm parasitemia was concomitant with the accumulation in blood of Bm-specific IgG antibodies, but not of Bm-specific IgM antibodies (FIG. 2). To test the hypothesis that IgG antibodies are critical for resolution of parasitemia, cd4-deficient mice that lack the aicda gene were generated. Lack of AICDA, the enzyme required for antibody class switching, including to the IgG class, prevented full resolution of Bm parasitemia in cd4-deficient mice (FIG. 3). Overall, these results strongly suggest that IgG antibodies are required for full resolution of Bm parasitemia in cd4-deficient mice.

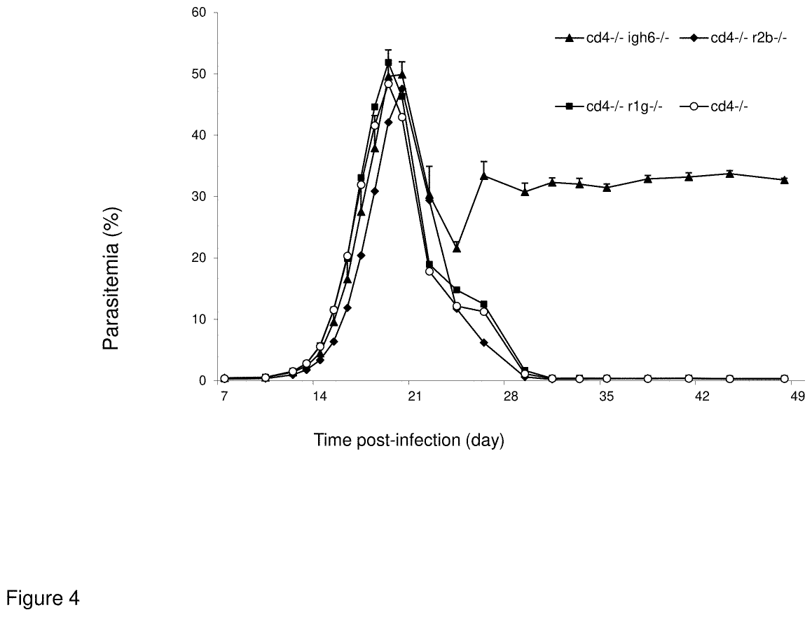

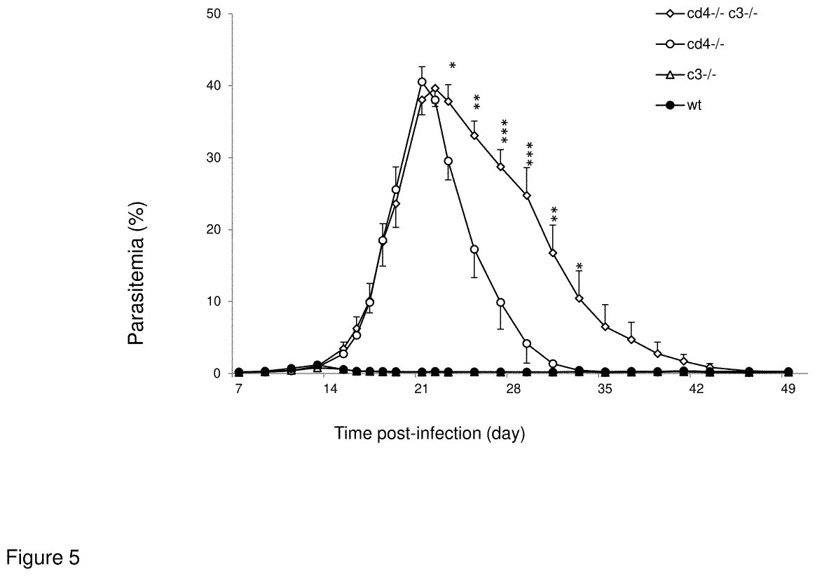

To assess whether IgG antibodies protect from persistent Bm parasitemia by engaging Fc receptors, cd4-deficient mice that lack fcerlg, the gene which encodes the chain common to all activating Fcy receptors, or fcgr2b, the inhibitory Fcy receptor, were generated. Lack of either receptor did not modify the resolution of Bm parasitemia in cd4-deficient mice (FIG. 4), indicating that the Fc portion of IgG antibodies is dispensable for resolution of Bm parasitemia. To test whether IgG antibodies protect from persistent Bm parasitemia by activating the complement system, cd4-deficient mice that lack c3, the gene that encodes the complement component C3, were generated. Lack of c3 delayed but did not prevent full resolution of Bm parasitemia in cd4-deficient mice (FIG. 5), indicating that the complement component C3 and the downstream effectors of the complement system which include C5 and the terminal membrane attack complex are not required for resolution of Bm parasitemia in cd4-deficient mice. Moreover, Bm parasitemia in C57BL/6 mice that lacked c3 was as low as that in wild-type C57BL/6 mice, indicating that neither the complement component C3 nor its downstream effectors are critical for host resistance to Bm. Taken together, these observations indicate that antibody-mediated neutralization of Bm antigens can protect a host from persistent Bm infection even when the Fc portion of the antibodies is removed or when the antibodies are designed to leave the complement system intact.

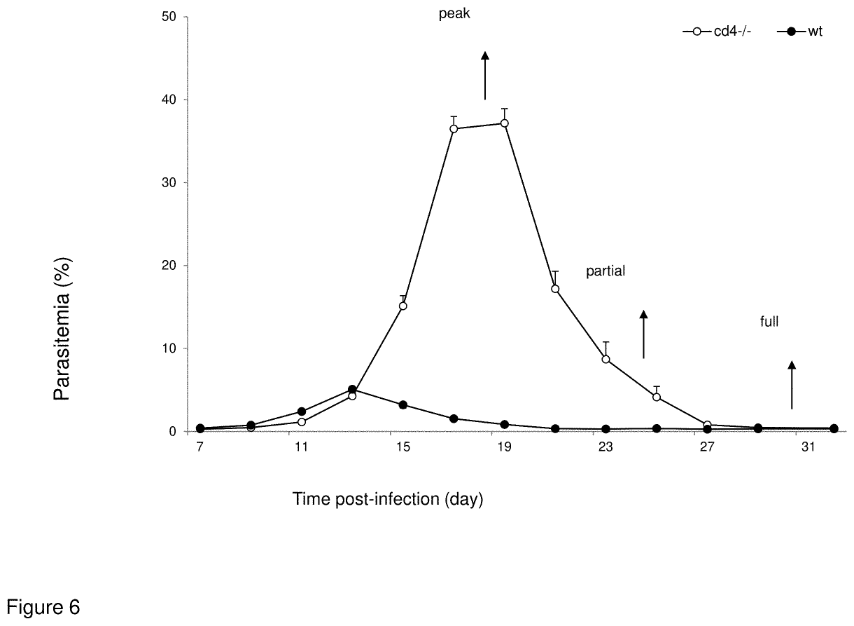

To characterize the IgG repertoire produced by cd4-deficient mice following Bm infection, peripheral blood was collected at times of peak parasitemia, partial resolution, and full resolution (FIG. 6). Peripheral blood was also collected from wild-type mice infected with Bm (FIG. 6). For each blood sample, plasma was separated and probed for IgG reactivity to Bm antigens using microarray chips, as described in Example 2, below.

### Example 2: A Few Bm Proteins are Targeted by Host IgG Antibodies in cd4-Deficient Mice

Proteome Microarray Chip Fabrication and Design

A large number of Bm genes (˜91% coverage of the LabS1 strain genome) were cloned into the expression vector pXT7. Included were the genes corresponding to SEQ ID NOs: 1-24. Custom polymerase chain reaction (PCR) primers comprising 20-bp gene-specific sequences tagged with 33-bp adapter sequences were used to amplify Bm genomic DNA. These adapter sequences, which flank the target amplicons, were homologous to adapter sequences found at the ends of the linearized T7 expression vector pXT7. Such homology allowed amplicons to be cloned by in vivo homologous recombination in competent DH5a cells. The clones were verified by amplifying the inserted genes using sequence-specific PCR primers and sequencing the resulting amplicons. For protein microarray chip fabrication, Bm proteins were expressed in an E. coli-based cell-free in vitro transcription and translation (IVTT) system (RTS 100 E. Coli HY Kit from BiotechRabbit, Berlin, Germany) according to manufacturer's instructions. Expressed Bm proteins were printed onto nitrocellulose-coated glass AVID slides (Grace Bio-Labs, Inc., Bend, Oreg.) using an OmniGrid Accent microarray printer (DigiLab, Inc., Marlborough, Mass.). Fabricated proteins microarray chips were QC'ed using monoclonal anti-polyhistidine (clone His-1; Sigma-Aldrich, St. Louis, Mo.) and anti-hemagglutinin (clone 3F10; Roche, Indianapolis, Ind.) antibodies.

Each chip was spotted with 4,333 peptide fragments representing proteins from 3,184 unique Bm genes as well as 176 IgG positive control spots and 61 spotted IVTT reactions without Bm ORFs (IVTT controls). The IgG positive control spots served as an assay control, while the IVTT control spots served as a sample-level normalization factor. For each chip, 3 replicates were printed on 3 nitrocellulose “pads.”

Plasma Probing

Plasma samples were diluted 1:100 in an E. coli lysate solution (3 mg/mL) in protein arraying buffer (Maine Manufacturing, Sanford, ME, USA) and incubated at room temperature for 30 min. Chips were rehydrated in blocking buffer for 30 min. Blocking buffer was removed, and chips were exposed to diluted plasma samples using sealed, fitted slide chambers to avoid cross-contamination between pads. Chips were incubated overnight at 4° C. with agitation. Chips were washed five times with TBS-0.05% Tween 20, and exposed at room temperature to biotin-conjugated goat anti-mouse IgM and Cy3-conjugated goat anti-mouse IgG (Jackson ImmunoResearch, West Grove, Pa., USA) diluted 1:500 and 1:200, respectively, in blocking buffer. Chips were washed three times with TBS-0.05% Tween 20 and exposed at room temperature to streptavidin-conjugated SureLight P-3 (Columbia Biosciences, Frederick, Md., USA) in the dark. Chips were washed three times with TBS-0.05% Tween 20, three times with TBS, and once with water. Chips were air-dried by centrifugation at 1,000×g for 4 min and scanned on a GenePix 4300A High-Resolution microarray scanner (Molecular Devices). Spot and background intensities were measured using an annotated grid file (.GAL).

Protein Microarray Data Analysis

Raw spot and local background fluorescence intensities, spot annotations and sample phenotypes were imported and merged in the R statistical environment, where all subsequent procedures were performed (www.r-project.org). Foreground spot intensities were adjusted for local background by subtraction, and negative values were converted to 1. All foreground values were transformed using the base 2 logarithm (log 2). The dataset was normalized to remove systematic effects by subtracting the median signal intensity of the IVTT controls for each sample. Given that the IVTT control spots carry the chip, sample and batch-level systematic effects, but also antibody background activity to the IVTT system, this procedure normalizes the data and provides a relative measure of specific antibody binding to non-specific antibody binding (a.k.a. background). For the normalized data, a value of 0.0 indicates that the intensity does not differ from background whereas a value of 1.0 (log 2) denotes an intensity that is twice that of background. Immunoreactive antigens were defined as those for which a mean immunoreactivity of at least 1.0 log 2 (i.e., at least twice that of background) was detected at time of peak infection, time of partial resolution, or time of full resolution of infection.

Results

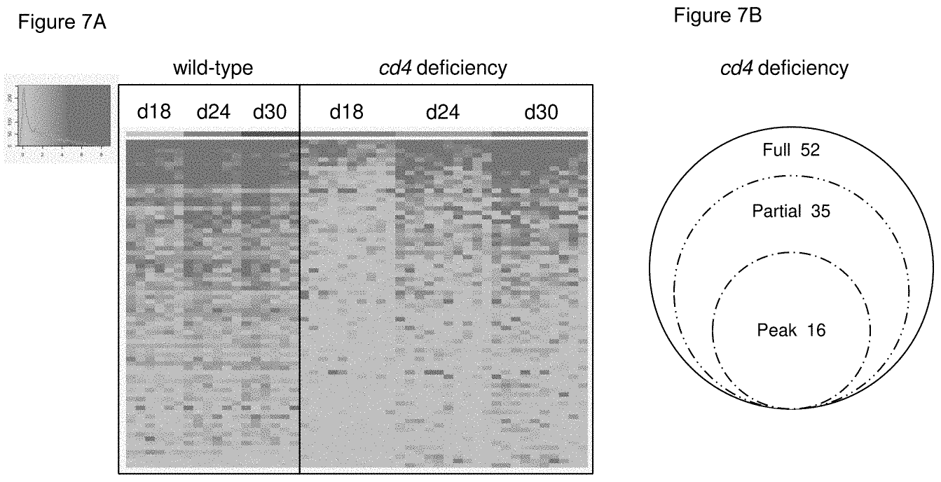

In cd4-deficient mice, as parasitemia resolves, the number of IgG reactive Bm antigens markedly increased (FIG. 7A). Sixteen were IgG reactive at time of peak infection, 35 at time of partial resolution, and 52 at time of full resolution (FIG. 7B).

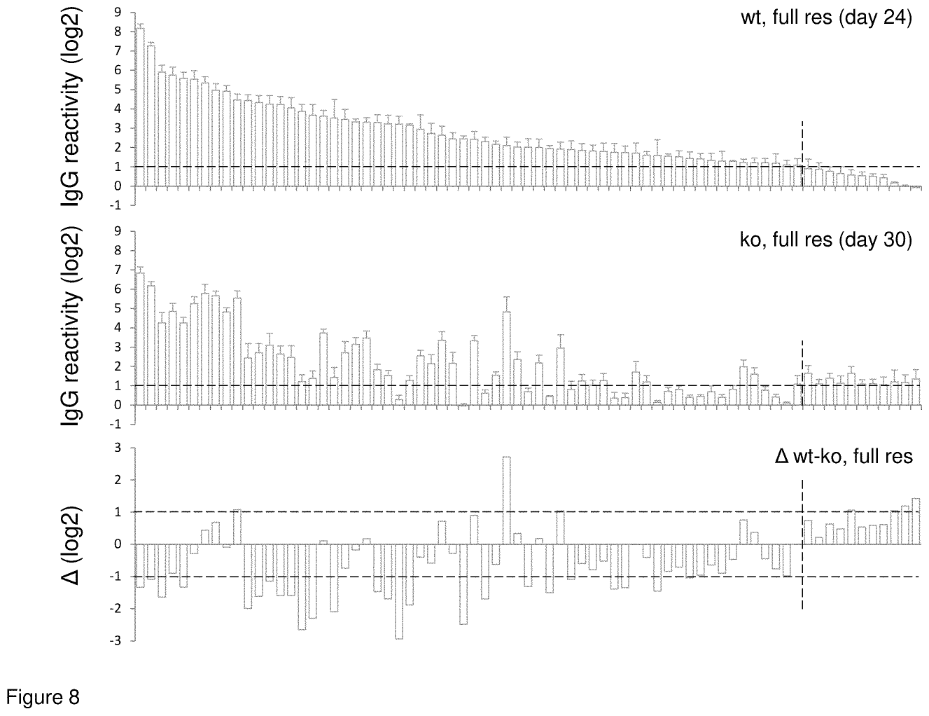

In WT mice, at time of full resolution, the IgG antibody repertoire was restricted to a set of 60 Bm antigens (FIG. 8, top panel, left of the vertical dashed line). Of these 60 antigens, only 41 were IgG reactive when plasma was obtained from cd4-deficient mice (FIG. 8, middle panel, left of the vertical dashed line and above the horizontal dashed line). Eleven additional antigens which were not reactive to plasma obtained from WT mice were reactive, although marginally, to plasma obtained from cd4-deficient mice (FIG. 8, middle panel, right of the vertical dashed line). Thus, cd4 deficiency results in the loss of IgG reactivity to 19 Bm antigens but in the gain of IgG reactivity to 11 Bm antigens. Such shift in the repertoire of cognate Bm antigens can be attributed to the fact that absence of CD4 curtails the T cell response to low-affinity antigens while promoting the T cell response to high-affinity antigens.

### Example 3: Characterization of Bm Antigens Associated with Protective Immunity

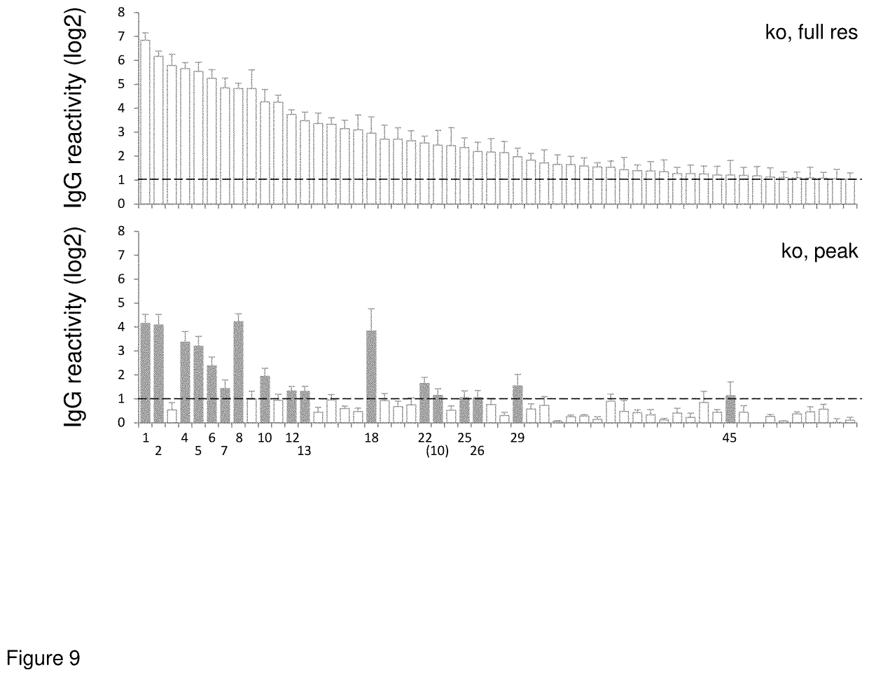

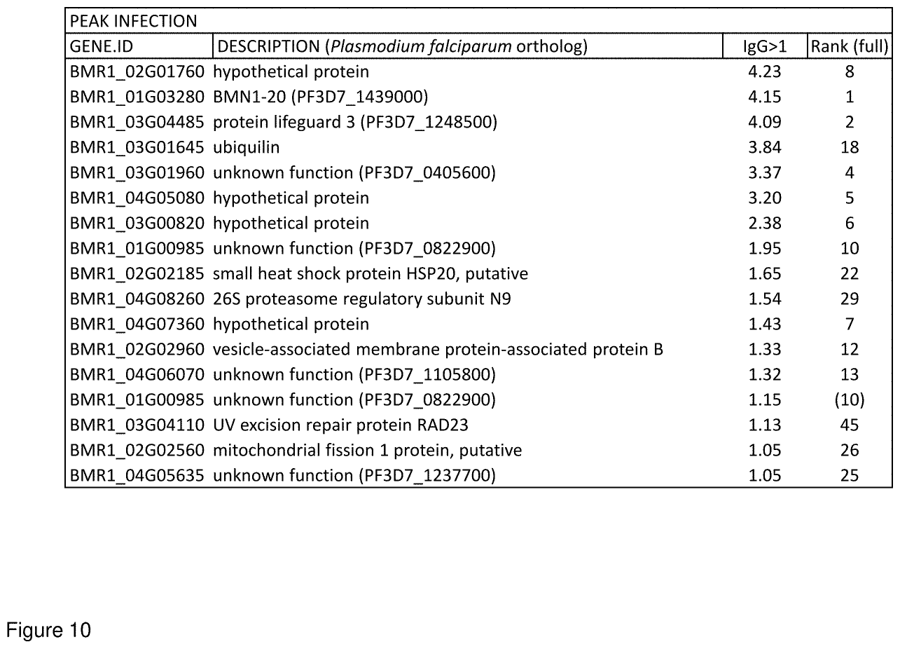

Sixteen distinct antigens were identified at time of peak infection as exhibiting an IgG reactivity that was above background by greater than 2.0-fold (>1.0 log 2) (FIG. 9). Neutralization of these Bm antigens by IgG antibodies may help curtail the rise in Bm parasitemia. Their gene ID and their name (if unnamed, a brief description of their characteristics) are provided in FIG. 10.

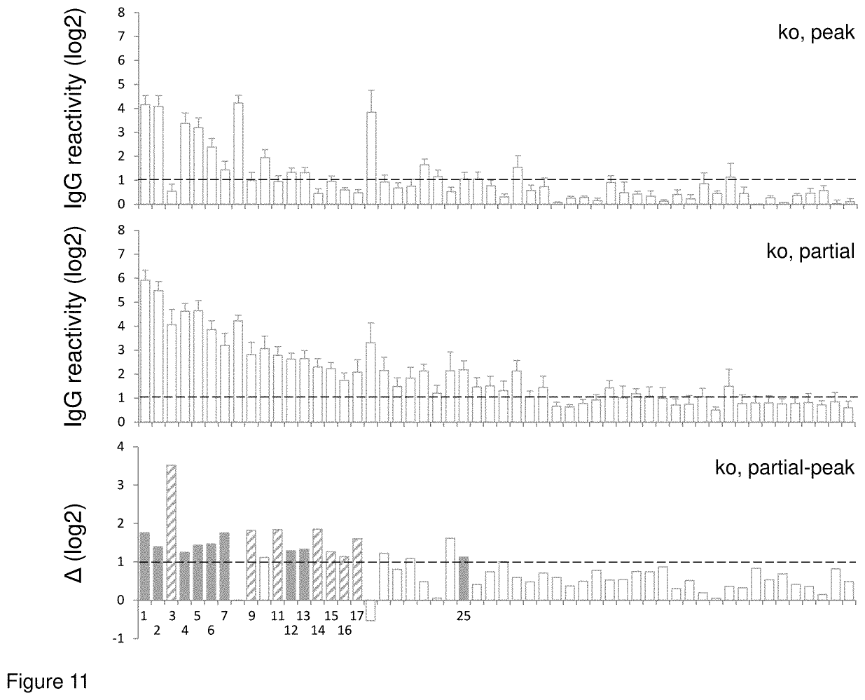

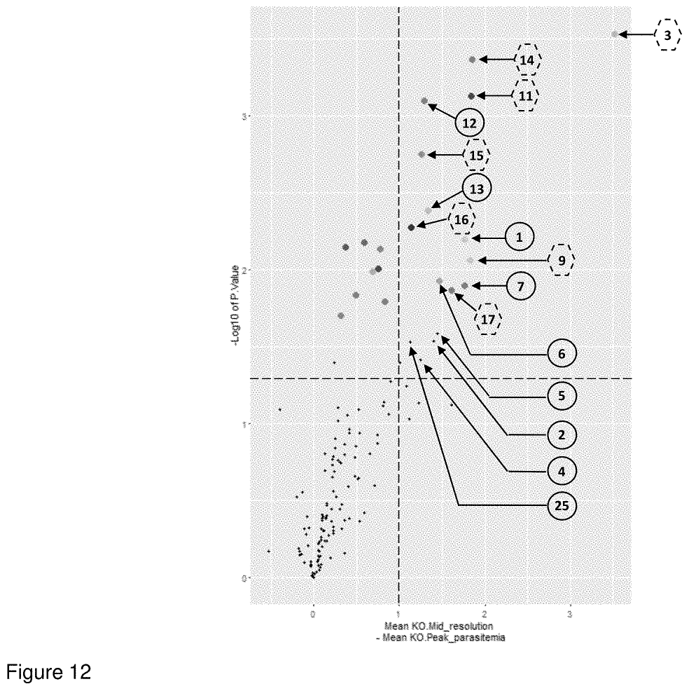

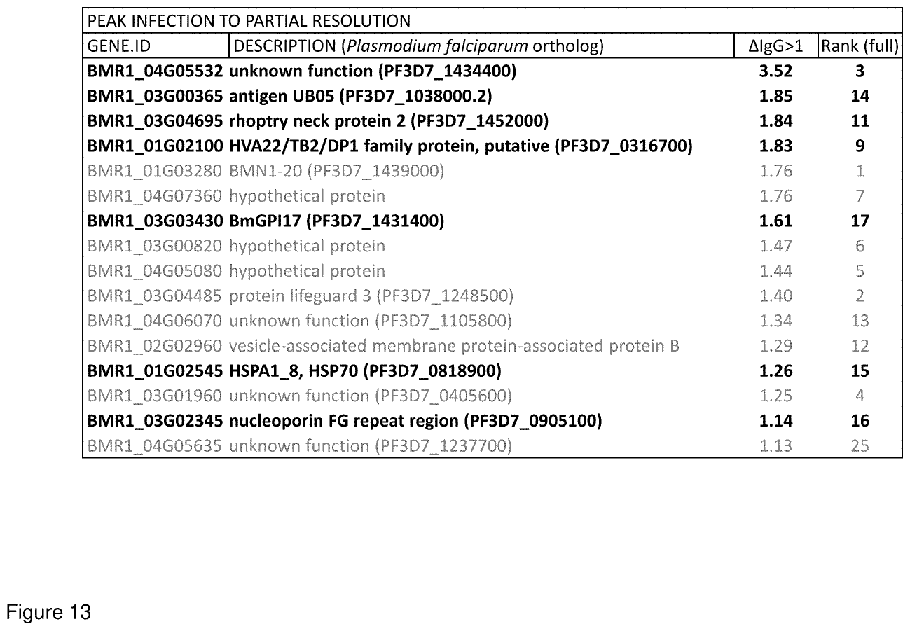

Sixteen antigens were identified as exhibiting a significant gain in IgG reactivity of greater than 2.0-fold (>1.0 log 2) from time of peak infection to time of partial resolution (FIGS. 11+12). Neutralization of these Bm antigens may help achieve partial resolution of Bm parasitemia. Their gene ID and their name (if unnamed, a brief description of their characteristics) are provided in FIG. 13. Of these 16 antigens, only seven were not identified as immunoreactive at time of peak infection (see hatched bars in FIG. 11; numbers highlighted by a hexagon in FIG. 12; antigen names written in black ink in FIG. 13).

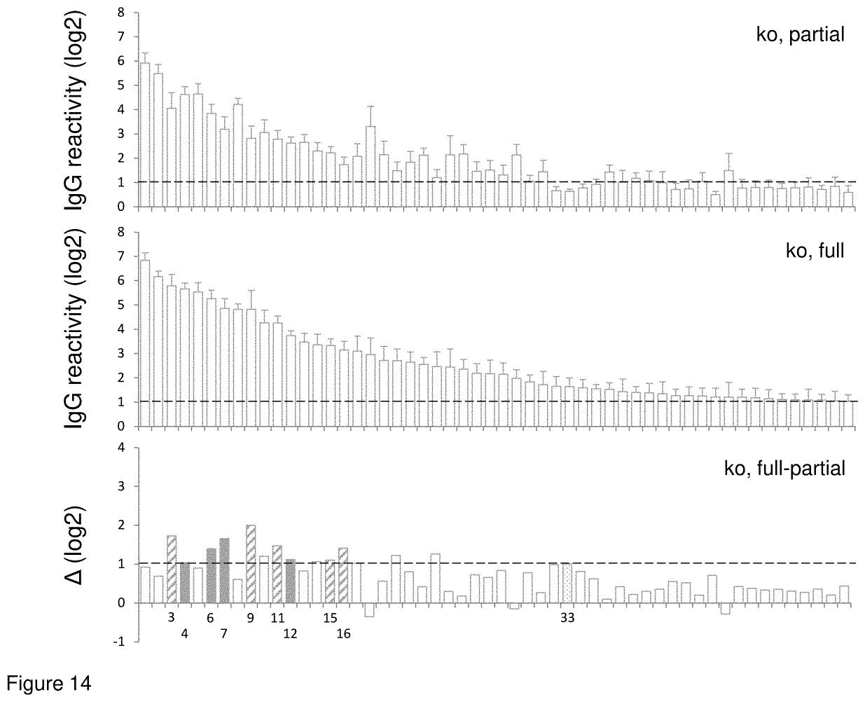

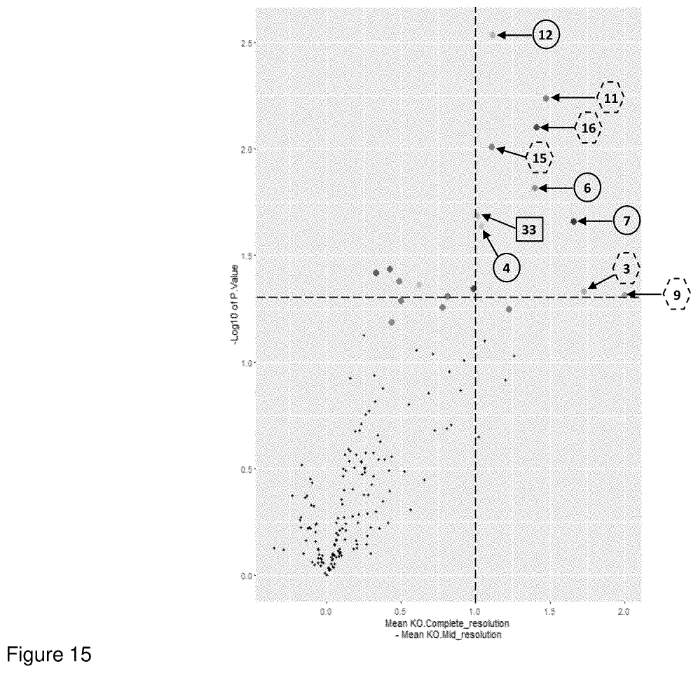

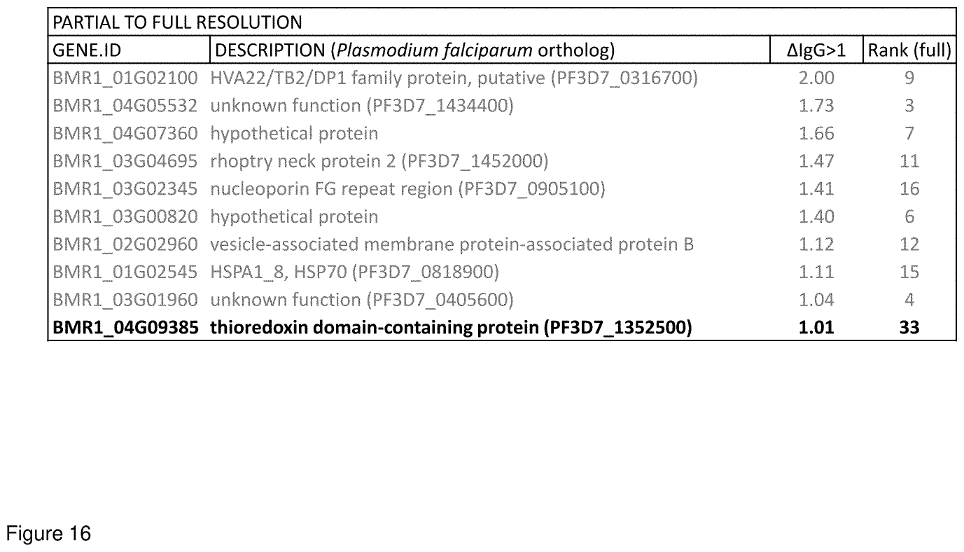

Ten antigens were identified as exhibiting a significant gain in IgG reactivity of greater than 2.0-fold (>1.0 log 2) from time of partial resolution to time of full resolution (FIGS. 14+15). Neutralization of these Bm antigens may help achieve full resolution of Bm parasitemia. Their gene ID and their name (if unnamed, a brief description of their characteristics) are provided in FIG. 16. Of these 10 antigens, only one was not identified as immunoreactive at time of peak infection or as gaining reactivity from time of peak infection to time of partial resolution (see dotted bar in FIG. 14; number highlighted by a rectangle in FIG. 15; antigen name written in black ink in FIG. 16).

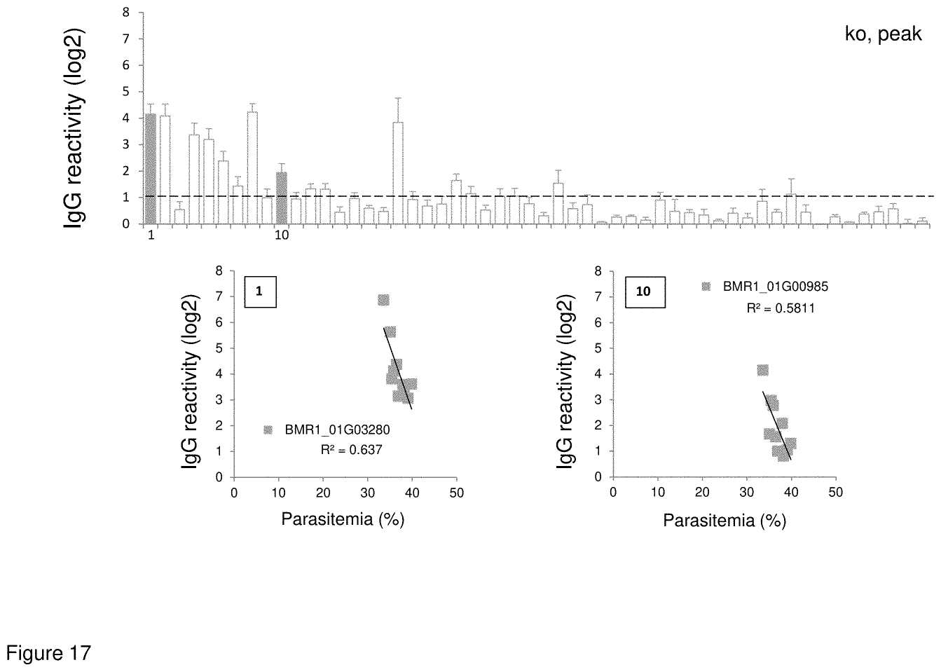

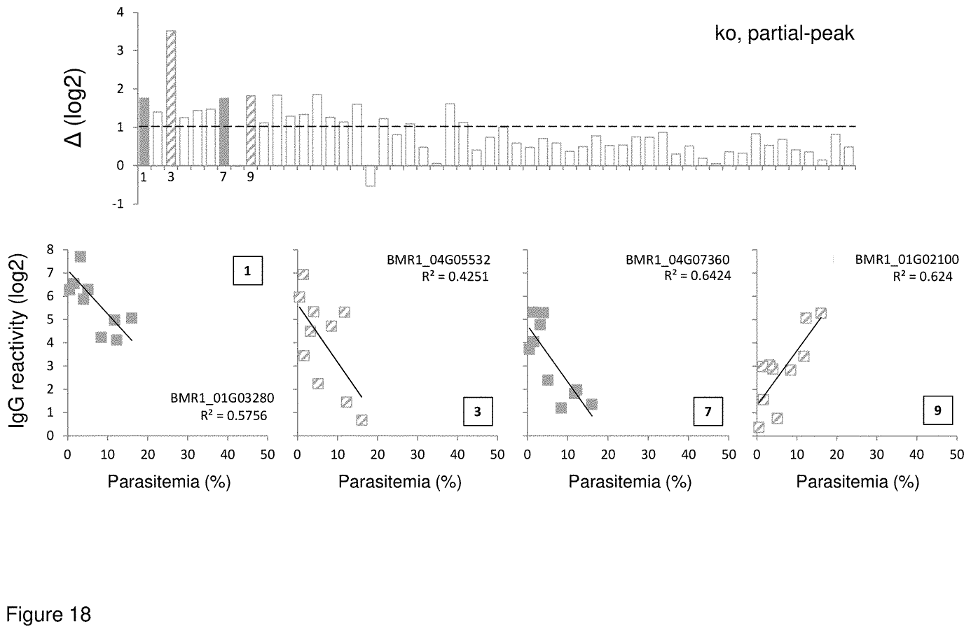

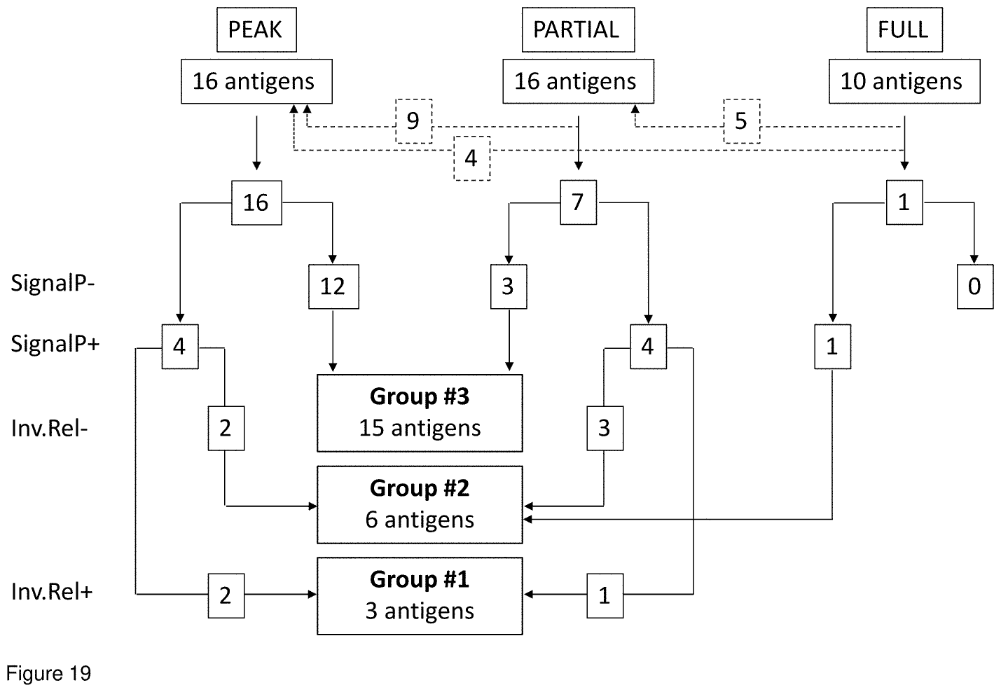

To characterize the 24 distinct antigens identified as exhibiting IgG reactivity at time of peak infection and/or gaining IgG reactivity from one time point to the next, we analyzed their amino acid sequence for the presence of a signal peptide. We also tested for an inverse relationship between IgG reactivity and Bm parasitemia at times of peak infection and/or partial resolution. Of the 24 antigens, nine exhibit a motif that can act as a signal peptide (FIG. 19). Of the eight antigens that contain a signal peptide and were immunoreactive at time of peak infection and/or had gained reactivity by the time of partial resolution, three displayed an immunoreactivity that was inversely correlated with Bm parasitemia at one of these two time points (FIGS. 17 and 18). These three antigens, namely BMR1_01G03280 (SEQ ID NO: 1), BMR1_04G05532 (SEQ ID NO: 2), and BMR1_01G00985 (SEQ ID NO: 3), are referred to as group #1 antigens (FIGS. 19+20).

Of the eight antigens that contain a signal peptide and were immunoreactive at time of peak infection and/or had gained significant immunoreactivity by the time of partial resolution, five displayed an immunoreactivity that was not inversely correlated with Bm parasitemia at one of these two time points. These five antigens, namely BMR1_02G01760 (SEQ ID NO: 4), BMR1_03G00365 (SEQ ID NO: 5), BMR1_03G04695 (SEQ ID NO: 6), BMR1_03G03430 (SEQ ID NO: 7) and BMR1_04G06070 (SEQ ID NO: 8), are referred to as group #2 antigens. This group of antigens also includes BMR1_04G09385 (SEQ ID NO: 9), an antigen which contains a signal peptide but for which a significant gain in IgG reactivity was only detected at time of full resolution, thereby precluding the testing of a relationship between IgG reactivity and Bm parasitemia at this time point.

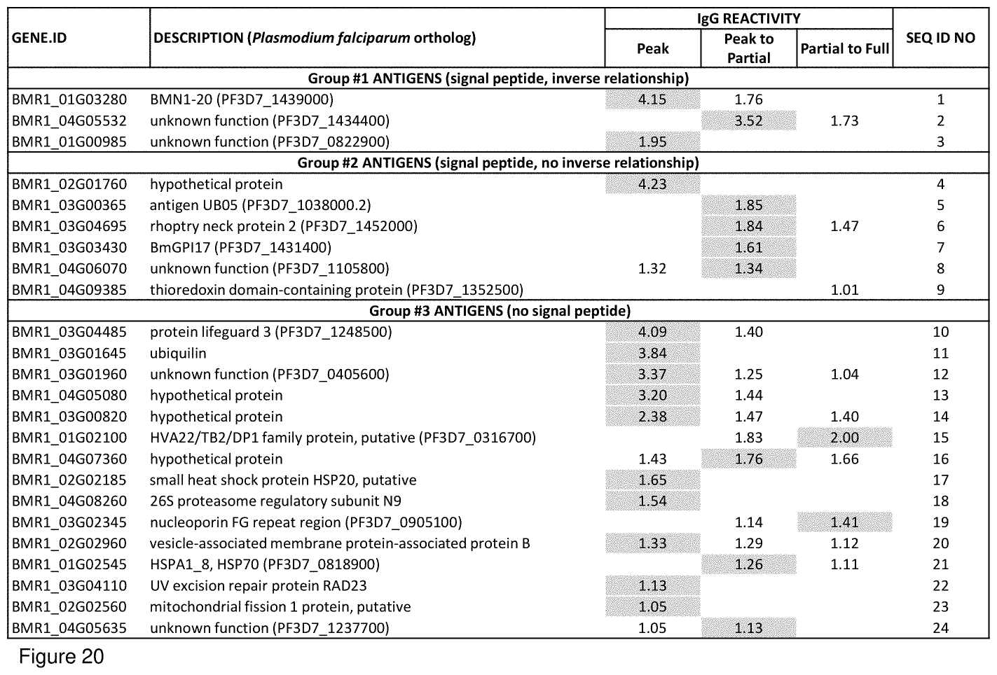

A third group of antigens comprise the 15 antigens that do not contain a signal peptide but were identified as immunoreactive at time of peak infection or as gaining immunoreactivity from one time point to the next. The gene ID of each of these 15 antigens is provided in FIG. 20. One of these 15 antigens, namely BMR1_04G07360 (SEQ ID NO: 16), displayed an immunoreactivity that was inversely correlated with Bm parasitemia at time of partial resolution of infection (FIG. 18).

Any of the 24 distinct antigens, which are listed in FIG. 20, may be used individually or in combination as a Bm antigen vaccine to confer protection from babesiosis, or may be targeted by a therapeutic antibody composition to resolve or help resolve babesiosis.

### Example 4: Screen of Ifngr1-Deficient Mice for Antibody Reactivity and Further Analysis of cd4-Deficient Mouse Screen

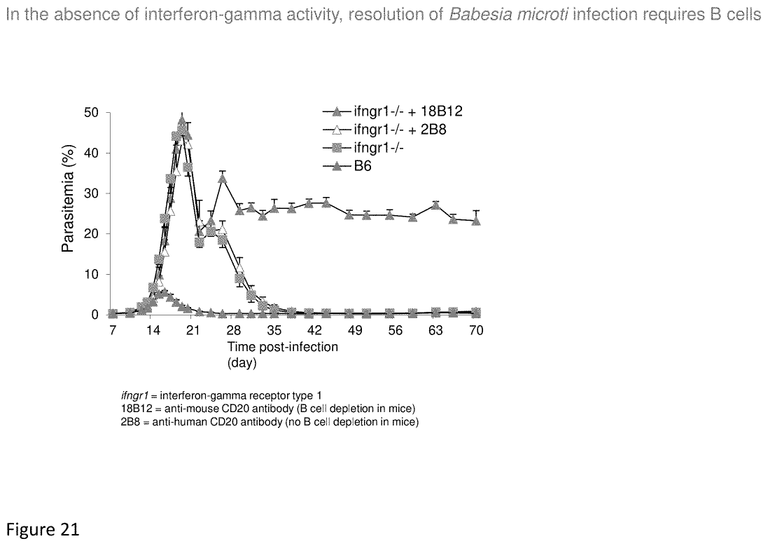

Experiments were carried out to identify antigens that are or become antibody reactive during the resolution of B. microti infection in a mouse model of interferon gamma receptor type 1 (ifngr1) deficiency. The results of these experiments are shown in FIGS. 21-X.

FIG. 21 shows that B cells are required for resolution of Babesia microti infection in ifngr1-deficient mice. The rationale is that B cells are required for resolution of infection in this second model as demonstrated by the lack of resolution in ifgnr1-deficient mice in which mature B cells have been depleted by chronic administration of the antibody 18B12 (gray triangles). As expected, the isotype control antibody 2B8 does not prevent the resolution of infection (open triangles).

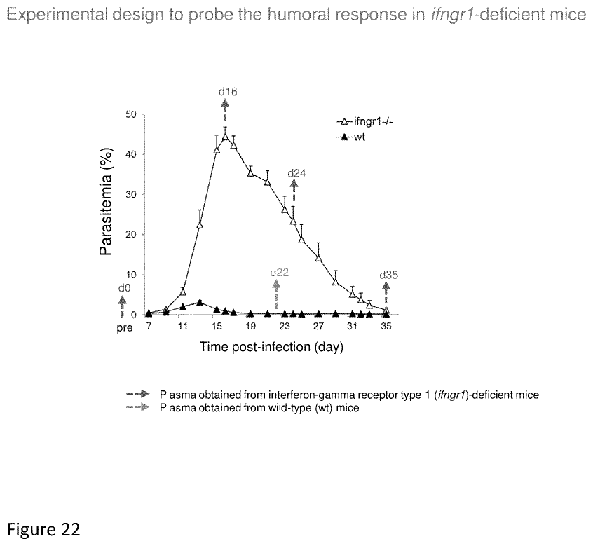

FIG. 22 shows an experimental design to probe the antibody response in ifngr1-deficient mice. We collected blood from B. microti infected ifngr1-deficient mice prior to infection (d0) and at three time points following infection, namely at time of peak infection (d16), mid resolution (d24), and full resolution (d35). We also collected blood from B. microti infected wild-type mice at time of full resolution (d22). Plasma was separated from blood and probed for IgG reactivity using B. microti whole proteome arrays.

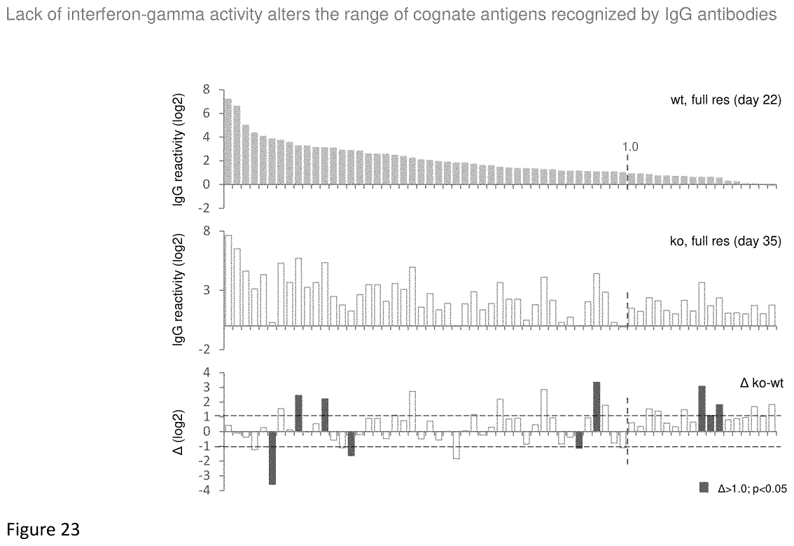

FIG. 23 shows that a lack of interferon-gamma activity alters the range of cognate antigens recognized by IgG antibodies.

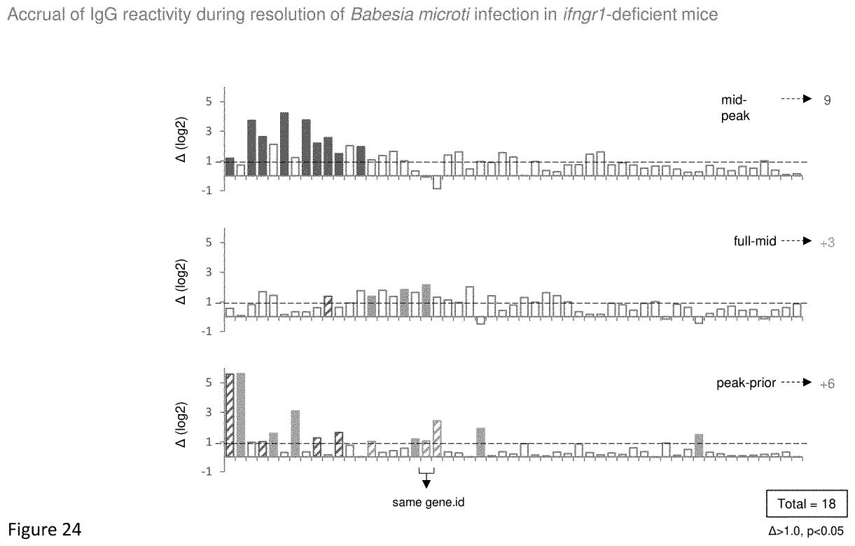

FIG. 24 shows the accrual of IgG reactivity during resolution of Babesia microti infection in ifngr1-deficient mice. We selected antigens for which IgG reactivity significantly increases by at least 2-fold between peak infection and mid-resolution (top panel) and/or between mid-resolution and full resolution (middle panel). Given that resolution may unfold only if other antigens are already neutralized, we selected antigens for which IgG reactivity significantly increases by at least 2-fold from prior infection to peak infection (bottom panel). Overall, the screen identified 18 distinct antigens for which neutralization may contribute to resolution of infection in ifngr1-deficient mice.

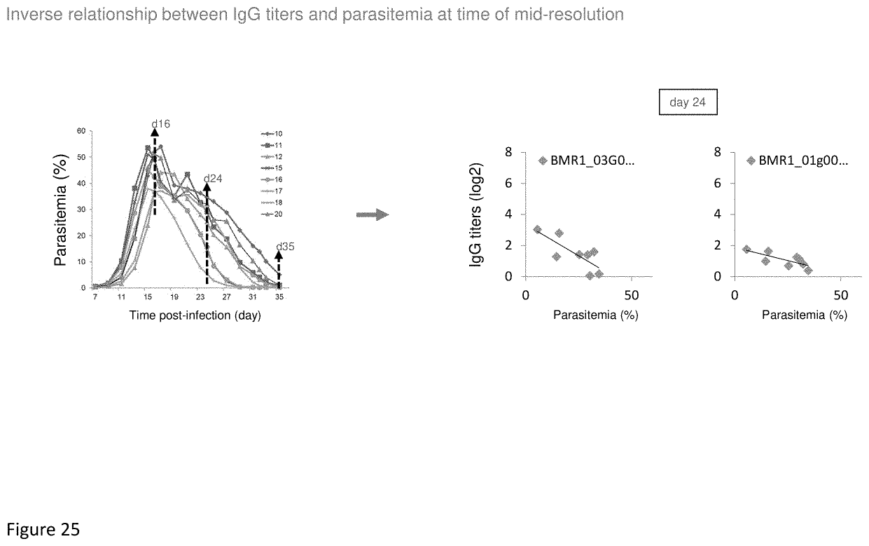

FIG. 25 shows the inverse relationship between IgG reactivity and parasitemia at time of mid-resolution. To gain evidence that neutralization of some antigens is effective in driving resolution of infection, we tested for an inverse relationship between IgG titers and parasitemia. No such inverse relationship was observed at time of peak infection. At time of mid-resolution, however, an inverse relationship was noted for two antigens, particularly for BMR1_03g00365.

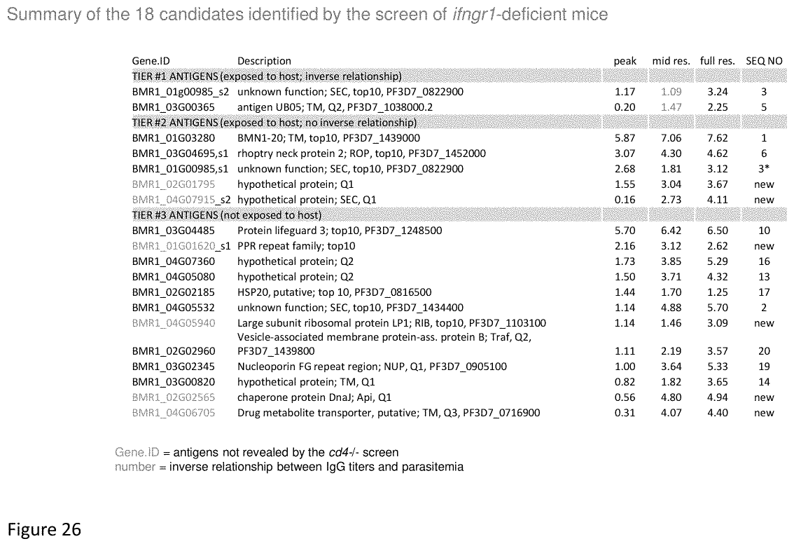

FIG. 26 shows a list of candidate antigens identified by the screen of ifngr1-deficient mice. We organized the 18 distinct antigens into three tiers. Tier #1 comprises 2 antigens which are predicted to be exposed to the host and for which an inverse relationship was noted. Tier #2 comprises 4 additional antigens which are predicted to be exposed to the host but for which no inverse relationship was noted. Tier #3 comprises 12 additional antigens which are predicted not to be exposed to the host. Of the 18 distinct antigens, 12 were identified by the screen of cd4-deficient mice but 6 are unique to the screen of ifgnr1-deficient mice. Of these 6 antigens, 2 belong to tier #2 whereas 4 belong to tier #3.

### Example 5: RNA Vaccines

RNA molecules encoding one or more of the Bm antigens described herein (or one or more antigenic variants and/or fragments thereof) are generated using standard methods (e.g., in vitro transcription of DNA templates) and formulated for administration to subjects. Optionally, modified nucleosides are used in the synthesis of the RNA molecules to reduce immunogenicity and/or to increase stability. RNA molecules are administered to subjects, e.g., by injection of lipid nanoparticles comprising the RNA.

### Example 6: DNA Vaccines

DNA vaccines can be prepared using expression vectors that are engineered to include sequences encoding one or more of the Bm antigens described herein (or one or more antigenic variants and/or fragments thereof). In addition to the antigen coding sequence(s), the vectors can include promoter sequences (for example, a viral promoter, e.g., a Rous Sarcoma Virus (RSV) promoter, a cytomegalovirus (CMV) immediate early promoter, or an SV40 promoter), a polyadenylation/transcription termination signal, and optionally other standard vector sequences, as are known in the art. The vectors can be mono- or poly-cistronic, as is known in the art. DNA vaccines can be administered to subjects by injection (e.g., intramuscular or intradermal injection), gene gun, or mucosal delivery.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

Other embodiments are within the scope of the claims.

1. A composition selected from the group consisting of compositions 1-112 of Tables 2-9, wherein the composition comprises each of the Bm antigens indicated as being present in the composition or one or more antigenic variants and/or antigenic fragments thereof.

2. The composition of claim 1, wherein the composition comprises one or more antigenic variant of one or more Bm antigen indicated as being present in the composition, and the sequence of the one or more variant has at least 80%, 85%, 90%, 95%, 97%, or 99% identity to the sequence of the corresponding Bm antigen indicated as being present in the composition, or an antigenic fragment thereof.

3. The composition of claim 1, wherein one or more (e.g., each) Bm antigen of the composition comprises a sequence having 100% identity to the sequence of a Bm antigen indicated as being present in the composition or an antigenic fragment thereof.

4. The composition of claim 1, further comprising a Bm antigen selected from the group consisting of Bm antigen of SEQ ID NO: 4, 8-24, 51-54, or an antigenic variant and/or antigenic fragment thereof.

5. The composition of claim 1, further comprising 1, 2, 3, 4, or 5 Bm antigens selected from the group consisting of Bm antigen(s) of SEQ ID NO: 4, 8-24, 51-54, or antigenic variant(s) and/or antigenic fragment(s) thereof.

6. A composition comprising: a Bm antigen of SEQ ID NO: 49, or an antigenic variant and/or antigenic fragment thereof; a Bm antigen of SEQ ID NO: 50, or an antigenic variant and/or antigenic fragment thereof; a Bm antigen of SEQ ID NO: 51, or an antigenic variant and/or antigenic fragment thereof; a Bm antigen of SEQ ID NO: 52, or an antigenic variant and/or antigenic fragment thereof; a Bm antigen of SEQ ID NO: 53, or an antigenic variant and/or antigenic fragment thereof; and/or a Bm antigen of SEQ ID NO: 54, or an antigenic variant and/or antigenic fragment thereof; or two or more of said Bm antigens, or antigenic variants and/or antigenic fragments thereof.

7. (canceled)

8. The composition of claim 6, comprising a Bm antigen of SEQ ID NO: 49, or an antigenic variant and/or antigenic fragment thereof; and/or a Bm antigen of SEQ ID NO: 50, or an antigenic variant and/or antigenic fragment thereof.

9. A composition comprising a nucleic acid molecule corresponding to each Bm antigen, antigenic variant, or antigenic fragment of a composition of claim 1.

10. A composition comprising an antibody that specifically binds to each Bm antigen, or antigenic variant and/or antigenic fragment thereof, of a composition of claim 1.

11. The composition of claim 1, further comprising one or more adjuvant, carrier, diluent, excipient, or preservative, and/or being formulated for administration by the oral route or a parenteral route, such as a parenteral route selected from the group consisting of the intravenous, intraperitoneal, subcutaneous, intramuscular, and topical routes.

12. (canceled)

13. A method of immunizing or conferring protective immunity against babesiosis to a subject, the method comprising administering a composition of claim 1 to the subject, and optionally wherein the etiology of babesiosis is Babesia microti.

14. The method of claim 13, wherein:
(a) the subject does not experience babesiosis
(b) the treatment reduces the severity of Babesia infection (e.g., parasitemia or parasite burden) upon a subsequent challenge;
(c) the treatment reduces the severity of babesiosis upon a subsequent challenge;
(d) the subject experiences babesiosis;
(e) the treatment reduces the severity of Babesia infection (e.g., parasitemia or parasite burden);
(f) the treatment reduces the severity of babesiosis; or (g) the subject experiences mild or severe babesiosis, including persistent or relapsing babesiosis.

15-20. (canceled)

21. A method of supplementing an immune response in a subject experiencing babesiosis or for treating babesiosis in a subject in need thereof, the method comprising administering a composition of claim 10 to the subject.

22-23. (canceled)

24. A method for determining whether a subject, prior to administration of a composition of claim 1, has protective immunity against Bm-induced babesiosis, the method comprising:
a. applying a body fluid sample from the subject to a solid support, wherein the solid support comprises two or more Bm antigens comprising an amino acid sequence having at least 80% sequence identity to the amino acid sequence of any one of SEQ ID NOs: 1-24 or 49-54, or one or more antigenic fragments thereof;
b. applying an antibody detection reagent to the solid support of (a); and
c. identifying the subject as likely to have protective immunity against Bm-induced babesiosis if the fluid sample from the subject is determined to have a sufficient titer of IgG antibodies that specifically react with two or more Bm antigens of (a) or antigenic fragments thereof of, that is, a titer in the fluid sample above a cutoff titer for the two or more Bm antigens.

25. A method for determining whether a subject, following administration of a composition of claim 1, has acquired protective immunity against Bm-induced babesiosis, the method comprising:
a. applying a body fluid sample from the subject to a solid support, wherein the solid support comprise two or more Bm antigens comprising an amino acid sequence having at least 80% sequence identity to the amino acid sequence of any one of SEQ ID NOs: 1-24 or 49-54, or one or more antigenic fragments thereof;
b. applying an antibody detection reagent to the solid support of (a); and
c. identifying the subject as likely to have protective immunity against Bm-induced babesiosis if the fluid sample from the subject is determined to have a sufficient titer of IgG antibodies that specifically react with two or more Bm antigens of (a) or antigenic fragments thereof of, that is, a titer in the fluid sample above a cutoff titer for the two or more Bm antigens.

26. A method for identifying a patient who experiences babesiosis and is likely to benefit from an anti-Bm antibody-based therapy, the method comprising:
a. applying a body fluid sample from the subject to a solid support, wherein the solid support comprise two or more Bm antigens comprising an amino acid sequence having at least 80% sequence identity to the amino acid sequence of any one of SEQ ID NOs: 1-24 or 49-54, or one or more antigenic fragments thereof;
b. applying an antibody detection reagent to the solid support of (a); and
c. identifying the patient as likely to benefit from a composition of claim 10 if the fluid sample from the subject is determined to have an insufficient titer of IgG antibodies that specifically react with two or more Bm antigens of (a) or antigenic fragments thereof, that is, a titer in the fluid sample below a cutoff titer for the two or more Bm antigens.

27. A method of optimizing the administration or therapeutic efficacy of an anti-Bm antibody-based therapy to a subject experiencing babesiosis, the method comprising:
a. applying a body fluid sample from the subject to a solid support, wherein the solid support comprise two or more Bm antigens comprising an amino acid sequence having at least 80% sequence identity to the amino acid sequence of any one of SEQ ID NOs: 1-24 or 49-54, or one or more antigenic fragments thereof;
b. applying an antibody detection reagent to the solid support of (a); and
c. administering to the subject the composition of claim 10 if the sample from the subject is determined to have an insufficient titer of IgG antibodies that specifically react with two or more Bm antigens of (a) or antigenic fragments thereof, that is, a titer below a cutoff titer for the two or more Bm antigens,
wherein optionally the method further comprises administering to the subject the composition described herein, wherein the subject has been determined as likely to benefit from an anti-Bm antibody-based therapy or from a modified dose or dosage of anti-Bm antibody-based therapy.

28. The method of claim 26, further comprising administering to the subject the composition described herein, wherein the subject has been determined as likely to benefit from an anti-Bm antibody-based therapy or from a modified dose or dosage of anti-Bm antibody-based therapy.

29. A kit comprising (a) two or more Bm antigens comprising an amino acid sequence having at least 80%, 85%, 90%, 92%, 95%, 97%, 98%, 99%, sequence identity to the amino acid sequence of any one of SEQ ID NOs: 1-24 or 49-54, or one or more antigenic fragments thereof, wherein two or more Bm antigens are immobilized on one or more solid supports; (b) an antibody detection reagent; and (c) a package insert comprising instructions for using the two or more Bm antigens and the antibody detection reagent in accordance with a method described herein, optionally wherein the kit comprises one or more Bm antigen comprising an amino acid sequence having at least 80% sequence identity to the amino acid sequence of any one of SEQ ID NOs: 49-54, or one or more antigenic fragments thereof.

30. (canceled)

31. A kit comprising (a) the composition of claim 1 and (b) a package insert comprising instructions for using the composition in accordance with a method described herein.


SEQUENCE LISTING (60 sequences)

SEQ ID NO: 1 - protein, 402 aa, Babesia microti
DVYEISSGNPPDIEPTSTSLETNVVTNYIPEPNADSESVHVEIQEHDNINPQDACDSEPLEQMDSDTRVLPESLDEGVPHQFSRLGHHSDMASDINDEEPSFKIGENDIIQPPWEDTAPYHSIDDEELDNLMRLTAQETSDDHEEGNGKLNTNKSEKTERKSHDTQTPQEIYEELDNLLRLTAQEIYEERKEGHGKPNTNKSEKAERKSHDTQTTQEICEECEEGHDKINKNKSGNAGIKSYDTQTPQETSDAHEEGHDEINTNKSEKAERKSHDTQTTQEICEECEEGHDKINKNKSGNAGIKSYDTQTPQETSDAHEEEHGNLNKNKSGKAGIKSHNTQTPLKKKDFCKEGCHGCNNKPEDNERDPSSPDDDGGCECGMTNHFVFDYKTTLLLKSLKTET

SEQ ID NO: 2 - protein, 217 aa, Babesia microti
IITDGLSAIGSVASEVGNTVKDVSSEALLGELQQIADGGKIIRDGTESNFVNSIANTVMKNVVGTAVLKASSGITHNGDFAFYNFMYPENDDYPWACICDESDYEEYIKGKKDKVRCRNYIDSSLQNAVLYCNPANHNSSINDNANNNPPKQIDNHVSIPQTAPANHTTVLSTEVDTNHNEQKQPNSPSVPSESQNSVSAPKDESVSSTVEGAKSSS

SEQ ID NO: 3 - protein, 925 aa, Babesia microti
TLVSKITTPNNLPGIDTCSNWEDVSVCTTKNTRNCISGEGKPKDCFAIGKSLFKTFPNCYEGVIIDQVTFSGFETLEIHYCDIDKILHKANEVVKSIHELKEKTNQLTEKIKDIPDLIDKVIKVNSEISKIFHQDKIKHMEKEANDFKNALKTTRNYIISYDSLDKTKQSNLLTSLGKLMNKIKTKLSEMDKTLHSTLDTNNTIIDLVNNNSSHVKHPNDFNKTMELYNETITKADAIKKNIEKLKEHRKISTHKTIFSNNIDKLIDNLTDYFENINRSIDAVRDKLSKYQLETGKMVLLFKNVNEIQKHIKNTDMHIRTCNYDFSDIEQKYSLITAKITVEDGQSITTSNKSTVDIPEEKVDRVNVNVDKAENSDNETSQENTSVKPTDHKEIEDSASEENAIGENGDYDSDEDIDTNDVKEDHENAIDSEYTVSSTGDVLEDEVVEENAIGENGDYDSDEDIDTNDVKEDHENAIDSEYTVSSTGDVLEDEVVEENAIGENGDYDSDEDIDTNDVKEDHENAIDSEYTVSSTGDVLEDEVVEENAIGENGDYDSDEDIDTNDVKEDHENAIDSEYTVSSTGDVLEDEVVEENAIGENGDYDSDEDIDTNDVKEDHENAIDSEYLVSSTGDVLEDEVVEENAIGENGDYDSDEDIDTNDVKEDHEDAIDSEYLVSLTGDVLEDEVVEENAIGENGDHDSDEDIDIEEVNEEDHEDAIDSDNSISNSENVDTTPTENVDTIPTKNANTTPTKNANATPTKNVDTIPTKNVDTIPTKNANTTPTKNVVTTPTKNANTTPTKNVVTTPTKNANTTPTKNVVTTPTKNANTTPTKNVVTTSTGNVRTKHTSTNSHVLAPDTDEYPAQISQHKTIDKYYQSLELEDEENADISSSDKPVSPINLEDKNSTYDHMHKTDNVKSAGIASYD

SEQ ID NO: 4 - protein, 187 aa, Babesia microti
NRSCPGNNGVGGGSGDNNSGIIPNDPHPCCNNLRQKPQYQTKPENELVNDDRDLNFNKIRGGKQIITFTVPSIDDLKNKRLSDSEFILSEKANPLISSGDSKNVIVFEVKNDNEKLMGSVEVGQWEVTITTSCIRRIVIFDSNEVSDNIPMYIYIVDYFEGGNSTVSKFFFANNRWNDFTNHTPNAA

SEQ ID NO: 5 - protein, 74 aa, Babesia microti
FLLNRSEFKWFKVGLIITTIFPFKHSFDYNLTHIFLFSICTLIFCVKPVDEESGAKKEGFDFKKMVPDKFKKYT

SEQ ID NO: 6 - protein, 1227 aa, Babesia microti
DSGNSSPQTPPETSSPINGVIGDENNGLEHLSSSGLEVDDNLPELLKTSPFSGQNSDVQSASTPVEPTTPVHSNDQSNPITNKVDTNSNDHTDIKNEGSSHRTSSNNSSVTTNTNNEIRNGGGPLDQNEDKAEDEGETDAEGRGWNERTKNKPTFNATNRDFVDDNLPELLKTSPFSGQNSDVQSASTPVEPTTPVHSNDQSNPITNKVDTNSNDHTDIKNEGSSHRTSSNNSSVTTNTNNEIRNGGGPLDQNEDKAEDEGETDAEGRGWNERTKNKPTFNATNRASPDGIGKMNMEEKQLENFINVSSNALELDISIGRDNFATKFLAQHVNIFGDRISGLSAAYVEGYNNLAKIMYNSHSVLFDRKFNGAVISDNLIGNIADFGSYFLEISPNTTRTNRSDYLKSVVLSKVQYLLSADFSTTDNIQRLTNLALALGYNNVKENNPGNSQHSITTSLSTELFWSFGNNIFLFGHLATLMLAYLESNAYFTSGATRPFFSWQTLVSTGGNEKFDKLDSMCGVIRGSKYSRKNNGFIKPHYKRLRRKTLLEGEPRLLCSMLEEALDTVDKAIKFKGEELNSQGANIENSVSNDINSKRLQAKLCSNLNDSLINVSCDFRSSKLDKHNKKLREAFDLLLACGNLNTGKKEAFPEYLRLISNPFEYGDIFSMTMWWDPREFDGKQGWVEIYKKLRKNIMKPELKNVDMQLKYDSAISYYKQLKESETYPKKNIPWARLYLYMSVIMSRSNAMSWAEDALRSFSNLYRMKPSLVMRGEGLETLLNYCAPDPVALSHIFLYHFLTKKDAGKDLEKDLRRLEKGTLLSRIVNSSSIFIPNKLKKFLKMGARGFFNKKLNTLRAKSTLLRLFPKNLLHSALGAIEFTTHSLATLQISKNMDMWESLAQTKNLDAGGFPGEIDSLFNHWSESGGYSGYITGKLENGDDLTGDDIKKMNIKAPINNDSLNWQKYINKKISEHFGKFLNLPFIQASGSQKNYIYQLVRDSKANLDDNLEQTVFFGKVLPPGKTNNVIKKLKRIADSFTSMLLRSSARPVDHAVWVGVKINVPIVIHITKKLYMIQRDMPRKEAWNLESAFLDLLQDLVIMVTNPGKRSPIGFETIGGNPGLPEISIRYPHMSIEERKIEFQHSQCADHCISIWRSLIAFTLNTLNNPAAIKQFEKSLSSNSSLNDMSKPEYINSFKYILKGDSVLHMYDNMLPRKVKREIKALKYGK

SEQ ID NO: 7 - protein, 433 aa, Babesia microti
LPDILSQNNTFKSFLEVNNVDQEDLICNKALCKSTDSINRNTSSYCYKYKLCSKCSVSNVPDHPVCYLLDNDHNYIHLMEGHLGSQPIGSANSHDNSSHDEHSSHSNNGDMMDEHEEENFLQEYESKSMKFIPTSNMSDFDHARRSCAVDSKGNVMISVRLIIQWYMSKDKSNNQQHHGNDDDSQNYDANYLQLTPMYSDDSVNSSMLEMDHDDSESSNSHKSRMANMAKNFQVLKNIHKSAVKRYKSPKAKIYLIFSNPKINSCRHPVIYNGKISPSSMFVAKLESTISQIDLTQDLIKSSIETIVSCEACDKLKYNSCIQVTCAKNTPGAASLAMGSAVYVPMTNTTIGVNAHNPNAVVAAGIPMGKIPVIPHPAAISGGNVGHLNNGLHKAVNNAVMMPNGTSLPVQSGVVIKSLYNCLAFLLTILYLNF

SEQ ID NO: 8 - protein, 219 aa, Babesia microti
NPIFASATSAPSRNRAQDMTKAECIELLKGIVKSQEETKIVMKKLTSDLINNPLRLEQVYTKASQMQPEDPMEQWGVTVIDLDHLIEKYQHDPIVKDYILKIMNSPGINDTTLSDDVRNITISQILAIHEYMLSELESVVREFKSLQNRQTMEIKTLTIAAQAIVAAKVEEKFNLTSDQVESAVIINHAELTASHAFTRLTMQMQTEMSELIGCQFPGW

SEQ ID NO: 9 - protein, 166 aa, Babesia microti
STNGKGGVDSVSKKSFVIELEDSTFERKTQASSGGTSGVWFVKFYAPWCGHCRSMENDWNELANILGKQINVAKIDATKHSVTAKRFGITSFPTLLLLKDGNFYQYENNNRTADALKQFALHGYKQVKSKPVPKEWSYFVRFKFFIKSGFYEVKRIYQLAYPGFIT

SEQ ID NO: 10 - protein, 251 aa, Babesia microti
MAKLHESTSSASASFDPEKSDYDDTYVLTETTPTYIRHGFVRKVFAILFAQLLVTLGFSLICYFYRESVHSFISKNIWIFPTLAILSFITSLILIFSPSLSRRYPLNYAILVIETLYFSFIVGLSCAFTKSPTAIVLSVSITLGIILLVVLFTLQTKIDFTRYIIYFILFSFVTLVFGFIGIFVPFDTPLRMFYYGLGVLGYSLWMVLDLQLIIGGKTYEWTVDDYVPASLSLYTDVIGIFLNVHGMFSDR

SEQ ID NO: 11 - protein, 320 aa, Babesia microti
MTITLNIKVNSETNFTVEAEPSFTVKELKILCESQSNIEAQNQRLICKGKLLKDTDILSDVGAVDGATVYLVRSQVNKTQSAAPKQNTVPQPTLQTTNQPAGQTQTSGLGFQQQGFQQGFSGSQQPGFQANPFQSMLAGGFPNLDPTQMMEILNSPMAQEAMQRLSQNPEVLRNILQNSSLMTPMLEQNPMLSEMLSNPELMRSMLRPEVLQAGLQMHQAMQQQQQQQPGTQTNPIGSQNPDFSNMMRQMMNVFQQNPSVAQPTAPQVYTDPRPPAERFATQLQALAEMGFIDTEKNITALIATNGDLNATVTRLLESNF

SEQ ID NO: 12 - protein, 309 aa, Babesia microti
MEEAERKFKEFNWADSQGWRIYWDNLYPTPPLSKVDKFKRSWFKRNVDSNISSTPLSEVGKQTQQTTPNQYRSQGNFAILTLEAGVRLLYLLITAPLFVLPLIGVRLSRYIYYHYYIDIALLLIFLLSGIVRERGLPKMDSVWLASAFYSDMTQYIMYTCILMMSAPRPVYLILPFLTCLIGLNSLAETNLSKLPKWLENIVGEIFKYTKDNIYWLMQTRGDVECYLLFYIVFGFLTKSSAVITLMAYINFMKLRIGIGDPFIMSAFSKLHGCIVKLLSYDMFGHIPLRMYLKISELLTKYMSGPRPQY

SEQ ID NO: 13 - protein, 966 aa, Babesia microti
MEDQNTTNESISNLHMENYFPKDIFSNLDNQKNLKTYHSKTFEKYFESAAKDNVERCRTSAVKEWLNRNLPSEYNERHKPTLKLNLSPNHLNSTYNKQISDYTRNAYNRIEQLKKLQEAYSLLRQKLQEKRRASCHKEVSEAEASVLNTQRLIISLKREDKDKIATELLIQNAEELGIPSKDLETLNGYTFNEALKTIIDIISNMINNKFMTNLKDRCESARRKAGSEYRDEMERIKVDLDMYKTKSEKLESTISTLNSYADSSKQNAEKVMELQHSLEDSNRQINQLKDSLYNMAKVNEELEKNEVKLEESLVELERYKLESQKLESVIKDLELLNQQKHDNEELIKMLHDELDQCKSKSLQLNKKIEDLENSNKENLIPLNNELAQAYQKLSELEHSYEQIEHQKNEADKKLESSIDEIEKQKQHSEELEQSITKLKQTIEEMEKETTDQVNDLQTCLIDANCKIESLEGTISQLKQLNLELEGGEHRIQELQSKLTESNSTIEKLEKTITELENVSSKYIDYDEKMDNLQKQLKEYTEKITVLENNASEFNYKDQAETLKTELDKSQLKIMELETMIKENSEKTERVPSPRKDNEEVDRLTSEILQLKNQVDILQNEKTDLEQKLSQQSPRKDNEEVDRLTSEILQLKNQVDILQNEKTDLEQKLSQQSPRKDNEEVDRLTSEILQLKNQVDILQNEKTDLEQKLSQQSPRKDNEEVDRLTSEILQLKNQVDILQNEKTDLEQKLSQQSPRKDNEEVDRLTSEILQLKNQVDILQNEKTDLEQKLSQISAVIEENRQYKEKIELLERKLNEINKEKPKDSFTNVEVINAAFEDVRSLPINSSNASDWTAMPSELELEEHKLYKKKSSRKGKKKSSREKETSRSDISSRSTSRHSKKDKPTIVDNNSTDVEASDSNEQMISDPVESITDQLNLVKQSTIQLTELGYDSISNVGSYLSDYIFGGN

SEQ ID NO: 14 - protein, 265 aa, Babesia microti
MDDLPGSLHQSTPNEKPMAPPTAPKSAPHVPISVESKELSKEIPKESPMTKEDKKDVAKSKVSAAVTEKKVVKEPVVPKITIEMKKFSMSRESTQYSIFIIFNLIFALLYIFKVRLMAIFCNLIIVAISIGAILSSIDPNRRKVDETNVNIIFISPTTVSDLAIVITAKLNQYISYFRRILLWQDFILSTRFTLCVYIMGILFKIIPLVALIYIMCWAFYLYMFICKEMADQLLDIIMVYLKRVSGNFDEFCCNIPKMKDVGKEL

SEQ ID NO: 15 - protein, 244 aa, Babesia microti
MFNENEIPQFQRPFTPSKDTEIDTSINFPANSILNNISFQKLLDWLDECTEGLPIVESFARRFNVTRGHIAAIFSAILILYFIFGWKINIFCNTIGLVYPAFKSHKVLLIHRAMTESPKATATITTGGKDKENDTNPQMPTCLNGIQGEIMFWLRYWIVYSLYLFISILIFPLISWLPLISIVRVGFILYLYHPYTRGANAIYYLVISPLLSKNQKIIEQAIDTLERVAMGEIRKFTEKQMKLH

SEQ ID NO: 16 - protein, 857 aa, Babesia microti
MDSIEECNKLVDAVTKLATSFDYQAQEYLYNLTRDENAINIALSFLLSNKYVLNYSSSYSNEILQYITDVRGPSSCCIWDNIQLDENTSRHLGHITSILFKEHMHIVLCFDDAKLIALIDTLVTIWQLQSTISLVTQSCFADNATDDHITRVISMIIFKKPQLLTHFINILNTPEVTDKYVADFSMKFKLRNCDRDDIYVTRYRHMIVYVLAQVLENFVSSNVTDKISFLPINFSDLVILSGNCNNRYLYSCALLIVNSLNFHISEELLHQLLVIVEKSGFDCDILEMWNFAISHLQYLKVDNFMMKSFRLVQNGLIESTNPINIVPVLNGITVYYVNNSSVPTEIVRAYRQLLSVESLDIPIEIGELFAAVRNACTESLITYDKIASFVSQFDIRMISSRLIELCNPNMQCWSWLNVNCCVEEFPTDYDIYIDSMKNVFKDLFFILPEQLLDTIISEILQSVNENTETSLIALICYDCLEVRHALAILDKIKSLFTLKLSSNYDFILTCMFCQSIFVPHLHRIKDILFQRFVINMKISNIWSKRLAKCIAILNDRDSVHSVIECVQYILETSKSKGVLSQNHYPSILSLSNLLDTLPQMPCDVNLTGSNYIFCKCLLALSISTRQLSQILPKIALNLDILYEGDVDNDLFCLSTSDSQAAVNLLMQLYIQNKVTDIPNELHEIRHPGLLMLCNLNENFEQFRMNKCVEFMSTVSQESCCHCAWACIAYASYVIEQKFNFEFSMEMVKVIYRVLPCLIKMVKSKNEPLHWDLDAILEYRINEIHPSIKCLKGTIWNIYYDGTVSVGSDACKYLSLVNKYAPRIIKSLSNPNVLQLVYQISMCTGSIEVIANKIAISL

SEQ ID NO: 17 - protein, 171 aa, Babesia microti
MSCILKCNNEDELVVDGEKPKVVEYVEKPSVIYKPTTVVPPNSLIEITAPKDLPQNPTFFPTIDTFFDNDVKQIVLLMELPGFVAGDIDLEVGEGEVCVCGPRSKEELYEKYGQNLDIHIRERKVGYFYRRFKLPHNALDNTVKASYQNGILEVRITCTEFSPKTRVEITS

SEQ ID NO: 18 - protein, 381 aa, Babesia microti
MNGSVEELLNRLSAINDRCYLLVDEIKNYMSNKLYHELTLALIELFTMSEISCNDRLLLFEMIVHPIKNDLNILKFSHILRLSSEHLEPLASLDQLSKYDNYLSTDTQASFIKIAKSYHHTRNQSYDQSLKLLEEVKPEIESGFGLDITVISAYYKVSANLNKATHKYNSWYQDSLMYLNYTPLDSISPTERDELALDIAIASIAAPDNYNFGAVLIQPLINTCLKQHSTFGWVYAILMALNDGDFTQYDEIISKYKVQISHSELNHHKEQLQRKITLMAFLKLVFRKAKKQRIFTFEEISQNCRIPIDEVEYLLLKAMCNNVVKGKINQVEQIVSFTWVQPRIIDSTKLTVLLDGVNEWNQQLKALINKLKEITPELLVS

SEQ ID NO: 19 - protein, 700 aa, Babesia microti
MSGLFGQSQQIGGGLFGQSNQQSGGGLFGSTSQQPTQTCSGLFGSSPAPANSSIFGSNTQSAASSGGIFGSSTAPVNSGGGIFGQSNTNVSSGSGLFGGGNTTGQSGGGIFGSSTTSAPASGGGLFGQTGTTTSGGGGLFTSSFAPAPSSGGLFGQPSTPATSGTGLFSSTSTTQPSSGAGLFGSSTTPASGSGGLFGQPSTSTTTSGGIFGSSTTSAPASGGGLFGQTGTPASGSSGIFGSTNTTTSASGTGLFGSTSTTPQPGSGSGLFGGGNTTGQSGGGIFGSSTTSAPASGGGLFGQTGTTTSGGNLFGTTSTTTPAPASGGLFGSSSTTSTTTPTQATTTVGGGGLFGTASATTAPASGGLFGTTSTTTPAPASGGLFGSSSTTSTTPTQATTTVGGGGLFGTASATTAPVSGGLFGTTSTTTPAPANNTTPANTTTVPTAILTTSTPSPATDGLFGSTDVTTTTDSTTKLVGTSPFEKQTDSGPDVTAATNDTPNAFITDKPTAGGDLKGQVELSFSSVEHECVQDLLSNWEKRMEVKIQRFTEFAQDIQRIDRDLILQTEKLQVLLDEQATVQERQNQVQEMIQVIEKEQQQVLESLDIMDTALETLLGPDKKITSKSGETIDFVSNKLRDLEAQLKAAHDVVDSVVKASQPEPLANVAKVFAFHQDTIENIQLQTSEIEKKLDAIKQQQAV

SEQ ID NO: 20 - protein, 226 aa, Babesia microti
MASLLKVSPQDNIEFPLVLYTPLNANLLLENLSGVHVAFKIKTTAPKGYLVRPSTGTIKPGEALTVQIILQPLSEVPNVVNDRFLVQCTAIANDELVSKDFWTTLDKASIQDHRLNVTFKKDIGLNIQTSQSNIGVPPHIAARILTPLGPNAGVAELRQKYEELVSYCLTAEKQKAALVKDNEKLRQRLHLGPNDPASGNKWPLEGWHLPVMVIILVIILKAIGYW

SEQ ID NO: 21 - protein, 644 aa, Babesia microti
MSQGPAIGIDLGTTYSCVGVWKNETVEIIANDQGNRTTPSYVAFTDVERLVGDAAKNQDARNPENTVFDAKRLIGRKINDPCIQSDIKHWPFTVAAGPNDKPVIKVQFQGETKSFHPEEISSMVLTKMKEIAESYLGKTISNAVITVPAYFNDSQRQATKDAGTIAGLNVMRIINEPTAAAIAYGMDKKGTSEKNVLIFDLGGGTFDVSILTIEDGIFEVKATQGDTHLGGEDFDNRLVNFCVDDFKRKNGGKNISTNRRALRRLRTQCERAKRTLSHSTQATIVVEAIFDGIDYSCNITRARFEELCAEMFKNTLIPVEKALADADMDKKQIHEVVLVGGSTRIPKIQQLIKDFFNGKEPCKSINPDEAVAYGAAVQAAILTGEQSSKVQDLLLLDVTPLSLGLETAGGVMTVLIPRNTTIPAKKEQEFTTNENNQTGVMIQVFEGERSMTCDNNLLGKFHLTGIPPAPRGVPQIKVTFDIDANGILTVSAADKSTGKTEHVTITNDKGRLSQQDIDRMVAEAEKFREDDEKKKRCVESKNELENYCYSMKNALEEEGVKSKLSSSELSEAQKLLQNTFSWIESNQLAEKEEFEAKLKEVQAVCTPLTAKLYQAGGGVPGGAAPGGFNAGGAAPSGPTVEEVD

SEQ ID NO: 22 - protein, 299 aa, Babesia microti
MKLIACTLKNVETCVEVDPSDTVDALTNKIGSSLNNASASKMRLIHAGKILKMEQKISDYSDIKDGDKIIVLFSKQSEASTIANPTPAPTSTPIADANTSPPKPIPTTDPNALLMGEELEKAINGIVEMGFDVESVKAAMSAAFNNPNRAIELLTRHEVDVSDHDTHQSVQTTGVLDELRQHPMFEQMRAIVRSNPQTLPQILSLIGQSDPSLLQAITENQEEFIQLLSEPVLGTSGDFIDAQSITLTPEEMESINRLEGLGFSRPAAVEAFLACDKNEEMAANYLLENIADYVSDNDN

SEQ ID NO: 23 - protein, 144 aa, Babesia microti
MDGQTEQQLIEENIERFRILYQLHLDNNEPSPTAQFNYACALVCSNQRSHNDTAIYLLDELVRIRYESEECFYQLALAHMKRRSFVKSKEYLDRIIALEGSNQRVMALKSVVVSLLAQDTFMGGLLGATAAFAIILFFTMKRNT

SEQ ID NO: 24 - protein, 184 aa, Babesia microti
MNQNSLCCSKGFTMFAGGAFFLVSFKPETVITNPLLLRPLLNVSWGYIFGSHLWAAISTYNKKYWENRIIPDYANSPSREIMQSRLKINESRIYRIYLENLIQTNVLANGILLVTTSALAPSNKFLRICSGAALLLSIGNVVFALPEDEKDDDVGVVKTSSTSCFLSEVLSFCTFGAIVPYVFA

SEQ ID NO: 25 - DNA, 1206 bp, Babesia microti
gatgtatatgagatatcttctggtaatccacccgacatagagccaacatctacttctctagaaacaaatgtagttaccaactatattccagaacccaatgcggattcagaatctgtacatgttgaaatccaggaacatgataacatcaatccacaagacgcttgcgatagtgagccgctcgaacaaatggattctgataccagggtgttgcccgaaagtttggatgagggggtaccacaccaattctctagattagggcaccactcagacatggcatctgatataaatgatgaagaaccatcatttaaaatcggcgagaatgacataattcaaccaccctgggaagatacagctccataccattcaatagatgatgaagagcttgacaacttaatgagactaacggcgcaagaaacaagtgacgatcatgaagaagggaatggcaaactcaatacgaataaaagtgagaagactgaaagaaaatcgcatgatactcagacaccgcaagaaatatatgaagagcttgacaacttactgagactaacggcacaagaaatatatgaagagcgtaaagaagggcatggcaaacccaatacgaataaaagtgagaaggctgaaagaaaatcgcatgatactcagacaacgcaagaaatatgtgaagagtgtgaagaagggcatgacaaaatcaataagaataaaagtggaaatgctggaataaaatcgtatgatactcagacaccgcaggaaacaagtgacgctcatgaagaagggcatgacgaaatcaatacgaataaaagtgagaaggctgaaagaaaatcgcatgatactcagacaacgcaagaaatatgtgaagagtgtgaagaagggcatgacaaaatcaataagaataaaagtggaaatgctggaataaaatcgtatgatactcagacaccgcaggaaacaagtgacgctcatgaagaagagcatggcaatctcaataagaataaaagtgggaaggctggaataaaatcgcataatactcagacaccgctgaaaaaaaaagacttttgtaaagaagggtgtcatggttgcaataataagcccgaggataatgaaagagacccgtcgtcgcctgatgatgatggtggctgcgaatgcggcatgacgaatcactttgtctttgactacaagacaacactcttgttaaagagcctcaagactgaaaca

SEQ ID NO: 26 - DNA, 651 bp, Babesia microti
ataattacggatggattgtccgctattggctctgtggcgagtgaagtgggaaatacagtgaaggatgtttccagtgaagcgctcttaggagaattacaacaaattgcagacggtggcaaaattataagagacggcactgaaagcaattttgtgaattcaatagctaatacggttatgaaaaatgtagttggcactgcagttctcaaagcttcatctggtattacacataatggtgatttcgccttttacaactttatgtacccggaaaatgatgattatccatgggcatgtatctgtgatgaatctgattatgaagagtatattaagggcaagaaggacaaggttaggtgcaggaactatattgattcatccttacaaaatgcagttttatattgtaacccagcaaatcataactcttccataaatgacaatgccaataacaatcccccaaagcagattgataaccatgtgtccataccacaaacagcgccagcaaatcataccacagttttatctacggaagtcgacactaatcacaatgaacaaaaacaaccaaattcgccttcagttccaagtgaatctcaaaattcagtgtccgctccgaaagatgaatctgttagtagcaccgtagaaggagccaaatcaagttca

SEQ ID NO: 27 - DNA, 2775 bp, Babesia microti
acacttgtatctaaaattacaactccaaacaatttacccggaatagatacttgctcgaactgggaagatgtatcagtgtgtactactaaaaacaccagaaattgcatttcaggtgaaggcaaaccaaaggattgttttgcgattgggaaatccttatttaaaacatttccaaattgttatgaaggtgttataattgatcaagtcacgttttctggatttgagacactagaaatccattattgcgatatagataagatcttacacaaagcaaatgaagttgttaaatcaatacacgaattgaaagagaaaacaaatcaattaaccgagaaaattaaggacatacctgatttgattgataaagttattaaggtaaattcggaaatttcaaaaatatttcatcaagataaaattaaacatatggaaaaggaggccaacgatttcaaaaacgccttgaaaaccactagaaactacataatcagttatgattccttagataaaaccaagcaaagcaatctactaacttcacttggaaaattaatgaataagattaaaacaaaactatcagaaatggataagacattacattctacacttgacacaaacaataccattatagatcttgtcaataacaatagttcacacgttaaacatcctaatgattttaacaaaacaatggaactctacaatgaaactattactaaagctgatgcgattaaaaaaaatattgaaaaacttaaagaacataggaaaatatctactcacaaaacaatattttcaaacaatatagataagttgattgacaatttgacagattattttgaaaatataaatcgctccattgatgctgttagagacaaactttccaaataccaacttgaaacgggcaagatggttttattgtttaagaacgtgaatgaaattcaaaagcatattaaaaatacagatatgcatataagaacgtgcaattatgatttctcagatatcgaacaaaaatactcactcattactgcaaaaattactgtcgaagatggacaatcaatcacaacttcaaataaatcaactgtggatataccagaagagaaggtagatagggtgaatgtcaatgttgacaaagcagagaactctgataatgaaacttctcaggaaaatacatctgttaaaccgactgatcacaaagaaattgaggattcagcttccgaagaaaatgccattggagaaaatggtgattatgattcagatgaagatattgatacaaatgacgttaaagaagatcacgaaaatgcaattgattccgaatacaccgtttcatcgactggggatgtattagaagatgaggtagttgaggaaaatgccattggagaaaatggtgattatgattcagatgaagatattgatacaaatgacgttaaagaagatcacgaaaatgcaattgattccgaatacaccgtttcatcgactggggatgtattagaagatgaggtagttgaggaaaatgccattggagaaaatggtgattatgattcagatgaagatattgatacaaatgacgttaaagaagatcacgaaaatgcaattgattccgaatacaccgtttcatcgactggggatgtattagaagatgaggtagttgaggaaaatgccattggagaaaatggtgattatgattcagatgaagatattgatacaaatgacgttaaagaagatcacgaaaatgcaattgattccgaatacaccgtttcatcgactggggatgtattagaagatgaggtagttgaggaaaatgccattggagaaaatggtgattatgattcagatgaagatattgatacaaatgacgttaaagaagatcacgaaaatgcaattgattccgaatacctcgtgtcatcgactggggatgtattagaagatgaggtagttgaggaaaatgccattggagaaaatggtgattatgattcagatgaagatattgatacaaatgacgttaaagaagatcacgaggatgcaattgattccgaatacctcgtgtcattgactggggatgtattagaagatgaggtagttgaggaaaatgccattggagaaaatggtgatcatgattcagatgaagatattgatatagaagaagttaacgaagaggatcatgaagatgcgattgattctgataactccatatcaaatagcgaaaatgtagatactacaccaactgagaatgtagatactataccaactaagaatgcaaatactacaccaactaagaatgcaaatgctacaccgactaagaatgtagatactataccaactaagaatgtagatactataccaactaagaatgcaaatactacaccaactaagaatgtagttacgacaccaactaagaatgcaaatactacaccaactaagaatgtagttacgacaccaactaagaatgcaaatactacaccaactaagaatgtagttacgacaccaactaagaatgcaaatactacaccaactaagaatgtagttacgacatcaactggaaatgtaagaaccaaacacacatcgacaaattctcatgttttagctccggatacagacgaatacccagcacaaatttcacaacataagacgattgataaatattatcaatcgttggaattggaggatgaagaaaatgcagatatatcaagctcagataaaccagtttctccaataaatttggaagataaaaattcgacatatgatcatatgcataaaaccgataatgttaaaagcgccggtattgcttcatatgat

SEQ ID NO: 28 - DNA, 564 bp, Babesia microti
aaccgctcttgtccaggaaacaatggcgttggcggtggatctggtgataataacagtggcataattccaaatgatccacacccctgttgtaacaatcttagacaaaaaccccaataccaaaccaagccagaaaatgaactagtcaatgatgatagagatttgaactttaataagatcagaggtggcaagcaaatcatcacctttactgtcccttccatagatgatctcaagaataaaagattatccgactctgaattcattttatcagaaaaggcaaatccattgatttcctccggcgacagtaaaaacgttattgtattcgaagtaaaaaacgataatgagaagttaatgggtagtgttgaagttggtcaatgggaagttactatcaccacatcatgcatcagacgtatcgttattttcgattccaatgaagtttcagataacattcccatgtatatatatatcgtggactactttgaaggaggtaatagcactgtttcaaaattctttttcgcgaataatagatggaacgctgatttcaccaatcacacacctaatgctgct

SEQ ID NO: 29 - DNA, 222 bp, Babesia microti
ttcctactgaaccgcagcgaatttaagtggtttaaagtgggtctaataattaccacgatattcccttttaagcattcatttgactataatttgacacacatatttctattttcaatatgcacgttaatattttgtgtaaaacctgtggatgaggagagcggtgccaaaaaggagggatttgacttcaagaagatggtgccagataagttcaaaaagtatacc

SEQ ID NO: 30 - DNA, 3681 bp, Babesia microti
gatagtggtaattcttccccccaaacgccgccagaaactagcagtcctataaatggtgtaatcggcgatgaaaataatggattggaacatttgagtagttctggtctggaagttgatgataacctgcccgaattactcaaaacttctccattttcaggccaaaattcagatgttcaatctgcttcaacaccagttgaacccactactcctgttcattccaatgaccaatctaatcctatcaccaataaagttgataccaattctaatgaccatactgatattaagaatgaaggttcatcccatcgtacttcatccaacaattcttctgtcacaaccaatactaataatgagattagaaatggcggaggaccattagatcagaatgaagataaggctgaagatgaaggtgaaactgatgccgaagggagaggatggaatgagagaacgaaaaataaacctacatttaatgccacaaatcgcgattttgttgatgataacctgcccgaattactcaaaacttctccattttcaggccaaaattcagatgttcaatctgcttcaacaccagttgaacccactactcctgttcattccaatgaccaatctaatcctatcaccaataaagttgataccaattctaatgaccatactgatattaagaatgaaggttcatcccatcgtacttcatccaacaattcttctgtcacaaccaatactaataatgagattagaaatggcggaggaccattagatcagaatgaagataaggctgaagatgaaggtgaaactgatgccgaagggagaggatggaatgagagaacgaaaaataaacctacatttaatgccacaaatcgcgcttctcccgatggaattggcaaaatgaacatggaagaaaaacagttggagaattttataaatgtatcatccaacgctcttgaattagatatctcaattggccgggataattttgctactaaattcctagcacaacacgttaatatttttggagacaggataagcggcttgagtgcggcgtatgtagagggctataataacttggctaaaattatgtacaatagtcactctgttttatttgatagaaaattcaatggagcagttatttccgacaatttgattggaaatattgcagattttggttcatacttcttagagatatctccaaatacaactagaaccaatagaagtgattatctcaaatcagtagtactttccaaagttcagtatctgttgtctgctgatttttccaccactgacaacatccaaaggctcacaaatttggcacttgccctaggctataacaatgtcaaagaaaataatcctggcaattcccaacattcaataacaacctccctctcaactgagctattttggtcctttggcaacaatatattcttatttggacacttggcaactctaatgcttgcttatctagaatctaatgcatatttcacatctggtgcgaccaggcctttcttttcatggcaaactctggtttccactggtggtaatgagaaatttgataaactggactctatgtgtggagtgattcgtggatcaaagtattcgcgaaaaaacaatggatttatcaagccacactacaaaagattacgaaggaagactttgctcgaaggtgaaccgcgtttgttgtgcagcatgctggaagaggctttggatactgtggataaagctattaaatttaagggcgaagagctaaattcacaaggcgcaaatattgaaaattcggtctccaatgacatcaacagtaaacgtttacaggcgaaactttgctcaaatttgaatgattcacttataaacgtgagctgtgattttagatcatcaaaattggacaaacacaataagaaattaagagaggcatttgacttattattagcttgtggcaatttgaacactggtaaaaaggaggcatttccggaatacttgagattgatctctaaccccttcgaatacggcgatattttttccatgactatgtggtgggatcctagggagtttgatggcaaacagggctgggtcgaaatatacaaaaagttgaggaagaatattatgaaacctgaactgaaaaatgtggacatgcaactcaaatacgactcggcaatttcttattacaaacaactgaaggaatcggaaacctaccccaagaaaaacattccatgggctagattatacctttacatgtcagtaatcatgtcaagatcgaacgctatgagttgggctgaggacgccctccgctcatttagcaatctatatcgcatgaaaccttcattagtgatgagaggggaagggttagagactttgctcaattactgcgctcctgatccagtggctttatcacacatattcctctaccactttttgacaaagaaagatgccggtaaagatttggaaaaagacttgagaagacttgagaaagggacactcctctcgcgcattgtgaattcatcgtcaatattcatacctaataaactaaagaaattccttaaaatgggcgctcgcggatttttcaataaaaaacttaacactttgagagcaaagagcacccttctacgattattccccaagaatttactacattctgcattgggcgcaattgaattcaccacacattcactagccacgttacaaatatctaagaatatggatatgtgggagagtttggcccagacaaagaatttagatgcaggcggtttccctggcgaaatcgattcactgtttaatcattggtcggaaagtggtggatacagtgggtatattaccggcaaattagagaatggagatgatctaacgggcgatgatatcaaaaaaatgaacataaaggcccctattaacaatgattcactcaattggcaaaagtatatcaataaaaaaatatccgagcattttggcaaatttctaaaccttccatttatccaagcctccggttcacaaaagaactacatatatcagcttgtacgggatagtaaggcaaaccttgacgataatttggaacaaactgtgttctttggcaaagtgcttcccccaggaaaaactaataatgttattaagaaattgaaaagaattgcagattcatttaccagcatgctgttacgttcttctgctcgtcctgttgaccatgctgtgtgggttggagtgaaaatcaacgtacctattgtaattcatattactaaaaagttgtacatgatacagcgtgatatgcctaggaaggaagcgtggaatttggaaagtgctttcttggacctgttgcaggatctggtgataatggttacaaacccggggaagagatctccgatagggtttgaaacgattgggggcaatcctggattaccagaaattagcattagataccctcatatgtcgattgaggaaaggaagattgaatttcaacattcacaatgtgcagatcattgtatatcaatttggagatccctaatcgcatttacacttaacacactaaacaatcctgcggcaataaagcaatttgagaaatccttatccagtaatagttctctaaacgacatgtcgaagcccgaatatatcaacagcttcaagtatatactaaaaggcgactcggtgttgcatatgtatgataacatgttgcctaggaaggtgaaaagggaaataaaggcactaaagtatggtaag

SEQ ID NO: 31 - DNA, 1299 bp, Babesia microti
ctgcctgacattctatcgcagaataacacgtttaaatcttttctcgaagtaaataacgtggatcaggaagatttgatttgtaataaggcactgtgtaaatccactgactctatcaacagaaatacaagttcttattgttataaatacaaactgtgtagtaagtgtagcgtatccaacgtcccagatcaccctgtttgctatcttctggataatgaccacaattacatccacttaatggaggggcatttaggctctcagcccataggatcagcgaatagtcacgataattcttctcatgacgaacattcatcgcattcgaataatggagatatgatggatgagcatgaagaggaaaattttctgcaggaatacgaatcaaaatcaatgaaattcatacctactagcaacatgagtgactttgatcatgctaggcgatcttgtgccgtggattccaaaggcaatgtaatgataagtgttagattgatcatccaatggtatatgtcaaaggataaatctaataatcagcagcaccatggcaatgatgatgattctcaaaattatgacgcaaattatttacaactcacccctatgtattctgacgactctgttaattcttctatgttagagatggaccacgacgatagtgaatcatcaaattcgcataaatctaggatggcaaatatggccaaaaacttccaagttttgaaaaacatccataaaagtgcagttaagcgttataaatctccaaaggccaagatatatctcatattttctaatccaaagatcaacagctgtagacacccggtaatatacaacggtaaaatctccccctcaagcatgtttgtagcgaaacttgagtcaacaatatcacaaattgatttgacacaagatctgatcaaatcgtcaatagaaactattgtctcttgtgaagcttgtgataagttaaaatacaacagttgtatacaggttacctgtgccaaaaatacaccaggcgcagcgtctttagctatgggtagtgctgtatacgttccaatgacgaatactacgattggagtaaatgcccacaaccctaatgcggtagtagctgccggtatccctatgggtaaaatacctgtaatcccccatccagcagctataagtggtggaaatgttggtcatttgaataatggtttgcacaaggcggttaataatgctgttatgatgccaaatggaacatcattgccagtgcaaagcggagttgttataaaatcattatacaattgtctcgcttttttactgacaattttgtatctcaatttc

SEQ ID NO: 32 - DNA, 657 bp, Babesia microti
aaccctatatttgcatctgcaacctcagctccaagtaggaatagagctcaagacatgaccaaagcggaatgtatagaactattaaaaggcatagtgaaatctcaagaggaaaccaaaattgttatgaagaagttgacctcagacctgataaacaatccactgcgcctagaacaggtatataccaaggccagtcaaatgcagccagaggatcctatggagcaatggggagtcactgtaattgatctcgaccatttgattgaaaagtatcaacatgatcctattgtgaaggattacattttgaagatcatgaattcgcccggcataaacgatacaactctgagtgacgatgtacgcaatattactattagtcaaatattagctatccatgaatatatgctcagcgaattggagtctgtagtccgcgaattcaaatcgctgcagaacagacaaactatggagattaaaacgttaactattgctgcgcaagcaatcgtagctgccaaagtagaggaaaaattcaatttaacatcagatcaggttgaatcggctgtgataataaaccatgccgaattaactgctagccatgcattcaccagattgactatgcaaatgcagacggagatgagtgagttgattgggtgccaatttcccgggtgg

SEQ ID NO: 33 - DNA, 498 bp, Babesia microti
agtacgaatggtaaggggggtgtagactctgtgtcgaagaaatctttcgtcattgaacttgaagactccacttttgaaagaaagacccaagcttcttctggtggcacttctggtgtatggtttgtcaagttttatgcaccatggtgtggacattgtagatcaatggagaatgattggaatgagttggccaatattttgggcaaacaaattaatgtagcaaaaattgatgctacaaaacacagtgttactgcgaaaagatttggaattacaagtttcccaacacttttgttgctgaaagatggaaatttctatcaatatgaaaataataatagaactgctgatgctcttaaacaattcgctttgcatggctataaacaggttaaatcaaagcctgttcctaaagaatggagttattttgttcggttcaaattctttatcaaatctggattttatgaagttaagcgcatataccagttagcatatcctgggttcataaca

SEQ ID NO: 34 - DNA, 753 bp, Babesia microti
atggcgaaattacatgagagtacatcaagtgcaagtgctagcttcgacccagagaagtcggattatgacgatacttatgttttaacagagacaacgccaacttacataaggcatggatttgttcgaaaggtctttgctattttgtttgctcaattactagtgacccttggattttcattgatctgttatttctaccgagaatctgttcattcgttcatatccaaaaatatctggatttttccaaccttagctatattatcatttataacatcccttatactcatattttcaccttcactctctaggagatatccgttgaattacgctattctcgttatagaaacattgtatttctcattcattgtcggattgtcatgcgcgtttactaaaagtccgactgccatagtattatccgtctctattactcttgggatcattcttcttgttgttctgttcacactccagaccaaaattgacttcactagatatattatctattttattttatttagttttgtaacgttggtttttggctttattggtatctttgtcccatttgacacccctctcaggatgttttattacggtttaggcgtacttggttattcactttggatggtacttgatcttcaactgattatcggtggcaagacttacgaatggacggttgatgattacgtccctgcatcactctcgctttatacagatgttattggaattttcttaaacgttcatggcatgttttctgataga

SEQ ID NO: 35 - DNA, 960 bp, Babesia microti
atgactataaccttgaatataaaggttaattctgagacaaattttacggtagaggctgaaccttcctttactgtaaaggaattaaagatattatgtgaatcacaatcgaatattgaggcgcaaaatcaacgcttaatttgtaagggcaagcttttgaaggatacagacattctttccgatgtaggggcagttgatggtgccacggtttacctggttcgtagccaagtcaacaaaacgcaatcagctgctcctaagcaaaatactgtccctcaacccacattacagactacaaatcaacctgctggtcaaacacaaacgtctggacttggatttcaacaacaagggtttcagcaaggattttctggatctcagcaaccaggatttcaagctaaccccttccaaagcatgcttgctggtggatttcccaatttagatcccactcaaatgatggagattttgaatagtccaatggcacaggaagctatgcagagattgagccagaatccagaagttttgaggaatattttgcagaactcatctttgatgactcccatgcttgaacaaaatcctatgctatcagaaatgctatcaaacccggaattgatgagaagtatgttgagacccgaggttttacaagccggattgcaaatgcaccaagccatgcagcagcagcaacaacaacaacctgggactcaaaccaatccaataggttcacaaaatccagacttcagcaacatgatgaggcaaatgatgaatgtatttcagcaaaatccatctgttgcacagcccacagcaccacaggtatataccgacccgaggcctccggctgaaagatttgcaactcagctacaggcattggctgagatgggattcattgacactgaaaaaaatatcactgcacttattgcgaccaatggcgatctcaacgctacagtgaccaggctgcttgaatccaatttt

SEQ ID NO: 36 - DNA, 927 bp, Babesia microti
atggaagaggctgaacgcaaattcaaggaatttaattgggccgatagtcagggatggcgaatttactgggacaacttgtatccaactccccccttatccaaagtagataagttcaaacgttcctggttcaagagaaacgtagattctaatatatcttcaacaccactgtctgaggttggcaaacaaactcaacaaactactccaaatcaatatcgttcgcagggcaactttgctattttaactctcgaggccggtgttagattgttgtacctgcttattacagcacctttatttgttctaccactaataggagtcaggctcagtagatatatatactaccactactacatagacatagcgctgttattgatctttctattgtctggcatcgtaagggaaagaggtctaccgaaaatggattccgtgtggctggcatctgctttctattcagacatgacacaatatataatgtatacatgcatcctgatgatgtccgcaccaaggcctgtttatctgattttaccctttctaacttgtctaatcgggctaaattctctggccgaaacaaatttatccaaactgcccaaatggcttgaaaacatagtgggtgaaatatttaagtatactaaggataatatatattggttaatgcaaactcgtggcgatgttgagtgttatttgttgttttatattgtattcgggttcctaacgaaatcaagcgcagttatcactttaatggcctacataaactttatgaagttaaggattggcattggtgatccctttataatgtctgccttctctaaattgcatggctgcatagtgaagttgttatcgtatgatatgtttggacatatacctttgaggatgtacttgaagatatcagagctcttaactaaatatatgtcggggccaaggccgcaatat

SEQ ID NO: 37 - DNA, 2898 bp, Babesia microti
atggaggaccaaaatactacaaatgaaagcatttcaaatttgcacatggaaaactacttccccaaagatattttttccaacctagataatcaaaaaaacttaaaaacatatcattcaaagacgtttgagaaatatttcgagtctgctgccaaagataatgttgaaaggtgtcgtacatctgctgtcaaagaatggctgaatagaaatcttcctagtgaatataatgaaaggcacaagcccacacttaaactaaacctgagccccaaccacttaaactcaacttacaacaaacaaatttccgattacacaaggaatgcctataatcgcatagaacagttgaagaagcttcaagaagcttactctctgttaagacagaaactacaagagaaacgcagggcctcttgtcataaggaggtctctgaggcagaggcttcagttcttaatactcaaagattgataattagcttaaagagagaagacaaagataaaatcgccacagagttacttatacaaaatgcagaagagttaggtataccatccaaagatttggaaactctaaacggctacacatttaatgaggctctcaaaactataatcgatataatttctaacatgataaacaacaaatttatgacaaatttgaaagataggtgtgaatcagctaggcgtaaggcaggatctgaatatcgcgatgaaatggaaaggataaaagtcgatttggacatgtacaaaacaaagtctgaaaaactcgaatccacaatatctacattgaattcttatgcagattctagcaaacaaaatgctgaaaaggtcatggaattgcagcattcgctagaagattccaatagacaaattaatcagctaaaagattcactgtacaatatggccaaagttaatgaggagttggaaaaaaacgaagttaagctagaggaaagtttggtagagttggagaggtataagttggaatctcaaaagcttgagtcagtcattaaagatttagagcttttgaaccaacaaaaacatgataatgaagaactcattaaaatgctgcatgatgagctagatcagtgtaagtctaagtcactgcaattgaataaaaagattgaagatcttgaaaattcaaacaaggaaaacctaatacccttgaacaatgaattggcccaggcttatcagaaactatccgaattggagcattcatatgaacagattgaacaccagaagaatgaagcagataaaaaacttgaatcttcaattgatgagattgagaaacagaaacaacactccgaagaattggagcaatccatcactaaattgaaacaaacaattgaagaaatggaaaaggaaaccacggaccaagtaaatgatctgcaaacgtgcctaatagatgcgaattgtaaaatcgaatcgctagagggaacgatttctcagttgaaacagctaaacttagagttggaagggggagaacataggatacaggaattacaatccaagcttaccgaatcaaattccacaattgaaaaacttgaaaagacgatcacagaattggaaaatgtgtctagtaaatatatcgactacgatgagaagatggataatttgcaaaagcagctgaaagaatacactgaaaagatcacagtgctggaaaacaacgcctctgaattcaactataaagaccaagctgaaacgttgaagacggaactggataagtcgcaactaaagataatggaactggagactatgattaaggagaacagcgaaaaaacggaaagagtgccatctcccagaaaggacaacgaggaggttgatagattaacttcggaaatattacagttgaagaatcaggtggacattctgcaaaatgaaaagactgaccttgaacaaaaattatcacaacaatctcccagaaaggacaacgaggaggttgatagattaacttcggaaatattacagttgaagaatcaggtggacattctgcaaaatgaaaagactgaccttgaacaaaaattatcacaacaatctcccagaaaggacaacgaggaagttgatagattaacttcggaaatattacagttgaagaatcaggtggacattctgcaaaatgaaaagactgaccttgaacaaaaattatcacaacaatctcccagaaaggacaacgaggaagttgatagattaacttcggaaatattacagttgaagaatcaggtggacattctgcaaaatgaaaagactgaccttgaacaaaaattatcacaacaatctcccagaaaggacaacgaggaggttgatagattaacttcggaaatattacagttgaagaatcaggtggacattctgcaaaatgaaaagactgaccttgaacaaaaattatcacaaattagtgcggttattgaagaaaatagacaatacaaggaaaaaatcgaattactcgagagaaaattgaatgaaataaataaagagaaaccaaaagactcatttacaaacgtagaagttatcaatgcagcttttgaagatgtaagatccctcccaatcaattcctccaatgcctctgattggaccgcaatgcccagtgagctggagctggaagagcataaattgtacaaaaaaaaatctagtagaaaggggaaaaagaaatcatcaagggagaaggagactagcagaagtgacatatcatccaggtcaactagcaggcactcaaagaaggataagcctacaatcgttgacaacaactctactgatgtagaagcatcagattcaaatgaacagatgattagtgatccagttgaatcgattacagaccagttgaacttggttaaacaatcaacaatacaactaactgagctggggtacgacagcatatccaacgttgggtcgtacttgtctgactacatatttggcggtaat

SEQ ID NO: 38 - DNA, 795 bp, Babesia microti
atggatgatctacccggttcattacatcaatcaacaccaaatgagaagccaatggctccacccacagcaccaaaatctgctccacacgttcctatttctgtcgaatcaaaggagttatcaaaagaaattccaaaagaatctcctatgactaaggaggacaagaaagacgtggccaagagtaaagtttctgctgctgtaactgagaaaaaagttgttaaggaaccagtggtacctaaaataacaattgagatgaaaaaattttctatgtccagagaatccacccaatactcaatcttcattatatttaaccttatctttgcactattatacattttcaaagtaagattgatggccatattttgcaatctaattattgtggctatttcaattggagctatcctttcctccatagaccctaatagacgcaaagttgatgagacaaatgttaatattatatttataagcccgacaacagtttcagatttagcaatcgtaatcacggcaaaactgaaccagtacatttcatactttagaagaattttgttatggcaggattttattctttcaaccagatttactttgtgtgtctacattatgggaatccttttcaaaattataccgctggttgctttaatatatataatgtgttgggcattttatctttatatgttcatatgcaaggagatggcagatcagttgttggacattataatggtttatttgaaaagggtgtcgggaaatttcgacgagttctgctgcaatatccccaagatgaaggatgtgggtaaggaactg

SEQ ID NO: 39 - DNA, 732 bp, Babesia microti
atgtttaacgaaaacgagatccctcagttccagcgccctttcaccccctccaaagacacggagattgacactagcatcaatttccctgccaattcaattctaaataacatatcatttcaaaaattattggattggttagatgaatgcacagaaggcttaccaatcgtggaaagttttgcgagacgattcaacgttactaggggtcatatagcggcaatattttctgctatattaattctgtactttattttcggctggaaaattaacatcttttgcaatacaatagggttggtatatccagcatttaaatctcataaggttttactgattcatagagccatgactgaaagtccaaaagcaacagctactatcacaactgggggaaaggacaaagaaaatgacacaaaccctcaaatgcctacttgtctcaatggaatccaaggggaaattatgttttggcttaggtactggattgtttactcgctttatttatttatttcgatattaattttccccttgatatcatggttgccactaatatctatagttcgggttggttttatcctatatctctaccatccatacactagaggggccaacgcgatctactacctcgtaatatcacctctgctaagcaaaaaccagaagatcatagaacaggccattgacaccctcgaaagggttgcgatgggtgaaatcaggaaatttacagaaaaacaaatgaagctacac

SEQ ID NO: 40 - DNA, 2571 bp, Babesia microti
atggatagcattgaagagtgtaataaattggtagatgcggtgaccaaattggctacatcatttgattaccaagcgcaagaatatctatacaatcttacccgcgatgaaaatgcaattaacattgcattatcctttctattatccaataaatacgtgttgaactattcaagcagttacagcaacgaaattttacaatatataaccgatgttcgtggcccaagcagttgttgcatctgggacaatatccaattggacgaaaataccagtaggcatctgggccacataacgtctattttgtttaaggagcatatgcatatagttttgtgtttcgacgatgcaaagcttattgcacttattgatacattggttactatttggcaattacaatcaactatatcactagtaactcaaagctgtttcgctgataatgctactgatgatcatataacgagagttatttctatgattatattcaaaaagccgcaattattaacccacttcatcaacatactaaacactcccgaggtaactgataagtatgtggctgatttctcaatgaaatttaaattaagaaattgcgatagagatgatatttatgtcacaaggtataggcatatgattgtctatgtgttagctcaggtgcttgaaaattttgtgtcttcgaacgttactgataagatatcttttttgccaattaacttctcagatttagtcatattgtcgggaaattgtaacaataggtatctttactcatgcgcgttattaattgtaaattcattgaactttcacatttcagaagagttattacatcaattattggtaattgttgagaaatctggatttgactgcgatatacttgagatgtggaattttgcaatcagtcatttacaatatttgaaagttgacaatttcatgatgaaatctttcaggttagtccaaaacgggctcattgaatccacaaatccaataaacattgtgcctgtattaaacggtataacagtatactatgttaataattcatcagttccaactgagatcgtaagagcttacaggcaattgctttccgtcgaatcccttgacattcccattgaaatcggtgagttatttgcagctgtccgaaatgcttgcactgaaagtctgattacgtatgataaaattgcttcatttgtgtcgcaatttgacataagaatgatttcaagtaggttaattgaactttgcaatcctaacatgcaatgttggtcttggttgaatgtaaattgttgtgtagaagaatttcccacggattatgacatatacattgattctatgaaaaatgtatttaaagacctatttttcatcttaccagaacaacttttagacactataataagtgagatattacaatctgtaaatgaaaatacggaaacgtcactaatagcattaatttgctatgattgcctagaagtaaggcatgcattggcaattctagacaagattaaatcattgtttacgctgaaattgtcttcaaactacgatttcatccttacttgtatgttttgccaatccatatttgtgcctcatttgcacaggataaaagatatattattccagagatttgttatcaacatgaaaatatcgaatatttggtctaaaagactggctaaatgtatagcaatattgaacgatagggattccgttcatagcgtgattgaatgtgttcagtatatattggaaacttctaaatccaagggtgtattgtcacaaaatcactatccttcaattctatcattgtcaaatcttttagatacgttgccacaaatgccttgtgatgttaatttgactggatccaattatattttttgcaaatgcttgctcgcgttaagtatttctactagacaattatcacaaatattgccaaaaatcgctctcaatttggatatactatatgaaggtgatgtggacaacgatttgttttgtttatcaacatctgatagccaagctgcagttaacttattgatgcaactatatatacaaaataaggtaaccgatatacctaatgaattgcacgagatacgtcaccccgggcttttaatgttatgtaatttgaatgaaaattttgaacaattcaggatgaataagtgtgtagagttcatgtcaacggtcagccaagaatcttgctgtcactgtgcatgggcctgtattgcctatgcatcttatgttatcgaacagaaattcaactttgaattttcaatggagatggttaaagtgatatatagggtgttaccatgtttaattaaaatggttaagtccaagaatgagcctttacattgggatttagatgcaattttggagtacagaattaatgaaatacatccttcaattaagtgtctaaaggggacaatttggaatatttactatgatggtacagttagtgtcggctctgacgcttgtaaatatttatcactagtaaataaatatgctccaagaatcattaaaagtttatcaaatccaaatgtattgcagttggtgtatcaaatttcaatgtgtactggatccattgaagttatagcaaataaaatcgcaatttcacta

SEQ ID NO: 41 - DNA, 513 bp, Babesia microti
atgagctgcatattgaagtgtaacaacgaggatgaattggttgtggatggggaaaaacccaaggtagtagagtacgtcgagaaaccatcggtaatttacaagcctacaaccgttgtcccccctaacagcctgatcgaaattactgctcccaaggatttgcctcaaaatccaacattcttcccaaccatagatacatttttcgacaatgacgtcaagcagatcgttttgttgatggaattgcccggatttgtggcgggagatattgatttggaggttggtgaaggtgaagtttgtgtttgcggccctaggtctaaggaggaactatatgagaagtatggtcaaaatttagatatacacattcgagaacgaaaggttggctacttttaccgtcgcttcaagctcccgcacaatgctcttgacaacaccgtaaaggcatcgtatcaaaatgggattctcgaagtgcgcattacctgtacagaattttcaccgaaaacgcgtgtggagatcacatct

SEQ ID NO: 42 - DNA, 1146 bp, Babesia microti
atgaacgggtcggtggaagaactattaaatcgcttatcggcgattaacgacaggtgttatctgctggtggatgaaataaaaaattacatgtcaaacaaactttaccatgagttaaccctagcactcatcgagttgtttaccatgtctgaaatatcttgcaatgacagacttcttttatttgaaatgatagtgcatcctatcaaaaatgatctgaacattttgaagttctcacatattttacgcctgtcctctgaacatctagagcctttagcgtcactagatcaattgtctaaatatgacaattacttatcaacagacactcaagccagttttatgattaaaattgcaaagtcataccaccatactcggaatcaatcctacgaccaaagtttgaagcttttggaggaagtaaaacctgagatagaatccggatttggcttggacataacggtaatctcagcctattacaaagtgtcagcgaatctaaataaggcaacgcacaaatacaattcatggtaccaggactcactcatgtacctaaactacactcctctggatagtatcagtccaactgaacgtgatgaattggcactggatatagcaattgcctcgattgcagcgccagataactacaattttggtgctgttctaattcagccactaatcaacacgtgtttgaaacagcactccacatttggctgggtatacgcaattttaatggctcttaatgatggcgacttcacacaatacgatgagataatttcaaaatataaagtccaaatcagccattccgaattaaatcatcacaaagagcaactccaaagaaaaattacacttatggcattcctgaaactcgtgtttagaaaggctaagaaacaacgcatatttacattcgaagaaatatcccagaattgtcggattccaatagatgaggtggaatacttgcttctaaaagctatgtgcaacaatgtggttaagggtaaaattaaccaggttgagcagatcgtcagttttacgtgggttcaacctaggataatcgactcaacaaaattgacagttctcctagatggggttaacgagtggaatcaacaactaaaagcgctcataaacaagctcaaagaaattactccagagttactagtgtcg

SEQ ID NO: 43 - DNA, 2100 bp, Babesia microti
atgtcgggactgtttggtcaatcgcagcagattggtggtggattatttggccaatctaaccaacagtctgggggagggttattcgggtcaacatcgcaacagccaacccaaacatgcagcggattatttggatcatctccagctcccgcaaatagtagtattttcggttcaaatacacagtctgctgcttctagcggtgggatttttggttcatccacagctccagttaatagcggaggcggcatttttggtcaatctaacactaatgttagcagtggtagtggattatttggcggtggaaatactacaggtcaatctggtgggggtatttttggttcatcaactacttcggctccagcatcaggaggagggttattcggccaaactggaaccactaccagtggaggaggtggattatttacctcatcttttgcgccagcgccaagtagtggcggactattcgggcagccatccacacctgcgacaagcggcactggattgttttctagtaccagtacgactcaaccaagttcgggagcggggctatttggttcaagtactactccagctagtggaagcggtggtctttttggtcaacctagcacttcaacgacaactagtggtggtatttttggttcatcaactacttcggctccagcatcaggaggagggttatttggccaaacaggaacccctgccagtggaagcagtggcatatttggctcaacaaataccacaacctcggcctcagggactggattgttcggttcgacgtcaacaactccacagcctggcagtggtagtggattatttggcggtggaaatactacaggtcaatctggtgggggtatttttggttcatcaactacttcggctccagcatcaggaggagggttattcggccaaactggaaccactaccagtggaggtaatttatttggcaccaccagcactacaacaccggcgcctgctagcggtggactattcggttcttcttctactacgtccacaacaactccaacacaagcaacaactacagtaggagggggagggttgtttggcactgcaagtgcaactactgcgccggcgtctggtggattatttggcaccaccagcaccacaacaccggcgcctgctagcggtggactattcggttcttcttctactacgtccacaactccaacacaagcaacaactacagtaggagggggagggttgtttggcactgcaagtgcaactactgcgccggtgtctggtggattatttggcaccaccagcactacaacaccggcgcctgctaacaacactactcctgctaacactacaactgtgccaactgccatactaacaactagtacaccgtcccctgctaccgatggtttatttggatcgaccgacgttacaactactactgattccacaactaaactcgttggcactagcccttttgaaaaacaaacagattcggggccggatgtcactgccgctactaacgatacgcctaacgcatttatcacagataagccaacagctggaggagatctcaaaggccaagttgagttgtcattttcgtctgtggagcatgaatgtgtgcaagacctactttctaactgggagaagagaatggaagtcaagatacaaaggtttactgagtttgctcaagacatacagaggatcgatcgcgatctaatactacaaacagaaaagttacaagtattgttggatgagcaagcaactgttcaggaaagacagaaccaagtacaggagatgatccaagttattgagaaggaacagcaacaggtgctagaatcgttggatataatggatactgcgcttgagacgttgttgggtccagataagaaaataaccagtaagagtggcgaaactatagatttcgtctccaacaaattgcgggatttagaagctcaattaaaggccgcacatgatgtggtggattctgtggttaaagcttcgcaaccggagcccctggctaatgttgctaaagtgtttgcattccaccaggacactattgaaaatatccagcttcagacatcggagattgagaagaaacttgacgctattaagcagcagcaagcagtc

SEQ ID NO: 44 - DNA, 678 bp, Babesia microti
atggcatcgctgctgaaagtatctccacaggacaatatcgagtttccactggtgttatacacgcctttaaatgcgaacttattactggaaaacttatctggtgtgcatgtggcctttaaaatcaagaccacagcacccaagggctatctagtccgcccctcaacagggacaattaaacctggagaggcacttactgttcaaatcattttgcagcctcttagtgaggttccaaatgtggtcaacgacaggttcctcgtccagtgcactgccatcgcaaatgacgaattggtttccaaggatttctggactacactcgacaaagccagtatccaagatcaccgcctcaatgtaacctttaagaaggatattggtctcaacatacagacaagccaatccaacattggcgtcccgcctcacatcgccgccagaattctcacccccttgggacccaatgcaggcgttgcagaactaagacaaaagtacgaggagctagtatcatactgtctaacggcagaaaagcaaaaggctgctctggttaaagacaacgagaaacttcgtcagcgcctccacctgggccccaacgaccctgcctctggaaacaaatggcccctagaaggttggcatctacccgtaatggtgatcatattagtaattattctcaaggccattggctactgg

SEQ ID NO: 45 - DNA, 1932 bp, Babesia microti
atgtcacaaggacctgctattgggattgatttgggaaccacttactcatgtgttggcgtttggaaaaatgaaactgtagaaattatagccaatgatcaaggcaaccgtacaactccgtcttatgttgccttcactgatgtggagcgattggttggcgacgccgccaaaaatcaggatgctagaaatcctgaaaacacagtttttgatgcaaaaagattgatagggagaaagatcaatgatccctgcatccaaagtgatataaaacactggccatttactgttgcggctggaccaaatgataagcctgtgatcaaggtacaatttcagggtgaaactaaatccttccacccagaggaaatatcttctatggtcctcaccaagatgaaggaaattgcagaatcttatttgggaaagacaatctctaatgctgttatcactgtacccgcttattttaacgattctcaacgacaggccactaaggacgctggtaccattgctgggttaaatgttatgcgtattatcaatgaaccaactgctgccgcaattgcctatggtatggacaagaaaggtacttctgaaaaaaatgtgttgattttcgatttgggcggtggcactttcgatgtatcaattttaactatcgaagatggcatttttgaggtaaaggccacacaaggtgatacccacttgggcggtgaggactttgacaacagattggtcaacttttgcgtagatgactttaagcgaaagaatggcgggaagaatatttcaaccaatagacgtgcattacgtagacttagaacacaatgtgaacgtgctaaacgcaccttatcccactcaacacaggccacgattgttgtagaagctatatttgatggcatcgactacagttgcaacatcactagggccagattcgaggagctctgtgccgaaatgttcaaaaacactttaatcccagtcgaaaaagccttagctgatgcagatatggataagaagcagatccatgaagtggtacttgtaggaggttcgacccgtatccctaagatacagcaattgattaaggactttttcaatggcaaagaaccctgcaaatcaattaacccagacgaagcagtagcctatggcgcggctgttcaggctgcaattttaactggcgaacaatctagcaaggtccaagatttactgttactagatgtcactccactgtcactaggacttgagaccgccggcggtgttatgaccgtgctgatacccagaaatactactatccccgccaagaaggaacaagaatttacaaccaatgaaaataaccaaacgggggttatgatccaggtcttcgaaggagaacgttctatgacatgtgacaacaatttactaggcaaattccatttgactggcatacctcctgctccaagaggagttccccagattaaagtcacctttgacatcgatgccaatggtatattgacagtgtctgctgccgacaagtcgacaggaaagactgaacatgtaactataacaaatgacaagggaagactttcacagcaagacattgataggatggttgctgaagcagagaagtttagggaagatgatgaaaagaagaagaggtgtgttgaatccaagaatgaacttgagaactattgttattcaatgaaaaatgccttggaagaggagggtgttaaatctaaattgtcatcttctgagctttccgaggctcagaaactgttacaaaacactttcagttggatagaatctaatcaattggctgagaaagaggaatttgaagctaaactcaaggaggtacaagctgtatgtacacctttgactgctaaattgtaccaagctggtggtggcgtcccaggtggcgctgccccaggtggatttaatgctggcggtgcagctccctcaggaccaactgtggaagaagttgat

SEQ ID NO: 46 - DNA, 897 bp, Babesia microti
atgaaacttatcgcctgtacccttaagaatgttgagacctgtgtcgaagtagacccatctgacaccgtagatgctttaaccaataaaattggatcgagcctgaacaatgctagtgccagcaaaatgcgattaatccatgctggtaaaattttgaaaatggaacaaaagatatcggactattctgacatcaaggatggggataaaattattgttctgttcagcaaacaatccgaagcaagtacaatagctaatcctacacctgcccctacctctactcccattgcagatgccaacaccagtcccccgaaacccatccctaccacggaccctaatgccttactgatgggcgaggagctagaaaaagccataaacggtatagtagaaatgggttttgatgttgaatcagtcaaagcggccatgagcgcagcattcaacaaccctaacagggctatagaactcctcacgcgtcatgaggtagacgtttcagaccatgatacgcatcaatctgttcaaacgacgggcgtgttggatgagctgcgacagcaccctatgtttgagcagatgcgggcgattgtgcgcagtaatccacagacgctgccacaaattttatcgctcatagggcagtcagacccatcattactccaagccatcactgaaaatcaggaagaatttatccagctactgagtgaaccggtactaggcacaagtggtgattttattgacgcccagtctataactttgacaccagaggaaatggagtctataaacagactggaggggctcgggttttcgagaccggcagctgtcgaggcatttttggcatgcgataaaaatgaggagatggcagcaaattatttgcttgaaaacatagcagattatgtctctgacaatgataat

SEQ ID NO: 47 - DNA, 432 bp, Babesia microti
atggatggacaaacggaacaacaattgattgaggaaaatatcgaaagatttaggatactttatcaattgcatttggacaacaatgaaccctctcccaccgctcaatttaattatgcatgtgctctagtctgctccaatcagcgatcacataatgacacagctatatatctcttagatgaattggttcgcattagatatgaaagtgaagaatgtttttatcaactcgctcttgcacatatgaaacgcagaagtttcgtcaagtcaaaggaatacttggatcgtatcattgcactggagggttctaatcaacgtgtaatggcacttaaatcagtagtcgtttcattactagctcaggatactttcatgggcggattacttggcgccactgcagcgttcgcaataattctcttcttcacaatgaaaaggaatacc

SEQ ID NO: 48 - DNA, 552 bp, Babesia microti
atgaaccagaattcactctgttgttccaagggttttaccatgtttgcaggtggtgcctttttcctagtttcatttaagccggaaactgttataacgaatccactgctgttacggccgctgttgaatgtgagctgggggtatatatttggatcccatctatgggcagcaatatctacctataacaaaaaatattgggaaaaccggataattcctgattatgctaattcaccatcacgggagattatgcaaagcagacttaagattaacgaatcgcgcatttatcgtatatacctagagaatctaatccaaacaaacgttttagcgaatgggatattattagttacaacaagcgcattggcgccttcaaacaaatttctaaggatttgctcgggagcagcactgcttttatccataggaaatgtagtatttgcgttgcctgaagacgaaaaggatgatgatgtgggagttgtaaagacatcttctacttcatgtttcctgtctgaagtactctctttttgcacattcggcgccatagtcccttatgtatttgca

SEQ ID NO: 49 - protein, 126 aa, Babesia microti
RNPRHTKFHKKHTPITDISPTANNLDDYELITYGNDEGLHDEPGLGSIVTDIEIRTPANFDGTAGNKGRKSKRTDKPVKKAKPVKTRNPVNITEINNANDEDTIDADLDEDLEDDTYTDKPTGFFM

SEQ ID NO: 50 - protein, 971 aa, Babesia microti
SIDSQKSDNDVANTSETSEKLNYYDARDDFLHTMDMVNLVGGSCTLIDKANQPSDFKQLLNASIFGYGRISALLTQTKSIYDVTVASTLAAVFGKLMQINLDDEGLAVEQWSARVCSMLEIAEASLLSTSFKILADRLTKHCGRIAEVFLSQKENDPIDKNKSSDSINDYSSGEGVDVVWSVSTECLMEFIAPNSPGGFTSKWTFVTFKHLLMQLSISIIMCEYQLRDPKLLDDEIEGVENLLFRSWDVLEHYSNIQFYTQLDGASGIMTFAQSNLMPLVKRDGDKINVSWNLQNEFEADPSSNSSGDLGLAAGDNASSDGEKPVATDPPFYSRRPRTYSMLSDYKRRLSLTYEMSDEDTPDEDDDFEREDEEVIVEKETESTPNKQIKNAKNVFGRRKTPQNKVFRPKLYPHDTVSTSSVTYAIKTNNENQIQFKSGAAQPIDSDIDSVPRAKASNPSIDSGNDSVPRAKASNPSIDSGNDSVPRAKASNPSNGSDIDSVPRAKASNPSNGPGNDSVPRAQASNPSNGPGNDSVPRAKASKPSNGSDIDSVPTAPASKPSNGSDIDSVPTAPASTSKSVRGTTGTGSNSKWSSVRNSVLKNKTEENDNNEGPSKASGSAGGGLKGEISFSDTVSILRFRSEPLRWTENTKVEANMHRKMVNKCQVEQLRPLSIDYYNKISPPNLGHPVMGRATASSSSTLLPKFMGIGAKVSKTSNNPSMDTLSNFWGMQRHDKELSGIRKLDKTVSQMAILDRFSRLADTCWDVASGRPSGYISPETMYRLGGNRKDDYIHHVTRVYPFNLVKAYRFLVTKEEPQVQSNGDDNFEFSLFLQAPEFVSILPNGDRAEALVIEQKLVSTIRTLNTNSTEDPPISVCGYVYKRNDVDRVLIGEFGVKLPSTILEHVYKIHTPYTILIHAKFTDSKFNKLKAVMQFLQGTHFGDFTLKFVYKGMVPSKNVVKDSCHECLIGHY

SEQ ID NO: 51 - protein, 1520 aa, Babesia microti
MDNEFTDFSLDKFGTTEYYINNHNHNSNDTVEDPFYLTHNHDSNDNVKPSILNPANASSCIPASYWQQQMEYNDSHLDNSRSSMMNENLIIKELGVDDSPQNDNEITSIDDITKLTEGELTTHTHEPYNLNYDSKTDLQNAFDTFYHDFSLFTPTETLPESQPCTIDSSGHLFNHKFDISNNDSYYQSNEYLNTLPQSFGHWTRTSQTNNCSMEDKHRDDDNNKLSTQLSTFLNPINSDNVDNSGHLYLDINASNTMATEYMHSNQPSGKPFDPLTLLNRVKLRDSTAQIALAQIFNEAQEQKQTLNNVLDSLFDNVGYEGNLLEGDFKFPFISDSLLTATISYSVRTANDEILSILVCYAITNKVNLRADLVADIIDIFFKSLRYSDAYKLIDYCYNDKTKLDSILKTDNFQHMFNMCKEITVDNKISFEIYQYLLQCNGEKEILCKIVESIDEAIDSDSHLASLLRTSSIPFIIRLFVERGDLNKLSYYLKIYLSKPSPSTHDLVEIIDVILETNRAQLQSVVQQISESNSFDLTILLYIFTFLHLKNKSNIIHMIYADAIDKQIQNIPSDLKAVLTSLSIPIFCFDFAPQYISDLLDSILRIYDTCNNSLVKRARAITSVHPATAMNEVDISNDVANEGDFDDLAISELKRMSLTPSGVDFAEIKSLIFSDDFLPRIIPLLTNENIFCLLQLSIASFDINSIEKISYSISLKDSNDLIIVAMINNALVNYGYVEKSKSLLKRAEKLICNVRLRLNNKYPSMPTGTGSPISDSSGIGVRTGGTKEDNFNFGVSDCSGSHLPLKLRDISWVSSISNLLCSKDLSNSESFHLLSFISSLYSHPFIVSTVLKKFIVAHHILIRMLKSKLHINQAMISAICQVLNNTCCINQLQQLLMMTDLLQYHMSNDFYSAILDACIRINSPDHLLESVNKYKKFGFHPDLQTYGLLIKFFSSSDNVMECFHLWNEMTSLYGYELNEVTYGCMFDALVSNNMLDEALSLLKDMKKNSNIKPNTIIYSTLIKGFGQTKQLNKALNIYLTMLDEGVVPNTITYNSIIDACARVGDMNKAANLLEDMLNNNIEPDLITFSTVIKGYCVQSNMDRSLQLLRAMSERGIKPDGILYNSLLDGCVKSGRPWLCQQLWDEMQENGIAPSNFTLTILIKMYGRLGQLDKAFQLMDELPRKYNIQTNTHVYTCLMSACITNGKYKMALDVFNCMNGNGIVPDSKTYETIIFGAIKGRLLYQVIDIIKAAYTLMSRGNGTGGRMNKSIFKIESRILKLFAQKVEASGDPLLLQQAQSLAENLKKFNIILPIRSNLVTQKTQIKKVSNSRAGHYDSFLLKNVTQSLRNDCTNNYRRNSVGATVFANNDGFKDNSVSDFREGINCFGSDDNLNSFKDEHFNAHTQRMHTFASDITCNYRNDERNFQKENRSASGGTTLKGVMWDKDIGGNINNAAYRFDASSIPYSAENAAIGSINSVFGGSYAGNGEDHFLSKNPQYSMQFNPAMVPASNCGNRRGKLKF

SEQ ID NO: 52 - protein, 120 aa, Babesia microti
MAQLAVEELSPFERQELLCVYSSLLLYDDELEISKENINKVLNSAGAKVEPYLPMLFAKALKGKDLNALFGSVASIGAPVASHAAATTSAAVDAPQAGKAQESNVEEEEDDDDMGFSLFD

SEQ ID NO: 53 - protein, 473 aa, Babesia microti
MRWKPIYVFTSALIRLFCIFSCNITFSECIRQSHGVDRRNINGLSLCTPTNVLFSTFACYIRPSKIGKSYNNLNKYKLGTSSSEVDGNEQSDISSGSDVERGNLIQGKRLKNYYRILNLDKYASGEDIKNQYENLIESLKPLENVDSNVIDMINEAYRILSDENTRRIYDELVAKKSLEKESGYNDSNIDQFYHDDGDYFPENLYNVLESDYDFTSDSDEMVLEMSTDDDYSDEDSNEMISLIPKLIKPNGTKLHTTLTIPFERAIMGGNETVTISRLENCKCLENLTTCKSCNGYGLDGKDKLGTGFVSSKECSECGGIGKSRAKKCDLCDNTGQVKVDNATIQVQVPRNVYDGARLLIRNQGNVYGTNGKAGDLVVTLRVKEHDHMYRMGKNIYSDVTVPYAAAILGTTIKLETCGGVVSLEIPPGTQHGDEIQVPNESIPMKHICRIEVALPKSVDKQERSLLEKIIQLK

SEQ ID NO: 54 - protein, 457 aa, Babesia microti
MDQLKDSIDQSNIDRKAALEAIESFLSSKIQAANAGSIRGKKNPAVNDFNNPPIVQSYLSEDEGVTSISEYKNGISDPFYNIAVGTNHSISSAKNIPVSYQPNHTDTNRETPLIQRSIAYFKNKTKFLSKMDTFTSALIPAAITLVITNSAQPLLIFLLRKYGGTPVGAYFFLFPTYLGMICVGLYPTKKPVWKENWIYPGVLAGIDFLHQLIEKAGLLYCGPCLYTVASSSNTLFLALFTSIYLNVKITRNTAISLTIISIAVSFSGSGKLCEINSTHLIGFTLNAFNFVIGEIILKENKIEGPNLVCIMGFISFISLTLWTCVWTIPNWSTIAHHTEMNVYAIFIILFVLFISNFIRSSVYWILIKRAGSLFTGVLKALRIVIVIVISHILFSHIDPMQRITFTKIFTALLCSTGIVIYSIDNNNKIDNYKNEMKGEREAVEDGEDEKIMVNDDV

SEQ ID NO: 55 - DNA, 378 bp, Babesia microti
aggaacccacgacacactaaatttcacaaaaaacacactccaataactgacatatcacccacagctaataatttggatgattatgaattaattacttatggaaatgacgaaggtctacacgatgaacctggccttggtagtatagttacagatatcgagataaggacacccgccaattttgatgggacagctggtaataaggggagaaagagcaagcgcactgataaaccagtgaaaaaggcaaagccagtgaaaacgaggaacccagtgaacatcaccgagataaataacgctaatgatgaagatactatcgacgcagacttagacgaagatctagaggatgatacttatactgataagcccaccggtttctttatg

SEQ ID NO: 56 - DNA, 2913 bp, Babesia microti
tccatagactcacaaaagtccgataatgatgtcgcaaacactagtgaaacaagtgagaaattgaattattacgatgcgcgagatgatttcctccatactatggatatggttaatctagtcggtggctcctgcactttgatagataaggcaaatcagcccagtgactttaaacaactactaaatgcttctatcttcggctacggtagaatctcagcccttctcacccaaaccaagtccatctacgacgtaacagtggccagtacattggccgctgtgtttggtaaactgatgcaaattaatttggatgatgaagggctggcggttgagcaatggtctgccagggtttgtagtatgttggagatagccgaggcgagtcttcttagtacatcattcaagatcctggctgataggcttaccaaacactgtgggaggatcgctgaggtgtttttgtcgcaaaaggaaaatgatcccatcgataaaaacaaatcatcagattctataaatgattattcgtccggtgaaggcgtggacgttgtttggagtgtgagtacggaatgcctaatggaattcatcgccccaaacagcccaggcgggttcacttctaaatggacctttgtgacattcaaacacctactaatgcagctgtccatctcaatcataatgtgtgaatatcagttgcgtgatccaaaattgttggatgatgaaattgaaggcgtagaaaatttgttatttcgctcatgggatgtgctggagcattatagcaacattcagttctacacgcaattagatggtgcatctgggataatgacttttgcccaaagcaatctaatgccccttgtgaaacgggatggggataaaattaacgtgtcatggaacctacagaatgaatttgaagctgatccatcgtctaatagcagtggggatttgggcttggccgccggtgataatgctagcagcgatggcgaaaagcccgttgctacagacccacccttctacagcaggaggcccaggacctatagcatgttgtcagactataaaaggaggctttctcttacctacgagatgtctgacgaggacacgcctgatgaggatgatgattttgagcgtgaagatgaggaagtaatagttgagaaggaaacggaatcaactcctaataaacaaattaaaaatgcaaaaaacgtgtttggcagaagaaaaactccgcaaaataaggtatttagacctaaactgtatccacatgatactgtatccacttcttctgtgacttatgccattaaaaccaataatgagaatcaaatccagttcaagtcgggggccgcccagcctattgactcagacatcgattcagtccctagggctaaagcttcaaatccatctattgactcaggcaacgattcagtccctagggctaaagcttcaaatccatctattgactcaggcaacgattcagtccctagggctaaagcttcaaacccatctaatggctcagacatcgattcagtccctagggctaaagcttcaaatccatctaatggcccaggcaacgattcagtccctagggctcaagcttcaaatccatctaatggcccaggcaacgattcagtccctagggctaaagcttcaaaaccatctaatggctcagacatcgattcagtccctacggctccagcttcaaaaccatctaatggctcagacatcgattcagtccctacggctccagcttcaacgtccaaatctgtaagaggcactaccggaactggcagtaacagtaaatggagcagtgtcagaaactcagtgctgaagaacaaaactgaagagaatgacaacaacgagggcccttccaaggctagtggtagtgctggtggtgggttgaaaggtgagatatccttctcggacacagtatcgatactcaggttcaggtcagaacctctgcggtggaccgaaaatactaaggtagaggctaacatgcataggaagatggtcaataagtgccaggtggagcagcttcgtcccctctcaatagactactacaacaagatctccccgcccaacctgggccatccagtcatgggacgggccaccgctagttcatcgtcgacccttctaccaaagttcatgggtatcggtgccaaggtatccaaaacgtctaacaacccttcgatggacacgctgtcaaacttttgggggatgcaacgccacgataaggagctgtcagggattcgtaaattggataaaactgttagccaaatggccattcttgaccgctttagtagactagctgacacctgctgggatgttgccagtgggaggccttcagggtatatttctccggagaccatgtaccgcctaggcggtaacaggaaagatgattacatccatcatgtaactagggtctatcccttcaatttagttaaggcctaccgtttcttagtgaccaaagaggagccccaagtgcagtcaaatggggacgataattttgaattctccctctttttacaggctccagaatttgtctcgattctacccaatggagatagggccgaagctctggttattgaacaaaaacttgtctcaactattaggaccctcaacactaatagcacagaagacccacctataagcgtctgtggatacgtttacaagcgaaacgacgtggatagggtgctcattggtgagtttggcgttaaattaccgtccactatcctagaacacgtctataaaatacatacgccatacactatactcattcacgccaagttcaccgatagcaagttcaataagctcaaggccgtcatgcagtttttgcaggggacacactttggtgacttcactttaaaatttgtctacaagggtatggtgcccagcaaaaatgtcgtaaaggacagttgccatgagtgtttgataggtcattac

SEQ ID NO: 57 - DNA, 4563 bp, Babesia microti
atggacaacgaattcactgatttctctttggataaatttggtaccacggaatattacattaataaccataatcacaattcaaatgatactgtagaagatccattctatcttacacataaccatgattctaatgataacgtaaaaccgagcatactaaaccctgctaatgctagttcttgcatccctgctagttattggcagcagcaaatggaatacaacgattctcatttggacaattcccgatcttccatgatgaacgagaatctcataatcaaagaactgggcgtcgatgattctcctcagaatgataatgagataacttctattgatgatattactaaactcactgaaggtgaactcactacccatacccatgaaccatacaatctaaattacgattctaaaacggacctacaaaatgcatttgatacattttaccatgatttttcactgtttactcctacggaaactttacctgaatcacagccatgcactattgattcctctggccaccttttcaaccacaaatttgatatatcaaacaatgactcgtattaccaatccaacgaatacctcaacacattgccacaatccttcgggcattggaccagaacttctcaaactaacaactgttcaatggaagataagcatcgggatgatgacaacaataaattatctacccagctttccacatttctaaatccaataaactccgacaacgtggacaacagtggtcatttataccttgatatcaacgcatcgaatacgatggcaacagagtatatgcactcaaatcaaccatctggcaaaccatttgaccccctaacccttctcaatagagtcaagttacgcgattcaacagcgcaaattgcattggcgcagatttttaacgaagcacaagaacagaaacaaacattgaacaatgtactggattcattatttgataatgtaggatatgaaggtaatcttttggagggcgacttcaaatttcctttcatatcggatagtttgttaactgcaactatttcatattcagtaaggacggcaaatgacgaaatcctttcaatacttgtatgttacgcaatcactaataaggttaatttacgtgcagatttggtggcagatataattgatattttcttcaaatcattaaggtactctgacgcctacaaactcatagattattgttataatgacaaaacaaagctcgacagcatcctcaaaactgataattttcagcacatgttcaacatgtgcaaggaaattacagttgataacaagattagttttgaaatatatcaatatttgttacagtgcaatggtgaaaaggagattttatgtaagatagttgaatccattgatgaggcaattgattctgattctcatttggcttcactgctcaggacatcatccattccatttattatcagattatttgtggaaaggggggatttgaataagttatcatattatttgaagatatatttgtcgaagccaagcccttctacacacgatttggttgagataatcgacgtcatactggagacgaatagagcccaacttcagtctgttgtacaacaaatatcagagtctaactcttttgatttgaccattttgctgtatattttcacatttttgcatttgaaaaataagtccaatatcattcatatgatctacgccgacgccattgataagcaaatccaaaacattccatctgaccttaaagcagtacttaccagcctttctataccaatattttgttttgattttgcgcctcagtatatttccgacttgctagattcaattctgaggatctatgatacatgcaataatagtttggtcaaacgcgcccgggccataacctcggttcatcctgcaacggctatgaatgaggtcgacatttcaaatgatgtggcaaacgaaggagattttgatgatttagcgatatccgagctgaaacgaatgtcattgactccttccggagttgattttgctgagattaaatctttgatattttcggacgattttttgcctcgaatcatacctttgttaacgaacgaaaatatattttgcctacttcaactatcaatagcttcttttgacataaattcaattgaaaaaataagctattcaatctcgcttaaggacagcaatgatttgattattgttgccatgattaacaatgcgttggtcaactacggttatgttgagaagtcaaagtcactgcttaaaagggcagagaagctgatttgcaatgtaagactaaggttgaataataagtatccatccatgccaactggtactggttctccaattagcgattcctctggtattggggtccgcactggtggtacgaaagaagataatttcaattttggcgtctctgactgttctggttcacacttaccattgaaactacgtgatatatcatgggtttcatctataagtaacttgttgtgttccaaagacctaagcaattctgagtcatttcacctgttatcttttatctccagtttgtacagtcatcccttcatcgtaagcactgtgctcaagaagttcattgtggcgcatcacattttgatacgtatgctcaaaagtaaattgcacataaatcaagcaatgatatctgccatatgtcaagtattgaataatacctgttgcataaatcaactgcaacaactacttatgatgaccgatttgttacagtaccacatgtcaaatgacttttattcggctattttggatgcttgtataagaattaattcacctgatcaccttttagagtctgttaataaatataaaaaatttggattccaccctgacctccagacatatggcttgttgatcaaattcttcagttcatcagacaatgtgatggagtgtttccacctttggaacgaaatgacgtccctttacggttatgagctgaatgaagtaacctatggttgcatgtttgatgcattagtctccaacaacatgttggacgaggcgttgagtttgctcaaagacatgaagaagaactctaatattaagccaaatacaattatttattcaacacttataaaggggtttgggcagacaaagcagttaaacaaggcgttgaatatctatttgaccatgttggatgaaggagtagtccccaatactattacgtataattctataattgatgcatgtgctcgtgttggggacatgaataaagctgccaatttgcttgaggatatgttaaacaataacatagaacctgacttaattactttctcaaccgtcattaagggatattgcgtacaatctaacatggatcgctccttgcaactacttagggcgatgtctgaaagagggattaaacccgatggaatactatacaattcacttctcgatggatgcgtaaaatctggcaggccttggttatgccaacaactttgggatgaaatgcaggagaacggtatagcaccaagtaactttaccctcactatactgattaaaatgtatggaagattaggccaactagacaaggcgttccagcttatggatgagttgcccagaaaatataacatccaaactaatacgcatgtatatacgtgcctaatgtctgcctgcatcacgaatggcaaatacaaaatggctttggacgtgttcaattgcatgaacggcaatggcatagttcccgattcaaagacttatgagacaataatatttggcgcaataaagggccgtttgctctaccaggtgatcgatattatcaaggctgcctataccctgatgagccgaggtaatggcacaggtgggcggatgaacaaatctattttcaagatagaatctaggatacttaagttgtttgcacagaaagtggaggccagtggcgaccctctgctgctacaacaagcccaatcattggcagagaatttgaaaaagttcaacatcattcttcccattcgtagcaatttggtcacacaaaaaacacagattaaaaaggtatctaacagtagggctggccattacgattcgtttttattgaagaatgttacgcagtcacttaggaatgattgtaccaacaactatcgccgaaacagcgttggtgccacagtatttgcaaataatgatgggtttaaggataatagtgtgagtgattttagggaaggaataaattgttttggaagcgatgataatttaaacagtttcaaagatgaacattttaacgcacacacgcaaagaatgcacacttttgcaagtgacattacatgcaattacagaaatgatgagaggaatttccaaaaggaaaaccggtcggcctccgggggaactacgttaaagggagtcatgtgggataaggatataggtggtaatattaacaatgcggcttatagatttgacgcctcgtctataccatacagcgctgaaaacgctgctataggctccataaacagcgtgttcgggggttcatatgcgggtaatggtgaggaccattttttatcgaagaacccgcagtattccatgcagtttaaccccgccatggtccctgcttcgaattgtggcaatcgcaggggtaagcttaaattttga

SEQ ID NO: 58 - DNA, 363 bp, Babesia microti
atggcgcaattagccgtagaggagcttagcccttttgaaagacaagaactgttatgtgtatattcatcactactattgtacgatgatgaattggagatatctaaggaaaacataaacaaagtgctaaacagtgccggtgctaaggtggagccctatcttccaatgctttttgccaaagctttgaagggtaaggatttgaatgctttgtttggttcagtagcctctattggtgctcctgttgcttcccatgctgctgccactactagtgcagcggtagatgcgcctcaggctggaaaggcccaggaatcaaatgttgaagaagaagaggatgatgatgacatgggattttcactctttgactaa

SEQ ID NO: 59 - DNA, 1422 bp, Babesia microti
atgaggtggaaaccaatttacgtatttacatctgctttaatacgcctcttttgcatatttagttgtaatataacattttccgagtgtataaggcaatcgcatggtgtggataggaggaatataaatggtttgtctttatgcactcctaccaatgttctattctcgactttcgcatgctatatacgaccatcaaagattggcaagagctacaacaatttgaataagtataaacttggcacaagttccagtgaggttgatggtaatgaacaatcagatatatcatctggttctgatgttgagcgtggaaatttaatacaagggaaaaggctgaagaattactatcgaattttaaatttggacaagtatgcatctggagaagacatcaagaatcagtatgaaaatctgattgaatcacttaaaccgcttgaaaatgtagattccaatgttatagatatgataaatgaagcctataggatcctatctgatgaaaatactcgacgcatttacgatgagttggtggcaaaaaaatctctagagaaggaaagcggatataatgatagtaatattgaccaattttatcacgatgatggtgattacttcccagaaaatctttacaacgttttagagtcagattacgactttaccagtgattctgacgaaatggttctagagatgagcacagatgatgattattctgatgaagattctaatgaaatgatcagcttgatacccaaattgattaaaccaaacggaacaaaattgcatacaacacttaccatcccctttgaaagagcaattatgggtggaaatgagacagtgacaatttcgaggcttgaaaattgtaaatgtttggaaaatttaactacatgtaagagttgcaatggatatggtctggatgggaaggataaattgggaacgggattcgtatcatcaaaggaatgttcggaatgtggaggaattggtaaatctagggcgaaaaaatgtgatttatgtgacaatactggacaagttaaagttgataatgcaactattcaagtgcaggttccgcgaaatgtatatgacggcgccaggcttttgattaggaaccagggaaatgtatatggcactaatggcaaagctggtgatttagttgtaactttacgagtaaaagagcatgatcatatgtatagaatgggtaaaaatatatattccgacgtcacagtgccatatgcagctgctatactcggtactactataaaattggaaacatgtggaggagttgtatcgcttgaaatccctcccggtactcaacatggagatgaaatacaagttccaaacgagtctattcccatgaaacacatttgtagaattgaggttgcgctgcctaaatctgtagataaacaagaaaggagtttgttggaaaagataatccagcttaagtag

SEQ ID NO: 60 - DNA, 1374 bp, Babesia microti
atggatcaattaaaagattccattgatcaatcaaatattgatcgaaaggcagctctggaggctattgaatcctttctttcctctaaaatacaagccgctaatgcggggagtattcgtgggaaaaagaatccagctgttaacgattttaataacccgccaatagtacagtcatatttatccgaggatgagggggtaaccagtatatctgaatataaaaatggaatatccgaccccttttacaatattgccgtaggtaccaatcattccatttcctcggcgaaaaatatacctgtatcgtatcagccgaatcacactgatacaaatagagaaacgcccctgatacaacgtagtattgcctattttaaaaataaaactaaattcctctccaaaatggacacgtttacctcggcgttgatccccgctgccattaccctggtcattactaactcagcacaaccactacttatattcttgcttagaaaatatggagggactccggtgggtgcctacttcttcctattcccaacctatctcggcatgatctgcgtcggcctatatccaaccaaaaaacccgtatggaaggaaaattggatttaccccggagtattagctggaattgacttcttgcatcagcttattgaaaaggctgggttgctatactgcggtccatgcctatacactgtggccagcagcagcaatactctgttcctagcgctatttacttccatttatttaaatgtaaaaataactagaaatactgcaatttcattgactattatttcaattgcagtgtcatttagtggttcgggaaaattgtgcgaaataaactcaacacatttgatcggctttacattgaacgcattcaactttgtgataggagagatcatactcaaagaaaataaaatcgaaggcccaaacctcgtctgtataatgggattcatatcctttatctctctcaccctctggacctgcgtttggaccattccaaactggagtacaatcgctcatcatacggaaatgaatgtatatgccattttcattattttatttgtcctgtttatatcaaatttcatacggtcttctgtatattggatactcataaaacgtgctggatctctattcacaggtgtgctaaaggcgctccgaattgttattgttattgtaataagtcacattttgttctcccacatagatcccatgcagaggatcacattcaccaaaatcttcaccgctctgctgtgttcaaccggtatcgtgatctattcaatagacaacaataataagattgataactataagaatgaaatgaagggggagagagaagcggttgaggatggcgaagacgaaaaaatcatggtaaatgatgatgtttaa